(19) United States
(12) Patent Application Publication
(10) Pub. No.: US 2024/0251664 A1
(43) Pub. Date: Jul. 25, 2024

(54) LIGHT EMITTING ELEMENT AND AMINE COMPOUND FOR THE SAME

(71) Applicant: Samsung Display Co., Ltd., Yongin-si, Gyeonggi-do (KR)

(72) Inventor: Takao SAKUMA, Yokohama (JP)

(21) Appl. No.: 18/394,637

(22) Filed: Dec. 22, 2023

(30) Foreign Application Priority Data
Jan. 3, 2023 (KR) 10-2023-0000738

(51) Int. Cl. H10K 85/60; C07C 211/54; C07D 209/88; C07D 307/91; C07D 333/76; C09K 11/06

(52) U.S. Cl. CPC H10K 85/633; C07C 211/54; C07D 209/88; C07D 307/91; C07D 333/76; C09K 11/06; H10K 85/636; H10K 50/15

(57) ABSTRACT

A light emitting element includes a first electrode, a second electrode on the first electrode, and at least one functional layer between the first electrode and the second electrode and including an amine compound including a aryl/heteroaryl group, an α-naphthyl group in which an aryl group is substituted at position 2 of the α-naphthyl group, and a β-naphthyl group, each of which is directly or indirectly connected with the nitrogen atom of the amine.

### CROSS-REFERENCE TO RELATED APPLICATION

This application claims priority to and the benefit of Korean Patent Application No. 10-2023-0000738, filed on Jan. 3, 2023, in the Korean Intellectual Property Office, the entire content of which is hereby incorporated by reference.

### BACKGROUND

One or more embodiments of the present disclosure relate to a light emitting element and an amine compound for a light emitting element, and particularly, to a light emitting element including the amine compound in a functional layer of the light emitting element.

Recently, the development of an organic electroluminescence display device as an image display device is being actively conducted. The organic electroluminescence display device is a display device including a self-luminescent-type or kind of light emitting element in which holes and electrons separately injected from a first electrode and a second electrode recombine in an emission layer of the organic electroluminescence display device so that a light emitting material in the emission layer emits light to achieve display (e.g., of an image).

In the application of a light emitting element to a display device, a long lifetime of the light emitting element is required and/or desired, and thus development on materials for a light emitting element, stably achieving the long lifetime, is being consistently required and/or pursued.

For example, in order to accomplish a light emitting element with long lifetime, development on materials for a hole transport region of the light emitting element, having excellent or suitable hole transport properties and stability, is being conducted and/or researched.

### SUMMARY

One or more aspects of the present disclosure are directed toward a light emitting element showing long-life characteristics and an amine compound which is included in the light emitting element.

Additional aspects will be set forth in part in the description which follows and, in part, will be apparent from the description, or may be learned by practice of the presented embodiments.

A light emitting element according to one or more embodiments of the present disclosure includes a first electrode, a second electrode on the first electrode, and at least one functional layer between the first electrode and the second electrode, wherein the at least one functional layer includes an amine compound represented by Formula 1.

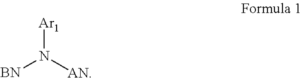

In Formula 1, Ar₁ may be a substituted or unsubstituted aryl group of 6 to 50 ring-forming carbon atoms, or a substituted or unsubstituted heteroaryl group of 5 to 50 ring-forming carbon atoms, any one (e.g., one) selected from among AN and BN may be represented by Formula A, and the remainder may be represented by Formula B.

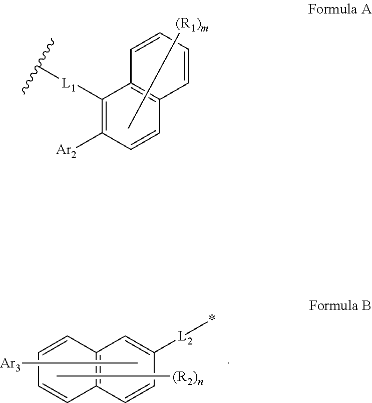

In Formula A and Formula B, L₁ and L₂ may each independently be a direct linkage, or a substituted or unsubstituted arylene group of 6 to 50 ring-forming carbon atoms. R₁ may be hydrogen, deuterium, a cyano group, or a substituted or unsubstituted alkyl group of 1 to 30 carbon atoms, R₂ may be hydrogen, deuterium, a cyano group, a substituted or unsubstituted alkyl group of 1 to 30 carbon atoms, or a substituted or unsubstituted aryl group of 6 to 30 ring-forming carbon atoms, and/or combined with an adjacent group to form a ring. Ar₂ is a substituted or unsubstituted aryl group of 6 to 50 ring-forming carbon atoms, and Ar₃ is hydrogen, deuterium, or a substituted or unsubstituted aryl group of 6 to 50 ring-forming carbon atoms. “n” and “m” may each independently be an integer of 0 to 6, an embodiment in which Ar₂ is a substituted or unsubstituted naphthyl group, is excluded, and “ [symbol] ” and “—*” are positions connected with Formula 1 (e.g., bonded to the nitrogen of the amine compound represented by Formula 1).

In one or more embodiments, Formula A may be represented by Formula A1 or Formula A2, and Formula B may be represented by Formula B1 or Formula B2.

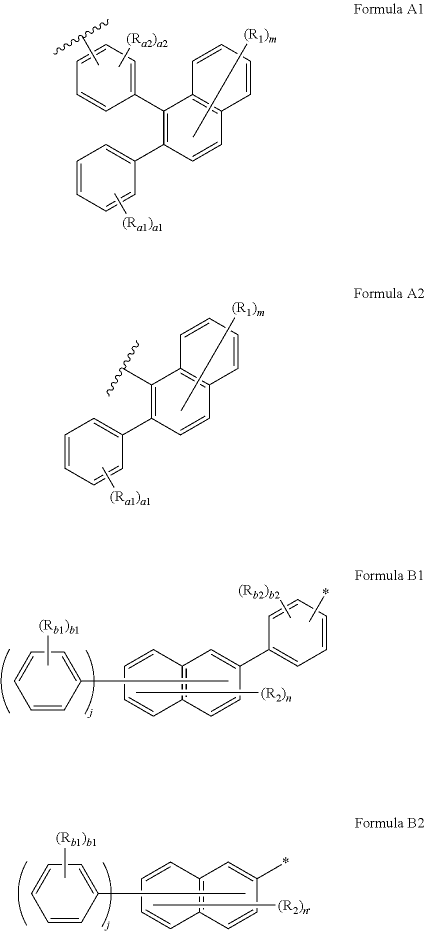

In Formula A1 and Formula A2, Rai may be hydrogen, deuterium, or a substituted or unsubstituted aryl group of 6 to 15 ring-forming carbon atoms, Rₐ₂ may be hydrogen or deuterium, a1 may be an integer of 0 to 5, a2 may be an integer of 0 to 4, and R₁ and “m” may each independently be the same as defined in Formula A.

In Formula B1 and Formula B2, R_{b1} and R_{b2} may each independently be hydrogen or deuterium, b1 may be an integer of 0 to 5, b2 is an integer of 0 to 4, “j” is 0 or 1, and R₂ and “n” may each independently be the same as defined in Formula B.

In one or more embodiments, the amine compound represented by Formula 1 may be represented by Formula 2.

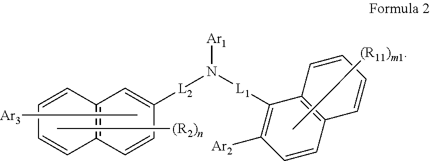

In Formula 2, R₁₁ is hydrogen or deuterium, m1 is an integer of 0 to 6, and Ar₁ to Ar₃, L₁, L₂, R₂, and “n” may each independently be the same as defined in Formula 1.

In one or more embodiments, the amine compound represented by Formula 1 may be represented by Formula 3-1 or Formula 3-2.

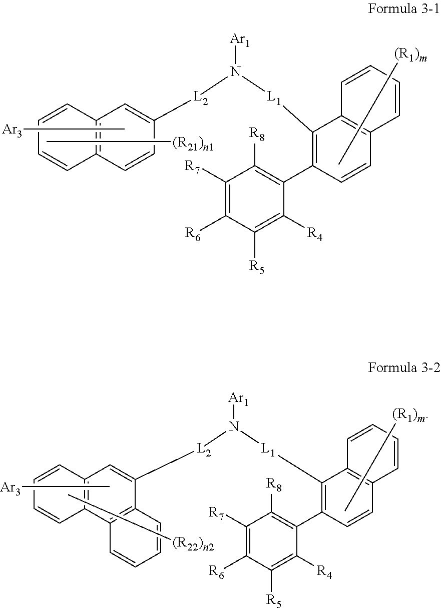

In Formula 3-1 and Formula 3-2, R₄ to R₈ may each independently be hydrogen, deuterium, or a substituted or unsubstituted aryl group of 6 to 15 ring-forming carbon atoms. R₂₁ and R₂₂ may each independently be hydrogen or deuterium, n1 may be an integer of 0 to 6, n2 may be an integer of 0 to 8, and Ar₁, Ar₃, L₁, L₂, R₁, and “m” may each independently be the same as defined in Formula 1.

In one or more embodiments, the amine compound represented by Formula 1 may be represented by Formula 4-1 or Formula 4-2.

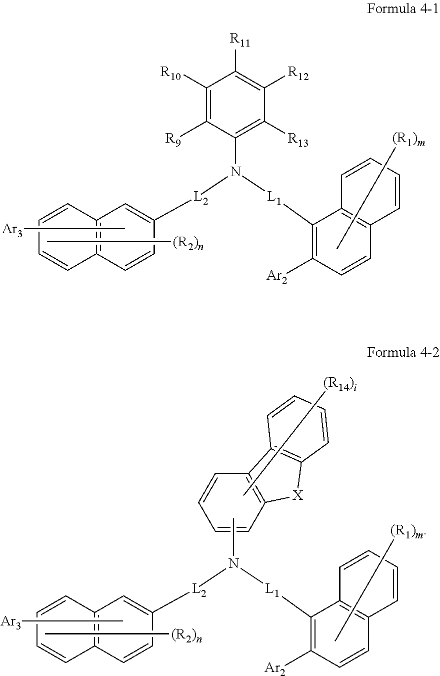

In Formula 4-1, R₉ to R₁₃ may each independently be hydrogen, deuterium, or a substituted or unsubstituted aryl group of 6 to 30 ring-forming carbon atoms, and/or substituents in which at least one pair selected from among adjacent R₉ and R₁₀, R₁₀ and R₁₁, R₁₁ and R₁₂, and R₁₂ and R₁₃ are combined with each other to form an aromatic hydrocarbon ring. In Formula 4-2, X may be O, S, NRₓ₁, or CRₓ₂Rₓ₃, Rₓ₁ may be a substituted or unsubstituted aryl group of 6 to 15 ring-forming carbon atoms, Rₓ₂ and Rₓ₃ may each independently be a substituted or unsubstituted alkyl group of 1 to 10 carbon atoms, or a substituted or unsubstituted aryl group of 6 to 15 ring-forming carbon atoms, or Rₓ₂ and Rₓ₃ are combined with each other to form a ring, R₁₄ may be hydrogen, deuterium, or a substituted or unsubstituted aryl group of 6 to 15 ring-forming carbon atoms, and/or combined with an adjacent group to form a ring, and “i” may be an integer of 0 to 7.

In Formula 4-1 and Formula 4-2, Ar₂, Ar₃, L₁, L₂, R₁, R₂, “m” and “n” may each independently be the same as defined in Formula 1.

In one or more embodiments, the amine compound represented by Formula 1 may be represented by any one selected from among Formula 5-1 to Formula 5-4.

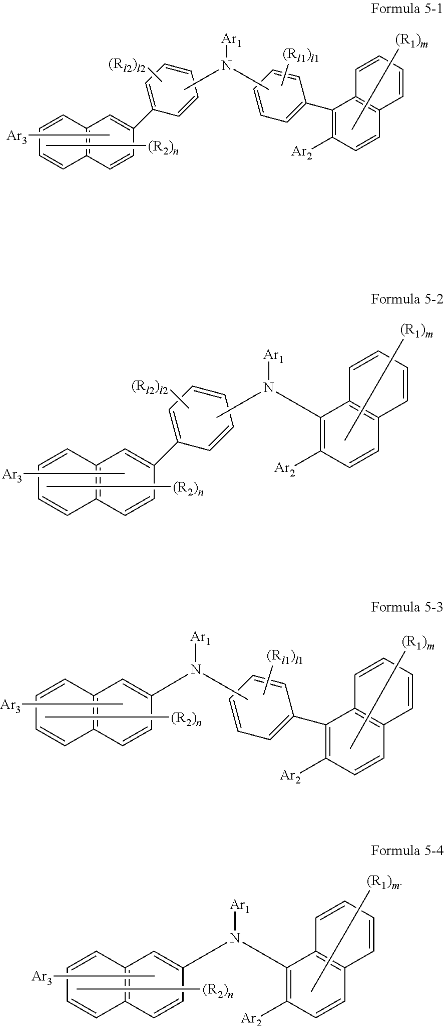

In Formula 5-1 to Formula 5-4, Rₗ₁ and Rₗ₂ may each independently be hydrogen or deuterium, l1 and l2 may each independently be an integer of 0 to 4, and Ar₁ to Ar₃, R₁, R₂, “m” and “n” may each independently be the same as defined in Formula 1.

In one or more embodiments, AN may be represented by any one in Substituent Group A, which will be explained later.

In one or more embodiments, BN may be represented by any one in Substituent Group B, which will be explained later.

In one or more embodiments, Ar₁ may be represented by any one in Substituent Group AR, which will be explained later.

In the light emitting element of one or more embodiments, the at least one functional layer may include a hole transport region on the first electrode, an emission layer on the hole transport region, and an electron transport region on the emission layer, and the hole transport region may include the amine compound.

In the light emitting element of one or more embodiments, the hole transport region may include a hole injection layer on the first electrode, and a hole transport layer on the hole injection layer, and the hole transport layer may include the amine compound.

### BRIEF DESCRIPTION OF THE FIGURES

The accompanying drawings are included to provide a further understanding of the present disclosure and are incorporated in and constitute a part of this specification. The drawings illustrate embodiments of the present disclosure and, together with the description, serve to explain principles of the present disclosure. Above and/or other aspects of the present disclosure should become apparent and appreciated from the following description of embodiments taken in conjunction with the accompanying drawings. In the drawings:

FIG. 1 is a plan view showing a display device according to one or more embodiments of the present disclosure;

FIG. 2 is a cross-sectional view of a display device according to one or more embodiments of the present disclosure;

FIG. 3 is a cross-sectional view schematically showing a light emitting element according to one or more embodiments of the present disclosure;

FIG. 4 is a cross-sectional view schematically showing a light emitting element according to one or more embodiments of the present disclosure;

FIG. 5 is a cross-sectional view schematically showing a light emitting element according to one or more embodiments of the present disclosure;

FIG. 6 is a cross-sectional view schematically showing a light emitting element according to one or more embodiments of the present disclosure;

FIG. 7 is a cross-sectional view of a display device according to one or more embodiments of the present disclosure;

FIG. 8 is a cross-sectional view of a display device according to one or more embodiments of the present disclosure;

FIG. 9 is a cross-sectional view showing a display device according to one or more embodiments of the present disclosure;

FIG. 10 is a cross-sectional view showing a display device according to one or more embodiments of the present disclosure; and

FIG. 11 is a diagram showing a vehicle in which a display device according to one or more embodiments is disposed.

### DETAILED DESCRIPTION

The present disclosure may be modified in one or more suitable manners and have many forms, and thus specific embodiments will be exemplified in the drawings and described in more detail in the detailed description of the present disclosure. It should be understood, however, that it is not intended to limit the present disclosure to the particular forms disclosed, but rather, is intended to cover all modifications, equivalents, and alternatives falling within the spirit and scope of the present disclosure.

When explaining each of drawings, like reference numbers are utilized for referring to like elements. In the accompanying drawings, the dimensions of each structure may be exaggeratingly illustrated for clarity of the present disclosure. It will be understood that, although the terms “first,” “second,” etc., may be utilized herein to describe one or more suitable components, these components should not be limited by these terms. These terms are only utilized to distinguish one component from another. For example, a first component could be termed a second component, and, similarly, a second component could be termed a first component, without departing from the scope of example embodiments of the present disclosure. As utilized herein, the singular forms, “a,” “an,” “one,” and “the” are intended to include the plural forms as well, unless the context clearly indicates otherwise. Further, the utilization of “may” when describing embodiments of the present disclosure refers to “one or more embodiments of the present disclosure”.

In the present disclosure, it will be understood that the terms “comprise(s)/include(s),” “have/has,” and/or the like specify the presence of features, numbers, steps, operations, component, parts, or combinations thereof disclosed in the disclosure, but do not exclude the possibility of presence or addition of one or more other features, numbers, steps, operations, component, parts, or combinations thereof.

In the present disclosure, when a layer, a film, a region, or a plate is referred to as being “on” or “in an upper portion of” another layer, film, region, or plate, it may be not only “directly on” the layer, film, region, or plate, but intervening layers, films, regions, or plates may also be present. In addition, when a layer, a film, a region, or a plate is referred to as being “below”, “in a lower portion of” another layer, film, region, or plate, it can be not only directly under the layer, film, region, or plate, but one or more intervening layers, films, regions, or plates may also be present. In some embodiments, it will be understood that when a part is referred to as being “on” another part, it can be disposed above the other part, or disposed under the other part as well.

As used herein, the terms “and”, “or”, and “and/or” may include any and all combinations of one or more of the associated listed items. Expressions such as “at least one of,” “one of,” and “selected from,” when preceding a list of elements, modify the entire list of elements and do not modify the individual elements of the list. For example, “at least one of a, b, or c”, “at least one selected from a, b, and c”, “at least one selected from among a to c”, etc., may indicate only a, only b, only c, both (e.g., simultaneously) a and b, both (e.g., simultaneously) a and c, both (e.g., simultaneously) b and c, all of a, b, and c, or variations thereof. The “/” utilized herein may be interpreted as “and” or as “or” depending on the situation.

In the present disclosure, the term “substituted or unsubstituted” may refer to substituted or unsubstituted with at least one substituent selected from the group consisting of deuterium, a halogen, a cyano group, a nitro group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, a hydrocarbon ring group, an aryl group, and a heterocyclic group. In some embodiments, each of the substituents exemplified above may be substituted or unsubstituted. For example, a biphenyl group may be interpreted as an aryl group or a phenyl group substituted with a phenyl group.

In the present disclosure, the phrase “bonded to an adjacent group to form a ring” may refer to that a group is bonded to an adjacent group to form a substituted or unsubstituted hydrocarbon ring, or a substituted or unsubstituted heterocycle. The hydrocarbon ring may include an aliphatic hydrocarbon ring and/or an aromatic hydrocarbon ring. The heterocycle may include an aliphatic heterocycle and/or an aromatic heterocycle. The hydrocarbon ring and the heterocycle may be monocyclic or polycyclic. In some embodiments, the rings formed by being bonded to each other may be connected to another ring to form a spiro structure.

In the present disclosure, the term “adjacent group” may refer to a substituent substituted for an atom which is directly linked to an atom substituted with a corresponding substituent, another substituent substituted for an atom which is substituted with a corresponding substituent, or a substituent sterically positioned at the nearest position to a corresponding substituent. For example, two methyl groups in 1,2-dimethylbenzene may be interpreted as “adjacent groups” to each other, and two ethyl groups in 1,1-diethylcyclopentane may be interpreted as “adjacent groups” to each other. In some embodiments, two methyl groups in 4,5-dimethylphenanthrene may be interpreted as “adjacent groups” to each other.

In the present disclosure, examples of a halogen may include fluorine, chlorine, bromine, or iodine.

In the present disclosure, an alkyl group may be linear or branched. The number of carbons in the alkyl group may be 1 to 50, 1 to 30, 1 to 20, 1 to 10, or 1 to 6. Examples of the alkyl group may include a methyl group, an ethyl group, an n-propyl group, an isopropyl group, an n-butyl group, an s-butyl group, a t-butyl group, an i-butyl group, a 2-ethylbutyl group, a 3,3-dimethylbutyl group, an n-pentyl group, an i-pentyl group, a neopentyl group, a t-pentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-heneicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, etc., but embodiments of the present disclosure are not limited thereto.

In the present disclosure, a cycloalkyl group may refer to a cyclic alkyl group. The number of carbons in the cycloalkyl group may be 3 to 50, 3 to 30, 3 to 20, or 3 to 10. Examples of the cycloalkyl group may include a cyclopropyl group, a cyclobutyl group, a cyclopentyl group, a cyclohexyl group, a 4-methylcyclohexyl group, a 4-t-butylcyclohexyl group, a cycloheptyl group, a cyclooctyl group, a cyclononyl group, a cyclodecyl group, a norbornyl group, a 1-adamantyl group, a 2-adamantyl group, an isobornyl group, a bicycloheptyl group, etc., but embodiments of the present disclosure are not limited thereto.

In the present disclosure, an alkenyl group may refer to a hydrocarbon group including at least one carbon-carbon double bond in the middle or terminal of an alkyl group having 2 or more carbon atoms. The alkenyl group may be linear or branched. The number of carbon atoms in the alkenyl group is not specifically limited, for example, may be 2 to 30, 2 to 20, or 2 to 10. Examples of the alkenyl group may include a vinyl group, a 1-butenyl group, a 1-pentenyl group, a 1,3-butadienyl group, a styrenyl group, a styryl vinyl group, etc., but embodiments of the present disclosure are not limited thereto.

In the present disclosure, an alkynyl group may refer to a hydrocarbon group including at least one carbon-carbon triple bond in the middle or terminal of an alkyl group having 2 or more carbon atoms. The alkynyl group may be linear or branched. Although the number of carbon atoms thereof is not specifically limited, it may be 2 to 30, 2 to 20, or 2 to 10. Examples of the alkynyl group may include an ethynyl group, a propynyl group, etc., but embodiments of the present disclosure are not limited thereto.

In the present disclosure, the hydrocarbon ring group may refer to any functional group or substituent derived from an aliphatic hydrocarbon ring. The hydrocarbon ring group may be a saturated hydrocarbon ring group having 5 to 20 ring-forming carbon atoms.

In the present disclosure, an aryl group may refer to any functional group or substituent derived from an aromatic hydrocarbon ring. The aryl group may be a monocyclic aryl group or a polycyclic aryl group. The number of ring-forming carbon atoms in the aryl group may be 6 to 30, 6 to 20, or 6 to 15. Examples of the aryl group may include a phenyl group, a naphthyl group, a fluorenyl group, an anthracenyl group, a phenanthryl group, a biphenyl group, a terphenyl group, a quaterphenyl group, a quinquephenyl group, a sexiphenyl group, a triphenylenyl group, a pyrenyl group, a benzofluoranthenyl group, a chrysenyl group, etc., but embodiments of the present disclosure are not limited thereto.

In the present disclosure, the fluorenyl group may be substituted, and two substituents may be bonded to each other to form a spiro structure. Examples of the substituted fluorenyl group may be as follows. However, embodiments of the present disclosure are not limited thereto.

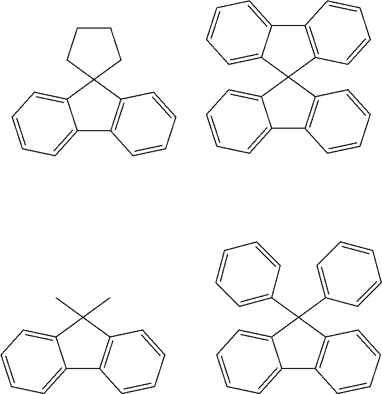

A heterocyclic group utilized herein may refer to any functional group or substituent derived from a ring containing at least one of B, O, N, P, Si, or Se as a heteroatom. The heterocyclic group may include an aliphatic heterocyclic group and/or an aromatic heterocyclic group. The aromatic heterocyclic group may be a heteroaryl group. The aliphatic heterocycle and the aromatic heterocycle may each be monocyclic or polycyclic.

In the present disclosure, the heterocyclic group may contain at least one of B, O, N, P, Si or S as a heteroatom. When the heterocyclic group contains two or more heteroatoms, the two or more heteroatoms may be the same as or different from each other. The heterocyclic group may be a monocyclic heterocyclic group or a polycyclic heterocyclic group, and may include a heteroaryl group. The number of ring-forming carbon atoms in the heterocyclic group may be 2 to 30, 2 to 20, or 2 to 10.

In the present disclosure, the aliphatic heterocyclic group may include at least one of B, O, N, P, Si, or S as a heteroatom. The number of ring-forming carbon atoms in the aliphatic heterocyclic group may be 2 to 30, 2 to 20, or 2 to 10. Examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, etc., but embodiments of the present disclosure are not limited thereto.

In the present disclosure, the heteroaryl group may contain at least one of B, O, N, P, Si, or S as a heteroatom. When the heteroaryl group contains two or more heteroatoms, the two or more heteroatoms may be the same as or different from each other. The heteroaryl group may be a monocyclic heterocyclic group or a polycyclic heterocyclic group. The number of ring-forming carbon atoms in the heteroaryl group may be 2 to 30, 2 to 20, or 2 to 10. Examples of the heteroaryl group may include a thiophene group, a furan group, a pyrrole group, an imidazole group, a pyridine group, a bipyridine group, a pyrimidine group, a triazine group, a triazole group, an acridyl group, a pyridazine group, a pyrazinyl group, a quinoline group, a quinazoline group, a quinoxaline group, a phenoxazine group, a phthalazine group, a pyrido pyrimidine group, a pyrido pyrazine group, a pyrazino pyrazine group, an isoquinoline group, an indole group, a carbazole group, an N-arylcarbazole group, an N-heteroarylcarbazole group, an N-alkylcarbazole group, a benzoxazole group, a benzimidazole group, a benzothiazole group, a benzocarbazole group, a benzothiophene group, a dibenzothiophene group, a thienothiophene group, a benzofuran group, a phenanthroline group, a thiazole group, an isoxazole group, an oxazole group, an oxadiazole group, a thiadiazole group, a phenothiazine group, a dibenzosilole group, a dibenzofuran group, etc., but embodiments of the present disclosure are not limited thereto.

In the present disclosure, the above description of the aryl group may be applied to an arylene group except that the arylene group is a divalent group. The above description of the heteroaryl group may be applied to a heteroarylene group except that the heteroarylene group is a divalent group.

In the present disclosure, a silyl group may include an alkylsilyl group and/or an arylsilyl group. Examples of the silyl group may include a trimethylsilyl group, a triethylsilyl group, a t-butyldimethylsilyl group, a propyldimethylsilyl group, a triphenylsilyl group, a diphenylsilyl group, a phenylsilyl group, etc., but embodiments of the present disclosure are not limited thereto.

In the present disclosure, the number of ring-forming carbon atoms in a carbonyl group is not specifically limited, for example, may be 1 to 40, 1 to 30, or 1 to 20. For example, the carbonyl group may have the following structures, but embodiments of the present disclosure are not limited thereto.

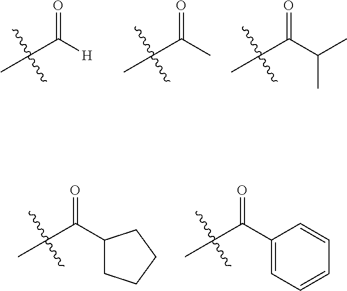

In the present disclosure, the number of carbon atoms in a sulfinyl group or a sulfonyl group is not particularly limited, for example, may be 1 to 30, 1 to 20, or 1 to 10. The sulfinyl group may include an alkyl sulfinyl group and/or an aryl sulfinyl group. The sulfonyl group may include an alkyl sulfonyl group and/or an aryl sulfonyl group.

In the present disclosure, a thio group may include an alkylthio group and/or an arylthio group. The thio group may refer to that a sulfur atom is bonded to the alkyl group or the aryl group as defined above. Examples of the thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, etc., but the embodiment of the present disclosure is not limited thereto.

In the present disclosure, an oxy group may refer to that an oxygen atom is bonded to the alkyl group or the aryl group as defined above. The oxy group may include an alkoxy group and/or an aryl oxy group. The alkoxy group may be a linear chain, a branched chain, or a ring. The number of carbon atoms in the alkoxy group is not specifically limited, for example, may be 1 to 20 or 1 to 10. Examples of the oxy group may include methoxy, ethoxy, n-propoxy, isopropoxy, butoxy, pentyloxy, hexyloxy, octyloxy, nonyloxy, decyloxy, benzyloxy, etc., but embodiments of the present disclosure are not limited thereto.

A boron group as utilized herein may refer to that a boron atom is bonded to the alkyl group or the aryl group as defined above. The boron group may include an alkyl boron group and/or an aryl boron group. Examples of the boron group may include a dimethylboron group, a trimethylboron group, a t-butyldimethylboron group, a diphenylboron group, a phenylboron group, etc., but embodiments of the present disclosure are not limited thereto.

In the present disclosure, the number of carbon atoms in an amine group is not specifically limited, for example, may be 1 to 30, 1 to 20, or 1 to 10. The amine group may include an alkyl amine group and/or an aryl amine group. Examples of the amine group may include a methylamine group, a dimethylamine group, a phenylamine group, a diphenylamine group, a naphthylamine group, a 9-methyl-anthracenylamine group, etc., but embodiments of the present disclosure are not limited thereto.

In the present disclosure, the alkyl group among an alkylthio group, an alkylsulfoxy group, an alkylaryl group, an alkylamino group, an alkyl boron group, an alkyl silyl group, and an alkyl amine group may be the same as the examples of the alkyl group described above.

In the present disclosure, the aryl group among an aryloxy group, an arylthio group, an arylsulfoxy group, an arylamino group, an arylboron group, an arylsilyl group, an arylamine group may be the same as the examples of the aryl group described above.

In the present disclosure, a direct linkage may refer to a single bond.

In the present disclosure, “ [symbol] ”, “—*”, “ [symbol] ”, and “ [symbol] ” may refer to a position to be connected.

Hereinafter, embodiments of the present disclosure will be described in more detail with reference to the accompanying drawings.

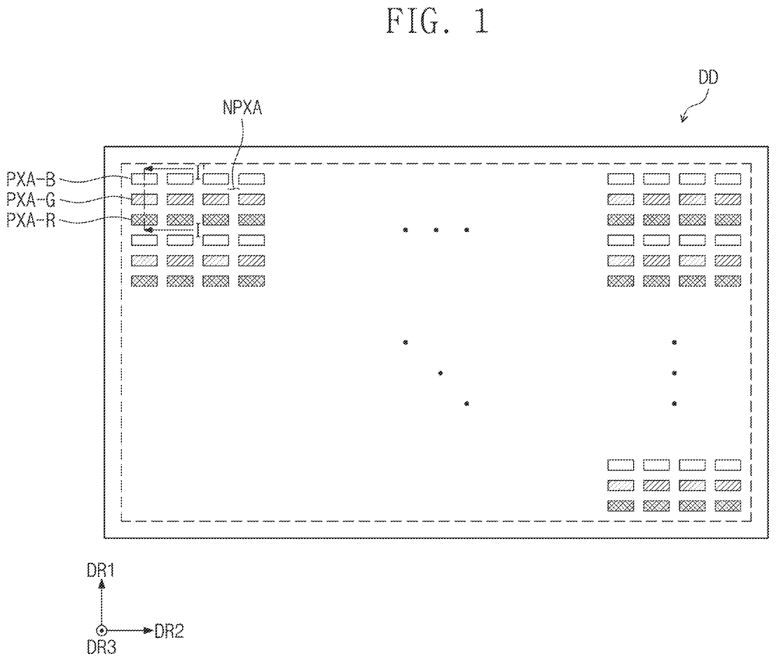

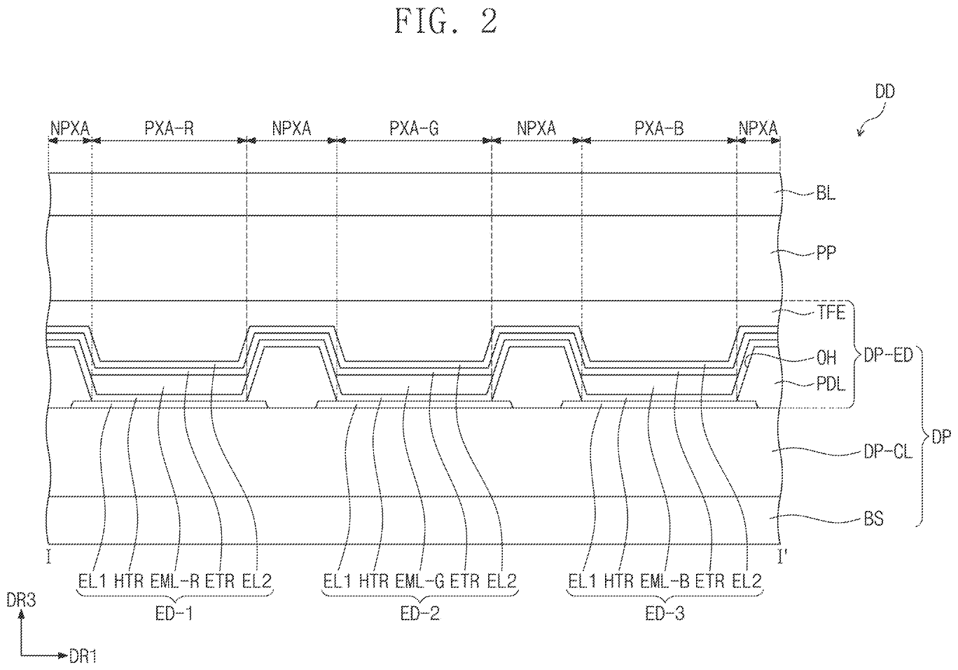

FIG. 1 is a plan view illustrating a display apparatus DD according to one or more embodiments of the present disclosure. FIG. 2 is a cross-sectional view of the display apparatus DD according to one or more embodiments of the present disclosure. FIG. 2 is a cross-sectional view illustrating a part taken along line I-I′ of FIG. 1.

The display apparatus DD may include a display panel DP and an optical layer PP disposed on the display panel DP. The display panel DP may include light emitting elements ED-1, ED-2, and ED-3. The display apparatus DD may include a plurality of light emitting elements ED-1, ED-2, and ED-3. The optical layer PP may be disposed on the display panel DP to control reflected light in the display panel DP due to external light. The optical layer PP may include, for example, a polarization layer and/or a color filter layer. In some embodiments, unlike the configuration illustrated in the drawing, the optical layer PP may not be provided in the display apparatus DD.

A base substrate BL may be disposed or provided on the optical layer PP. The base substrate BL may be a member which provides a base surface on which the optical layer PP disposed. The base substrate BL may be a glass substrate, a metal substrate, a plastic substrate, etc. However, embodiments of the present disclosure are not limited thereto, and the base substrate BL may be an inorganic layer, an organic layer, or a composite material layer. In some embodiments, unlike the configuration illustrated, the base substrate BL may not be provided.

The display apparatus DD according to one or more embodiments may further include a filling layer. The filling layer may be disposed between a display device layer DP-ED and the base substrate BL. The filling layer may be an organic material layer. The filling layer may include at least one of an acrylic-based resin, a silicone-based resin, or an epoxy-based resin.

The display panel DP may include a base layer BS, a circuit layer DP-CL provided on the base layer BS, and the display device layer DP-ED. The display device layer DP-ED may include a pixel defining film PDL, the light emitting elements ED-1, ED-2, and ED-3 disposed between portions of the pixel defining film PDL, and an encapsulation layer TFE disposed on the light emitting elements ED-1, ED-2, and ED-3.

The base layer BS may be a member which provides a base surface on which the display device layer DP-ED is disposed. The base layer BS may be a glass substrate, a metal substrate, a plastic substrate, etc. However, embodiments of the present disclosure are not limited thereto, and the base layer BS may be an inorganic layer, an organic layer, or a composite material layer.

In one or more embodiments, the circuit layer DP-CL may be disposed on the base layer BS, and the circuit layer DP-CL may include a plurality of transistors. Each of the transistors may include a control electrode, an input electrode, and an output electrode. For example, the circuit layer DP-CL may include switching transistor(s) and driving transistor(s) for driving the light emitting elements ED-1, ED-2, and ED-3 of the display device layer DP-ED.

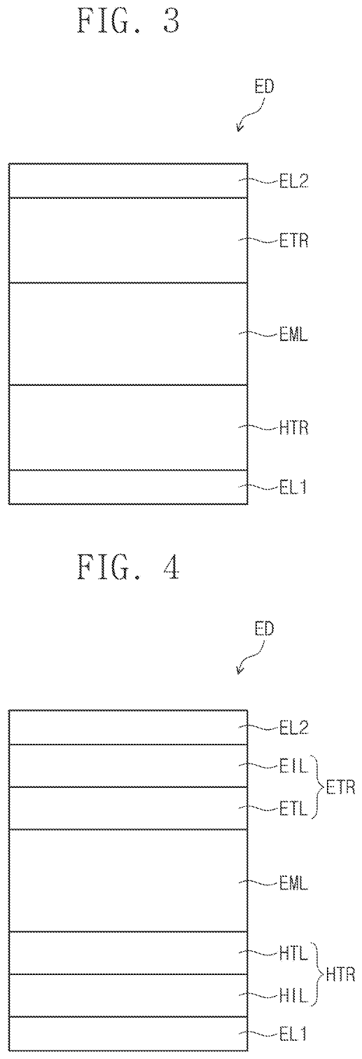

Each of the light emitting elements ED-1, ED-2, and ED-3 may have a structure of a light emitting element ED of one or more embodiments according to FIGS. 3 to 6, which will be described later. Each of the light emitting elements ED-1, ED-2, and ED-3 may include a first electrode EL1, a hole transport region HTR, a respective one selected from emission layers EML-R, EML-G, and EML-B, an electron transport region ETR, and a second electrode EL2.

FIG. 2 illustrates an embodiment in which the respective emission layers EML-R, EML-G, and EML-B of the light emitting elements ED-1, ED-2, and ED-3 are disposed in openings OH defined in the pixel defining film PDL, and the hole transport region HTR, the electron transport region ETR, and the second electrode EL2 are provided as a common layer throughout the entire light emitting elements ED-1, ED-2, and ED-3. However, embodiments of the present disclosure are not limited thereto, in one or more embodiments, the hole transport region HTR and the electron transport region ETR may be provided by being patterned inside the openings OH defined in the pixel defining film PDL. For example, in some embodiments, the hole transport region HTR, the respective emission layers EML-R, EML-G, and EML-B, and the electron transport region ETR of the light emitting elements ED-1, ED-2, and ED-3 may be provided by being patterned in an inkjet printing method.

The encapsulation layer TFE may cover the light emitting elements ED-1, ED-2, and ED-3. The encapsulation layer TFE may seal the display device layer DP-ED. The encapsulation layer TFE may be a thin film encapsulation layer. The encapsulation layer TFE may be formed by laminating one layer or a plurality of layers. The encapsulation layer TFE may include at least one insulation layer. The encapsulation layer TFE according to an embodiment may include at least one inorganic film (hereinafter, an encapsulation-inorganic film). The encapsulation layer TFE according to an embodiment may also include at least one organic film (hereinafter, an encapsulation-organic film) and at least one encapsulation-inorganic film.

The encapsulation-inorganic film protects the display device layer DP-ED from moisture/oxygen, and the encapsulation-organic film protects the display device layer DP-ED from foreign substances such as dust particles. The encapsulation-inorganic film may include silicon nitride, silicon oxynitride, silicon oxide, titanium oxide, aluminum oxide, and/or the like, but embodiments of the present disclosure are not particularly limited thereto. The encapsulation-organic film may include an acrylic-based compound, an epoxy-based compound, and/or the like. In some embodiments, the encapsulation-organic film may include a photopolymerizable organic material, but embodiments of the present disclosure are not particularly limited thereto.

The encapsulation layer TFE may be disposed on the second electrode EL2 and may be disposed filling the opening OH.

Referring to FIG. 1 and FIG. 2, the display apparatus DD may include a non-light emitting region NPXA and light emitting regions PXA-R, PXA-G, and PXA-B. The light emitting regions PXA-R, PXA-G, and PXA-B may be regions in which light generated by the respective light emitting elements ED-1, ED-2, and ED-3 is emitted. The light emitting regions PXA-R, PXA-G, and PXA-B may be spaced apart from each other on a plane (e.g., in a plan view).

Each of the light emitting regions PXA-R, PXA-G, and PXA-B may be a region divided by the pixel defining film PDL. The non-light emitting regions NPXA may be regions between the adjacent light emitting regions PXA-R, PXA-G, and PXA-B, and may correspond to the pixel defining film PDL. In one or more embodiments, the light emitting regions PXA-R, PXA-G, and PXA-B may respectively correspond to pixels. The pixel defining film PDL may divide the light emitting elements ED-1, ED-2, and ED-3. The respective emission layers EML-R, EML-G, and EML-B of the light emitting elements ED-1, ED-2, and ED-3 may be disposed in openings OH defined in the pixel defining film PDL and separated from each other.

The light emitting regions PXA-R, PXA-G, and PXA-B may be divided into a plurality of groups according to the color of each light generated from the light emitting elements ED-1, ED-2, and ED-3. In the display apparatus DD of an embodiment illustrated in FIGS. 1 and 2, three light emitting regions PXA-R, PXA-G, and PXA-B, which emit red light, green light, and blue light, respectively, are exemplarily illustrated. For example, the display apparatus DD of an embodiment may include a red light emitting region PXA-R, a green light emitting region PXA-G, and a blue light emitting region PXA-B that are separated from each other.

In the display apparatus DD according to one or more embodiments, the plurality of light emitting elements ED-1, ED-2, and ED-3 may be to emit light beams having wavelengths different from each other. For example, in an embodiment, the display apparatus DD may include a first light emitting element ED-1 that emits red light, a second light emitting element ED-2 that emits green light, and a third light emitting element ED-3 that emits blue light. For example, in some embodiments, the red light emitting region PXA-R, the green light emitting region PXA-G, and the blue light emitting region PXA-B of the display apparatus DD may correspond to the first light emitting element ED-1, the second light emitting element ED-2, and the third light emitting element ED-3, respectively.

However, embodiments of the present disclosure are not limited thereto, and the first to third light emitting elements ED-1, ED-2, and ED-3 may be to emit light beams in substantially the same wavelength range or at least one light emitting element may be to emit a light beam in a wavelength range different from the others. For example, in some embodiments, the first to third light emitting elements ED-1, ED-2, and ED-3 may all emit blue light.

The light emitting regions PXA-R, PXA-G, and PXA-B in the display apparatus DD according to an embodiment may be arranged in a stripe form. Referring to FIG. 1, a plurality of red light emitting regions PXA-R may be arranged with each other along a second direction axis DR2, a plurality of green light emitting regions PXA-G may be arranged with each other along the second direction axis DR2, and a plurality of blue light emitting regions PXA-B may be arranged with each other along the second direction axis DR2. In some embodiments, the red light emitting region PXA-R, the green light emitting region PXA-G, and the blue light emitting region PXA-B may be alternately arranged in the stated order along a first direction axis DR1.

FIG. 1 and FIG. 2 illustrate that all the light emitting regions PXA-R, PXA-G, and PXA-B have similar area, but embodiments of the present disclosure are not limited thereto. Thus, in some embodiments, the light emitting regions PXA-R, PXA-G, and PXA-B may have different areas from each other according to the wavelength range of the emitted light. In these embodiments, the areas of the light emitting regions PXA-R, PXA-G, and PXA-B may refer to areas when viewed on a plane defined by the first direction axis DR1 and the second direction axis DR2 (e.g., areas in a plan view).

In some embodiments, an arrangement form of the light emitting regions PXA-R, PXA-G, and PXA-B is not limited to the configuration illustrated in FIG. 1, and the order in which the red light emitting region PXA-R, the green light emitting region PXA-G, and the blue light emitting region PXA-B are arranged may be provided in one or more suitable combinations according to the characteristics of display quality required in the display apparatus DD. For example, the arrangement form of the light emitting regions PXA-R, PXA-G, and PXA-B may be a pentile (PENTILE®) arrangement form (for example, an RGBG matrix, an RGBG structure, or an RGBG matrix structure) or a diamond (Diamond Pixel™) arrangement form (e.g., a display (e.g., an OLED display) containing red, blue, and green (RGB) light-emitting regions arranged in the shape of diamonds). PENTILE® is a duly registered trademark of Samsung Display Co., Ltd. Diamond Pixel™ is a trademark of Samsung Display Co., Ltd.

In some embodiments, the areas of the light emitting regions PXA-R, PXA-G, and PXA-B may be different from each other. For example, in an embodiment, the area of the green light emitting region PXA-G may be smaller than that of the blue light emitting region PXA-B, but embodiments of the present disclosure are not limited thereto.

Hereinafter, FIG. 3 to FIG. 6 are cross-sectional views schematically showing light emitting elements according to one or more embodiments of the present disclosure. The light emitting element ED of an embodiment may include a first electrode EL1, a hole transport region HTR, an emission layer EML, an electron transport region ETR, and a second electrode EL2 stacked in order (e.g., in the stated order).

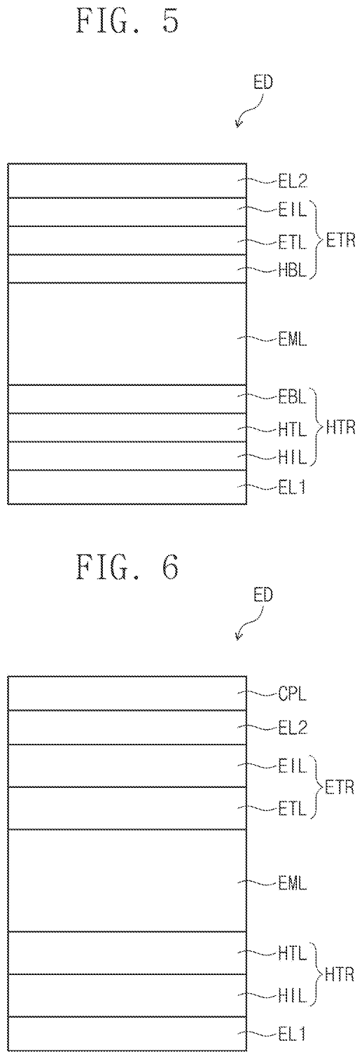

Compared with FIG. 3, FIG. 4 illustrates a cross-sectional view of a light emitting element ED of an embodiment, in which a hole transport region HTR may include a hole injection layer HIL and a hole transport layer HTL, and an electron transport region ETR may include an electron injection layer EIL and an electron transport layer ETL. In some embodiments, compared with FIG. 3, FIG. 5 illustrates a cross-sectional view of a light emitting element ED of an embodiment, in which a hole transport region HTR may include a hole injection layer HIL, a hole transport layer HTL, and an electron blocking layer EBL, and an electron transport region ETR may include an electron injection layer EIL, an electron transport layer ETL, and a hole blocking layer HBL. Compared with FIG. 4, FIG. 6 illustrates a cross-sectional view of a light emitting element ED of an embodiment including a capping layer CPL disposed on a second electrode EL2.

The light emitting element ED of an embodiment may include the amine compound of one or more embodiments of the present disclosure, which will be further explained herein, in the hole transport region HTR. The light emitting element ED of an embodiment may include the amine compound of one or more embodiments in at least one selected from among the hole injection layer HIL, hole transport layer HTL, and electron blocking layer EBL of the hole transport region HTR. For example, in the light emitting element ED of an embodiment, the hole transport layer HTL may include the amine compound of an embodiment.

The first electrode EL1 has conductivity (e.g., is a conductor). The first electrode EL1 may be formed of a metal material, a metal alloy, and/or a conductive compound. The first electrode EL1 may be an anode or a cathode. However, embodiments of the present disclosure are not limited thereto. In some embodiments, the first electrode EL1 may be a pixel electrode. The first electrode EL1 may be a transmissive electrode, a transflective electrode, or a reflective electrode. The first electrode EL1 may include at least one selected from among silver (Ag), magnesium (Mg), copper (Cu), aluminum (Al), platinum (Pt), palladium (Pd), gold (Au), nickel (Ni), neodymium (Nd), iridium (Ir), chromium (Cr), lithium (Li), calcium (Ca), lithium fluoride (LiF), molybdenum (Mo), titanium (Ti), tungsten (W), indium (In), tin (Sn), and zinc (Zn), a compound of two or more selected from among these, a mixture of two or more selected from among these, and/or an oxide thereof.

When the first electrode EL1 is a transmissive electrode, the first electrode EL1 may include a transparent metal oxide such as indium tin oxide (ITO), indium zinc oxide (IZO), zinc oxide (ZnO), and/or indium tin zinc oxide (ITZO). When the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, a compound or mixture thereof (e.g., a mixture of Ag and Mg). In some embodiments, the first electrode EL1 may have a multilayer structure including a reflective film or a transflective film formed of the above-described materials, and a transparent conductive film formed of ITO, IZO, ZnO, ITZO, etc. For example, in some embodiments, the first electrode EL1 may have a three-layer structure of ITO/Ag/ITO, but embodiments of the present disclosure are not limited thereto. In some embodiments, the first electrode EL1 may include the above-described metal materials, combinations of at least two metal materials of the above-described metal materials, oxides of the above-described metal materials, and/or the like. A thickness of the first electrode EL1 may be from about 700 Å to about 10,000 Å. For example, in some embodiments, the thickness of the first electrode EL1 may be from about 1,000 Å to about 3,000 Å.

The hole transport region HTR may be provided on the first electrode EL1. The hole transport region HTR may include at least one of a hole injection layer HIL, a hole transport layer HTL, a buffer layer or an emission-auxiliary layer, or an electron blocking layer EBL.

The hole transport region HTR may have a single layer formed of a single material, a single layer formed of a plurality of different materials, or a multilayer structure including a plurality of layers formed of a plurality of different materials.

For example, in one or more embodiments, the hole transport region HTR may have a single layer structure of a hole injection layer HIL or a hole transport layer HTL, or may have a single layer structure formed of a hole injection material and a hole transport material. In some embodiments, the hole transport region HTR may have a single layer structure formed of a plurality of different materials, or a structure in which a hole injection layer HIL/hole transport layer HTL, a hole injection layer HIL/hole transport layer HTL/buffer layer, a hole injection layer HIL/buffer layer, a hole transport layer HTL/buffer layer, or a hole injection layer HIL/hole transport layer HTL/electron blocking layer EBL are stacked in order (e.g., in each stated order) from the first electrode EL1, but embodiments of the present disclosure are not limited thereto.

A thickness of the hole transport region HTR may be, for example, from about 50 Å to about 15,000 Å. The hole transport region HTR may be formed utilizing one or more suitable methods such as a vacuum deposition method, a spin coating method, a casting method, a Langmuir-Blodgett (LB) method, an inkjet printing method, a laser printing method, and/or a laser induced thermal imaging (LITI) method.

The light emitting element ED of an embodiment may include the amine compound of one or more embodiments in the hole transport region HTR. In the light emitting element ED of an embodiment, the hole transport region HTR may include a hole injection layer HIL and a hole transport layer HTL, and the amine compound of one or more embodiments may be included in at least one selected from among the hole injection layer HIL and the hole transport layer HTL. For example, in one or more embodiments, the hole transport layer HTL may include the amine compound of one or more embodiments.

The amine compound of an embodiment may include a structure in which a first substituent, a second substituent, and a third substituent are connected with the nitrogen atom of an amine. In the amine compound of an embodiment, the first substituent to the third substituent may be directly or indirectly combined with the nitrogen atom of the amine. The first substituent may be a substituted or unsubstituted aryl group or a substituted or unsubstituted heteroaryl group, directly connected with the nitrogen atom of the amine.

The second substituent may include an α-naphthyl moiety. The α-naphthyl moiety may be an α-naphthyl moiety in which an aryl group is connected at position 2 of the α-naphthyl moiety. The second substituent may be directly connected with the nitrogen atom of the amine at the alpha position (carbon position 1) of a naphthalene, or may be indirectly connected with the nitrogen atom of the amine compound through a substituted or unsubstituted arylene linker.

The third substituent may include a β-naphthyl moiety. The third substituent may be directly connected with the nitrogen atom of the amine at the beta position (carbon position 2) of a naphthalene (β-naphthyl moiety), or may be indirectly connected with the nitrogen atom of the amine through a substituted or unsubstituted arylene linker.

The amine compound of an embodiment may be a monoamine compound including a single amine group. The amine compound of an embodiment may be a monoamine compound in which only one amine group is present in a state not forming a ring in a molecular structure.

In one or more embodiments, the amine compound may be represented by Formula 1. In Formula 1, Ar₁ may correspond to the first substituent. In Formula 1, any one selected from among AN and BN may correspond to the second substituent, and the remainder may correspond to the third substituent.

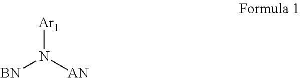

In Formula 1, Ar₁ may be a substituted or unsubstituted aryl group of 6 to 50 ring-forming carbon atoms, or a substituted or unsubstituted heteroaryl group of 5 to 50 ring-forming carbon atoms. For example, in one or more embodiments, Ar₁ may be a substituted or unsubstituted phenyl group, a substituted or unsubstituted biphenyl group, a substituted or unsubstituted terphenyl group, a substituted or unsubstituted quaterphenyl group, a substituted or unsubstituted naphthyl group, a substituted or unsubstituted dibenzofuran group, a substituted or unsubstituted naphthobenzofuran group, a substituted or unsubstituted dibenzothiophene group, a substituted or unsubstituted 9-phenylcarbazole group, or a substituted or unsubstituted fluorenyl group.

In one or more embodiments, Ar₁ may be represented by any one in Substituent Group AR. In Substituent Group AR, “D” is deuterium. “ [symbol] ” is a position connected with the nitrogen atom of an amine in the amine compound of an embodiment, represented by Formula 1.

### Substituent Group AR

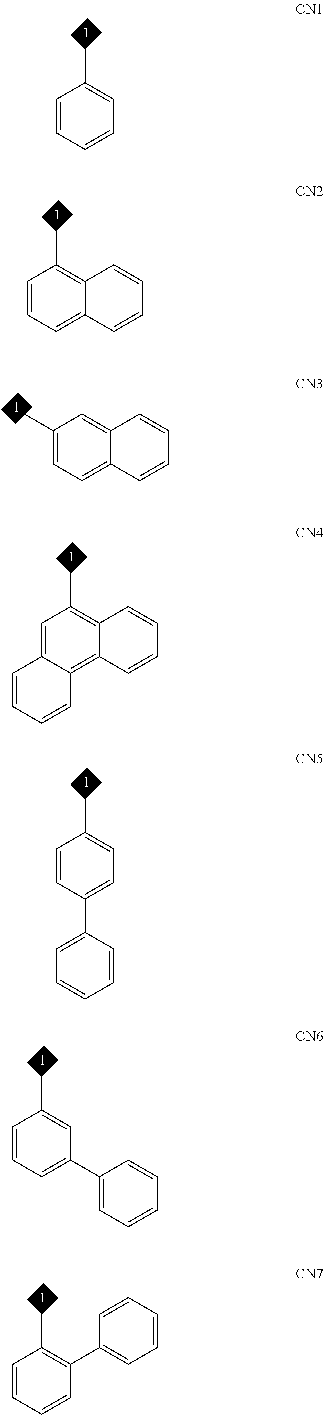

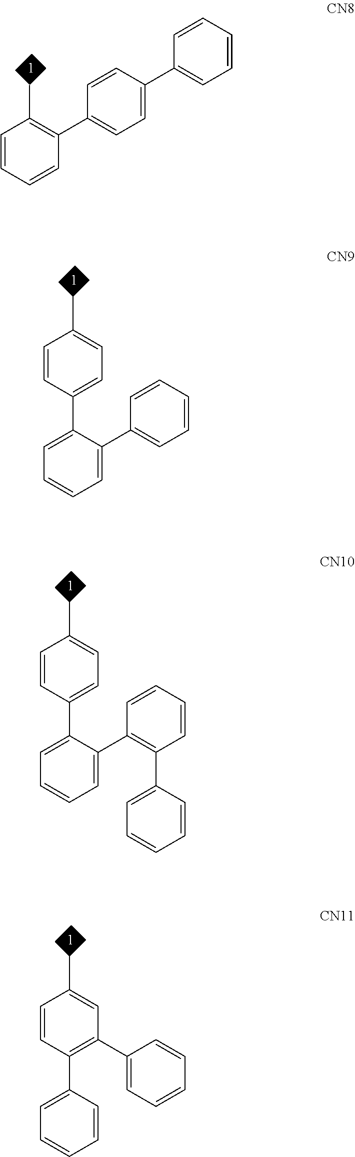

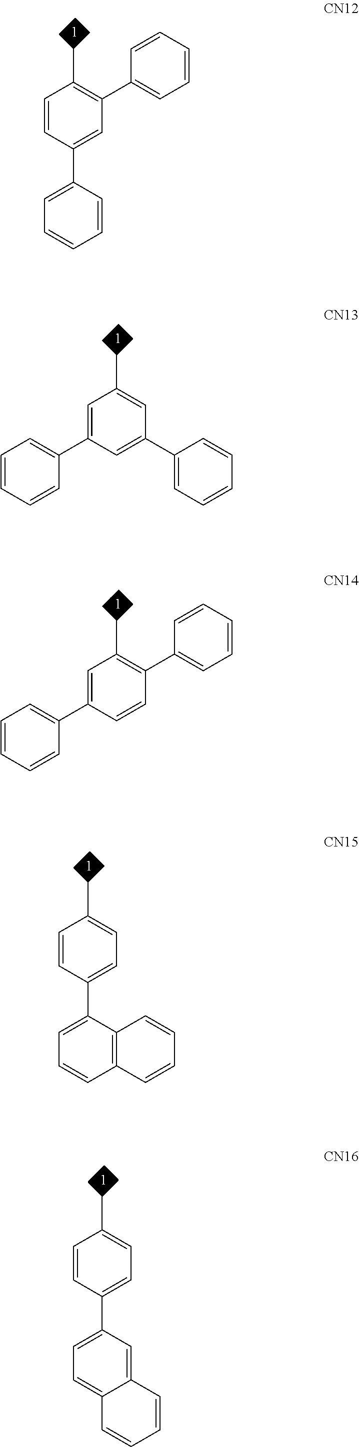

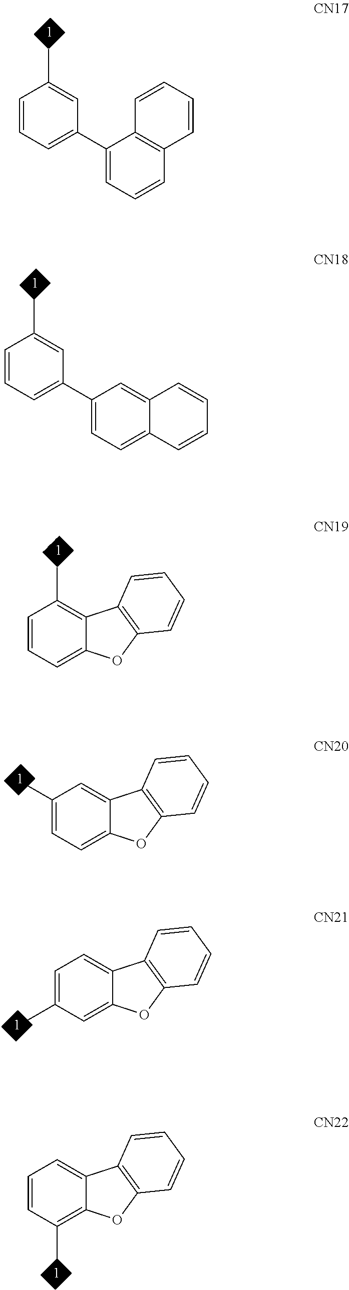

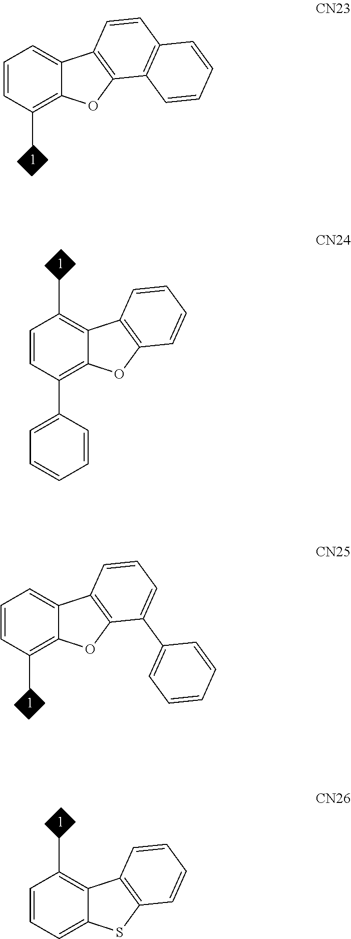 [SMILES: Cc1cccc2c1oc1c3ccccc3ccc21 ; Cc1ccc(-c2ccccc2)c2oc3ccccc3c12 ; Cc1cccc2c1oc1c(-c3ccccc3)cccc12 ; Cc1cccc2sc3ccccc3c12]

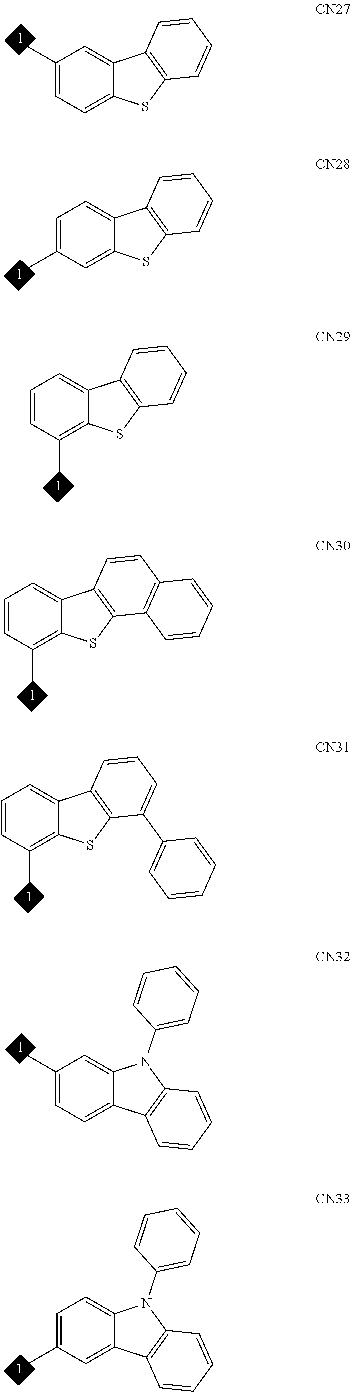

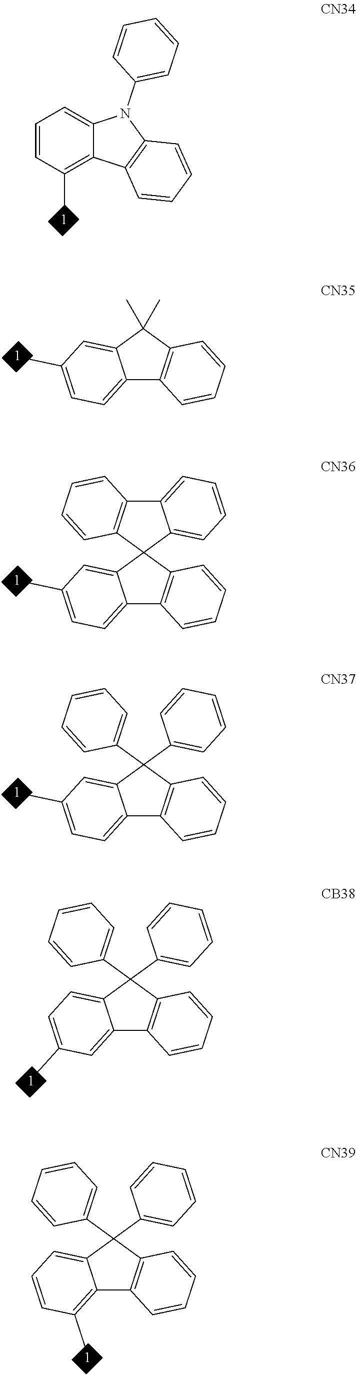

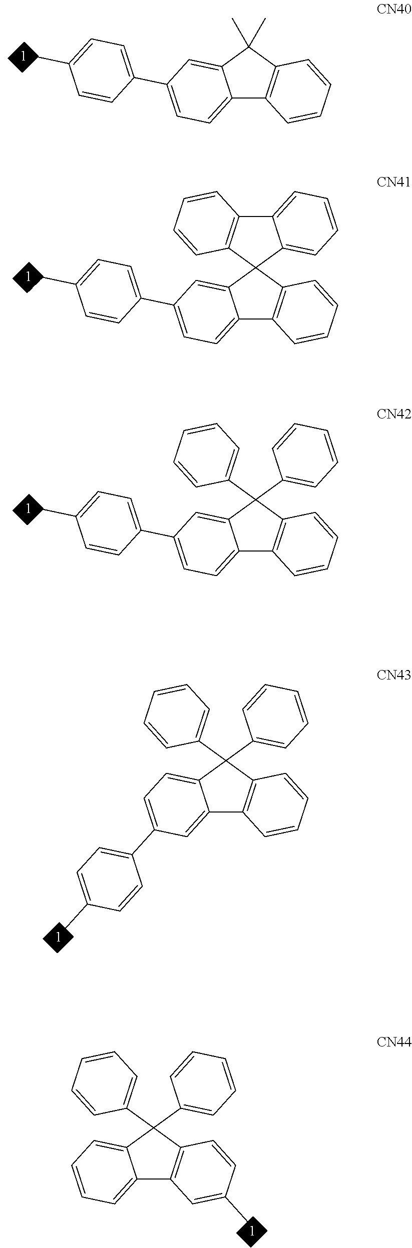

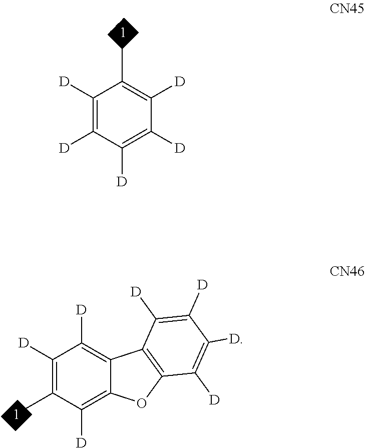

In Formula 1, any one selected from among AN and BN may be represented by Formula A, and the remainder not represented by Formula A may be represented by Formula B. For example, in one or more embodiments, AN may be represented by Formula A, and BN may be represented by Formula B. In one or more embodiments, Formula A may correspond to the second substituent, and Formula B may correspond to the third substituent.

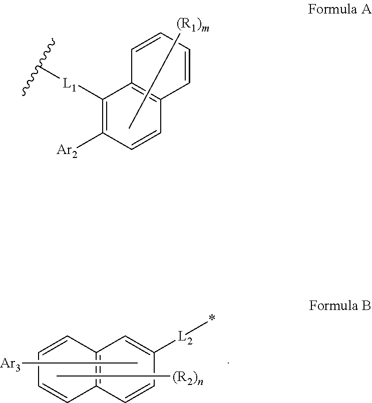

In Formula A and Formula B, L₁ and L₂ may each independently be a direct linkage, or a substituted or unsubstituted arylene group of 6 to 50 ring-forming carbon atoms. For example, in one or more embodiments, L₁ and L₂ may each independently be a direct linkage, or a substituted or unsubstituted phenylene group. When L₁ and L₂ are substituted phenylene groups, L₁ and L₂ may be substituted with one or more deuterium atoms.

In Formula A, R₁ may be hydrogen, deuterium, a cyano group, or a

substituted or unsubstituted alkyl group of 1 to 30 carbon atoms. For example, in some embodiments, R₁ may be hydrogen or deuterium.

In Formula A, “m” may be an integer of 0 to 6. In Formula A, when “m” is 0, Formula A may be unsubstituted with R₁. For example, when “m” is 0 in Formula A, the amine compound of an embodiment may include the second substituent which is unsubstituted with R₁. In Formula A, an embodiment in which “m” is 6, and six R₁(s) are all hydrogen, may be the same as Formula A where “m” is 0. When “m” is an integer of 2 or more, multiple R₁(s) may be all the same, or at least one selected from among multiple R₁(s) may be different from the remainder.

In Formula A, Ar₂ may be a substituted or unsubstituted aryl group of 6 to 50 ring-forming carbon atoms. Ar₂ may be a substituted or unsubstituted monocyclic aryl group of 6 to 50 ring-forming carbon atoms. For example, in one or more embodiments, Ar₂ may be a substituted or unsubstituted phenyl group or a substituted or unsubstituted biphenyl group. In some embodiments, embodiments in which Ar₂ is a substituted or unsubstituted naphthyl group, may be excluded.

In Formula B, R₂ may be hydrogen, deuterium, a cyano group, a substituted or unsubstituted alkyl group of 1 to 30 carbon atoms, or a substituted or unsubstituted aryl group of 6 to 30 ring-forming carbon atoms. For example, in some embodiments, R₂ may be hydrogen or deuterium. In some embodiments, R₂ may be combined with an adjacent group to form a ring. In some embodiments, multiple R₂(s) may be provided, and elements in a pair of adjacent R₂(s) may be combined with each other to form an aromatic hydrocarbon ring.

In Formula B, “n” may be an integer of 0 to 6. In Formula B, when “n” is 0, Formula B may be unsubstituted with R₂. For example, when “n” is 0 in Formula B, the amine compound of an embodiment may include the third substituent which is unsubstituted with R₂. In Formula B, an embodiment in which “n” is 6, and six R₂(s) are all hydrogen, may be the same as Formula B where “n” is 0. When “n” is an integer of 2 or more, multiple R₂(s) may be all the same, or at least one selected from among multiple R₂(s) may be different from the remainder.

In Formula B, Ar₃ may be hydrogen, deuterium, or a substituted or unsubstituted aryl group of 6 to 50 ring-forming carbon atoms. For example, in one or more embodiments, Ar₃ may be hydrogen, deuterium, or a substituted or unsubstituted phenyl group.

For example, in one or more embodiments, when AN is represented by Formula A, and BN is represented by Formula B, the amine compound represented by Formula 1 may be represented by Formula 1a. The same contents as those explained in Formula 1, Formula A, and Formula B may be applied for Ar₁ to Ar₃, R₁, R₂, L₁, L₂, “n” and “m” in Formula 1a.

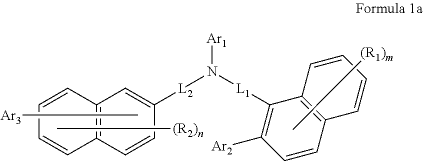

In the present disclosure, Formula 1 may include a structure in which an optional hydrogen atom is substituted with deuterium. In one or more embodiments, at least one selected from among Ar₁, AN, and BN of Formula 1 may include deuterium, or a substituent including deuterium. Formula 1 may have a structure not including deuterium, or a structure of which partial or whole hydrogen atoms are substituted with deuterium.

In one or more embodiments, Formula A may be represented by Formula A1 or Formula A2. In Formula A1 and Formula A2, the same contents as those explained in Formula A may be applied for R₁ and “m”.

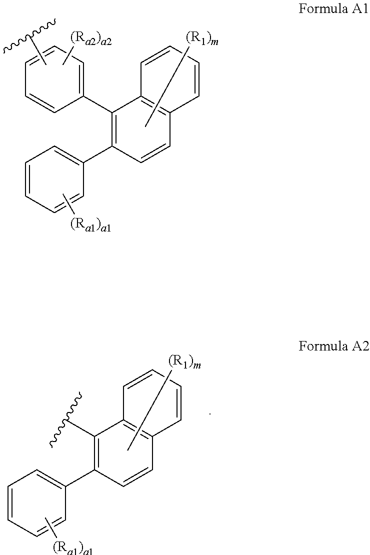

In Formula A1 and Formula A2, Rₐ₁ may be hydrogen, deuterium, or a substituted or unsubstituted aryl group of 6 to 15 carbon atoms for forming a ring. For example, in one or more embodiments, Rₐ₁ may be hydrogen, deuterium, or a substituted or unsubstituted phenyl group. For example, in one or more embodiments, the second substituent may include an α-naphthyl moiety in which an aryl group such as a substituted or unsubstituted phenyl group and/or a substituted or unsubstituted biphenyl group is combined/bonded at position 2 of the α-naphthyl moiety.

In Formula A1 and Formula A2, a1 may be an integer of 0 to 5. In Formula A1 and Formula A2, when a1 is 0, the amine compound of an embodiment may be unsubstituted with Rₐ₁. An embodiment in which a1 is 5, and five Rₐ₁(s) are all hydrogen, may be the same as an embodiment in which a1 is 0. When a1 is an integer of 2 or more, multiple Rₐ₁(s) may be all the same, or at least one selected from among multiple Rₐ₁(s) may be different from the remainder.

In Formula A1, Rₐ₂ may be hydrogen or deuterium, and a2 may be an integer of 0 to 4. In Formula A1, when a2 is 0, the amine compound of an embodiment may be unsubstituted with Rₐ₂. An embodiment in which a2 is 4, and four Rₐ₂(s) are all hydrogen, may be the same as an embodiment in which a2 is 0. When a2 is an integer of 2 or more, multiple Rₐ₂(s) may be all the same, or at least one selected from among multiple Rₐ₂(s) may be different from the remainder.

In one or more embodiments, Formula B may be represented by Formula B1 or Formula B2. In Formula B1 and Formula B2, the same contents explained in Formula B may be applied for R₂ and “n”.

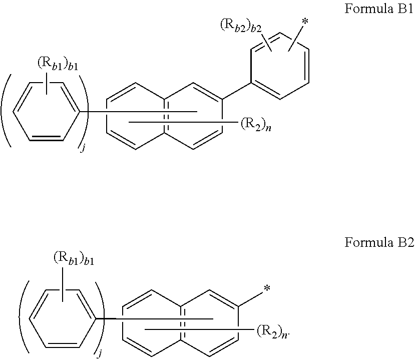

In Formula B1 and Formula B2, R_{b1} and R_{b2} may each independently be hydrogen or deuterium. b1 may be an integer of 0 to 5, and b2 may be an integer of 0 to 4. In Formula B1 and Formula B2, when b1 and b2 are 0, the amine compound of an embodiment may be unsubstituted with R_{b1} and R_{b2}, respectively. An embodiment in which b1 is 5, and five R_{b1}(s) are all hydrogen, may be the same as an embodiment in which b1 is 0. An embodiment in which b2 is 4, and four R_{b2}(s) are all hydrogen, may be the same as an embodiment in which b2 is 0. When b1 and b2 are integers of 2 or more, each of multiple R_{b1}(s) and R_{b2}(s) may be all the same, or at least one selected from among each of multiple R_{b1}(s) and R_{b2}(s) may be different from the remainder.

In Formula B1 and Formula B2, “j” may be 0 or 1. For example, in some embodiments, in the third substituent, a phenyl group having the substituent of R_{b1} may be connected or unconnected to the β-naphthyl.

In one or more embodiments, Formula B1 may be represented by Formula B1-1 or Formula B1-2. Formula B2 may be represented by Formula B2-1 or Formula B2-2. In Formula B1-1, Formula B1-2, Formula B2-1, and Formula B2-2, the same contents as those explained in Formula B1 and Formula B2 may be applied for R_{b1}, R_{b2}, b1, b2, and “j”.

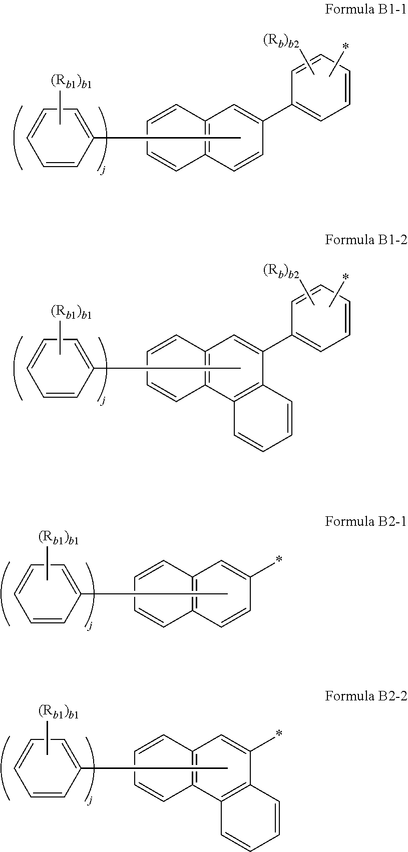

In one or more embodiments, AN in Formula 1 may be represented by any one in Substituent Group A. The substituents illustrated in Substituent Group A may be mere example embodiments of Formula A. However, embodiments of the present disclosure are not limited thereto.

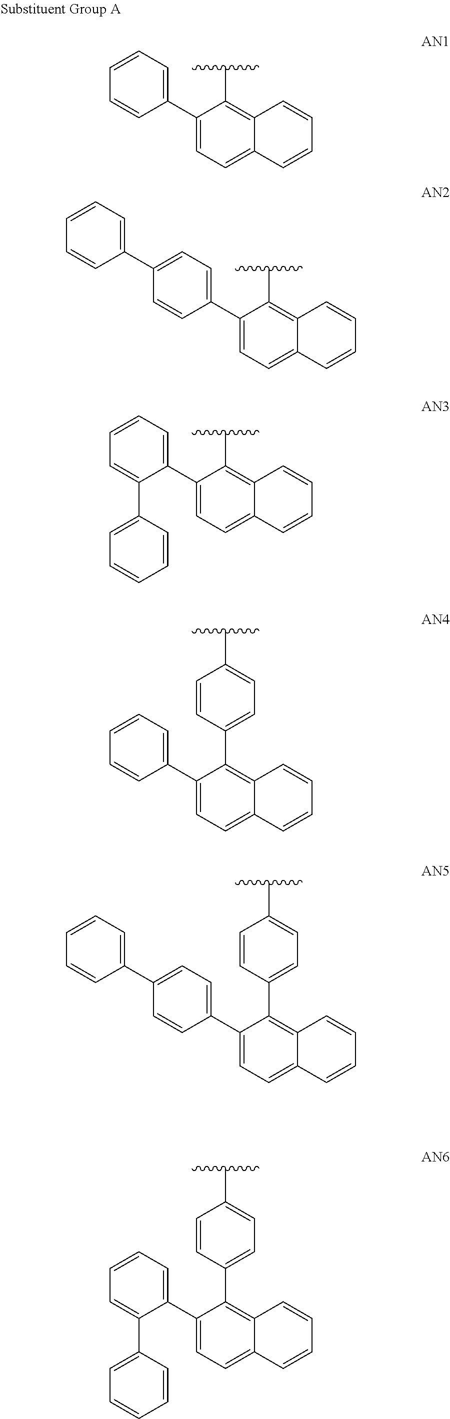 [SMILES: *c1c(-c2ccccc2)ccc2ccccc12 ; *c1c(-c2ccc(-c3ccccc3)cc2)ccc2ccccc12 ; *c1c(-c2ccccc2-c2ccccc2)ccc2ccccc12 ; *c1ccc(-c2c(-c3ccccc3)ccc3ccccc23)cc1 ; *c1ccc(-c2c(-c3ccc(-c4ccccc4)cc3)ccc3ccccc23)cc1 ; *c1ccc(-c2c(-c3ccccc3-c3ccccc3)ccc3ccccc23)cc1]

In one or more embodiments, BN in Formula 1 may be represented by any one in Substituent Group B. The substituents illustrated in Substituent Group B may be mere example embodiments of Formula B. However, embodiments of the present disclosure are not limited thereto.

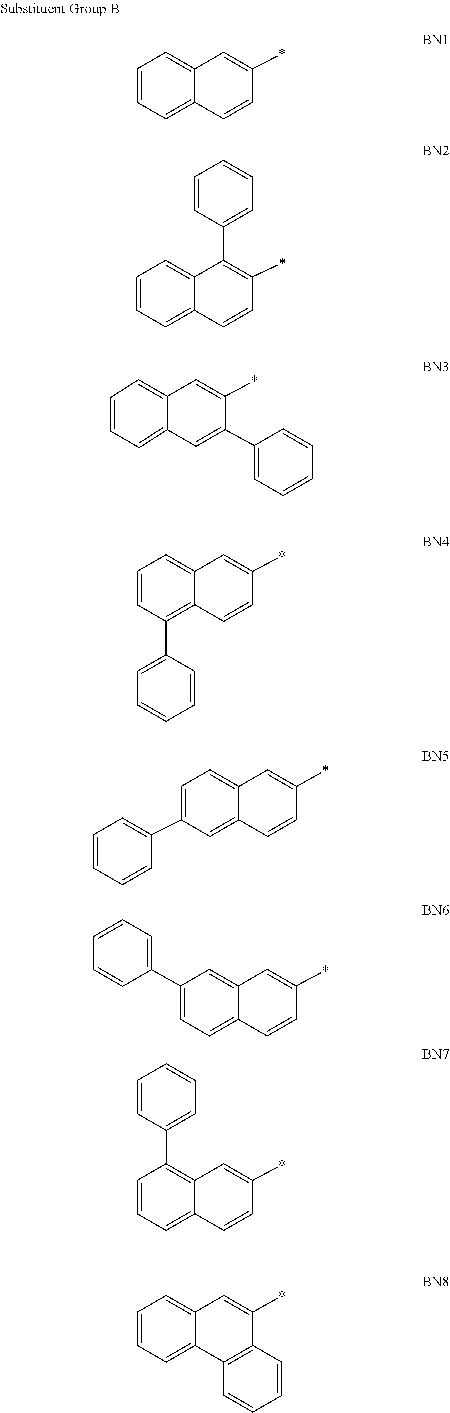

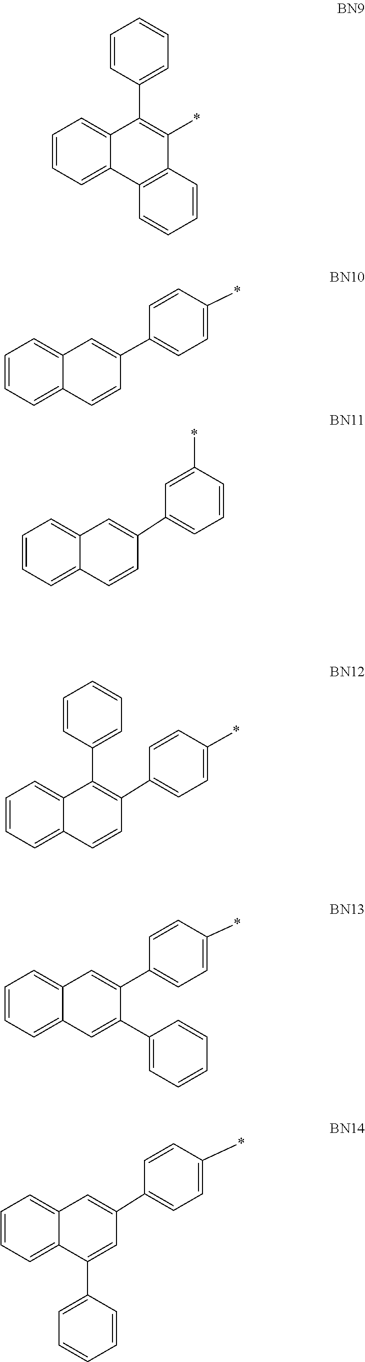 [SMILES: *c1c(-c2ccccc2)c2ccccc2c2ccccc12 ; *c1ccc(-c2ccc3ccccc3c2)cc1 ; *c1cccc(-c2ccc3ccccc3c2)c1 ; *c1ccc(-c2ccc3ccccc3c2-c2ccccc2)cc1 ; *c1ccc(-c2cc3ccccc3cc2-c2ccccc2)cc1 ; *Cc1ccc(-c2cc(-c3ccccc3)c3ccccc3c2)cc1]

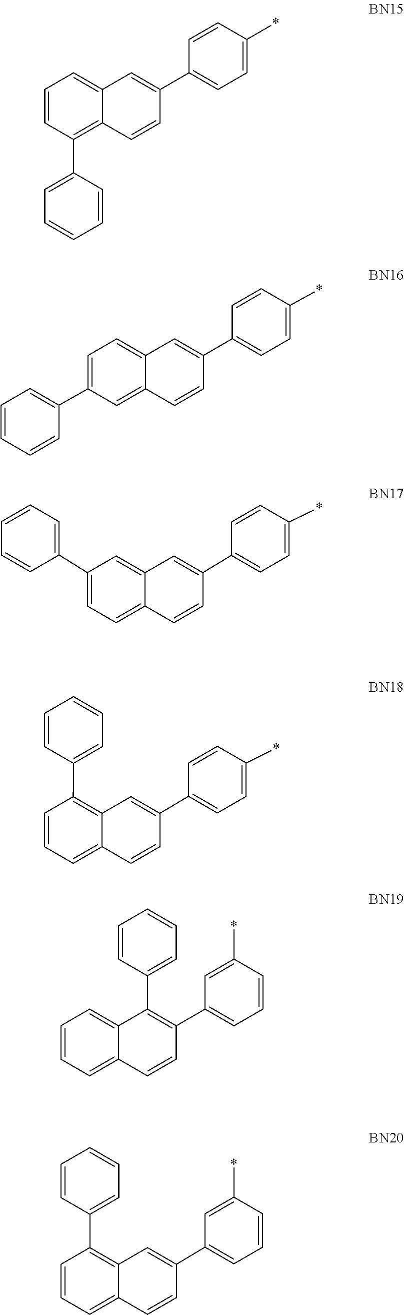 [SMILES: *c1ccc(-c2ccc3c(-c4ccccc4)cccc3c2)cc1 ; *c1ccc(-c2ccc3cc(-c4ccccc4)ccc3c2)cc1 ; *c1ccc(-c2ccc3ccc(-c4ccccc4)cc3c2)cc1 ; *c1ccc(-c2ccc3cccc(-c4ccccc4)c3c2)cc1 ; *c1cccc(-c2ccc3ccccc3c2-c2ccccc2)c1 ; *c1cccc(-c2ccc3cccc(-c4ccccc4)c3c2)c1]

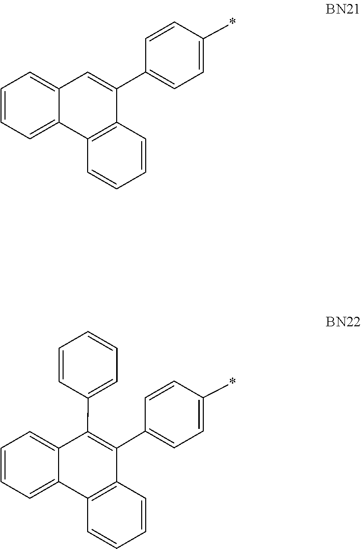 [SMILES: *c1ccc(-c2cc3ccccc3c3ccccc23)cc1 ; *c1ccc(-c2c(-c3ccccc3)c3ccccc3c3ccccc23)cc1]

In one or more embodiments, the amine compound represented by Formula may be represented by Formula 2. In Formula 2, the same contents as those explained in Formula 1 may each independently be applied for Ar₁ to Ar₃, L₁, L₂, R₂, and “n”.

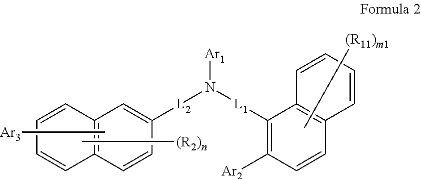

In Formula 2, R₁₁ may be hydrogen or deuterium, and m1 may be an integer of 0 to 6. In Formula 2, when m1 is 0, Formula 2 may be unsubstituted with R₁₁. In Formula 2, an embodiment in which m1 is 6, and six R₁₁(s) are all hydrogen, may be the same as an embodiment in which m1 is 0. When m1 is an integer of 2 or more, multiple R₁₁(s) may be all the same, or at least one selected from among multiple R₁₁(s) may be different from the remainder.

In one or more embodiments, the amine compound represented by Formula may be represented by Formula 3-1 or Formula 3-2. Formula 3-1 and Formula 3-2 correspond to Formula 1 where AN is represented by Formula A, and Ar₂ is embodied. In some embodiments, Formula 3-1 and Formula 3-2 correspond to Formula 1 where BN is represented by Formula B, and R₂ is embodied. In Formula 3-1 and Formula 3-2, the same contents as those explained in Formula 1 may each independently be applied for Ar₁, Ar₃, L₁, L₂, R₁, and “m”.

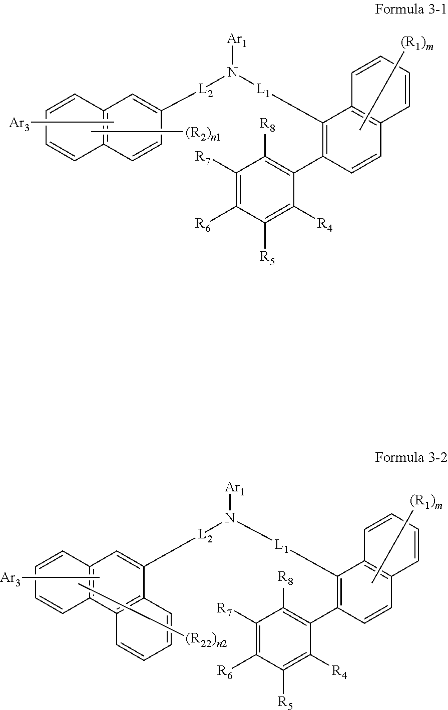

In Formula 3-1 and Formula 3-2, R₄ to R₈ may each independently be hydrogen, deuterium, or a substituted or unsubstituted aryl group of 6 to 15 ring-forming carbon atoms. For example, in some embodiments, R₄ to R₈ may each independently be hydrogen, deuterium, or a substituted or unsubstituted phenyl group.

In Formula 3-1 and Formula 3-2, R₂₁ and R₂₂ may each independently be hydrogen or deuterium. n1 may be an integer of 0 to 6, and n2 may be an integer of 0 to 8. In Formula 3-1 and Formula 3-2, when n1 and n2 are 0, the amine compound of an embodiment may be unsubstituted with R₂₁ and R₂₂, respectively. An embodiment in which n1 is 6, and six R₂₁(s) are all hydrogen, may be the same as an embodiment in which n1 is 0. An embodiment in which n2 is 8, and eight R₂₂(s) are all hydrogen, may be the same as an embodiment in which n2 is 0. When n1 and n2 are integers of 2 or more, each of multiple R₂₁(s) and R₂₂(s) may be all the same, or at least one selected among each of multiple R₂₁(s) and R₂₂(s) may be different from the remainder.

In one or more embodiments, the amine compound represented by Formula may be represented by Formula 4-1 or Formula 4-2. Formula 4-1 or Formula 4-2 represents amine compounds of embodiments of Formula 1 where AN is represented by Formula A, BN is represented by Formula B, and the substituent represented by Ar₁ is embodied. In Formula 4-1 or Formula 4-2, the same contents as those explained in Formula 1 may each independently be applied for Ar₂, Ar₃, L₁, L₂, R₁, R₂, “m” and “n”.

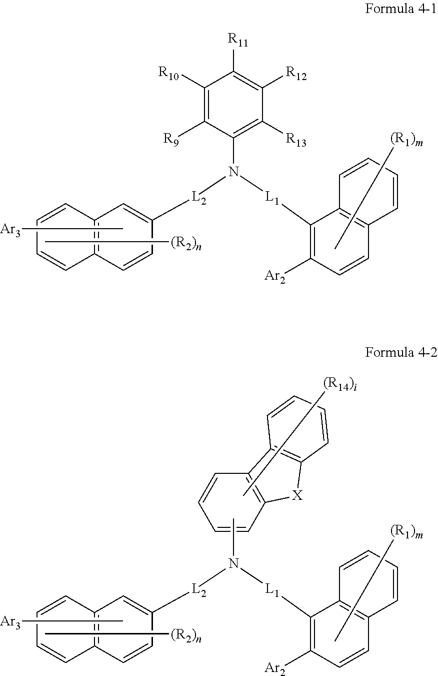

In Formula 4-1, R₉ to R₁₃ may each independently be hydrogen, deuterium, or a substituted or unsubstituted aryl group of 6 to 30 ring-forming carbon atoms. In some embodiments, substituents in at least one pair selected from among adjacent R₉ and R₁₀, R₁₀ and R₁₁, R₁₁ and R₁₂, and R₁₂ and R₁₃ may be combined with each other to form an aromatic hydrocarbon ring. For example, in some embodiments, R₉ to R₁₃ may each independently be hydrogen, deuterium, or a substituted or unsubstituted phenyl group, a substituted or unsubstituted biphenyl group, a substituted or unsubstituted terphenyl group, a substituted or unsubstituted naphthyl group, or a substituted or unsubstituted fluorenyl group. In some embodiments, in R₉ to R₁₃, any one pair or two pairs selected from among adjacent R₉ and R₁₀, R₁₀ and R₁₁, R₁₁ and R₁₂, and R₁₂ and R₁₃ may be combined to form aromatic rings. In these embodiments, a benzene ring where R₉ to R₁₃ are substituted may be a naphthyl group or a phenanthryl group.

In Formula 4-2, X may be O, S, NRₓ₁, or CRₓ₂Rₓ₃. For example, when X is O, the amine compound of an embodiment may include a dibenzofuran moiety, and when X is S, the amine compound of an embodiment may include a dibenzothiophene moiety. In some embodiments, when X is NRₓ₁, the amine compound of an embodiment may include a carbazole moiety, and when X is CRₓ₂Rₓ₃, the amine compound of an embodiment may include a fluorene moiety.

In Formula 4-2, Rₓ₁ may be a substituted or unsubstituted aryl group of 6 to 15 ring-forming carbon atoms. For example, in one or more embodiments, Rₓ₁ may be a substituted or unsubstituted phenyl group.

In Formula 4-2, Rₓ₂ and Rₓ₃ may each independently be a substituted or unsubstituted alkyl group of 1 to 10 carbon atoms, or a substituted or unsubstituted aryl group of 6 to 15 ring-forming carbon atoms. For example, in one or more embodiments, Rₓ₂ and Rₓ₃ may each independently be a substituted or unsubstituted methyl group, or a substituted or unsubstituted phenyl group. In some embodiments, Rₓ₂ and Rₓ₃ may be combined with each other to form a ring. For example, in some embodiments, Rₓ₂ and Rₓ₃ may be combined with each other to form a spiro structure.

In Formula 4-2, R₁₄ may be hydrogen, deuterium, or a substituted or unsubstituted aryl group of 6 to 15 ring-forming carbon atoms. For example, in one or more embodiments, R₁₄ may be hydrogen, deuterium, or a substituted or unsubstituted phenyl group. In some embodiments, R₁₄ may be combined with an adjacent group to form a ring. For example, in some embodiments, multiple R₁₄(s) may be provided, and adjacent two R₁₄(s) may be combined with each other to form a ring.

In Formula 4-2, “i” may be an integer of 0 to 7. In Formula 4-2, when “i” is 0, the amine compound of an embodiment may be unsubstituted with R₁₄. An embodiment in which “i” is 7, and seven R₁₄(s) are all hydrogen, may be the same as an embodiment in which “i” is 0. When “i” is an integer of 2 or more, multiple R₁₄(s) may be all the same, or at least one selected from among multiple R₁₄(s) may be different from the remainder.

In one or more embodiments, the amine compound represented by Formula may be represented by any one selected from among Formula 5-1 to Formula 5-4. Formula 5-1 to Formula 5-4 represent Formula 1 where AN is represented by Formula A, BN is represented by Formula B, and L₁ and L₂ are specified. Formula 5-1 represents embodiments of the amine compound represented by Formula 1 where L₁ and L₂ are substituted or unsubstituted phenylene groups. Formula 5-2 represents embodiments of the amine compound represented by Formula 1 where L₁ is a direct linkage. Formula 5-3 represents embodiments of the amine compound represented by Formula 1 where L₂ is a direct linkage. Formula 5-4 represents embodiments of the amine compound represented by Formula 1 where L₁ and L₂ are all direct linkages.

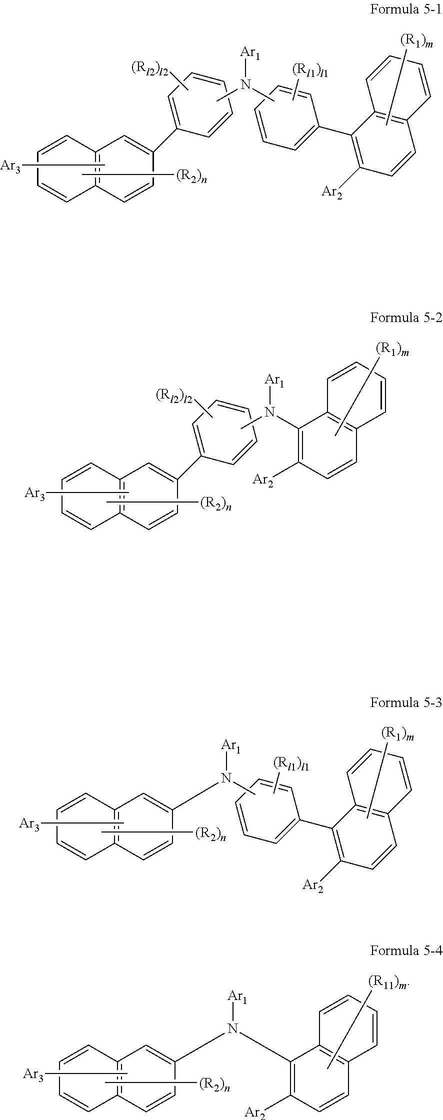

In Formula 5-1 to Formula 5-4, Rₗ₁ and Rₗ₂ may each independently be hydrogen, or deuterium. l1 and l2 may each independently be an integer of 0 to 4. In Formula 5-1 to Formula 5-4, when I1 and 12 are 0, the amine compound of an embodiment may be unsubstituted with Rₗ₁ and Rₗ₂, respectively. Embodiments in which l1 and l2 are 4, and four Rₗ₁(s) and Rₗ₂(s) are all hydrogen, may be the same as embodiments in which l1 and l2 are 0, respectively. When l1 and l2 are integers of 2 or more, each of multiple Rₗ₁(s) and Rₗ₂(s) may be all the same, or at least one selected from among each of multiple Rₗ₁ and Rₗ₂ may be different from the remainder.

In Formula 5-1 to Formula 5-4, the same contents as those explained in Formula 1 may each independently be applied for Ar₁ to Ar₃, R₁, R₂, “m” and “n”.

In one or more embodiments, the amine compound represented by Formula may be a compound satisfying any one selected from among substituent combinations represented in Compound Combination Table 1. In Compound Combination Table 1, AN may be selected from Substituent Group A, BN may be selected from Substituent Group B, and Ar₁ may be selected from Substituent Group AR. In other words, the amine compound represented by Formula 1 may be a compound satisfying any one selected from among the substituent combinations represented in Compound Combination Table 1, where AN is represented by any one in Substituent Group A, BN is represented by any one in Substituent Group B, and Ar₁ is represented by any one in Substituent Group AR.

Compound Combination Table 1

| Compound | | | |
|---|---|---|---|
| NO. | AN | BN | AR₁ |
| AA1 | AN1 | BN1 | CN1 |
| AA2 | AN1 | BN1 | CN2 |
| AA3 | AN1 | BN1 | CN3 |
| AA4 | AN1 | BN1 | CN4 |
| AA5 | AN1 | BN1 | CN5 |
| AA6 | AN1 | BN1 | CN6 |
| AA7 | AN1 | BN1 | CN7 |
| AA8 | AN1 | BN1 | CN8 |
| AA9 | AN1 | BN1 | CN9 |
| AA10 | AN1 | BN1 | CN10 |
| AA11 | AN1 | BN1 | CN11 |
| AA12 | AN1 | BN1 | CN12 |
| AA13 | AN1 | BN1 | CN13 |
| AA14 | AN1 | BN1 | CN14 |
| AA15 | AN1 | BN1 | CN15 |
| AA16 | AN1 | BN1 | CN16 |
| AA17 | AN1 | BN1 | CN17 |
| AA18 | AN1 | BN1 | CN18 |
| AA19 | AN1 | BN1 | CN19 |
| AA20 | AN1 | BN1 | CN20 |
| AA21 | AN1 | BN1 | CN21 |
| AA22 | AN1 | BN1 | CN22 |
| AA23 | AN1 | BN1 | CN23 |
| AA24 | AN1 | BN1 | CN24 |
| AA25 | AN1 | BN1 | CN25 |
| AA26 | AN1 | BN1 | CN26 |
| AA27 | AN1 | BN1 | CN27 |
| AA28 | AN1 | BN1 | CN28 |
| AA29 | AN1 | BN1 | CN29 |
| AA30 | AN1 | BN1 | CN30 |
| AA31 | AN1 | BN1 | CN31 |
| AA32 | AN1 | BN1 | CN32 |
| AA33 | AN1 | BN1 | CN33 |
| AA34 | AN1 | BN1 | CN34 |
| AA35 | AN1 | BN1 | CN35 |
| AA36 | AN1 | BN1 | CN36 |
| AB1 | AN1 | BN2 | CN1 |
| AB2 | AN1 | BN2 | CN2 |
| AB3 | AN1 | BN2 | CN3 |
| AB4 | AN1 | BN2 | CN4 |
| AB5 | AN1 | BN2 | CN5 |
| AB6 | AN1 | BN2 | CN6 |
| AB7 | AN1 | BN2 | CN7 |
| AB8 | AN1 | BN2 | CN8 |
| AB9 | AN1 | BN2 | CN9 |
| AB10 | AN1 | BN2 | CN10 |
| AB11 | AN1 | BN2 | CN11 |
| AB12 | AN1 | BN2 | CN12 |
| AB13 | AN1 | BN2 | CN13 |
| AB14 | AN1 | BN2 | CN14 |
| AB15 | AN1 | BN2 | CN15 |
| AB16 | AN1 | BN2 | CN16 |
| AB17 | AN1 | BN2 | CN17 |
| AB18 | AN1 | BN2 | CN18 |
| AB19 | AN1 | BN2 | CN19 |
| AB20 | AN1 | BN2 | CN20 |
| AB21 | AN1 | BN2 | CN21 |
| AB22 | AN1 | BN2 | CN22 |
| AB23 | AN1 | BN2 | CN23 |
| AB24 | AN1 | BN2 | CN24 |
| AB25 | AN1 | BN2 | CN25 |
| AB26 | AN1 | BN2 | CN26 |
| AB27 | AN1 | BN2 | CN27 |
| AB28 | AN1 | BN2 | CN28 |
| AB29 | AN1 | BN2 | CN29 |
| AB30 | AN1 | BN2 | CN30 |
| AB31 | AN1 | BN2 | CN31 |
| AB32 | AN1 | BN2 | CN32 |
| AB33 | AN1 | BN2 | CN33 |
| AB34 | AN1 | BN2 | CN34 |
| AB35 | AN1 | BN2 | CN35 |
| AB36 | AN1 | BN2 | CN36 |
| AC1 | AN1 | BN3 | CN1 |
| AC2 | AN1 | BN3 | CN2 |
| AC3 | AN1 | BN3 | CN3 |
| AC4 | AN1 | BN3 | CN4 |
| AC5 | AN1 | BN3 | CN5 |
| AC6 | AN1 | BN3 | CN6 |
| AC7 | AN1 | BN3 | CN7 |
| AC8 | AN1 | BN3 | CN8 |
| AC9 | AN1 | BN3 | CN9 |
| AC10 | AN1 | BN3 | CN10 |
| AC11 | AN1 | BN3 | CN11 |
| AC12 | AN1 | BN3 | CN12 |
| AC13 | AN1 | BN3 | CN13 |
| AC14 | AN1 | BN3 | CN14 |
| AC15 | AN1 | BN3 | CN15 |
| AC16 | AN1 | BN3 | CN16 |
| AC17 | AN1 | BN3 | CN17 |
| AC18 | AN1 | BN3 | CN18 |
| AC19 | AN1 | BN3 | CN19 |
| AC20 | AN1 | BN3 | CN20 |
| AC21 | AN1 | BN3 | CN21 |
| AC22 | AN1 | BN3 | CN22 |
| AC23 | AN1 | BN3 | CN23 |
| AC24 | AN1 | BN3 | CN24 |
| AC25 | AN1 | BN3 | CN25 |
| AC26 | AN1 | BN3 | CN26 |
| AC27 | AN1 | BN3 | CN27 |
| AC28 | AN1 | BN3 | CN28 |
| AC29 | AN1 | BN3 | CN29 |
| AC30 | AN1 | BN3 | CN30 |
| AC31 | AN1 | BN3 | CN31 |
| AC32 | AN1 | BN3 | CN32 |
| AC33 | AN1 | BN3 | CN33 |
| AC34 | AN1 | BN3 | CN34 |
| AC35 | AN1 | BN3 | CN35 |
| AC36 | AN1 | BN3 | CN36 |
| AD1 | AN1 | BN4 | CN1 |
| AD2 | AN1 | BN4 | CN2 |
| AD3 | AN1 | BN4 | CN3 |
| AD4 | AN1 | BN4 | CN4 |
| AD5 | AN1 | BN4 | CN5 |
| AD6 | AN1 | BN4 | CN6 |
| AD7 | AN1 | BN4 | CN7 |
| AD8 | AN1 | BN4 | CN8 |
| AD9 | AN1 | BN4 | CN9 |
| AD10 | AN1 | BN4 | CN10 |
| AD11 | AN1 | BN4 | CN11 |
| AD12 | AN1 | BN4 | CN12 |
| AD13 | AN1 | BN4 | CN13 |
| AD14 | AN1 | BN4 | CN14 |
| AD15 | AN1 | BN4 | CN15 |
| AD16 | AN1 | BN4 | CN16 |
| AD17 | AN1 | BN4 | CN17 |
| AD18 | AN1 | BN4 | CN18 |
| AD19 | AN1 | BN4 | CN19 |
| AD20 | AN1 | BN4 | CN20 |
| AD21 | AN1 | BN4 | CN21 |
| AD22 | AN1 | BN4 | CN22 |
| AD23 | AN1 | BN4 | CN23 |
| AD24 | AN1 | BN4 | CN24 |
| AD25 | AN1 | BN4 | CN25 |
| AD26 | AN1 | BN4 | CN26 |
| AD27 | AN1 | BN4 | CN27 |
| AD28 | AN1 | BN4 | CN28 |
| AD29 | AN1 | BN4 | CN29 |
| AD30 | AN1 | BN4 | CN30 |
| AD31 | AN1 | BN4 | CN31 |
| AD32 | AN1 | BN4 | CN32 |
| AD33 | AN1 | BN4 | CN33 |
| AD34 | AN1 | BN4 | CN34 |
| AD35 | AN1 | BN4 | CN35 |
| AD36 | AN1 | BN4 | CN36 |
| AE1 | AN1 | BN5 | CN1 |
| AE2 | AN1 | BN5 | CN2 |
| AE3 | AN1 | BN5 | CN3 |
| AE4 | AN1 | BN5 | CN4 |
| AE5 | AN1 | BN5 | CN5 |
| AE6 | AN1 | BN5 | CN6 |
| AE7 | AN1 | BN5 | CN7 |
| AE8 | AN1 | BN5 | CN8 |
| AE9 | AN1 | BN5 | CN9 |
| AE10 | AN1 | BN5 | CN10 |
| AE11 | AN1 | BN5 | CN11 |
| AE12 | AN1 | BN5 | CN12 |
| AE13 | AN1 | BN5 | CN13 |
| AE14 | AN1 | BN5 | CN14 |
| AE15 | AN1 | BN5 | CN15 |
| AE16 | AN1 | BN5 | CN16 |
| AE17 | AN1 | BN5 | CN17 |
| AE18 | AN1 | BN5 | CN18 |
| AE19 | AN1 | BN5 | CN19 |
| AE20 | AN1 | BN5 | CN20 |
| AE21 | AN1 | BN5 | CN21 |
| AE22 | AN1 | BN5 | CN22 |
| AE23 | AN1 | BN5 | CN23 |
| AE24 | AN1 | BN5 | CN24 |
| AE25 | AN1 | BN5 | CN25 |
| AE26 | AN1 | BN5 | CN26 |
| AE27 | AN1 | BN5 | CN27 |
| AE28 | AN1 | BN5 | CN28 |
| AE29 | AN1 | BN5 | CN29 |
| AE30 | AN1 | BN5 | CN30 |
| AE31 | AN1 | BN5 | CN31 |
| AE32 | AN1 | BN5 | CN32 |
| AE33 | AN1 | BN5 | CN33 |
| AE34 | AN1 | BN5 | CN34 |
| AE35 | AN1 | BN5 | CN35 |
| AE36 | AN1 | BN5 | CN36 |
| AF1 | AN1 | BN6 | CN1 |
| AF2 | AN1 | BN6 | CN2 |
| AF3 | AN1 | BN6 | CN3 |
| AF4 | AN1 | BN6 | CN4 |
| AF5 | AN1 | BN6 | CN5 |
| AF6 | AN1 | BN6 | CN6 |
| AF7 | AN1 | BN6 | CN7 |
| AF8 | AN1 | BN6 | CN8 |
| AF9 | AN1 | BN6 | CN9 |
| AF10 | AN1 | BN6 | CN10 |
| AF11 | AN1 | BN6 | CN11 |
| AF12 | AN1 | BN6 | CN12 |
| AF13 | AN1 | BN6 | CN13 |
| AF14 | AN1 | BN6 | CN14 |
| AF15 | AN1 | BN6 | CN15 |
| AF16 | AN1 | BN6 | CN16 |
| AF17 | AN1 | BN6 | CN17 |
| AF18 | AN1 | BN6 | CN18 |
| AF19 | AN1 | BN6 | CN19 |
| AF20 | AN1 | BN6 | CN20 |
| AF21 | AN1 | BN6 | CN21 |
| AF22 | AN1 | BN6 | CN22 |
| AF23 | AN1 | BN6 | CN23 |
| AF24 | AN1 | BN6 | CN24 |
| AF25 | AN1 | BN6 | CN25 |
| AF26 | AN1 | BN6 | CN26 |
| AF27 | AN1 | BN6 | CN27 |
| AF28 | AN1 | BN6 | CN28 |
| AF29 | AN1 | BN6 | CN29 |
| AF30 | AN1 | BN6 | CN30 |
| AF31 | AN1 | BN6 | CN31 |
| AF32 | AN1 | BN6 | CN32 |
| AF33 | AN1 | BN6 | CN33 |
| AF34 | AN1 | BN6 | CN34 |
| AF35 | AN1 | BN6 | CN35 |
| AF36 | AN1 | BN6 | CN36 |
| AG1 | AN1 | BN7 | CN1 |
| AG2 | AN1 | BN7 | CN2 |
| AG3 | AN1 | BN7 | CN3 |
| AG4 | AN1 | BN7 | CN4 |
| AG5 | AN1 | BN7 | CN5 |
| AG6 | AN1 | BN7 | CN6 |
| AG7 | AN1 | BN7 | CN7 |
| AG8 | AN1 | BN7 | CN8 |
| AG9 | AN1 | BN7 | CN9 |
| AG10 | AN1 | BN7 | CN10 |
| AG11 | AN1 | BN7 | CN11 |
| AG12 | AN1 | BN7 | CN12 |
| AG13 | AN1 | BN7 | CN13 |
| AG14 | AN1 | BN7 | CN14 |
| AG15 | AN1 | BN7 | CN15 |
| AG16 | AN1 | BN7 | CN16 |
| AG17 | AN1 | BN7 | CN17 |
| AG18 | AN1 | BN7 | CN18 |
| AG19 | AN1 | BN7 | CN19 |
| AG20 | AN1 | BN7 | CN20 |
| AG21 | AN1 | BN7 | CN21 |
| AG22 | AN1 | BN7 | CN22 |
| AG23 | AN1 | BN7 | CN23 |
| AG24 | AN1 | BN7 | CN24 |
| AG25 | AN1 | BN7 | CN25 |
| AG26 | AN1 | BN7 | CN26 |
| AG27 | AN1 | BN7 | CN27 |
| AG28 | AN1 | BN7 | CN28 |
| AG29 | AN1 | BN7 | CN29 |
| AG30 | AN1 | BN7 | CN30 |
| AG31 | AN1 | BN7 | CN31 |
| AG32 | AN1 | BN7 | CN32 |
| AG33 | AN1 | BN7 | CN33 |
| AG34 | AN1 | BN7 | CN34 |
| AG35 | AN1 | BN7 | CN35 |
| AG36 | AN1 | BN7 | CN36 |
| AH1 | AN1 | BN8 | CN1 |
| AH2 | AN1 | BN8 | CN2 |
| AH3 | AN1 | BN8 | CN3 |
| AH4 | AN1 | BN8 | CN4 |
| AH5 | AN1 | BN8 | CN5 |
| AH6 | AN1 | BN8 | CN6 |
| AH7 | AN1 | BN8 | CN7 |
| AH8 | AN1 | BN8 | CN8 |
| AH9 | AN1 | BN8 | CN9 |
| AH10 | AN1 | BN8 | CN10 |
| AH11 | AN1 | BN8 | CN11 |
| AH12 | AN1 | BN8 | CN12 |
| AH13 | AN1 | BN8 | CN13 |
| AH14 | AN1 | BN8 | CN14 |
| AH15 | AN1 | BN8 | CN15 |
| AH16 | AN1 | BN8 | CN16 |
| AH17 | AN1 | BN8 | CN17 |
| AH18 | AN1 | BN8 | CN18 |
| AH19 | AN1 | BN8 | CN19 |
| AH20 | AN1 | BN8 | CN20 |
| AH21 | AN1 | BN8 | CN21 |
| AH22 | AN1 | BN8 | CN22 |
| AH23 | AN1 | BN8 | CN23 |
| AH24 | AN1 | BN8 | CN24 |
| AH25 | AN1 | BN8 | CN25 |
| AH26 | AN1 | BN8 | CN26 |
| AH27 | AN1 | BN8 | CN27 |
| AH28 | AN1 | BN8 | CN28 |
| AH29 | AN1 | BN8 | CN29 |
| AH30 | AN1 | BN8 | CN30 |
| AH31 | AN1 | BN8 | CN31 |
| AH32 | AN1 | BN8 | CN32 |
| AH33 | AN1 | BN8 | CN33 |
| AH34 | AN1 | BN8 | CN34 |
| AH35 | AN1 | BN8 | CN35 |
| AH36 | AN1 | BN8 | CN36 |
| AI1 | AN1 | BN9 | CN1 |
| AI2 | AN1 | BN9 | CN2 |
| AI3 | AN1 | BN9 | CN3 |
| AI4 | AN1 | BN9 | CN4 |
| AI5 | AN1 | BN9 | CN5 |
| AI6 | AN1 | BN9 | CN6 |
| AI7 | AN1 | BN9 | CN7 |
| AI8 | AN1 | BN9 | CN8 |
| AI9 | AN1 | BN9 | CN9 |
| AI10 | AN1 | BN9 | CN10 |
| AI11 | AN1 | BN9 | CN11 |
| AI12 | AN1 | BN9 | CN12 |
| AI13 | AN1 | BN9 | CN13 |
| AI14 | AN1 | BN9 | CN14 |
| AI15 | AN1 | BN9 | CN15 |
| AI16 | AN1 | BN9 | CN16 |
| AI17 | AN1 | BN9 | CN17 |
| AI18 | AN1 | BN9 | CN18 |
| AI19 | AN1 | BN9 | CN19 |
| AI20 | AN1 | BN9 | CN20 |
| AI21 | AN1 | BN9 | CN21 |
| AI22 | AN1 | BN9 | CN22 |
| AI23 | AN1 | BN9 | CN23 |
| AI24 | AN1 | BN9 | CN24 |
| AI25 | AN1 | BN9 | CN25 |
| AI26 | AN1 | BN9 | CN26 |
| AI27 | AN1 | BN9 | CN27 |
| AI28 | AN1 | BN9 | CN28 |
| AI29 | AN1 | BN9 | CN29 |
| AI30 | AN1 | BN9 | CN30 |
| AI31 | AN1 | BN9 | CN31 |
| AI32 | AN1 | BN9 | CN32 |
| AI33 | AN1 | BN9 | CN33 |
| AI34 | AN1 | BN9 | CN34 |
| AI35 | AN1 | BN9 | CN35 |
| AI36 | AN1 | BN9 | CN36 |
| AJ1 | AN1 | BN10 | CN1 |
| AJ2 | AN1 | BN10 | CN2 |
| AJ3 | AN1 | BN10 | CN3 |
| AJ4 | AN1 | BN10 | CN4 |
| AJ5 | AN1 | BN10 | CN5 |
| AJ6 | AN1 | BN10 | CN6 |
| AJ7 | AN1 | BN10 | CN7 |
| AJ8 | AN1 | BN10 | CN8 |
| AJ9 | AN1 | BN10 | CN9 |
| AJ10 | AN1 | BN10 | CN10 |
| AJ11 | AN1 | BN10 | CN11 |
| AJ12 | AN1 | BN10 | CN12 |
| AJ13 | AN1 | BN10 | CN13 |
| AJ14 | AN1 | BN10 | CN14 |
| AJ15 | AN1 | BN10 | CN15 |
| AJ16 | AN1 | BN10 | CN16 |
| AJ17 | AN1 | BN10 | CN17 |
| AJ18 | AN1 | BN10 | CN18 |
| AJ19 | AN1 | BN10 | CN19 |
| AJ20 | AN1 | BN10 | CN20 |
| AJ21 | AN1 | BN10 | CN21 |
| AJ22 | AN1 | BN10 | CN22 |
| AJ23 | AN1 | BN10 | CN23 |
| AJ24 | AN1 | BN10 | CN24 |
| AJ25 | AN1 | BN10 | CN25 |
| AJ26 | AN1 | BN10 | CN26 |
| AJ27 | AN1 | BN10 | CN27 |
| AJ28 | AN1 | BN10 | CN28 |
| AJ29 | AN1 | BN10 | CN29 |
| AJ30 | AN1 | BN10 | CN30 |
| AJ31 | AN1 | BN10 | CN31 |
| AJ32 | AN1 | BN10 | CN32 |
| AJ33 | AN1 | BN10 | CN33 |
| AJ34 | AN1 | BN10 | CN34 |
| AJ35 | AN1 | BN10 | CN35 |
| AJ36 | AN1 | BN10 | CN36 |
| AK1 | AN1 | BN11 | CN1 |
| AK2 | AN1 | BN11 | CN2 |
| AK3 | AN1 | BN11 | CN3 |
| AK4 | AN1 | BN11 | CN4 |
| AK5 | AN1 | BN11 | CN5 |
| AK6 | AN1 | BN11 | CN6 |
| AK7 | AN1 | BN11 | CN7 |
| AK8 | AN1 | BN11 | CN8 |
| AK9 | AN1 | BN11 | CN9 |
| AK10 | AN1 | BN11 | CN10 |
| AK11 | AN1 | BN11 | CN11 |
| AK12 | AN1 | BN11 | CN12 |
| AK13 | AN1 | BN11 | CN13 |
| AK14 | AN1 | BN11 | CN14 |
| AK15 | AN1 | BN11 | CN15 |
| AK16 | AN1 | BN11 | CN16 |
| AK17 | AN1 | BN11 | CN17 |
| AK18 | AN1 | BN11 | CN18 |
| AK19 | AN1 | BN11 | CN19 |
| AK20 | AN1 | BN11 | CN20 |
| AK21 | AN1 | BN11 | CN21 |
| AK22 | AN1 | BN11 | CN22 |
| AK23 | AN1 | BN11 | CN23 |
| AK24 | AN1 | BN11 | CN24 |
| AK25 | AN1 | BN11 | CN25 |
| AK26 | AN1 | BN11 | CN26 |
| AK27 | AN1 | BN11 | CN27 |
| AK28 | AN1 | BN11 | CN28 |
| AK29 | AN1 | BN11 | CN29 |
| AK30 | AN1 | BN11 | CN30 |
| AK31 | AN1 | BN11 | CN31 |
| AK32 | AN1 | BN11 | CN32 |
| AK33 | AN1 | BN11 | CN33 |
| AK34 | AN1 | BN11 | CN34 |
| AK35 | AN1 | BN11 | CN35 |
| AK36 | AN1 | BN11 | CN36 |
| AL1 | AN1 | BN12 | CN1 |
| AL2 | AN1 | BN12 | CN2 |
| AL3 | AN1 | BN12 | CN3 |
| AL4 | AN1 | BN12 | CN4 |
| AL5 | AN1 | BN12 | CN5 |
| AL6 | AN1 | BN12 | CN6 |
| AL7 | AN1 | BN12 | CN7 |
| AL8 | AN1 | BN12 | CN8 |
| AL9 | AN1 | BN12 | CN9 |
| AL10 | AN1 | BN12 | CN10 |
| AL11 | AN1 | BN12 | CN11 |
| AL12 | AN1 | BN12 | CN12 |
| AL13 | AN1 | BN12 | CN13 |
| AL14 | AN1 | BN12 | CN14 |
| AL15 | AN1 | BN12 | CN15 |
| AL16 | AN1 | BN12 | CN16 |
| AL17 | AN1 | BN12 | CN17 |
| AL18 | AN1 | BN12 | CN18 |
| AL19 | AN1 | BN12 | CN19 |
| AL20 | AN1 | BN12 | CN20 |
| AL21 | AN1 | BN12 | CN21 |
| AL22 | AN1 | BN12 | CN22 |
| AL23 | AN1 | BN12 | CN23 |
| AL24 | AN1 | BN12 | CN24 |
| AL25 | AN1 | BN12 | CN25 |
| AL26 | AN1 | BN12 | CN26 |
| AL27 | AN1 | BN12 | CN27 |
| AL28 | AN1 | BN12 | CN28 |
| AL29 | AN1 | BN12 | CN29 |
| AL30 | AN1 | BN12 | CN30 |
| AL31 | AN1 | BN12 | CN31 |
| AL32 | AN1 | BN12 | CN32 |
| AL33 | AN1 | BN12 | CN33 |
| AL34 | AN1 | BN12 | CN34 |
| AL35 | AN1 | BN12 | CN35 |
| AL36 | AN1 | BN12 | CN36 |
| AM1 | AN1 | BN13 | CN1 |
| AM2 | AN1 | BN13 | CN2 |
| AM3 | AN1 | BN13 | CN3 |
| AM4 | AN1 | BN13 | CN4 |
| AM5 | AN1 | BN13 | CN5 |
| AM6 | AN1 | BN13 | CN6 |
| AM7 | AN1 | BN13 | CN7 |
| AM8 | AN1 | BN13 | CN8 |
| AM9 | AN1 | BN13 | CN9 |
| AM10 | AN1 | BN13 | CN10 |
| AM11 | AN1 | BN13 | CN11 |
| AM12 | AN1 | BN13 | CN12 |
| AM13 | AN1 | BN13 | CN13 |
| AM14 | AN1 | BN13 | CN14 |
| AM15 | AN1 | BN13 | CN15 |
| AM16 | AN1 | BN13 | CN16 |
| AM17 | AN1 | BN13 | CN17 |
| AM18 | AN1 | BN13 | CN18 |
| AM19 | AN1 | BN13 | CN19 |
| AM20 | AN1 | BN13 | CN20 |
| AM21 | AN1 | BN13 | CN21 |
| AM22 | AN1 | BN13 | CN22 |
| AM23 | AN1 | BN13 | CN23 |
| AM24 | AN1 | BN13 | CN24 |
| AM25 | AN1 | BN13 | CN25 |
| AM26 | AN1 | BN13 | CN26 |
| AM27 | AN1 | BN13 | CN27 |
| AM28 | AN1 | BN13 | CN28 |
| AM29 | AN1 | BN13 | CN29 |
| AM30 | AN1 | BN13 | CN30 |
| AM31 | AN1 | BN13 | CN31 |
| AM32 | AN1 | BN13 | CN32 |
| AM33 | AN1 | BN13 | CN33 |
| AM34 | AN1 | BN13 | CN34 |
| AM35 | AN1 | BN13 | CN35 |
| AM36 | AN1 | BN13 | CN36 |
| AO1 | AN1 | BN14 | CN1 |
| AO2 | AN1 | BN14 | CN2 |
| AO3 | AN1 | BN14 | CN3 |
| AO4 | AN1 | BN14 | CN4 |
| AO5 | AN1 | BN14 | CN5 |
| AO6 | AN1 | BN14 | CN6 |
| AO7 | AN1 | BN14 | CN7 |
| AO8 | AN1 | BN14 | CN8 |
| AO9 | AN1 | BN14 | CN9 |
| AO10 | AN1 | BN14 | CN10 |
| AO11 | AN1 | BN14 | CN11 |
| AO12 | AN1 | BN14 | CN12 |
| AO13 | AN1 | BN14 | CN13 |
| AO14 | AN1 | BN14 | CN14 |
| AO15 | AN1 | BN14 | CN15 |
| AO16 | AN1 | BN14 | CN16 |
| AO17 | AN1 | BN14 | CN17 |
| AO18 | AN1 | BN14 | CN18 |
| AO19 | AN1 | BN14 | CN19 |
| AO20 | AN1 | BN14 | CN20 |
| AO21 | AN1 | BN14 | CN21 |
| AO22 | AN1 | BN14 | CN22 |
| AO23 | AN1 | BN14 | CN23 |
| AO24 | AN1 | BN14 | CN24 |
| AO25 | AN1 | BN14 | CN25 |
| AO26 | AN1 | BN14 | CN26 |
| AO27 | AN1 | BN14 | CN27 |
| AO28 | AN1 | BN14 | CN28 |
| AO29 | AN1 | BN14 | CN29 |
| AO30 | AN1 | BN14 | CN30 |
| AO31 | AN1 | BN14 | CN31 |
| AO32 | AN1 | BN14 | CN32 |
| AO33 | AN1 | BN14 | CN33 |
| AO34 | AN1 | BN14 | CN34 |
| AO35 | AN1 | BN14 | CN35 |
| AO36 | AN1 | BN14 | CN36 |
| AP1 | AN1 | BN15 | CN1 |
| AP2 | AN1 | BN15 | CN2 |
| AP3 | AN1 | BN15 | CN3 |
| AP4 | AN1 | BN15 | CN4 |
| AP5 | AN1 | BN15 | CN5 |
| AP6 | AN1 | BN15 | CN6 |
| AP7 | AN1 | BN15 | CN7 |
| AP8 | AN1 | BN15 | CN8 |
| AP9 | AN1 | BN15 | CN9 |
| AP10 | AN1 | BN15 | CN10 |
| AP11 | AN1 | BN15 | CN11 |
| AP12 | AN1 | BN15 | CN12 |
| AP13 | AN1 | BN15 | CN13 |
| AP14 | AN1 | BN15 | CN14 |
| AP15 | AN1 | BN15 | CN15 |
| AP16 | AN1 | BN15 | CN16 |
| AP17 | AN1 | BN15 | CN17 |
| AP18 | AN1 | BN15 | CN18 |
| AP19 | AN1 | BN15 | CN19 |
| AP20 | AN1 | BN15 | CN20 |
| AP21 | AN1 | BN15 | CN21 |
| AP22 | AN1 | BN15 | CN22 |
| AP23 | AN1 | BN15 | CN23 |
| AP24 | AN1 | BN15 | CN24 |
| AP25 | AN1 | BN15 | CN25 |
| AP26 | AN1 | BN15 | CN26 |
| AP27 | AN1 | BN15 | CN27 |
| AP28 | AN1 | BN15 | CN28 |
| AP29 | AN1 | BN15 | CN29 |
| AP30 | AN1 | BN15 | CN30 |
| AP31 | AN1 | BN15 | CN31 |
| AP32 | AN1 | BN15 | CN32 |
| AP33 | AN1 | BN15 | CN33 |
| AP34 | AN1 | BN15 | CN34 |
| AP35 | AN1 | BN15 | CN35 |
| AP36 | AN1 | BN15 | CN36 |
| AQ1 | AN1 | BN16 | CN1 |
| AQ2 | AN1 | BN16 | CN2 |
| AQ3 | AN1 | BN16 | CN3 |
| AQ4 | AN1 | BN16 | CN4 |
| AQ5 | AN1 | BN16 | CN5 |
| AQ6 | AN1 | BN16 | CN6 |
| AQ7 | AN1 | BN16 | CN7 |
| AQ8 | AN1 | BN16 | CN8 |
| AQ9 | AN1 | BN16 | CN9 |
| AQ10 | AN1 | BN16 | CN10 |
| AQ11 | AN1 | BN16 | CN11 |
| AQ12 | AN1 | BN16 | CN12 |
| AQ13 | AN1 | BN16 | CN13 |
| AQ14 | AN1 | BN16 | CN14 |
| AQ15 | AN1 | BN16 | CN15 |
| AQ16 | AN1 | BN16 | CN16 |
| AQ17 | AN1 | BN16 | CN17 |
| AQ18 | AN1 | BN16 | CN18 |
| AQ19 | AN1 | BN16 | CN19 |
| AQ20 | AN1 | BN16 | CN20 |
| AQ21 | AN1 | BN16 | CN21 |
| AQ22 | AN1 | BN16 | CN22 |
| AQ23 | AN1 | BN16 | CN23 |
| AQ24 | AN1 | BN16 | CN24 |
| AQ25 | AN1 | BN16 | CN25 |
| AQ26 | AN1 | BN16 | CN26 |
| AQ27 | AN1 | BN16 | CN27 |
| AQ28 | AN1 | BN16 | CN28 |
| AQ29 | AN1 | BN16 | CN29 |
| AQ30 | AN1 | BN16 | CN30 |
| AQ31 | AN1 | BN16 | CN31 |
| AQ32 | AN1 | BN16 | CN32 |
| AQ33 | AN1 | BN16 | CN33 |
| AQ34 | AN1 | BN16 | CN34 |
| AQ35 | AN1 | BN16 | CN35 |
| AQ36 | AN1 | BN16 | CN36 |
| AR1 | AN1 | BN17 | CN1 |
| AR2 | AN1 | BN17 | CN2 |
| AR3 | AN1 | BN17 | CN3 |
| AR4 | AN1 | BN17 | CN4 |
| AR5 | AN1 | BN17 | CN5 |
| AR6 | AN1 | BN17 | CN6 |
| AR7 | AN1 | BN17 | CN7 |
| AR8 | AN1 | BN17 | CN8 |
| AR9 | AN1 | BN17 | CN9 |
| AR10 | AN1 | BN17 | CN10 |
| AR11 | AN1 | BN17 | CN11 |
| AR12 | AN1 | BN17 | CN12 |
| AR13 | AN1 | BN17 | CN13 |
| AR14 | AN1 | BN17 | CN14 |
| AR15 | AN1 | BN17 | CN15 |
| AR16 | AN1 | BN17 | CN16 |
| AR17 | AN1 | BN17 | CN17 |
| AR18 | AN1 | BN17 | CN18 |
| AR19 | AN1 | BN17 | CN19 |
| AR20 | AN1 | BN17 | CN20 |
| AR21 | AN1 | BN17 | CN21 |
| AR22 | AN1 | BN17 | CN22 |
| AR23 | AN1 | BN17 | CN23 |
| AR24 | AN1 | BN17 | CN24 |
| AR25 | AN1 | BN17 | CN25 |
| AR26 | AN1 | BN17 | CN26 |
| AR27 | AN1 | BN17 | CN27 |
| AR28 | AN1 | BN17 | CN28 |
| AR29 | AN1 | BN17 | CN29 |
| AR30 | AN1 | BN17 | CN30 |
| AR31 | AN1 | BN17 | CN31 |
| AR32 | AN1 | BN17 | CN32 |
| AR33 | AN1 | BN17 | CN33 |
| AR34 | AN1 | BN17 | CN34 |
| AR35 | AN1 | BN17 | CN35 |
| AR36 | AN1 | BN17 | CN36 |
| AS1 | AN1 | BN18 | CN1 |
| AS2 | AN1 | BN18 | CN2 |
| AS3 | AN1 | BN18 | CN3 |
| AS4 | AN1 | BN18 | CN4 |
| AS5 | AN1 | BN18 | CN5 |
| AS6 | AN1 | BN18 | CN6 |
| AS7 | AN1 | BN18 | CN7 |
| AS8 | AN1 | BN18 | CN8 |
| AS9 | AN1 | BN18 | CN9 |
| AS10 | AN1 | BN18 | CN10 |
| AS11 | AN1 | BN18 | CN11 |
| AS12 | AN1 | BN18 | CN12 |
| AS13 | AN1 | BN18 | CN13 |
| AS14 | AN1 | BN18 | CN14 |
| AS15 | AN1 | BN18 | CN15 |
| AS16 | AN1 | BN18 | CN16 |
| AS17 | AN1 | BN18 | CN17 |
| AS18 | AN1 | BN18 | CN18 |
| AS19 | AN1 | BN18 | CN19 |
| AS20 | AN1 | BN18 | CN20 |
| AS21 | AN1 | BN18 | CN21 |
| AS22 | AN1 | BN18 | CN22 |
| AS23 | AN1 | BN18 | CN23 |
| AS24 | AN1 | BN18 | CN24 |
| AS25 | AN1 | BN18 | CN25 |
| AS26 | AN1 | BN18 | CN26 |
| AS27 | AN1 | BN18 | CN27 |
| AS28 | AN1 | BN18 | CN28 |
| AS29 | AN1 | BN18 | CN29 |
| AS30 | AN1 | BN18 | CN30 |
| AS31 | AN1 | BN18 | CN31 |
| AS32 | AN1 | BN18 | CN32 |
| AS33 | AN1 | BN18 | CN33 |
| AS34 | AN1 | BN18 | CN34 |
| AS35 | AN1 | BN18 | CN35 |
| AS36 | AN1 | BN18 | CN36 |
| AT1 | AN1 | BN19 | CN1 |
| AT2 | AN1 | BN19 | CN2 |
| AT3 | AN1 | BN19 | CN3 |
| AT4 | AN1 | BN19 | CN4 |
| AT5 | AN1 | BN19 | CN5 |
| AT6 | AN1 | BN19 | CN6 |
| AT7 | AN1 | BN19 | CN7 |
| AT8 | AN1 | BN19 | CN8 |
| AT9 | AN1 | BN19 | CN9 |
| AT10 | AN1 | BN19 | CN10 |
| AT11 | AN1 | BN19 | CN11 |
| AT12 | AN1 | BN19 | CN12 |
| AT13 | AN1 | BN19 | CN13 |
| AT14 | AN1 | BN19 | CN14 |
| AT15 | AN1 | BN19 | CN15 |
| AT16 | AN1 | BN19 | CN16 |
| AT17 | AN1 | BN19 | CN17 |
| AT18 | AN1 | BN19 | CN18 |
| AT19 | AN1 | BN19 | CN19 |
| AT20 | AN1 | BN19 | CN20 |
| AT21 | AN1 | BN19 | CN21 |
| AT22 | AN1 | BN19 | CN22 |
| AT23 | AN1 | BN19 | CN23 |
| AT24 | AN1 | BN19 | CN24 |
| AT25 | AN1 | BN19 | CN25 |
| AT26 | AN1 | BN19 | CN26 |
| AT27 | AN1 | BN19 | CN27 |
| AT28 | AN1 | BN19 | CN28 |
| AT29 | AN1 | BN19 | CN29 |
| AT30 | AN1 | BN19 | CN30 |
| AT31 | AN1 | BN19 | CN31 |
| AT32 | AN1 | BN19 | CN32 |
| AT33 | AN1 | BN19 | CN33 |
| AT34 | AN1 | BN19 | CN34 |
| AT35 | AN1 | BN19 | CN35 |
| AT36 | AN1 | BN19 | CN36 |
| AU1 | AN1 | BN20 | CN1 |
| AU2 | AN1 | BN20 | CN2 |
| AU3 | AN1 | BN20 | CN3 |
| AU4 | AN1 | BN20 | CN4 |
| AU5 | AN1 | BN20 | CN5 |
| AU6 | AN1 | BN20 | CN6 |
| AU7 | AN1 | BN20 | CN7 |
| AU8 | AN1 | BN20 | CN8 |
| AU9 | AN1 | BN20 | CN9 |
| AU10 | AN1 | BN20 | CN10 |
| AU11 | AN1 | BN20 | CN11 |
| AU12 | AN1 | BN20 | CN12 |
| AU13 | AN1 | BN20 | CN13 |
| AU14 | AN1 | BN20 | CN14 |
| AU15 | AN1 | BN20 | CN15 |
| AU16 | AN1 | BN20 | CN16 |
| AU17 | AN1 | BN20 | CN17 |
| AU18 | AN1 | BN20 | CN18 |
| AU19 | AN1 | BN20 | CN19 |
| AU20 | AN1 | BN20 | CN20 |
| AU21 | AN1 | BN20 | CN21 |
| AU22 | AN1 | BN20 | CN22 |
| AU23 | AN1 | BN20 | CN23 |
| AU24 | AN1 | BN20 | CN24 |
| AU25 | AN1 | BN20 | CN25 |
| AU26 | AN1 | BN20 | CN26 |
| AU27 | AN1 | BN20 | CN27 |
| AU28 | AN1 | BN20 | CN28 |
| AU29 | AN1 | BN20 | CN29 |
| AU30 | AN1 | BN20 | CN30 |
| AU31 | AN1 | BN20 | CN31 |
| AU32 | AN1 | BN20 | CN32 |
| AU33 | AN1 | BN20 | CN33 |
| AU34 | AN1 | BN20 | CN34 |
| AU35 | AN1 | BN20 | CN35 |
| AU36 | AN1 | BN20 | CN36 |
| AV1 | AN1 | BN21 | CN1 |
| AV2 | AN1 | BN21 | CN2 |
| AV3 | AN1 | BN21 | CN3 |
| AV4 | AN1 | BN21 | CN4 |
| AV5 | AN1 | BN21 | CN5 |
| AV6 | AN1 | BN21 | CN6 |
| AV7 | AN1 | BN21 | CN7 |
| AV8 | AN1 | BN21 | CN8 |
| AV9 | AN1 | BN21 | CN9 |
| AV10 | AN1 | BN21 | CN10 |
| AV11 | AN1 | BN21 | CN11 |
| AV12 | AN1 | BN21 | CN12 |
| AV13 | AN1 | BN21 | CN13 |
| AV14 | AN1 | BN21 | CN14 |
| AV15 | AN1 | BN21 | CN15 |
| AV16 | AN1 | BN21 | CN16 |
| AV17 | AN1 | BN21 | CN17 |
| AV18 | AN1 | BN21 | CN18 |
| AV19 | AN1 | BN21 | CN19 |
| AV20 | AN1 | BN21 | CN20 |
| AV21 | AN1 | BN21 | CN21 |
| AV22 | AN1 | BN21 | CN22 |
| AV23 | AN1 | BN21 | CN23 |
| AV24 | AN1 | BN21 | CN24 |
| AV25 | AN1 | BN21 | CN25 |
| AV26 | AN1 | BN21 | CN26 |
| AV27 | AN1 | BN21 | CN27 |
| AV28 | AN1 | BN21 | CN28 |
| AV29 | AN1 | BN21 | CN29 |
| AV30 | AN1 | BN21 | CN30 |
| AV31 | AN1 | BN21 | CN31 |
| AV32 | AN1 | BN21 | CN32 |
| AV33 | AN1 | BN21 | CN33 |
| AV34 | AN1 | BN21 | CN34 |
| AV35 | AN1 | BN21 | CN35 |
| AV36 | AN1 | BN21 | CN36 |
| AW1 | AN1 | BN22 | CN1 |
| AW2 | AN1 | BN22 | CN2 |
| AW3 | AN1 | BN22 | CN3 |
| AW4 | AN1 | BN22 | CN4 |
| AW5 | AN1 | BN22 | CN5 |
| AW6 | AN1 | BN22 | CN6 |
| AW7 | AN1 | BN22 | CN7 |
| AW8 | AN1 | BN22 | CN8 |
| AW9 | AN1 | BN22 | CN9 |
| AW10 | AN1 | BN22 | CN10 |
| AW11 | AN1 | BN22 | CN11 |
| AW12 | AN1 | BN22 | CN12 |
| AW13 | AN1 | BN22 | CN13 |
| AW14 | AN1 | BN22 | CN14 |
| AW15 | AN1 | BN22 | CN15 |
| AW16 | AN1 | BN22 | CN16 |
| AW17 | AN1 | BN22 | CN17 |
| AW18 | AN1 | BN22 | CN18 |
| AW19 | AN1 | BN22 | CN19 |
| AW20 | AN1 | BN22 | CN20 |
| AW21 | AN1 | BN22 | CN21 |
| AW22 | AN1 | BN22 | CN22 |
| AW23 | AN1 | BN22 | CN23 |
| AW24 | AN1 | BN22 | CN24 |
| AW25 | AN1 | BN22 | CN25 |
| AW26 | AN1 | BN22 | CN26 |
| AW27 | AN1 | BN22 | CN27 |
| AW28 | AN1 | BN22 | CN28 |
| AW29 | AN1 | BN22 | CN29 |
| AW30 | AN1 | BN22 | CN30 |
| AW31 | AN1 | BN22 | CN31 |
| AW32 | AN1 | BN22 | CN32 |
| AW33 | AN1 | BN22 | CN33 |
| AW34 | AN1 | BN22 | CN34 |
| AW35 | AN1 | BN22 | CN35 |
| AW36 | AN1 | BN22 | CN36 |
| BA1 | AN2 | BN1 | CN1 |
| BA2 | AN2 | BN1 | CN2 |
| BA3 | AN2 | BN1 | CN3 |
| BA4 | AN2 | BN1 | CN4 |
| BA5 | AN2 | BN1 | CN5 |
| BA6 | AN2 | BN1 | CN6 |
| BA7 | AN2 | BN1 | CN7 |
| BA8 | AN2 | BN1 | CN8 |
| BA9 | AN2 | BN1 | CN9 |
| BA10 | AN2 | BN1 | CN10 |
| BA11 | AN2 | BN1 | CN11 |
| BA12 | AN2 | BN1 | CN12 |
| BA13 | AN2 | BN1 | CN13 |
| BA14 | AN2 | BN1 | CN14 |
| BA15 | AN2 | BN1 | CN15 |
| BA16 | AN2 | BN1 | CN16 |
| BA17 | AN2 | BN1 | CN17 |
| BA18 | AN2 | BN1 | CN18 |
| BA19 | AN2 | BN1 | CN19 |
| BA20 | AN2 | BN1 | CN20 |
| BA21 | AN2 | BN1 | CN21 |
| BA22 | AN2 | BN1 | CN22 |
| BA23 | AN2 | BN1 | CN23 |
| BA24 | AN2 | BN1 | CN24 |
| BA25 | AN2 | BN1 | CN25 |
| BA26 | AN2 | BN1 | CN26 |
| BA27 | AN2 | BN1 | CN27 |
| BA28 | AN2 | BN1 | CN28 |
| BA29 | AN2 | BN1 | CN29 |
| BA30 | AN2 | BN1 | CN30 |
| BA31 | AN2 | BN1 | CN31 |
| BA32 | AN2 | BN1 | CN32 |
| BA33 | AN2 | BN1 | CN33 |
| BA34 | AN2 | BN1 | CN34 |
| BA35 | AN2 | BN1 | CN35 |
| BA36 | AN2 | BN1 | CN36 |
| BB1 | AN2 | BN2 | CN1 |
| BB2 | AN2 | BN2 | CN2 |
| BB3 | AN2 | BN2 | CN3 |
| BB4 | AN2 | BN2 | CN4 |
| BB5 | AN2 | BN2 | CN5 |
| BB6 | AN2 | BN2 | CN6 |
| BB7 | AN2 | BN2 | CN7 |
| BB8 | AN2 | BN2 | CN8 |
| BB9 | AN2 | BN2 | CN9 |
| BB10 | AN2 | BN2 | CN10 |
| BB11 | AN2 | BN2 | CN11 |
| BB12 | AN2 | BN2 | CN12 |
| BB13 | AN2 | BN2 | CN13 |
| BB14 | AN2 | BN2 | CN14 |
| BB15 | AN2 | BN2 | CN15 |
| BB16 | AN2 | BN2 | CN16 |
| BB17 | AN2 | BN2 | CN17 |
| BB18 | AN2 | BN2 | CN18 |
| BB19 | AN2 | BN2 | CN19 |
| BB20 | AN2 | BN2 | CN20 |
| BB21 | AN2 | BN2 | CN21 |
| BB22 | AN2 | BN2 | CN22 |
| BB23 | AN2 | BN2 | CN23 |
| BB24 | AN2 | BN2 | CN24 |
| BB25 | AN2 | BN2 | CN25 |
| BB26 | AN2 | BN2 | CN26 |
| BB27 | AN2 | BN2 | CN27 |
| BB28 | AN2 | BN2 | CN28 |
| BB29 | AN2 | BN2 | CN29 |
| BB30 | AN2 | BN2 | CN30 |
| BB31 | AN2 | BN2 | CN31 |
| BB32 | AN2 | BN2 | CN32 |
| BB33 | AN2 | BN2 | CN33 |
| BB34 | AN2 | BN2 | CN34 |
| BB35 | AN2 | BN2 | CN35 |
| BB36 | AN2 | BN2 | CN36 |
| BC1 | AN2 | BN3 | CN1 |
| BC2 | AN2 | BN3 | CN2 |
| BC3 | AN2 | BN3 | CN3 |
| BC4 | AN2 | BN3 | CN4 |
| BC5 | AN2 | BN3 | CN5 |
| BC6 | AN2 | BN3 | CN6 |
| BC7 | AN2 | BN3 | CN7 |
| BC8 | AN2 | BN3 | CN8 |
| BC9 | AN2 | BN3 | CN9 |
| BC10 | AN2 | BN3 | CN10 |
| BC11 | AN2 | BN3 | CN11 |
| BC12 | AN2 | BN3 | CN12 |
| BC13 | AN2 | BN3 | CN13 |
| BC14 | AN2 | BN3 | CN14 |
| BC15 | AN2 | BN3 | CN15 |
| BC16 | AN2 | BN3 | CN16 |
| BC17 | AN2 | BN3 | CN17 |
| BC18 | AN2 | BN3 | CN18 |
| BC19 | AN2 | BN3 | CN19 |
| BC20 | AN2 | BN3 | CN20 |
| BC21 | AN2 | BN3 | CN21 |
| BC22 | AN2 | BN3 | CN22 |
| BC23 | AN2 | BN3 | CN23 |
| BC24 | AN2 | BN3 | CN24 |
| BC25 | AN2 | BN3 | CN25 |
| BC26 | AN2 | BN3 | CN26 |
| BC27 | AN2 | BN3 | CN27 |
| BC28 | AN2 | BN3 | CN28 |
| BC29 | AN2 | BN3 | CN29 |
| BC30 | AN2 | BN3 | CN30 |
| BC31 | AN2 | BN3 | CN31 |
| BC32 | AN2 | BN3 | CN32 |
| BC33 | AN2 | BN3 | CN33 |
| BC34 | AN2 | BN3 | CN34 |
| BC35 | AN2 | BN3 | CN35 |
| BC36 | AN2 | BN3 | CN36 |
| BD1 | AN2 | BN4 | CN1 |
| BD2 | AN2 | BN4 | CN2 |
| BD3 | AN2 | BN4 | CN3 |
| BD4 | AN2 | BN4 | CN4 |
| BD5 | AN2 | BN4 | CN5 |
| BD6 | AN2 | BN4 | CN6 |
| BD7 | AN2 | BN4 | CN7 |
| BD8 | AN2 | BN4 | CN8 |
| BD9 | AN2 | BN4 | CN9 |
| BD10 | AN2 | BN4 | CN10 |
| BD11 | AN2 | BN4 | CN11 |
| BD12 | AN2 | BN4 | CN12 |
| BD13 | AN2 | BN4 | CN13 |
| BD14 | AN2 | BN4 | CN14 |
| BD15 | AN2 | BN4 | CN15 |
| BD16 | AN2 | BN4 | CN16 |
| BD17 | AN2 | BN4 | CN17 |
| BD18 | AN2 | BN4 | CN18 |
| BD19 | AN2 | BN4 | CN19 |
| BD20 | AN2 | BN4 | CN20 |
| BD21 | AN2 | BN4 | CN21 |
| BD22 | AN2 | BN4 | CN22 |
| BD23 | AN2 | BN4 | CN23 |
| BD24 | AN2 | BN4 | CN24 |
| BD25 | AN2 | BN4 | CN25 |
| BD26 | AN2 | BN4 | CN26 |
| BD27 | AN2 | BN4 | CN27 |
| BD28 | AN2 | BN4 | CN28 |
| BD29 | AN2 | BN4 | CN29 |
| BD30 | AN2 | BN4 | CN30 |
| BD31 | AN2 | BN4 | CN31 |
| BD32 | AN2 | BN4 | CN32 |
| BD33 | AN2 | BN4 | CN33 |
| BD34 | AN2 | BN4 | CN34 |
| BD35 | AN2 | BN4 | CN35 |
| BD36 | AN2 | BN4 | CN36 |
| BE1 | AN2 | BN5 | CN1 |
| BE2 | AN2 | BN5 | CN2 |
| BE3 | AN2 | BN5 | CN3 |
| BE4 | AN2 | BN5 | CN4 |
| BE5 | AN2 | BN5 | CN5 |
| BE6 | AN2 | BN5 | CN6 |
| BE7 | AN2 | BN5 | CN7 |
| BE8 | AN2 | BN5 | CN8 |
| BE9 | AN2 | BN5 | CN9 |
| BE10 | AN2 | BN5 | CN10 |
| BE11 | AN2 | BN5 | CN11 |
| BE12 | AN2 | BN5 | CN12 |
| BE13 | AN2 | BN5 | CN13 |
| BE14 | AN2 | BN5 | CN14 |
| BE15 | AN2 | BN5 | CN15 |
| BE16 | AN2 | BN5 | CN16 |
| BE17 | AN2 | BN5 | CN17 |
| BE18 | AN2 | BN5 | CN18 |
| BE19 | AN2 | BN5 | CN19 |
| BE20 | AN2 | BN5 | CN20 |
| BE21 | AN2 | BN5 | CN21 |
| BE22 | AN2 | BN5 | CN22 |
| BE23 | AN2 | BN5 | CN23 |
| BE24 | AN2 | BN5 | CN24 |
| BE25 | AN2 | BN5 | CN25 |
| BE26 | AN2 | BN5 | CN26 |
| BE27 | AN2 | BN5 | CN27 |
| BE28 | AN2 | BN5 | CN28 |
| BE29 | AN2 | BN5 | CN29 |
| BE30 | AN2 | BN5 | CN30 |
| BE31 | AN2 | BN5 | CN31 |
| BE32 | AN2 | BN5 | CN32 |
| BE33 | AN2 | BN5 | CN33 |
| BE34 | AN2 | BN5 | CN34 |
| BE35 | AN2 | BN5 | CN35 |
| BE36 | AN2 | BN5 | CN36 |
| BF1 | AN2 | BN6 | CN1 |
| BF2 | AN2 | BN6 | CN2 |
| BF3 | AN2 | BN6 | CN3 |
| BF4 | AN2 | BN6 | CN4 |
| BF5 | AN2 | BN6 | CN5 |
| BF6 | AN2 | BN6 | CN6 |
| BF7 | AN2 | BN6 | CN7 |
| BF8 | AN2 | BN6 | CN8 |
| BF9 | AN2 | BN6 | CN9 |
| BF10 | AN2 | BN6 | CN10 |
| BF11 | AN2 | BN6 | CN11 |
| BF12 | AN2 | BN6 | CN12 |
| BF13 | AN2 | BN6 | CN13 |
| BF14 | AN2 | BN6 | CN14 |
| BF15 | AN2 | BN6 | CN15 |
| BF16 | AN2 | BN6 | CN16 |
| BF17 | AN2 | BN6 | CN17 |
| BF18 | AN2 | BN6 | CN18 |
| BF19 | AN2 | BN6 | CN19 |
| BF20 | AN2 | BN6 | CN20 |
| BF21 | AN2 | BN6 | CN21 |
| BF22 | AN2 | BN6 | CN22 |
| BF23 | AN2 | BN6 | CN23 |
| BF24 | AN2 | BN6 | CN24 |
| BF25 | AN2 | BN6 | CN25 |
| BF26 | AN2 | BN6 | CN26 |
| BF27 | AN2 | BN6 | CN27 |
| BF28 | AN2 | BN6 | CN28 |
| BF29 | AN2 | BN6 | CN29 |
| BF30 | AN2 | BN6 | CN30 |
| BF31 | AN2 | BN6 | CN31 |
| BF32 | AN2 | BN6 | CN32 |
| BF33 | AN2 | BN6 | CN33 |
| BF34 | AN2 | BN6 | CN34 |
| BF35 | AN2 | BN6 | CN35 |
| BF36 | AN2 | BN6 | CN36 |
| BG1 | AN2 | BN7 | CN1 |
| BG2 | AN2 | BN7 | CN2 |
| BG3 | AN2 | BN7 | CN3 |
| BG4 | AN2 | BN7 | CN4 |
| BG5 | AN2 | BN7 | CN5 |
| BG6 | AN2 | BN7 | CN6 |
| BG7 | AN2 | BN7 | CN7 |
| BG8 | AN2 | BN7 | CN8 |
| BG9 | AN2 | BN7 | CN9 |
| BG10 | AN2 | BN7 | CN10 |
| BG11 | AN2 | BN7 | CN11 |
| BG12 | AN2 | BN7 | CN12 |
| BG13 | AN2 | BN7 | CN13 |
| BG14 | AN2 | BN7 | CN14 |
| BG15 | AN2 | BN7 | CN15 |
| BG16 | AN2 | BN7 | CN16 |
| BG17 | AN2 | BN7 | CN17 |
| BG18 | AN2 | BN7 | CN18 |
| BG19 | AN2 | BN7 | CN19 |
| BG20 | AN2 | BN7 | CN20 |
| BG21 | AN2 | BN7 | CN21 |
| BG22 | AN2 | BN7 | CN22 |
| BG23 | AN2 | BN7 | CN23 |
| BG24 | AN2 | BN7 | CN24 |
| BG25 | AN2 | BN7 | CN25 |
| BG26 | AN2 | BN7 | CN26 |
| BG27 | AN2 | BN7 | CN27 |
| BG28 | AN2 | BN7 | CN28 |
| BG29 | AN2 | BN7 | CN29 |
| BG30 | AN2 | BN7 | CN30 |
| BG31 | AN2 | BN7 | CN31 |
| BG32 | AN2 | BN7 | CN32 |
| BG33 | AN2 | BN7 | CN33 |
| BG34 | AN2 | BN7 | CN34 |
| BG35 | AN2 | BN7 | CN35 |
| BG36 | AN2 | BN7 | CN36 |
| BH1 | AN2 | BN8 | CN1 |
| BH2 | AN2 | BN8 | CN2 |
| BH3 | AN2 | BN8 | CN3 |
| BH4 | AN2 | BN8 | CN4 |
| BH5 | AN2 | BN8 | CN5 |
| BH6 | AN2 | BN8 | CN6 |
| BH7 | AN2 | BN8 | CN7 |
| BH8 | AN2 | BN8 | CN8 |
| BH9 | AN2 | BN8 | CN9 |
| BH10 | AN2 | BN8 | CN10 |
| BH11 | AN2 | BN8 | CN11 |
| BH12 | AN2 | BN8 | CN12 |
| BH13 | AN2 | BN8 | CN13 |
| BH14 | AN2 | BN8 | CN14 |
| BH15 | AN2 | BN8 | CN15 |
| BH16 | AN2 | BN8 | CN16 |
| BH17 | AN2 | BN8 | CN17 |
| BH18 | AN2 | BN8 | CN18 |
| BH19 | AN2 | BN8 | CN19 |
| BH20 | AN2 | BN8 | CN20 |
| BH21 | AN2 | BN8 | CN21 |
| BH22 | AN2 | BN8 | CN22 |
| BH23 | AN2 | BN8 | CN23 |
| BH24 | AN2 | BN8 | CN24 |
| BH25 | AN2 | BN8 | CN25 |
| BH26 | AN2 | BN8 | CN26 |
| BH27 | AN2 | BN8 | CN27 |
| BH28 | AN2 | BN8 | CN28 |
| BH29 | AN2 | BN8 | CN29 |
| BH30 | AN2 | BN8 | CN30 |
| BH31 | AN2 | BN8 | CN31 |
| BH32 | AN2 | BN8 | CN32 |
| BH33 | AN2 | BN8 | CN33 |
| BH34 | AN2 | BN8 | CN34 |
| BH35 | AN2 | BN8 | CN35 |
| BH36 | AN2 | BN8 | CN36 |
| BI1 | AN2 | BN9 | CN1 |
| BI2 | AN2 | BN9 | CN2 |
| BI3 | AN2 | BN9 | CN3 |
| BI4 | AN2 | BN9 | CN4 |
| BI5 | AN2 | BN9 | CN5 |
| BI6 | AN2 | BN9 | CN6 |
| BI7 | AN2 | BN9 | CN7 |
| BI8 | AN2 | BN9 | CN8 |
| BI9 | AN2 | BN9 | CN9 |
| BI10 | AN2 | BN9 | CN10 |
| BI11 | AN2 | BN9 | CN11 |
| BI12 | AN2 | BN9 | CN12 |
| BI13 | AN2 | BN9 | CN13 |
| BI14 | AN2 | BN9 | CN14 |
| BI15 | AN2 | BN9 | CN15 |
| BI16 | AN2 | BN9 | CN16 |
| BI17 | AN2 | BN9 | CN17 |
| BI18 | AN2 | BN9 | CN18 |
| BI19 | AN2 | BN9 | CN19 |
| BI20 | AN2 | BN9 | CN20 |
| BI21 | AN2 | BN9 | CN21 |
| BI22 | AN2 | BN9 | CN22 |
| BI23 | AN2 | BN9 | CN23 |
| BI24 | AN2 | BN9 | CN24 |
| BI25 | AN2 | BN9 | CN25 |
| BI26 | AN2 | BN9 | CN26 |
| BI27 | AN2 | BN9 | CN27 |
| BI28 | AN2 | BN9 | CN28 |
| BI29 | AN2 | BN9 | CN29 |
| BI30 | AN2 | BN9 | CN30 |
| BI31 | AN2 | BN9 | CN31 |
| BI32 | AN2 | BN9 | CN32 |
| BI33 | AN2 | BN9 | CN33 |
| BI34 | AN2 | BN9 | CN34 |
| BI35 | AN2 | BN9 | CN35 |
| BI36 | AN2 | BN9 | CN36 |
| BJ1 | AN2 | BN10 | CN1 |
| BJ2 | AN2 | BN10 | CN2 |
| BJ3 | AN2 | BN10 | CN3 |
| BJ4 | AN2 | BN10 | CN4 |
| BJ5 | AN2 | BN10 | CN5 |
| BJ6 | AN2 | BN10 | CN6 |
| BJ7 | AN2 | BN10 | CN7 |
| BJ8 | AN2 | BN10 | CN8 |
| BJ9 | AN2 | BN10 | CN9 |
| BJ10 | AN2 | BN10 | CN10 |
| BJ11 | AN2 | BN10 | CN11 |
| BJ12 | AN2 | BN10 | CN12 |
| BJ13 | AN2 | BN10 | CN13 |
| BJ14 | AN2 | BN10 | CN14 |
| BJ15 | AN2 | BN10 | CN15 |
| BJ16 | AN2 | BN10 | CN16 |
| BJ17 | AN2 | BN10 | CN17 |
| BJ18 | AN2 | BN10 | CN18 |
| BJ19 | AN2 | BN10 | CN19 |
| BJ20 | AN2 | BN10 | CN20 |
| BJ21 | AN2 | BN10 | CN21 |
| BJ22 | AN2 | BN10 | CN22 |
| BJ23 | AN2 | BN10 | CN23 |
| BJ24 | AN2 | BN10 | CN24 |
| BJ25 | AN2 | BN10 | CN25 |
| BJ26 | AN2 | BN10 | CN26 |
| BJ27 | AN2 | BN10 | CN27 |
| BJ28 | AN2 | BN10 | CN28 |
| BJ29 | AN2 | BN10 | CN29 |
| BJ30 | AN2 | BN10 | CN30 |
| BJ31 | AN2 | BN10 | CN31 |
| BJ32 | AN2 | BN10 | CN32 |
| BJ33 | AN2 | BN10 | CN33 |
| BJ34 | AN2 | BN10 | CN34 |
| BJ35 | AN2 | BN10 | CN35 |
| BJ36 | AN2 | BN10 | CN36 |
| BK1 | AN2 | BN11 | CN1 |
| BK2 | AN2 | BN11 | CN2 |
| BK3 | AN2 | BN11 | CN3 |
| BK4 | AN2 | BN11 | CN4 |
| BK5 | AN2 | BN11 | CN5 |
| BK6 | AN2 | BN11 | CN6 |
| BK7 | AN2 | BN11 | CN7 |
| BK8 | AN2 | BN11 | CN8 |
| BK9 | AN2 | BN11 | CN9 |
| BK10 | AN2 | BN11 | CN10 |
| BK11 | AN2 | BN11 | CN11 |
| BK12 | AN2 | BN11 | CN12 |
| BK13 | AN2 | BN11 | CN13 |
| BK14 | AN2 | BN11 | CN14 |
| BK15 | AN2 | BN11 | CN15 |
| BK16 | AN2 | BN11 | CN16 |
| BK17 | AN2 | BN11 | CN17 |
| BK18 | AN2 | BN11 | CN18 |
| BK19 | AN2 | BN11 | CN19 |
| BK20 | AN2 | BN11 | CN20 |
| BK21 | AN2 | BN11 | CN21 |
| BK22 | AN2 | BN11 | CN22 |
| BK23 | AN2 | BN11 | CN23 |
| BK24 | AN2 | BN11 | CN24 |
| BK25 | AN2 | BN11 | CN25 |
| BK26 | AN2 | BN11 | CN26 |
| BK27 | AN2 | BN11 | CN27 |
| BK28 | AN2 | BN11 | CN28 |
| BK29 | AN2 | BN11 | CN29 |
| BK30 | AN2 | BN11 | CN30 |
| BK31 | AN2 | BN11 | CN31 |
| BK32 | AN2 | BN11 | CN32 |
| BK33 | AN2 | BN11 | CN33 |
| BK34 | AN2 | BN11 | CN34 |
| BK35 | AN2 | BN11 | CN35 |
| BK36 | AN2 | BN11 | CN36 |
| BL1 | AN2 | BN12 | CN1 |
| BL2 | AN2 | BN12 | CN2 |
| BL3 | AN2 | BN12 | CN3 |
| BL4 | AN2 | BN12 | CN4 |
| BL5 | AN2 | BN12 | CN5 |
| BL6 | AN2 | BN12 | CN6 |
| BL7 | AN2 | BN12 | CN7 |
| BL8 | AN2 | BN12 | CN8 |
| BL9 | AN2 | BN12 | CN9 |
| BL10 | AN2 | BN12 | CN10 |
| BL11 | AN2 | BN12 | CN11 |
| BL12 | AN2 | BN12 | CN12 |
| BL13 | AN2 | BN12 | CN13 |
| BL14 | AN2 | BN12 | CN14 |
| BL15 | AN2 | BN12 | CN15 |
| BL16 | AN2 | BN12 | CN16 |
| BL17 | AN2 | BN12 | CN17 |
| BL18 | AN2 | BN12 | CN18 |
| BL19 | AN2 | BN12 | CN19 |
| BL20 | AN2 | BN12 | CN20 |
| BL21 | AN2 | BN12 | CN21 |
| BL22 | AN2 | BN12 | CN22 |
| BL23 | AN2 | BN12 | CN23 |
| BL24 | AN2 | BN12 | CN24 |
| BL25 | AN2 | BN12 | CN25 |
| BL26 | AN2 | BN12 | CN26 |
| BL27 | AN2 | BN12 | CN27 |
| BL28 | AN2 | BN12 | CN28 |
| BL29 | AN2 | BN12 | CN29 |
| BL30 | AN2 | BN12 | CN30 |
| BL31 | AN2 | BN12 | CN31 |
| BL32 | AN2 | BN12 | CN32 |
| BL33 | AN2 | BN12 | CN33 |
| BL34 | AN2 | BN12 | CN34 |
| BL35 | AN2 | BN12 | CN35 |
| BL36 | AN2 | BN12 | CN36 |
| BM1 | AN2 | BN13 | CN1 |
| BM2 | AN2 | BN13 | CN2 |
| BM3 | AN2 | BN13 | CN3 |
| BM4 | AN2 | BN13 | CN4 |
| BM5 | AN2 | BN13 | CN5 |
| BM6 | AN2 | BN13 | CN6 |
| BM7 | AN2 | BN13 | CN7 |
| BM8 | AN2 | BN13 | CN8 |
| BM9 | AN2 | BN13 | CN9 |
| BM10 | AN2 | BN13 | CN10 |
| BM11 | AN2 | BN13 | CN11 |
| BM12 | AN2 | BN13 | CN12 |
| BM13 | AN2 | BN13 | CN13 |
| BM14 | AN2 | BN13 | CN14 |
| BM15 | AN2 | BN13 | CN15 |
| BM16 | AN2 | BN13 | CN16 |
| BM17 | AN2 | BN13 | CN17 |
| BM18 | AN2 | BN13 | CN18 |
| BM19 | AN2 | BN13 | CN19 |
| BM20 | AN2 | BN13 | CN20 |
| BM21 | AN2 | BN13 | CN21 |
| BM22 | AN2 | BN13 | CN22 |
| BM23 | AN2 | BN13 | CN23 |
| BM24 | AN2 | BN13 | CN24 |
| BM25 | AN2 | BN13 | CN25 |
| BM26 | AN2 | BN13 | CN26 |
| BM27 | AN2 | BN13 | CN27 |
| BM28 | AN2 | BN13 | CN28 |
| BM29 | AN2 | BN13 | CN29 |
| BM30 | AN2 | BN13 | CN30 |
| BM31 | AN2 | BN13 | CN31 |
| BM32 | AN2 | BN13 | CN32 |
| BM33 | AN2 | BN13 | CN33 |
| BM34 | AN2 | BN13 | CN34 |
| BM35 | AN2 | BN13 | CN35 |
| BM36 | AN2 | BN13 | CN36 |
| BO1 | AN2 | BN14 | CN1 |
| BO2 | AN2 | BN14 | CN2 |
| BO3 | AN2 | BN14 | CN3 |
| BO4 | AN2 | BN14 | CN4 |
| BO5 | AN2 | BN14 | CN5 |
| BO6 | AN2 | BN14 | CN6 |
| BO7 | AN2 | BN14 | CN7 |
| BO8 | AN2 | BN14 | CN8 |
| BO9 | AN2 | BN14 | CN9 |
| BO10 | AN2 | BN14 | CN10 |
| BO11 | AN2 | BN14 | CN11 |
| BO12 | AN2 | BN14 | CN12 |
| BO13 | AN2 | BN14 | CN13 |
| BO14 | AN2 | BN14 | CN14 |
| BO15 | AN2 | BN14 | CN15 |
| BO16 | AN2 | BN14 | CN16 |
| BO17 | AN2 | BN14 | CN17 |
| BO18 | AN2 | BN14 | CN18 |
| BO19 | AN2 | BN14 | CN19 |
| BO20 | AN2 | BN14 | CN20 |
| BO21 | AN2 | BN14 | CN21 |
| BO22 | AN2 | BN14 | CN22 |
| BO23 | AN2 | BN14 | CN23 |
| BO24 | AN2 | BN14 | CN24 |
| BO25 | AN2 | BN14 | CN25 |
| BO26 | AN2 | BN14 | CN26 |
| BO27 | AN2 | BN14 | CN27 |
| BO28 | AN2 | BN14 | CN28 |
| BO29 | AN2 | BN14 | CN29 |
| BO30 | AN2 | BN14 | CN30 |
| BO31 | AN2 | BN14 | CN31 |
| BO32 | AN2 | BN14 | CN32 |
| BO33 | AN2 | BN14 | CN33 |
| BO34 | AN2 | BN14 | CN34 |
| BO35 | AN2 | BN14 | CN35 |
| BO36 | AN2 | BN14 | CN36 |
| BP1 | AN2 | BN15 | CN1 |
| BP2 | AN2 | BN15 | CN2 |
| BP3 | AN2 | BN15 | CN3 |
| BP4 | AN2 | BN15 | CN4 |
| BP5 | AN2 | BN15 | CN5 |
| BP6 | AN2 | BN15 | CN6 |
| BP7 | AN2 | BN15 | CN7 |
| BP8 | AN2 | BN15 | CN8 |
| BP9 | AN2 | BN15 | CN9 |
| BP10 | AN2 | BN15 | CN10 |
| BP11 | AN2 | BN15 | CN11 |
| BP12 | AN2 | BN15 | CN12 |
| BP13 | AN2 | BN15 | CN13 |
| BP14 | AN2 | BN15 | CN14 |
| BP15 | AN2 | BN15 | CN15 |
| BP16 | AN2 | BN15 | CN16 |
| BP17 | AN2 | BN15 | CN17 |
| BP18 | AN2 | BN15 | CN18 |
| BP19 | AN2 | BN15 | CN19 |
| BP20 | AN2 | BN15 | CN20 |
| BP21 | AN2 | BN15 | CN21 |
| BP22 | AN2 | BN15 | CN22 |
| BP23 | AN2 | BN15 | CN23 |
| BP24 | AN2 | BN15 | CN24 |
| BP25 | AN2 | BN15 | CN25 |
| BP26 | AN2 | BN15 | CN26 |
| BP27 | AN2 | BN15 | CN27 |
| BP28 | AN2 | BN15 | CN28 |
| BP29 | AN2 | BN15 | CN29 |
| BP30 | AN2 | BN15 | CN30 |
| BP31 | AN2 | BN15 | CN31 |
| BP32 | AN2 | BN15 | CN32 |
| BP33 | AN2 | BN15 | CN33 |
| BP34 | AN2 | BN15 | CN34 |
| BP35 | AN2 | BN15 | CN35 |
| BP36 | AN2 | BN15 | CN36 |
| BQ1 | AN2 | BN16 | CN1 |
| BQ2 | AN2 | BN16 | CN2 |
| BQ3 | AN2 | BN16 | CN3 |
| BQ4 | AN2 | BN16 | CN4 |
| BQ5 | AN2 | BN16 | CN5 |
| BQ6 | AN2 | BN16 | CN6 |
| BQ7 | AN2 | BN16 | CN7 |
| BQ8 | AN2 | BN16 | CN8 |
| BQ9 | AN2 | BN16 | CN9 |
| BQ10 | AN2 | BN16 | CN10 |
| BQ11 | AN2 | BN16 | CN11 |
| BQ12 | AN2 | BN16 | CN12 |
| BQ13 | AN2 | BN16 | CN13 |
| BQ14 | AN2 | BN16 | CN14 |
| BQ15 | AN2 | BN16 | CN15 |
| BQ16 | AN2 | BN16 | CN16 |
| BQ17 | AN2 | BN16 | CN17 |
| BQ18 | AN2 | BN16 | CN18 |
| BQ19 | AN2 | BN16 | CN19 |
| BQ20 | AN2 | BN16 | CN20 |
| BQ21 | AN2 | BN16 | CN21 |
| BQ22 | AN2 | BN16 | CN22 |
| BQ23 | AN2 | BN16 | CN23 |
| BQ24 | AN2 | BN16 | CN24 |
| BQ25 | AN2 | BN16 | CN25 |
| BQ26 | AN2 | BN16 | CN26 |
| BQ27 | AN2 | BN16 | CN27 |
| BQ28 | AN2 | BN16 | CN28 |
| BQ29 | AN2 | BN16 | CN29 |
| BQ30 | AN2 | BN16 | CN30 |
| BQ31 | AN2 | BN16 | CN31 |
| BQ32 | AN2 | BN16 | CN32 |
| BQ33 | AN2 | BN16 | CN33 |
| BQ34 | AN2 | BN16 | CN34 |
| BQ35 | AN2 | BN16 | CN35 |
| BQ36 | AN2 | BN16 | CN36 |
| BR1 | AN2 | BN17 | CN1 |
| BR2 | AN2 | BN17 | CN2 |
| BR3 | AN2 | BN17 | CN3 |
| BR4 | AN2 | BN17 | CN4 |
| BR5 | AN2 | BN17 | CN5 |
| BR6 | AN2 | BN17 | CN6 |
| BR7 | AN2 | BN17 | CN7 |
| BR8 | AN2 | BN17 | CN8 |
| BR9 | AN2 | BN17 | CN9 |
| BR10 | AN2 | BN17 | CN10 |
| BR11 | AN2 | BN17 | CN11 |
| BR12 | AN2 | BN17 | CN12 |
| BR13 | AN2 | BN17 | CN13 |
| BR14 | AN2 | BN17 | CN14 |
| BR15 | AN2 | BN17 | CN15 |
| BR16 | AN2 | BN17 | CN16 |
| BR17 | AN2 | BN17 | CN17 |
| BR18 | AN2 | BN17 | CN18 |
| BR19 | AN2 | BN17 | CN19 |
| BR20 | AN2 | BN17 | CN20 |
| BR21 | AN2 | BN17 | CN21 |
| BR22 | AN2 | BN17 | CN22 |
| BR23 | AN2 | BN17 | CN23 |
| BR24 | AN2 | BN17 | CN24 |
| BR25 | AN2 | BN17 | CN25 |
| BR26 | AN2 | BN17 | CN26 |
| BR27 | AN2 | BN17 | CN27 |
| BR28 | AN2 | BN17 | CN28 |
| BR29 | AN2 | BN17 | CN29 |
| BR30 | AN2 | BN17 | CN30 |
| BR31 | AN2 | BN17 | CN31 |
| BR32 | AN2 | BN17 | CN32 |
| BR33 | AN2 | BN17 | CN33 |
| BR34 | AN2 | BN17 | CN34 |
| BR35 | AN2 | BN17 | CN35 |
| BR36 | AN2 | BN17 | CN36 |
| BS1 | AN2 | BN18 | CN1 |
| BS2 | AN2 | BN18 | CN2 |
| BS3 | AN2 | BN18 | CN3 |
| BS4 | AN2 | BN18 | CN4 |
| BS5 | AN2 | BN18 | CN5 |
| BS6 | AN2 | BN18 | CN6 |
| BS7 | AN2 | BN18 | CN7 |
| BS8 | AN2 | BN18 | CN8 |
| BS9 | AN2 | BN18 | CN9 |
| BS10 | AN2 | BN18 | CN10 |
| BS11 | AN2 | BN18 | CN11 |
| BS12 | AN2 | BN18 | CN12 |
| BS13 | AN2 | BN18 | CN13 |
| BS14 | AN2 | BN18 | CN14 |
| BS15 | AN2 | BN18 | CN15 |
| BS16 | AN2 | BN18 | CN16 |
| BS17 | AN2 | BN18 | CN17 |
| BS18 | AN2 | BN18 | CN18 |
| BS19 | AN2 | BN18 | CN19 |
| BS20 | AN2 | BN18 | CN20 |
| BS21 | AN2 | BN18 | CN21 |
| BS22 | AN2 | BN18 | CN22 |
| BS23 | AN2 | BN18 | CN23 |
| BS24 | AN2 | BN18 | CN24 |
| BS25 | AN2 | BN18 | CN25 |
| BS26 | AN2 | BN18 | CN26 |
| BS27 | AN2 | BN18 | CN27 |
| BS28 | AN2 | BN18 | CN28 |
| BS29 | AN2 | BN18 | CN29 |
| BS30 | AN2 | BN18 | CN30 |
| BS31 | AN2 | BN18 | CN31 |
| BS32 | AN2 | BN18 | CN32 |
| BS33 | AN2 | BN18 | CN33 |
| BS34 | AN2 | BN18 | CN34 |
| BS35 | AN2 | BN18 | CN35 |
| BS36 | AN2 | BN18 | CN36 |
| BT1 | AN2 | BN19 | CN1 |
| BT2 | AN2 | BN19 | CN2 |
| BT3 | AN2 | BN19 | CN3 |
| BT4 | AN2 | BN19 | CN4 |
| BT5 | AN2 | BN19 | CN5 |
| BT6 | AN2 | BN19 | CN6 |
| BT7 | AN2 | BN19 | CN7 |
| BT8 | AN2 | BN19 | CN8 |
| BT9 | AN2 | BN19 | CN9 |
| BT10 | AN2 | BN19 | CN10 |
| BT11 | AN2 | BN19 | CN11 |
| BT12 | AN2 | BN19 | CN12 |
| BT13 | AN2 | BN19 | CN13 |
| BT14 | AN2 | BN19 | CN14 |
| BT15 | AN2 | BN19 | CN15 |
| BT16 | AN2 | BN19 | CN16 |
| BT17 | AN2 | BN19 | CN17 |
| BT18 | AN2 | BN19 | CN18 |
| BT19 | AN2 | BN19 | CN19 |
| BT20 | AN2 | BN19 | CN20 |
| BT21 | AN2 | BN19 | CN21 |
| BT22 | AN2 | BN19 | CN22 |
| BT23 | AN2 | BN19 | CN23 |
| BT24 | AN2 | BN19 | CN24 |
| BT25 | AN2 | BN19 | CN25 |
| BT26 | AN2 | BN19 | CN26 |
| BT27 | AN2 | BN19 | CN27 |
| BT28 | AN2 | BN19 | CN28 |
| BT29 | AN2 | BN19 | CN29 |
| BT30 | AN2 | BN19 | CN30 |
| BT31 | AN2 | BN19 | CN31 |
| BT32 | AN2 | BN19 | CN32 |
| BT33 | AN2 | BN19 | CN33 |
| BT34 | AN2 | BN19 | CN34 |
| BT35 | AN2 | BN19 | CN35 |
| BT36 | AN2 | BN19 | CN36 |
| BU1 | AN2 | BN20 | CN1 |
| BU2 | AN2 | BN20 | CN2 |
| BU3 | AN2 | BN20 | CN3 |
| BU4 | AN2 | BN20 | CN4 |
| BU5 | AN2 | BN20 | CN5 |
| BU6 | AN2 | BN20 | CN6 |
| BU7 | AN2 | BN20 | CN7 |
| BU8 | AN2 | BN20 | CN8 |
| BU9 | AN2 | BN20 | CN9 |
| BU10 | AN2 | BN20 | CN10 |
| BU11 | AN2 | BN20 | CN11 |
| BU12 | AN2 | BN20 | CN12 |
| BU13 | AN2 | BN20 | CN13 |
| BU14 | AN2 | BN20 | CN14 |
| BU15 | AN2 | BN20 | CN15 |
| BU16 | AN2 | BN20 | CN16 |
| BU17 | AN2 | BN20 | CN17 |
| BU18 | AN2 | BN20 | CN18 |
| BU19 | AN2 | BN20 | CN19 |
| BU20 | AN2 | BN20 | CN20 |
| BU21 | AN2 | BN20 | CN21 |
| BU22 | AN2 | BN20 | CN22 |
| BU23 | AN2 | BN20 | CN23 |
| BU24 | AN2 | BN20 | CN24 |
| BU25 | AN2 | BN20 | CN25 |
| BU26 | AN2 | BN20 | CN26 |
| BU27 | AN2 | BN20 | CN27 |
| BU28 | AN2 | BN20 | CN28 |
| BU29 | AN2 | BN20 | CN29 |
| BU30 | AN2 | BN20 | CN30 |
| BU31 | AN2 | BN20 | CN31 |
| BU32 | AN2 | BN20 | CN32 |
| BU33 | AN2 | BN20 | CN33 |
| BU34 | AN2 | BN20 | CN34 |
| BU35 | AN2 | BN20 | CN35 |
| BU36 | AN2 | BN20 | CN36 |
| BV1 | AN2 | BN21 | CN1 |
| BV2 | AN2 | BN21 | CN2 |
| BV3 | AN2 | BN21 | CN3 |
| BV4 | AN2 | BN21 | CN4 |
| BV5 | AN2 | BN21 | CN5 |
| BV6 | AN2 | BN21 | CN6 |
| BV7 | AN2 | BN21 | CN7 |
| BV8 | AN2 | BN21 | CN8 |
| BV9 | AN2 | BN21 | CN9 |
| BV10 | AN2 | BN21 | CN10 |
| BV11 | AN2 | BN21 | CN11 |
| BV12 | AN2 | BN21 | CN12 |
| BV13 | AN2 | BN21 | CN13 |
| BV14 | AN2 | BN21 | CN14 |
| BV15 | AN2 | BN21 | CN15 |
| BV16 | AN2 | BN21 | CN16 |
| BV17 | AN2 | BN21 | CN17 |
| BV18 | AN2 | BN21 | CN18 |
| BV19 | AN2 | BN21 | CN19 |
| BV20 | AN2 | BN21 | CN20 |
| BV21 | AN2 | BN21 | CN21 |
| BV22 | AN2 | BN21 | CN22 |
| BV23 | AN2 | BN21 | CN23 |
| BV24 | AN2 | BN21 | CN24 |
| BV25 | AN2 | BN21 | CN25 |
| BV26 | AN2 | BN21 | CN26 |
| BV27 | AN2 | BN21 | CN27 |
| BV28 | AN2 | BN21 | CN28 |
| BV29 | AN2 | BN21 | CN29 |
| BV30 | AN2 | BN21 | CN30 |
| BV31 | AN2 | BN21 | CN31 |
| BV32 | AN2 | BN21 | CN32 |
| BV33 | AN2 | BN21 | CN33 |
| BV34 | AN2 | BN21 | CN34 |
| BV35 | AN2 | BN21 | CN35 |
| BV36 | AN2 | BN21 | CN36 |
| BW1 | AN2 | BN22 | CN1 |
| BW2 | AN2 | BN22 | CN2 |
| BW3 | AN2 | BN22 | CN3 |
| BW4 | AN2 | BN22 | CN4 |
| BW5 | AN2 | BN22 | CN5 |
| BW6 | AN2 | BN22 | CN6 |
| BW7 | AN2 | BN22 | CN7 |
| BW8 | AN2 | BN22 | CN8 |
| BW9 | AN2 | BN22 | CN9 |
| BW10 | AN2 | BN22 | CN10 |
| BW11 | AN2 | BN22 | CN11 |
| BW12 | AN2 | BN22 | CN12 |
| BW13 | AN2 | BN22 | CN13 |
| BW14 | AN2 | BN22 | CN14 |
| BW15 | AN2 | BN22 | CN15 |
| BW16 | AN2 | BN22 | CN16 |
| BW17 | AN2 | BN22 | CN17 |
| BW18 | AN2 | BN22 | CN18 |
| BW19 | AN2 | BN22 | CN19 |
| BW20 | AN2 | BN22 | CN20 |
| BW21 | AN2 | BN22 | CN21 |
| BW22 | AN2 | BN22 | CN22 |
| BW23 | AN2 | BN22 | CN23 |
| BW24 | AN2 | BN22 | CN24 |
| BW25 | AN2 | BN22 | CN25 |
| BW26 | AN2 | BN22 | CN26 |
| BW27 | AN2 | BN22 | CN27 |
| BW28 | AN2 | BN22 | CN28 |
| BW29 | AN2 | BN22 | CN29 |
| BW30 | AN2 | BN22 | CN30 |
| BW31 | AN2 | BN22 | CN31 |
| BW32 | AN2 | BN22 | CN32 |
| BW33 | AN2 | BN22 | CN33 |
| BW34 | AN2 | BN22 | CN34 |
| BW35 | AN2 | BN22 | CN35 |
| BW36 | AN2 | BN22 | CN36 |
| CA1 | AN3 | BN1 | CN1 |
| CA2 | AN3 | BN1 | CN2 |
| CA3 | AN3 | BN1 | CN3 |
| CA4 | AN3 | BN1 | CN4 |
| CA5 | AN3 | BN1 | CN5 |
| CA6 | AN3 | BN1 | CN6 |
| CA7 | AN3 | BN1 | CN7 |
| CA8 | AN3 | BN1 | CN8 |
| CA9 | AN3 | BN1 | CN9 |
| CA10 | AN3 | BN1 | CN10 |
| CA11 | AN3 | BN1 | CN11 |
| CA12 | AN3 | BN1 | CN12 |
| CA13 | AN3 | BN1 | CN13 |
| CA14 | AN3 | BN1 | CN14 |
| CA15 | AN3 | BN1 | CN15 |
| CA16 | AN3 | BN1 | CN16 |
| CA17 | AN3 | BN1 | CN17 |
| CA18 | AN3 | BN1 | CN18 |
| CA19 | AN3 | BN1 | CN19 |
| CA20 | AN3 | BN1 | CN20 |
| CA21 | AN3 | BN1 | CN21 |
| CA22 | AN3 | BN1 | CN22 |
| CA23 | AN3 | BN1 | CN23 |
| CA24 | AN3 | BN1 | CN24 |
| CA25 | AN3 | BN1 | CN25 |
| CA26 | AN3 | BN1 | CN26 |
| CA27 | AN3 | BN1 | CN27 |
| CA28 | AN3 | BN1 | CN28 |
| CA29 | AN3 | BN1 | CN29 |
| CA30 | AN3 | BN1 | CN30 |
| CA31 | AN3 | BN1 | CN31 |
| CA32 | AN3 | BN1 | CN32 |
| CA33 | AN3 | BN1 | CN33 |
| CA34 | AN3 | BN1 | CN34 |
| CA35 | AN3 | BN1 | CN35 |
| CA36 | AN3 | BN1 | CN36 |
| CB1 | AN3 | BN2 | CN1 |
| CB2 | AN3 | BN2 | CN2 |
| CB3 | AN3 | BN2 | CN3 |
| CB4 | AN3 | BN2 | CN4 |
| CB5 | AN3 | BN2 | CN5 |
| CB6 | AN3 | BN2 | CN6 |
| CB7 | AN3 | BN2 | CN7 |
| CB8 | AN3 | BN2 | CN8 |
| CB9 | AN3 | BN2 | CN9 |
| CB10 | AN3 | BN2 | CN10 |
| CB11 | AN3 | BN2 | CN11 |
| CB12 | AN3 | BN2 | CN12 |
| CB13 | AN3 | BN2 | CN13 |
| CB14 | AN3 | BN2 | CN14 |
| CB15 | AN3 | BN2 | CN15 |
| CB16 | AN3 | BN2 | CN16 |
| CB17 | AN3 | BN2 | CN17 |
| CB18 | AN3 | BN2 | CN18 |
| CB19 | AN3 | BN2 | CN19 |
| CB20 | AN3 | BN2 | CN20 |
| CB21 | AN3 | BN2 | CN21 |
| CB22 | AN3 | BN2 | CN22 |
| CB23 | AN3 | BN2 | CN23 |
| CB24 | AN3 | BN2 | CN24 |
| CB25 | AN3 | BN2 | CN25 |
| CB26 | AN3 | BN2 | CN26 |
| CB27 | AN3 | BN2 | CN27 |
| CB28 | AN3 | BN2 | CN28 |
| CB29 | AN3 | BN2 | CN29 |
| CB30 | AN3 | BN2 | CN30 |
| CB31 | AN3 | BN2 | CN31 |
| CB32 | AN3 | BN2 | CN32 |
| CB33 | AN3 | BN2 | CN33 |
| CB34 | AN3 | BN2 | CN34 |
| CB35 | AN3 | BN2 | CN35 |
| CB36 | AN3 | BN2 | CN36 |
| CC1 | AN3 | BN3 | CN1 |
| CC2 | AN3 | BN3 | CN2 |
| CC3 | AN3 | BN3 | CN3 |
| CC4 | AN3 | BN3 | CN4 |
| CC5 | AN3 | BN3 | CN5 |
| CC6 | AN3 | BN3 | CN6 |
| CC7 | AN3 | BN3 | CN7 |
| CC8 | AN3 | BN3 | CN8 |
| CC9 | AN3 | BN3 | CN9 |
| CC10 | AN3 | BN3 | CN10 |
| CC11 | AN3 | BN3 | CN11 |
| CC12 | AN3 | BN3 | CN12 |
| CC13 | AN3 | BN3 | CN13 |
| CC14 | AN3 | BN3 | CN14 |
| CC15 | AN3 | BN3 | CN15 |
| CC16 | AN3 | BN3 | CN16 |
| CC17 | AN3 | BN3 | CN17 |
| CC18 | AN3 | BN3 | CN18 |
| CC19 | AN3 | BN3 | CN19 |
| CC20 | AN3 | BN3 | CN20 |
| CC21 | AN3 | BN3 | CN21 |
| CC22 | AN3 | BN3 | CN22 |
| CC23 | AN3 | BN3 | CN23 |
| CC24 | AN3 | BN3 | CN24 |
| CC25 | AN3 | BN3 | CN25 |
| CC26 | AN3 | BN3 | CN26 |
| CC27 | AN3 | BN3 | CN27 |
| CC28 | AN3 | BN3 | CN28 |
| CC29 | AN3 | BN3 | CN29 |
| CC30 | AN3 | BN3 | CN30 |
| CC31 | AN3 | BN3 | CN31 |
| CC32 | AN3 | BN3 | CN32 |
| CC33 | AN3 | BN3 | CN33 |
| CC34 | AN3 | BN3 | CN34 |
| CC35 | AN3 | BN3 | CN35 |
| CC36 | AN3 | BN3 | CN36 |
| CD1 | AN3 | BN4 | CN1 |
| CD2 | AN3 | BN4 | CN2 |
| CD3 | AN3 | BN4 | CN3 |
| CD4 | AN3 | BN4 | CN4 |
| CD5 | AN3 | BN4 | CN5 |
| CD6 | AN3 | BN4 | CN6 |
| CD7 | AN3 | BN4 | CN7 |
| CD8 | AN3 | BN4 | CN8 |
| CD9 | AN3 | BN4 | CN9 |
| CD10 | AN3 | BN4 | CN10 |
| CD11 | AN3 | BN4 | CN11 |
| CD12 | AN3 | BN4 | CN12 |
| CD13 | AN3 | BN4 | CN13 |
| CD14 | AN3 | BN4 | CN14 |
| CD15 | AN3 | BN4 | CN15 |
| CD16 | AN3 | BN4 | CN16 |
| CD17 | AN3 | BN4 | CN17 |
| CD18 | AN3 | BN4 | CN18 |
| CD19 | AN3 | BN4 | CN19 |
| CD20 | AN3 | BN4 | CN20 |
| CD21 | AN3 | BN4 | CN21 |
| CD22 | AN3 | BN4 | CN22 |
| CD23 | AN3 | BN4 | CN23 |
| CD24 | AN3 | BN4 | CN24 |
| CD25 | AN3 | BN4 | CN25 |
| CD26 | AN3 | BN4 | CN26 |
| CD27 | AN3 | BN4 | CN27 |
| CD28 | AN3 | BN4 | CN28 |
| CD29 | AN3 | BN4 | CN29 |
| CD30 | AN3 | BN4 | CN30 |
| CD31 | AN3 | BN4 | CN31 |
| CD32 | AN3 | BN4 | CN32 |
| CD33 | AN3 | BN4 | CN33 |
| CD34 | AN3 | BN4 | CN34 |
| CD35 | AN3 | BN4 | CN35 |
| CD36 | AN3 | BN4 | CN36 |
| CE1 | AN3 | BN5 | CN1 |
| CE2 | AN3 | BN5 | CN2 |
| CE3 | AN3 | BN5 | CN3 |
| CE4 | AN3 | BN5 | CN4 |
| CE5 | AN3 | BN5 | CN5 |
| CE6 | AN3 | BN5 | CN6 |
| CE7 | AN3 | BN5 | CN7 |
| CE8 | AN3 | BN5 | CN8 |
| CE9 | AN3 | BN5 | CN9 |
| CE10 | AN3 | BN5 | CN10 |
| CE11 | AN3 | BN5 | CN11 |
| CE12 | AN3 | BN5 | CN12 |
| CE13 | AN3 | BN5 | CN13 |
| CE14 | AN3 | BN5 | CN14 |
| CE15 | AN3 | BN5 | CN15 |
| CE16 | AN3 | BN5 | CN16 |
| CE17 | AN3 | BN5 | CN17 |
| CE18 | AN3 | BN5 | CN18 |
| CE19 | AN3 | BN5 | CN19 |
| CE20 | AN3 | BN5 | CN20 |
| CE21 | AN3 | BN5 | CN21 |
| CE22 | AN3 | BN5 | CN22 |
| CE23 | AN3 | BN5 | CN23 |
| CE24 | AN3 | BN5 | CN24 |
| CE25 | AN3 | BN5 | CN25 |
| CE26 | AN3 | BN5 | CN26 |
| CE27 | AN3 | BN5 | CN27 |
| CE28 | AN3 | BN5 | CN28 |
| CE29 | AN3 | BN5 | CN29 |
| CE30 | AN3 | BN5 | CN30 |
| CE31 | AN3 | BN5 | CN31 |
| CE32 | AN3 | BN5 | CN32 |
| CE33 | AN3 | BN5 | CN33 |
| CE34 | AN3 | BN5 | CN34 |
| CE35 | AN3 | BN5 | CN35 |
| CE36 | AN3 | BN5 | CN36 |
| CF1 | AN3 | BN6 | CN1 |
| CF2 | AN3 | BN6 | CN2 |
| CF3 | AN3 | BN6 | CN3 |
| CF4 | AN3 | BN6 | CN4 |
| CF5 | AN3 | BN6 | CN5 |
| CF6 | AN3 | BN6 | CN6 |
| CF7 | AN3 | BN6 | CN7 |
| CF8 | AN3 | BN6 | CN8 |
| CF9 | AN3 | BN6 | CN9 |
| CF10 | AN3 | BN6 | CN10 |
| CF11 | AN3 | BN6 | CN11 |
| CF12 | AN3 | BN6 | CN12 |
| CF13 | AN3 | BN6 | CN13 |
| CF14 | AN3 | BN6 | CN14 |
| CF15 | AN3 | BN6 | CN15 |
| CF16 | AN3 | BN6 | CN16 |
| CF17 | AN3 | BN6 | CN17 |
| CF18 | AN3 | BN6 | CN18 |
| CF19 | AN3 | BN6 | CN19 |
| CF20 | AN3 | BN6 | CN20 |
| CF21 | AN3 | BN6 | CN21 |
| CF22 | AN3 | BN6 | CN22 |
| CF23 | AN3 | BN6 | CN23 |
| CF24 | AN3 | BN6 | CN24 |
| CF25 | AN3 | BN6 | CN25 |
| CF26 | AN3 | BN6 | CN26 |
| CF27 | AN3 | BN6 | CN27 |
| CF28 | AN3 | BN6 | CN28 |
| CF29 | AN3 | BN6 | CN29 |
| CF30 | AN3 | BN6 | CN30 |
| CF31 | AN3 | BN6 | CN31 |
| CF32 | AN3 | BN6 | CN32 |
| CF33 | AN3 | BN6 | CN33 |
| CF34 | AN3 | BN6 | CN34 |
| CF35 | AN3 | BN6 | CN35 |
| CF36 | AN3 | BN6 | CN36 |
| CG1 | AN3 | BN7 | CN1 |
| CG2 | AN3 | BN7 | CN2 |
| CG3 | AN3 | BN7 | CN3 |
| CG4 | AN3 | BN7 | CN4 |
| CG5 | AN3 | BN7 | CN5 |
| CG6 | AN3 | BN7 | CN6 |
| CG7 | AN3 | BN7 | CN7 |
| CG8 | AN3 | BN7 | CN8 |
| CG9 | AN3 | BN7 | CN9 |
| CG10 | AN3 | BN7 | CN10 |
| CG11 | AN3 | BN7 | CN11 |
| CG12 | AN3 | BN7 | CN12 |
| CG13 | AN3 | BN7 | CN13 |
| CG14 | AN3 | BN7 | CN14 |
| CG15 | AN3 | BN7 | CN15 |
| CG16 | AN3 | BN7 | CN16 |
| CG17 | AN3 | BN7 | CN17 |
| CG18 | AN3 | BN7 | CN18 |
| CG19 | AN3 | BN7 | CN19 |
| CG20 | AN3 | BN7 | CN20 |
| CG21 | AN3 | BN7 | CN21 |
| CG22 | AN3 | BN7 | CN22 |
| CG23 | AN3 | BN7 | CN23 |
| CG24 | AN3 | BN7 | CN24 |
| CG25 | AN3 | BN7 | CN25 |
| CG26 | AN3 | BN7 | CN26 |
| CG27 | AN3 | BN7 | CN27 |
| CG28 | AN3 | BN7 | CN28 |
| CG29 | AN3 | BN7 | CN29 |
| CG30 | AN3 | BN7 | CN30 |
| CG31 | AN3 | BN7 | CN31 |
| CG32 | AN3 | BN7 | CN32 |
| CG33 | AN3 | BN7 | CN33 |
| CG34 | AN3 | BN7 | CN34 |
| CG35 | AN3 | BN7 | CN35 |
| CG36 | AN3 | BN7 | CN36 |
| CH1 | AN3 | BN8 | CN1 |
| CH2 | AN3 | BN8 | CN2 |
| CH3 | AN3 | BN8 | CN3 |
| CH4 | AN3 | BN8 | CN4 |
| CH5 | AN3 | BN8 | CN5 |
| CH6 | AN3 | BN8 | CN6 |
| CH7 | AN3 | BN8 | CN7 |
| CH8 | AN3 | BN8 | CN8 |
| CH9 | AN3 | BN8 | CN9 |
| CH10 | AN3 | BN8 | CN10 |
| CH11 | AN3 | BN8 | CN11 |
| CH12 | AN3 | BN8 | CN12 |
| CH13 | AN3 | BN8 | CN13 |
| CH14 | AN3 | BN8 | CN14 |
| CH15 | AN3 | BN8 | CN15 |
| CH16 | AN3 | BN8 | CN16 |
| CH17 | AN3 | BN8 | CN17 |
| CH18 | AN3 | BN8 | CN18 |
| CH19 | AN3 | BN8 | CN19 |
| CH20 | AN3 | BN8 | CN20 |
| CH21 | AN3 | BN8 | CN21 |
| CH22 | AN3 | BN8 | CN22 |
| CH23 | AN3 | BN8 | CN23 |
| CH24 | AN3 | BN8 | CN24 |
| CH25 | AN3 | BN8 | CN25 |
| CH26 | AN3 | BN8 | CN26 |
| CH27 | AN3 | BN8 | CN27 |
| CH28 | AN3 | BN8 | CN28 |
| CH29 | AN3 | BN8 | CN29 |
| CH30 | AN3 | BN8 | CN30 |
| CH31 | AN3 | BN8 | CN31 |
| CH32 | AN3 | BN8 | CN32 |
| CH33 | AN3 | BN8 | CN33 |
| CH34 | AN3 | BN8 | CN34 |
| CH35 | AN3 | BN8 | CN35 |
| CH36 | AN3 | BN8 | CN36 |
| CI1 | AN3 | BN9 | CN1 |
| CI2 | AN3 | BN9 | CN2 |
| CI3 | AN3 | BN9 | CN3 |
| CI4 | AN3 | BN9 | CN4 |
| CI5 | AN3 | BN9 | CN5 |
| CI6 | AN3 | BN9 | CN6 |
| CI7 | AN3 | BN9 | CN7 |
| CI8 | AN3 | BN9 | CN8 |
| CI9 | AN3 | BN9 | CN9 |
| CI10 | AN3 | BN9 | CN10 |
| CI11 | AN3 | BN9 | CN11 |
| CI12 | AN3 | BN9 | CN12 |
| CI13 | AN3 | BN9 | CN13 |
| CI14 | AN3 | BN9 | CN14 |
| CI15 | AN3 | BN9 | CN15 |
| CI16 | AN3 | BN9 | CN16 |
| CI17 | AN3 | BN9 | CN17 |
| CI18 | AN3 | BN9 | CN18 |
| CI19 | AN3 | BN9 | CN19 |
| CI20 | AN3 | BN9 | CN20 |
| CI21 | AN3 | BN9 | CN21 |
| CI22 | AN3 | BN9 | CN22 |
| CI23 | AN3 | BN9 | CN23 |
| CI24 | AN3 | BN9 | CN24 |
| CI25 | AN3 | BN9 | CN25 |
| CI26 | AN3 | BN9 | CN26 |
| CI27 | AN3 | BN9 | CN27 |
| CI28 | AN3 | BN9 | CN28 |
| CI29 | AN3 | BN9 | CN29 |
| CI30 | AN3 | BN9 | CN30 |
| CI31 | AN3 | BN9 | CN31 |
| CI32 | AN3 | BN9 | CN32 |
| CI33 | AN3 | BN9 | CN33 |
| CI34 | AN3 | BN9 | CN34 |
| CI35 | AN3 | BN9 | CN35 |
| CI36 | AN3 | BN9 | CN36 |
| CJ1 | AN3 | BN10 | CN1 |
| CJ2 | AN3 | BN10 | CN2 |
| CJ3 | AN3 | BN10 | CN3 |
| CJ4 | AN3 | BN10 | CN4 |
| CJ5 | AN3 | BN10 | CN5 |
| CJ6 | AN3 | BN10 | CN6 |
| CJ7 | AN3 | BN10 | CN7 |
| CJ8 | AN3 | BN10 | CN8 |
| CJ9 | AN3 | BN10 | CN9 |
| CJ10 | AN3 | BN10 | CN10 |
| CJ11 | AN3 | BN10 | CN11 |
| CJ12 | AN3 | BN10 | CN12 |
| CJ13 | AN3 | BN10 | CN13 |
| CJ14 | AN3 | BN10 | CN14 |
| CJ15 | AN3 | BN10 | CN15 |
| CJ16 | AN3 | BN10 | CN16 |
| CJ17 | AN3 | BN10 | CN17 |
| CJ18 | AN3 | BN10 | CN18 |
| CJ19 | AN3 | BN10 | CN19 |
| CJ20 | AN3 | BN10 | CN20 |
| CJ21 | AN3 | BN10 | CN21 |
| CJ22 | AN3 | BN10 | CN22 |
| CJ23 | AN3 | BN10 | CN23 |
| CJ24 | AN3 | BN10 | CN24 |
| CJ25 | AN3 | BN10 | CN25 |
| CJ26 | AN3 | BN10 | CN26 |
| CJ27 | AN3 | BN10 | CN27 |
| CJ28 | AN3 | BN10 | CN28 |
| CJ29 | AN3 | BN10 | CN29 |
| CJ30 | AN3 | BN10 | CN30 |
| CJ31 | AN3 | BN10 | CN31 |
| CJ32 | AN3 | BN10 | CN32 |
| CJ33 | AN3 | BN10 | CN33 |
| CJ34 | AN3 | BN10 | CN34 |
| CJ35 | AN3 | BN10 | CN35 |
| CJ36 | AN3 | BN10 | CN36 |
| CK1 | AN3 | BN11 | CN1 |
| CK2 | AN3 | BN11 | CN2 |
| CK3 | AN3 | BN11 | CN3 |
| CK4 | AN3 | BN11 | CN4 |
| CK5 | AN3 | BN11 | CN5 |
| CK6 | AN3 | BN11 | CN6 |
| CK7 | AN3 | BN11 | CN7 |
| CK8 | AN3 | BN11 | CN8 |
| CK9 | AN3 | BN11 | CN9 |
| CK10 | AN3 | BN11 | CN10 |
| CK11 | AN3 | BN11 | CN11 |
| CK12 | AN3 | BN11 | CN12 |
| CK13 | AN3 | BN11 | CN13 |
| CK14 | AN3 | BN11 | CN14 |
| CK15 | AN3 | BN11 | CN15 |
| CK16 | AN3 | BN11 | CN16 |
| CK17 | AN3 | BN11 | CN17 |
| CK18 | AN3 | BN11 | CN18 |
| CK19 | AN3 | BN11 | CN19 |
| CK20 | AN3 | BN11 | CN20 |
| CK21 | AN3 | BN11 | CN21 |
| CK22 | AN3 | BN11 | CN22 |
| CK23 | AN3 | BN11 | CN23 |
| CK24 | AN3 | BN11 | CN24 |
| CK25 | AN3 | BN11 | CN25 |
| CK26 | AN3 | BN11 | CN26 |
| CK27 | AN3 | BN11 | CN27 |
| CK28 | AN3 | BN11 | CN28 |
| CK29 | AN3 | BN11 | CN29 |
| CK30 | AN3 | BN11 | CN30 |
| CK31 | AN3 | BN11 | CN31 |
| CK32 | AN3 | BN11 | CN32 |
| CK33 | AN3 | BN11 | CN33 |
| CK34 | AN3 | BN11 | CN34 |
| CK35 | AN3 | BN11 | CN35 |
| CK36 | AN3 | BN11 | CN36 |
| CL1 | AN3 | BN12 | CN1 |
| CL2 | AN3 | BN12 | CN2 |
| CL3 | AN3 | BN12 | CN3 |
| CL4 | AN3 | BN12 | CN4 |
| CL5 | AN3 | BN12 | CN5 |
| CL6 | AN3 | BN12 | CN6 |
| CL7 | AN3 | BN12 | CN7 |
| CL8 | AN3 | BN12 | CN8 |
| CL9 | AN3 | BN12 | CN9 |
| CL10 | AN3 | BN12 | CN10 |
| CL11 | AN3 | BN12 | CN11 |
| CL12 | AN3 | BN12 | CN12 |
| CL13 | AN3 | BN12 | CN13 |
| CL14 | AN3 | BN12 | CN14 |
| CL15 | AN3 | BN12 | CN15 |
| CL16 | AN3 | BN12 | CN16 |
| CL17 | AN3 | BN12 | CN17 |
| CL18 | AN3 | BN12 | CN18 |
| CL19 | AN3 | BN12 | CN19 |
| CL20 | AN3 | BN12 | CN20 |
| CL21 | AN3 | BN12 | CN21 |
| CL22 | AN3 | BN12 | CN22 |
| CL23 | AN3 | BN12 | CN23 |
| CL24 | AN3 | BN12 | CN24 |
| CL25 | AN3 | BN12 | CN25 |
| CL26 | AN3 | BN12 | CN26 |
| CL27 | AN3 | BN12 | CN27 |
| CL28 | AN3 | BN12 | CN28 |
| CL29 | AN3 | BN12 | CN29 |
| CL30 | AN3 | BN12 | CN30 |
| CL31 | AN3 | BN12 | CN31 |
| CL32 | AN3 | BN12 | CN32 |
| CL33 | AN3 | BN12 | CN33 |
| CL34 | AN3 | BN12 | CN34 |
| CL35 | AN3 | BN12 | CN35 |
| CL36 | AN3 | BN12 | CN36 |
| CM1 | AN3 | BN13 | CN1 |
| CM2 | AN3 | BN13 | CN2 |
| CM3 | AN3 | BN13 | CN3 |
| CM4 | AN3 | BN13 | CN4 |
| CM5 | AN3 | BN13 | CN5 |
| CM6 | AN3 | BN13 | CN6 |
| CM7 | AN3 | BN13 | CN7 |
| CM8 | AN3 | BN13 | CN8 |
| CM9 | AN3 | BN13 | CN9 |
| CM10 | AN3 | BN13 | CN10 |
| CM11 | AN3 | BN13 | CN11 |
| CM12 | AN3 | BN13 | CN12 |
| CM13 | AN3 | BN13 | CN13 |
| CM14 | AN3 | BN13 | CN14 |
| CM15 | AN3 | BN13 | CN15 |
| CM16 | AN3 | BN13 | CN16 |
| CM17 | AN3 | BN13 | CN17 |
| CM18 | AN3 | BN13 | CN18 |
| CM19 | AN3 | BN13 | CN19 |
| CM20 | AN3 | BN13 | CN20 |
| CM21 | AN3 | BN13 | CN21 |
| CM22 | AN3 | BN13 | CN22 |
| CM23 | AN3 | BN13 | CN23 |
| CM24 | AN3 | BN13 | CN24 |
| CM25 | AN3 | BN13 | CN25 |
| CM26 | AN3 | BN13 | CN26 |
| CM27 | AN3 | BN13 | CN27 |
| CM28 | AN3 | BN13 | CN28 |
| CM29 | AN3 | BN13 | CN29 |
| CM30 | AN3 | BN13 | CN30 |
| CM31 | AN3 | BN13 | CN31 |
| CM32 | AN3 | BN13 | CN32 |
| CM33 | AN3 | BN13 | CN33 |
| CM34 | AN3 | BN13 | CN34 |
| CM35 | AN3 | BN13 | CN35 |
| CM36 | AN3 | BN13 | CN36 |
| CO1 | AN3 | BN14 | CN1 |
| CO2 | AN3 | BN14 | CN2 |
| CO3 | AN3 | BN14 | CN3 |
| CO4 | AN3 | BN14 | CN4 |
| CO5 | AN3 | BN14 | CN5 |
| CO6 | AN3 | BN14 | CN6 |
| CO7 | AN3 | BN14 | CN7 |
| CO8 | AN3 | BN14 | CN8 |
| CO9 | AN3 | BN14 | CN9 |
| CO10 | AN3 | BN14 | CN10 |
| CO11 | AN3 | BN14 | CN11 |
| CO12 | AN3 | BN14 | CN12 |
| CO13 | AN3 | BN14 | CN13 |
| CO14 | AN3 | BN14 | CN14 |
| CO15 | AN3 | BN14 | CN15 |
| CO16 | AN3 | BN14 | CN16 |
| CO17 | AN3 | BN14 | CN17 |
| CO18 | AN3 | BN14 | CN18 |
| CO19 | AN3 | BN14 | CN19 |
| CO20 | AN3 | BN14 | CN20 |
| CO21 | AN3 | BN14 | CN21 |
| CO22 | AN3 | BN14 | CN22 |
| CO23 | AN3 | BN14 | CN23 |
| CO24 | AN3 | BN14 | CN24 |
| CO25 | AN3 | BN14 | CN25 |
| CO26 | AN3 | BN14 | CN26 |
| CO27 | AN3 | BN14 | CN27 |
| CO28 | AN3 | BN14 | CN28 |
| CO29 | AN3 | BN14 | CN29 |
| CO30 | AN3 | BN14 | CN30 |
| CO31 | AN3 | BN14 | CN31 |
| CO32 | AN3 | BN14 | CN32 |
| CO33 | AN3 | BN14 | CN33 |
| CO34 | AN3 | BN14 | CN34 |
| CO35 | AN3 | BN14 | CN35 |
| CO36 | AN3 | BN14 | CN36 |
| CP1 | AN3 | BN15 | CN1 |
| CP2 | AN3 | BN15 | CN2 |
| CP3 | AN3 | BN15 | CN3 |
| CP4 | AN3 | BN15 | CN4 |
| CP5 | AN3 | BN15 | CN5 |
| CP6 | AN3 | BN15 | CN6 |
| CP7 | AN3 | BN15 | CN7 |
| CP8 | AN3 | BN15 | CN8 |
| CP9 | AN3 | BN15 | CN9 |
| CP10 | AN3 | BN15 | CN10 |
| CP11 | AN3 | BN15 | CN11 |
| CP12 | AN3 | BN15 | CN12 |
| CP13 | AN3 | BN15 | CN13 |
| CP14 | AN3 | BN15 | CN14 |
| CP15 | AN3 | BN15 | CN15 |
| CP16 | AN3 | BN15 | CN16 |
| CP17 | AN3 | BN15 | CN17 |
| CP18 | AN3 | BN15 | CN18 |
| CP19 | AN3 | BN15 | CN19 |
| CP20 | AN3 | BN15 | CN20 |
| CP21 | AN3 | BN15 | CN21 |
| CP22 | AN3 | BN15 | CN22 |
| CP23 | AN3 | BN15 | CN23 |
| CP24 | AN3 | BN15 | CN24 |
| CP25 | AN3 | BN15 | CN25 |
| CP26 | AN3 | BN15 | CN26 |
| CP27 | AN3 | BN15 | CN27 |
| CP28 | AN3 | BN15 | CN28 |
| CP29 | AN3 | BN15 | CN29 |
| CP30 | AN3 | BN15 | CN30 |
| CP31 | AN3 | BN15 | CN31 |
| CP32 | AN3 | BN15 | CN32 |
| CP33 | AN3 | BN15 | CN33 |
| CP34 | AN3 | BN15 | CN34 |
| CP35 | AN3 | BN15 | CN35 |
| CP36 | AN3 | BN15 | CN36 |
| CQ1 | AN3 | BN16 | CN1 |
| CQ2 | AN3 | BN16 | CN2 |
| CQ3 | AN3 | BN16 | CN3 |
| CQ4 | AN3 | BN16 | CN4 |
| CQ5 | AN3 | BN16 | CN5 |
| CQ6 | AN3 | BN16 | CN6 |
| CQ7 | AN3 | BN16 | CN7 |
| CQ8 | AN3 | BN16 | CN8 |
| CQ9 | AN3 | BN16 | CN9 |
| CQ10 | AN3 | BN16 | CN10 |
| CQ11 | AN3 | BN16 | CN11 |
| CQ12 | AN3 | BN16 | CN12 |
| CQ13 | AN3 | BN16 | CN13 |
| CQ14 | AN3 | BN16 | CN14 |
| CQ15 | AN3 | BN16 | CN15 |
| CQ16 | AN3 | BN16 | CN16 |
| CQ17 | AN3 | BN16 | CN17 |
| CQ18 | AN3 | BN16 | CN18 |
| CQ19 | AN3 | BN16 | CN19 |
| CQ20 | AN3 | BN16 | CN20 |
| CQ21 | AN3 | BN16 | CN21 |
| CQ22 | AN3 | BN16 | CN22 |
| CQ23 | AN3 | BN16 | CN23 |
| CQ24 | AN3 | BN16 | CN24 |
| CQ25 | AN3 | BN16 | CN25 |
| CQ26 | AN3 | BN16 | CN26 |
| CQ27 | AN3 | BN16 | CN27 |
| CQ28 | AN3 | BN16 | CN28 |
| CQ29 | AN3 | BN16 | CN29 |
| CQ30 | AN3 | BN16 | CN30 |
| CQ31 | AN3 | BN16 | CN31 |
| CQ32 | AN3 | BN16 | CN32 |
| CQ33 | AN3 | BN16 | CN33 |
| CQ34 | AN3 | BN16 | CN34 |
| CQ35 | AN3 | BN16 | CN35 |
| CQ36 | AN3 | BN16 | CN36 |
| CR1 | AN3 | BN17 | CN1 |
| CR2 | AN3 | BN17 | CN2 |
| CR3 | AN3 | BN17 | CN3 |
| CR4 | AN3 | BN17 | CN4 |
| CR5 | AN3 | BN17 | CN5 |
| CR6 | AN3 | BN17 | CN6 |
| CR7 | AN3 | BN17 | CN7 |
| CR8 | AN3 | BN17 | CN8 |
| CR9 | AN3 | BN17 | CN9 |
| CR10 | AN3 | BN17 | CN10 |
| CR11 | AN3 | BN17 | CN11 |
| CR12 | AN3 | BN17 | CN12 |
| CR13 | AN3 | BN17 | CN13 |
| CR14 | AN3 | BN17 | CN14 |
| CR15 | AN3 | BN17 | CN15 |
| CR16 | AN3 | BN17 | CN16 |
| CR17 | AN3 | BN17 | CN17 |
| CR18 | AN3 | BN17 | CN18 |
| CR19 | AN3 | BN17 | CN19 |
| CR20 | AN3 | BN17 | CN20 |
| CR21 | AN3 | BN17 | CN21 |
| CR22 | AN3 | BN17 | CN22 |
| CR23 | AN3 | BN17 | CN23 |
| CR24 | AN3 | BN17 | CN24 |
| CR25 | AN3 | BN17 | CN25 |
| CR26 | AN3 | BN17 | CN26 |
| CR27 | AN3 | BN17 | CN27 |
| CR28 | AN3 | BN17 | CN28 |
| CR29 | AN3 | BN17 | CN29 |
| CR30 | AN3 | BN17 | CN30 |
| CR31 | AN3 | BN17 | CN31 |
| CR32 | AN3 | BN17 | CN32 |
| CR33 | AN3 | BN17 | CN33 |
| CR34 | AN3 | BN17 | CN34 |
| CR35 | AN3 | BN17 | CN35 |
| CR36 | AN3 | BN17 | CN36 |
| CS1 | AN3 | BN18 | CN1 |
| CS2 | AN3 | BN18 | CN2 |
| CS3 | AN3 | BN18 | CN3 |
| CS4 | AN3 | BN18 | CN4 |
| CS5 | AN3 | BN18 | CN5 |
| CS6 | AN3 | BN18 | CN6 |
| CS7 | AN3 | BN18 | CN7 |
| CS8 | AN3 | BN18 | CN8 |
| CS9 | AN3 | BN18 | CN9 |
| CS10 | AN3 | BN18 | CN10 |
| CS11 | AN3 | BN18 | CN11 |
| CS12 | AN3 | BN18 | CN12 |
| CS13 | AN3 | BN18 | CN13 |
| CS14 | AN3 | BN18 | CN14 |
| CS15 | AN3 | BN18 | CN15 |
| CS16 | AN3 | BN18 | CN16 |
| CS17 | AN3 | BN18 | CN17 |
| CS18 | AN3 | BN18 | CN18 |
| CS19 | AN3 | BN18 | CN19 |
| CS20 | AN3 | BN18 | CN20 |
| CS21 | AN3 | BN18 | CN21 |
| CS22 | AN3 | BN18 | CN22 |
| CS23 | AN3 | BN18 | CN23 |
| CS24 | AN3 | BN18 | CN24 |
| CS25 | AN3 | BN18 | CN25 |
| CS26 | AN3 | BN18 | CN26 |
| CS27 | AN3 | BN18 | CN27 |
| CS28 | AN3 | BN18 | CN28 |
| CS29 | AN3 | BN18 | CN29 |
| CS30 | AN3 | BN18 | CN30 |
| CS31 | AN3 | BN18 | CN31 |
| CS32 | AN3 | BN18 | CN32 |
| CS33 | AN3 | BN18 | CN33 |
| CS34 | AN3 | BN18 | CN34 |
| CS35 | AN3 | BN18 | CN35 |
| CS36 | AN3 | BN18 | CN36 |
| CT1 | AN3 | BN19 | CN1 |
| CT2 | AN3 | BN19 | CN2 |
| CT3 | AN3 | BN19 | CN3 |
| CT4 | AN3 | BN19 | CN4 |
| CT5 | AN3 | BN19 | CN5 |
| CT6 | AN3 | BN19 | CN6 |
| CT7 | AN3 | BN19 | CN7 |
| CT8 | AN3 | BN19 | CN8 |
| CT9 | AN3 | BN19 | CN9 |
| CT10 | AN3 | BN19 | CN10 |
| CT11 | AN3 | BN19 | CN11 |
| CT12 | AN3 | BN19 | CN12 |
| CT13 | AN3 | BN19 | CN13 |
| CT14 | AN3 | BN19 | CN14 |
| CT15 | AN3 | BN19 | CN15 |
| CT16 | AN3 | BN19 | CN16 |
| CT17 | AN3 | BN19 | CN17 |
| CT18 | AN3 | BN19 | CN18 |
| CT19 | AN3 | BN19 | CN19 |
| CT20 | AN3 | BN19 | CN20 |
| CT21 | AN3 | BN19 | CN21 |
| CT22 | AN3 | BN19 | CN22 |
| CT23 | AN3 | BN19 | CN23 |
| CT24 | AN3 | BN19 | CN24 |
| CT25 | AN3 | BN19 | CN25 |
| CT26 | AN3 | BN19 | CN26 |
| CT27 | AN3 | BN19 | CN27 |
| CT28 | AN3 | BN19 | CN28 |
| CT29 | AN3 | BN19 | CN29 |
| CT30 | AN3 | BN19 | CN30 |
| CT31 | AN3 | BN19 | CN31 |
| CT32 | AN3 | BN19 | CN32 |
| CT33 | AN3 | BN19 | CN33 |
| CT34 | AN3 | BN19 | CN34 |
| CT35 | AN3 | BN19 | CN35 |
| CT36 | AN3 | BN19 | CN36 |
| CU1 | AN3 | BN20 | CN1 |
| CU2 | AN3 | BN20 | CN2 |
| CU3 | AN3 | BN20 | CN3 |
| CU4 | AN3 | BN20 | CN4 |
| CU5 | AN3 | BN20 | CN5 |
| CU6 | AN3 | BN20 | CN6 |
| CU7 | AN3 | BN20 | CN7 |
| CU8 | AN3 | BN20 | CN8 |
| CU9 | AN3 | BN20 | CN9 |
| CU10 | AN3 | BN20 | CN10 |
| CU11 | AN3 | BN20 | CN11 |
| CU12 | AN3 | BN20 | CN12 |
| CU13 | AN3 | BN20 | CN13 |
| CU14 | AN3 | BN20 | CN14 |
| CU15 | AN3 | BN20 | CN15 |
| CU16 | AN3 | BN20 | CN16 |
| CU17 | AN3 | BN20 | CN17 |
| CU18 | AN3 | BN20 | CN18 |
| CU19 | AN3 | BN20 | CN19 |
| CU20 | AN3 | BN20 | CN20 |
| CU21 | AN3 | BN20 | CN21 |
| CU22 | AN3 | BN20 | CN22 |
| CU23 | AN3 | BN20 | CN23 |
| CU24 | AN3 | BN20 | CN24 |
| CU25 | AN3 | BN20 | CN25 |
| CU26 | AN3 | BN20 | CN26 |
| CU27 | AN3 | BN20 | CN27 |
| CU28 | AN3 | BN20 | CN28 |
| CU29 | AN3 | BN20 | CN29 |
| CU30 | AN3 | BN20 | CN30 |
| CU31 | AN3 | BN20 | CN31 |
| CU32 | AN3 | BN20 | CN32 |
| CU33 | AN3 | BN20 | CN33 |
| CU34 | AN3 | BN20 | CN34 |
| CU35 | AN3 | BN20 | CN35 |
| CU36 | AN3 | BN20 | CN36 |
| CV1 | AN3 | BN21 | CN1 |
| CV2 | AN3 | BN21 | CN2 |
| CV3 | AN3 | BN21 | CN3 |
| CV4 | AN3 | BN21 | CN4 |
| CV5 | AN3 | BN21 | CN5 |
| CV6 | AN3 | BN21 | CN6 |
| CV7 | AN3 | BN21 | CN7 |
| CV8 | AN3 | BN21 | CN8 |
| CV9 | AN3 | BN21 | CN9 |
| CV10 | AN3 | BN21 | CN10 |
| CV11 | AN3 | BN21 | CN11 |
| CV12 | AN3 | BN21 | CN12 |
| CV13 | AN3 | BN21 | CN13 |
| CV14 | AN3 | BN21 | CN14 |
| CV15 | AN3 | BN21 | CN15 |
| CV16 | AN3 | BN21 | CN16 |
| CV17 | AN3 | BN21 | CN17 |
| CV18 | AN3 | BN21 | CN18 |
| CV19 | AN3 | BN21 | CN19 |
| CV20 | AN3 | BN21 | CN20 |
| CV21 | AN3 | BN21 | CN21 |
| CV22 | AN3 | BN21 | CN22 |
| CV23 | AN3 | BN21 | CN23 |
| CV24 | AN3 | BN21 | CN24 |
| CV25 | AN3 | BN21 | CN25 |
| CV26 | AN3 | BN21 | CN26 |
| CV27 | AN3 | BN21 | CN27 |
| CV28 | AN3 | BN21 | CN28 |
| CV29 | AN3 | BN21 | CN29 |
| CV30 | AN3 | BN21 | CN30 |
| CV31 | AN3 | BN21 | CN31 |
| CV32 | AN3 | BN21 | CN32 |
| CV33 | AN3 | BN21 | CN33 |
| CV34 | AN3 | BN21 | CN34 |
| CV35 | AN3 | BN21 | CN35 |
| CV36 | AN3 | BN21 | CN36 |
| CW1 | AN3 | BN22 | CN1 |
| CW2 | AN3 | BN22 | CN2 |
| CW3 | AN3 | BN22 | CN3 |
| CW4 | AN3 | BN22 | CN4 |
| CW5 | AN3 | BN22 | CN5 |
| CW6 | AN3 | BN22 | CN6 |
| CW7 | AN3 | BN22 | CN7 |
| CW8 | AN3 | BN22 | CN8 |
| CW9 | AN3 | BN22 | CN9 |
| CW10 | AN3 | BN22 | CN10 |
| CW11 | AN3 | BN22 | CN11 |
| CW12 | AN3 | BN22 | CN12 |
| CW13 | AN3 | BN22 | CN13 |
| CW14 | AN3 | BN22 | CN14 |
| CW15 | AN3 | BN22 | CN15 |
| CW16 | AN3 | BN22 | CN16 |
| CW17 | AN3 | BN22 | CN17 |
| CW18 | AN3 | BN22 | CN18 |
| CW19 | AN3 | BN22 | CN19 |
| CW20 | AN3 | BN22 | CN20 |
| CW21 | AN3 | BN22 | CN21 |
| CW22 | AN3 | BN22 | CN22 |
| CW23 | AN3 | BN22 | CN23 |
| CW24 | AN3 | BN22 | CN24 |
| CW25 | AN3 | BN22 | CN25 |
| CW26 | AN3 | BN22 | CN26 |
| CW27 | AN3 | BN22 | CN27 |
| CW28 | AN3 | BN22 | CN28 |
| CW29 | AN3 | BN22 | CN29 |
| CW30 | AN3 | BN22 | CN30 |
| CW31 | AN3 | BN22 | CN31 |
| CW32 | AN3 | BN22 | CN32 |
| CW33 | AN3 | BN22 | CN33 |
| CW34 | AN3 | BN22 | CN34 |
| CW35 | AN3 | BN22 | CN35 |
| CW36 | AN3 | BN22 | CN36 |
| DA1 | AN4 | BN1 | CN1 |
| DA2 | AN4 | BN1 | CN2 |
| DA3 | AN4 | BN1 | CN3 |
| DA4 | AN4 | BN1 | CN4 |
| DA5 | AN4 | BN1 | CN5 |
| DA6 | AN4 | BN1 | CN6 |
| DA7 | AN4 | BN1 | CN7 |
| DA8 | AN4 | BN1 | CN8 |
| DA9 | AN4 | BN1 | CN9 |
| DA10 | AN4 | BN1 | CN10 |
| DA11 | AN4 | BN1 | CN11 |
| DA12 | AN4 | BN1 | CN12 |
| DA13 | AN4 | BN1 | CN13 |
| DA14 | AN4 | BN1 | CN14 |
| DA15 | AN4 | BN1 | CN15 |
| DA16 | AN4 | BN1 | CN16 |
| DA17 | AN4 | BN1 | CN17 |
| DA18 | AN4 | BN1 | CN18 |
| DA19 | AN4 | BN1 | CN19 |
| DA20 | AN4 | BN1 | CN20 |
| DA21 | AN4 | BN1 | CN21 |
| DA22 | AN4 | BN1 | CN22 |
| DA23 | AN4 | BN1 | CN23 |
| DA24 | AN4 | BN1 | CN24 |
| DA25 | AN4 | BN1 | CN25 |
| DA26 | AN4 | BN1 | CN26 |
| DA27 | AN4 | BN1 | CN27 |
| DA28 | AN4 | BN1 | CN28 |
| DA29 | AN4 | BN1 | CN29 |
| DA30 | AN4 | BN1 | CN30 |
| DA31 | AN4 | BN1 | CN31 |
| DA32 | AN4 | BN1 | CN32 |
| DA33 | AN4 | BN1 | CN33 |
| DA34 | AN4 | BN1 | CN34 |
| DA35 | AN4 | BN1 | CN35 |
| DA36 | AN4 | BN1 | CN36 |
| DB1 | AN4 | BN2 | CN1 |
| DB2 | AN4 | BN2 | CN2 |
| DB3 | AN4 | BN2 | CN3 |
| DB4 | AN4 | BN2 | CN4 |
| DB5 | AN4 | BN2 | CN5 |
| DB6 | AN4 | BN2 | CN6 |
| DB7 | AN4 | BN2 | CN7 |
| DB8 | AN4 | BN2 | CN8 |
| DB9 | AN4 | BN2 | CN9 |
| DB10 | AN4 | BN2 | CN10 |
| DB11 | AN4 | BN2 | CN11 |
| DB12 | AN4 | BN2 | CN12 |
| DB13 | AN4 | BN2 | CN13 |
| DB14 | AN4 | BN2 | CN14 |
| DB15 | AN4 | BN2 | CN15 |
| DB16 | AN4 | BN2 | CN16 |
| DB17 | AN4 | BN2 | CN17 |
| DB18 | AN4 | BN2 | CN18 |
| DB19 | AN4 | BN2 | CN19 |
| DB20 | AN4 | BN2 | CN20 |
| DB21 | AN4 | BN2 | CN21 |
| DB22 | AN4 | BN2 | CN22 |
| DB23 | AN4 | BN2 | CN23 |
| DB24 | AN4 | BN2 | CN24 |
| DB25 | AN4 | BN2 | CN25 |
| DB26 | AN4 | BN2 | CN26 |
| DB27 | AN4 | BN2 | CN27 |
| DB28 | AN4 | BN2 | CN28 |
| DB29 | AN4 | BN2 | CN29 |
| DB30 | AN4 | BN2 | CN30 |
| DB31 | AN4 | BN2 | CN31 |
| DB32 | AN4 | BN2 | CN32 |
| DB33 | AN4 | BN2 | CN33 |
| DB34 | AN4 | BN2 | CN34 |
| DB35 | AN4 | BN2 | CN35 |
| DB36 | AN4 | BN2 | CN36 |
| DC1 | AN4 | BN3 | CN1 |
| DC2 | AN4 | BN3 | CN2 |
| DC3 | AN4 | BN3 | CN3 |
| DC4 | AN4 | BN3 | CN4 |
| DC5 | AN4 | BN3 | CN5 |
| DC6 | AN4 | BN3 | CN6 |
| DC7 | AN4 | BN3 | CN7 |
| DC8 | AN4 | BN3 | CN8 |
| DC9 | AN4 | BN3 | CN9 |
| DC10 | AN4 | BN3 | CN10 |
| DC11 | AN4 | BN3 | CN11 |
| DC12 | AN4 | BN3 | CN12 |
| DC13 | AN4 | BN3 | CN13 |
| DC14 | AN4 | BN3 | CN14 |
| DC15 | AN4 | BN3 | CN15 |
| DC16 | AN4 | BN3 | CN16 |
| DC17 | AN4 | BN3 | CN17 |
| DC18 | AN4 | BN3 | CN18 |
| DC19 | AN4 | BN3 | CN19 |
| DC20 | AN4 | BN3 | CN20 |
| DC21 | AN4 | BN3 | CN21 |
| DC22 | AN4 | BN3 | CN22 |
| DC23 | AN4 | BN3 | CN23 |
| DC24 | AN4 | BN3 | CN24 |
| DC25 | AN4 | BN3 | CN25 |
| DC26 | AN4 | BN3 | CN26 |
| DC27 | AN4 | BN3 | CN27 |
| DC28 | AN4 | BN3 | CN28 |
| DC29 | AN4 | BN3 | CN29 |
| DC30 | AN4 | BN3 | CN30 |
| DC31 | AN4 | BN3 | CN31 |
| DC32 | AN4 | BN3 | CN32 |
| DC33 | AN4 | BN3 | CN33 |
| DC34 | AN4 | BN3 | CN34 |
| DC35 | AN4 | BN3 | CN35 |
| DC36 | AN4 | BN3 | CN36 |
| DD1 | AN4 | BN4 | CN1 |
| DD2 | AN4 | BN4 | CN2 |
| DD3 | AN4 | BN4 | CN3 |
| DD4 | AN4 | BN4 | CN4 |
| DD5 | AN4 | BN4 | CN5 |
| DD6 | AN4 | BN4 | CN6 |
| DD7 | AN4 | BN4 | CN7 |
| DD8 | AN4 | BN4 | CN8 |
| DD9 | AN4 | BN4 | CN9 |
| DD10 | AN4 | BN4 | CN10 |
| DD11 | AN4 | BN4 | CN11 |
| DD12 | AN4 | BN4 | CN12 |
| DD13 | AN4 | BN4 | CN13 |
| DD14 | AN4 | BN4 | CN14 |
| DD15 | AN4 | BN4 | CN15 |
| DD16 | AN4 | BN4 | CN16 |
| DD17 | AN4 | BN4 | CN17 |
| DD18 | AN4 | BN4 | CN18 |
| DD19 | AN4 | BN4 | CN19 |
| DD20 | AN4 | BN4 | CN20 |
| DD21 | AN4 | BN4 | CN21 |
| DD22 | AN4 | BN4 | CN22 |
| DD23 | AN4 | BN4 | CN23 |
| DD24 | AN4 | BN4 | CN24 |
| DD25 | AN4 | BN4 | CN25 |
| DD26 | AN4 | BN4 | CN26 |
| DD27 | AN4 | BN4 | CN27 |
| DD28 | AN4 | BN4 | CN28 |
| DD29 | AN4 | BN4 | CN29 |
| DD30 | AN4 | BN4 | CN30 |
| DD31 | AN4 | BN4 | CN31 |
| DD32 | AN4 | BN4 | CN32 |
| DD33 | AN4 | BN4 | CN33 |
| DD34 | AN4 | BN4 | CN34 |
| DD35 | AN4 | BN4 | CN35 |
| DD36 | AN4 | BN4 | CN36 |
| DE1 | AN4 | BN5 | CN1 |
| DE2 | AN4 | BN5 | CN2 |
| DE3 | AN4 | BN5 | CN3 |
| DE4 | AN4 | BN5 | CN4 |
| DE5 | AN4 | BN5 | CN5 |
| DE6 | AN4 | BN5 | CN6 |
| DE7 | AN4 | BN5 | CN7 |
| DE8 | AN4 | BN5 | CN8 |
| DE9 | AN4 | BN5 | CN9 |
| DE10 | AN4 | BN5 | CN10 |
| DE11 | AN4 | BN5 | CN11 |
| DE12 | AN4 | BN5 | CN12 |
| DE13 | AN4 | BN5 | CN13 |
| DE14 | AN4 | BN5 | CN14 |
| DE15 | AN4 | BN5 | CN15 |
| DE16 | AN4 | BN5 | CN16 |
| DE17 | AN4 | BN5 | CN17 |
| DE18 | AN4 | BN5 | CN18 |
| DE19 | AN4 | BN5 | CN19 |
| DE20 | AN4 | BN5 | CN20 |
| DE21 | AN4 | BN5 | CN21 |
| DE22 | AN4 | BN5 | CN22 |
| DE23 | AN4 | BN5 | CN23 |
| DE24 | AN4 | BN5 | CN24 |
| DE25 | AN4 | BN5 | CN25 |
| DE26 | AN4 | BN5 | CN26 |
| DE27 | AN4 | BN5 | CN27 |
| DE28 | AN4 | BN5 | CN28 |
| DE29 | AN4 | BN5 | CN29 |
| DE30 | AN4 | BN5 | CN30 |
| DE31 | AN4 | BN5 | CN31 |
| DE32 | AN4 | BN5 | CN32 |
| DE33 | AN4 | BN5 | CN33 |
| DE34 | AN4 | BN5 | CN34 |
| DE35 | AN4 | BN5 | CN35 |
| DE36 | AN4 | BN5 | CN36 |
| DF1 | AN4 | BN6 | CN1 |
| DF2 | AN4 | BN6 | CN2 |
| DF3 | AN4 | BN6 | CN3 |
| DF4 | AN4 | BN6 | CN4 |
| DF5 | AN4 | BN6 | CN5 |
| DF6 | AN4 | BN6 | CN6 |
| DF7 | AN4 | BN6 | CN7 |
| DF8 | AN4 | BN6 | CN8 |
| DF9 | AN4 | BN6 | CN9 |
| DF10 | AN4 | BN6 | CN10 |
| DF11 | AN4 | BN6 | CN11 |
| DF12 | AN4 | BN6 | CN12 |
| DF13 | AN4 | BN6 | CN13 |
| DF14 | AN4 | BN6 | CN14 |
| DF15 | AN4 | BN6 | CN15 |
| DF16 | AN4 | BN6 | CN16 |
| DF17 | AN4 | BN6 | CN17 |
| DF18 | AN4 | BN6 | CN18 |
| DF19 | AN4 | BN6 | CN19 |
| DF20 | AN4 | BN6 | CN20 |
| DF21 | AN4 | BN6 | CN21 |
| DF22 | AN4 | BN6 | CN22 |
| DF23 | AN4 | BN6 | CN23 |
| DF24 | AN4 | BN6 | CN24 |
| DF25 | AN4 | BN6 | CN25 |
| DF26 | AN4 | BN6 | CN26 |
| DF27 | AN4 | BN6 | CN27 |
| DF28 | AN4 | BN6 | CN28 |
| DF29 | AN4 | BN6 | CN29 |
| DF30 | AN4 | BN6 | CN30 |
| DF31 | AN4 | BN6 | CN31 |
| DF32 | AN4 | BN6 | CN32 |
| DF33 | AN4 | BN6 | CN33 |
| DF34 | AN4 | BN6 | CN34 |
| DF35 | AN4 | BN6 | CN35 |
| DF36 | AN4 | BN6 | CN36 |
| DG1 | AN4 | BN7 | CN1 |
| DG2 | AN4 | BN7 | CN2 |
| DG3 | AN4 | BN7 | CN3 |
| DG4 | AN4 | BN7 | CN4 |
| DG5 | AN4 | BN7 | CN5 |
| DG6 | AN4 | BN7 | CN6 |
| DG7 | AN4 | BN7 | CN7 |
| DG8 | AN4 | BN7 | CN8 |
| DG9 | AN4 | BN7 | CN9 |
| DG10 | AN4 | BN7 | CN10 |
| DG11 | AN4 | BN7 | CN11 |
| DG12 | AN4 | BN7 | CN12 |
| DG13 | AN4 | BN7 | CN13 |
| DG14 | AN4 | BN7 | CN14 |
| DG15 | AN4 | BN7 | CN15 |
| DG16 | AN4 | BN7 | CN16 |
| DG17 | AN4 | BN7 | CN17 |
| DG18 | AN4 | BN7 | CN18 |
| DG19 | AN4 | BN7 | CN19 |
| DG20 | AN4 | BN7 | CN20 |
| DG21 | AN4 | BN7 | CN21 |
| DG22 | AN4 | BN7 | CN22 |
| DG23 | AN4 | BN7 | CN23 |
| DG24 | AN4 | BN7 | CN24 |
| DG25 | AN4 | BN7 | CN25 |
| DG26 | AN4 | BN7 | CN26 |
| DG27 | AN4 | BN7 | CN27 |
| DG28 | AN4 | BN7 | CN28 |
| DG29 | AN4 | BN7 | CN29 |
| DG30 | AN4 | BN7 | CN30 |
| DG31 | AN4 | BN7 | CN31 |
| DG32 | AN4 | BN7 | CN32 |
| DG33 | AN4 | BN7 | CN33 |
| DG34 | AN4 | BN7 | CN34 |
| DG35 | AN4 | BN7 | CN35 |
| DG36 | AN4 | BN7 | CN36 |
| DH1 | AN4 | BN8 | CN1 |
| DH2 | AN4 | BN8 | CN2 |
| DH3 | AN4 | BN8 | CN3 |
| DH4 | AN4 | BN8 | CN4 |
| DH5 | AN4 | BN8 | CN5 |
| DH6 | AN4 | BN8 | CN6 |
| DH7 | AN4 | BN8 | CN7 |
| DH8 | AN4 | BN8 | CN8 |
| DH9 | AN4 | BN8 | CN9 |
| DH10 | AN4 | BN8 | CN10 |
| DH11 | AN4 | BN8 | CN11 |
| DH12 | AN4 | BN8 | CN12 |
| DH13 | AN4 | BN8 | CN13 |
| DH14 | AN4 | BN8 | CN14 |
| DH15 | AN4 | BN8 | CN15 |
| DH16 | AN4 | BN8 | CN16 |
| DH17 | AN4 | BN8 | CN17 |
| DH18 | AN4 | BN8 | CN18 |
| DH19 | AN4 | BN8 | CN19 |
| DH20 | AN4 | BN8 | CN20 |
| DH21 | AN4 | BN8 | CN21 |
| DH22 | AN4 | BN8 | CN22 |
| DH23 | AN4 | BN8 | CN23 |
| DH24 | AN4 | BN8 | CN24 |
| DH25 | AN4 | BN8 | CN25 |
| DH26 | AN4 | BN8 | CN26 |
| DH27 | AN4 | BN8 | CN27 |
| DH28 | AN4 | BN8 | CN28 |
| DH29 | AN4 | BN8 | CN29 |
| DH30 | AN4 | BN8 | CN30 |
| DH31 | AN4 | BN8 | CN31 |
| DH32 | AN4 | BN8 | CN32 |
| DH33 | AN4 | BN8 | CN33 |
| DH34 | AN4 | BN8 | CN34 |
| DH35 | AN4 | BN8 | CN35 |
| DH36 | AN4 | BN8 | CN36 |
| DI1 | AN4 | BN9 | CN1 |
| DI2 | AN4 | BN9 | CN2 |
| DI3 | AN4 | BN9 | CN3 |
| DI4 | AN4 | BN9 | CN4 |
| DI5 | AN4 | BN9 | CN5 |
| DI6 | AN4 | BN9 | CN6 |
| DI7 | AN4 | BN9 | CN7 |
| DI8 | AN4 | BN9 | CN8 |
| DI9 | AN4 | BN9 | CN9 |
| DI10 | AN4 | BN9 | CN10 |
| DI11 | AN4 | BN9 | CN11 |
| DI12 | AN4 | BN9 | CN12 |
| DI13 | AN4 | BN9 | CN13 |
| DI14 | AN4 | BN9 | CN14 |
| DI15 | AN4 | BN9 | CN15 |
| DI16 | AN4 | BN9 | CN16 |
| DI17 | AN4 | BN9 | CN17 |
| DI18 | AN4 | BN9 | CN18 |
| DI19 | AN4 | BN9 | CN19 |
| DI20 | AN4 | BN9 | CN20 |
| DI21 | AN4 | BN9 | CN21 |
| DI22 | AN4 | BN9 | CN22 |
| DI23 | AN4 | BN9 | CN23 |
| DI24 | AN4 | BN9 | CN24 |
| DI25 | AN4 | BN9 | CN25 |
| DI26 | AN4 | BN9 | CN26 |
| DI27 | AN4 | BN9 | CN27 |
| DI28 | AN4 | BN9 | CN28 |
| DI29 | AN4 | BN9 | CN29 |
| DI30 | AN4 | BN9 | CN30 |
| DI31 | AN4 | BN9 | CN31 |
| DI32 | AN4 | BN9 | CN32 |
| DI33 | AN4 | BN9 | CN33 |
| DI34 | AN4 | BN9 | CN34 |
| DI35 | AN4 | BN9 | CN35 |
| DI36 | AN4 | BN9 | CN36 |
| DJ1 | AN4 | BN10 | CN1 |
| DJ2 | AN4 | BN10 | CN2 |
| DJ3 | AN4 | BN10 | CN3 |
| DJ4 | AN4 | BN10 | CN4 |
| DJ5 | AN4 | BN10 | CN5 |
| DJ6 | AN4 | BN10 | CN6 |
| DJ7 | AN4 | BN10 | CN7 |
| DJ8 | AN4 | BN10 | CN8 |
| DJ9 | AN4 | BN10 | CN9 |
| DJ10 | AN4 | BN10 | CN10 |
| DJ11 | AN4 | BN10 | CN11 |
| DJ12 | AN4 | BN10 | CN12 |
| DJ13 | AN4 | BN10 | CN13 |
| DJ14 | AN4 | BN10 | CN14 |
| DJ15 | AN4 | BN10 | CN15 |
| DJ16 | AN4 | BN10 | CN16 |
| DJ17 | AN4 | BN10 | CN17 |
| DJ18 | AN4 | BN10 | CN18 |
| DJ19 | AN4 | BN10 | CN19 |
| DJ20 | AN4 | BN10 | CN20 |
| DJ21 | AN4 | BN10 | CN21 |
| DJ22 | AN4 | BN10 | CN22 |
| DJ23 | AN4 | BN10 | CN23 |
| DJ24 | AN4 | BN10 | CN24 |
| DJ25 | AN4 | BN10 | CN25 |
| DJ26 | AN4 | BN10 | CN26 |
| DJ27 | AN4 | BN10 | CN27 |
| DJ28 | AN4 | BN10 | CN28 |
| DJ29 | AN4 | BN10 | CN29 |
| DJ30 | AN4 | BN10 | CN30 |
| DJ31 | AN4 | BN10 | CN31 |
| DJ32 | AN4 | BN10 | CN32 |
| DJ33 | AN4 | BN10 | CN33 |
| DJ34 | AN4 | BN10 | CN34 |
| DJ35 | AN4 | BN10 | CN35 |
| DJ36 | AN4 | BN10 | CN36 |
| DK1 | AN4 | BN11 | CN1 |
| DK2 | AN4 | BN11 | CN2 |
| DK3 | AN4 | BN11 | CN3 |
| DK4 | AN4 | BN11 | CN4 |
| DK5 | AN4 | BN11 | CN5 |
| DK6 | AN4 | BN11 | CN6 |
| DK7 | AN4 | BN11 | CN7 |
| DK8 | AN4 | BN11 | CN8 |
| DK9 | AN4 | BN11 | CN9 |
| DK10 | AN4 | BN11 | CN10 |
| DK11 | AN4 | BN11 | CN11 |
| DK12 | AN4 | BN11 | CN12 |
| DK13 | AN4 | BN11 | CN13 |
| DK14 | AN4 | BN11 | CN14 |
| DK15 | AN4 | BN11 | CN15 |
| DK16 | AN4 | BN11 | CN16 |
| DK17 | AN4 | BN11 | CN17 |
| DK18 | AN4 | BN11 | CN18 |
| DK19 | AN4 | BN11 | CN19 |
| DK20 | AN4 | BN11 | CN20 |
| DK21 | AN4 | BN11 | CN21 |
| DK22 | AN4 | BN11 | CN22 |
| DK23 | AN4 | BN11 | CN23 |
| DK24 | AN4 | BN11 | CN24 |
| DK25 | AN4 | BN11 | CN25 |
| DK26 | AN4 | BN11 | CN26 |
| DK27 | AN4 | BN11 | CN27 |
| DK28 | AN4 | BN11 | CN28 |
| DK29 | AN4 | BN11 | CN29 |
| DK30 | AN4 | BN11 | CN30 |
| DK31 | AN4 | BN11 | CN31 |
| DK32 | AN4 | BN11 | CN32 |
| DK33 | AN4 | BN11 | CN33 |
| DK34 | AN4 | BN11 | CN34 |
| DK35 | AN4 | BN11 | CN35 |
| DK36 | AN4 | BN11 | CN36 |
| DL1 | AN4 | BN12 | CN1 |
| DL2 | AN4 | BN12 | CN2 |
| DL3 | AN4 | BN12 | CN3 |
| DL4 | AN4 | BN12 | CN4 |
| DL5 | AN4 | BN12 | CN5 |
| DL6 | AN4 | BN12 | CN6 |
| DL7 | AN4 | BN12 | CN7 |
| DL8 | AN4 | BN12 | CN8 |
| DL9 | AN4 | BN12 | CN9 |
| DL10 | AN4 | BN12 | CN10 |
| DL11 | AN4 | BN12 | CN11 |
| DL12 | AN4 | BN12 | CN12 |
| DL13 | AN4 | BN12 | CN13 |
| DL14 | AN4 | BN12 | CN14 |
| DL15 | AN4 | BN12 | CN15 |
| DL16 | AN4 | BN12 | CN16 |
| DL17 | AN4 | BN12 | CN17 |
| DL18 | AN4 | BN12 | CN18 |
| DL19 | AN4 | BN12 | CN19 |
| DL20 | AN4 | BN12 | CN20 |
| DL21 | AN4 | BN12 | CN21 |
| DL22 | AN4 | BN12 | CN22 |
| DL23 | AN4 | BN12 | CN23 |
| DL24 | AN4 | BN12 | CN24 |
| DL25 | AN4 | BN12 | CN25 |
| DL26 | AN4 | BN12 | CN26 |
| DL27 | AN4 | BN12 | CN27 |
| DL28 | AN4 | BN12 | CN28 |
| DL29 | AN4 | BN12 | CN29 |
| DL30 | AN4 | BN12 | CN30 |
| DL31 | AN4 | BN12 | CN31 |
| DL32 | AN4 | BN12 | CN32 |
| DL33 | AN4 | BN12 | CN33 |
| DL34 | AN4 | BN12 | CN34 |
| DL35 | AN4 | BN12 | CN35 |
| DL36 | AN4 | BN12 | CN36 |
| DM1 | AN4 | BN13 | CN1 |
| DM2 | AN4 | BN13 | CN2 |
| DM3 | AN4 | BN13 | CN3 |
| DM4 | AN4 | BN13 | CN4 |
| DM5 | AN4 | BN13 | CN5 |
| DM6 | AN4 | BN13 | CN6 |
| DM7 | AN4 | BN13 | CN7 |
| DM8 | AN4 | BN13 | CN8 |
| DM9 | AN4 | BN13 | CN9 |
| DM10 | AN4 | BN13 | CN10 |
| DM11 | AN4 | BN13 | CN11 |
| DM12 | AN4 | BN13 | CN12 |
| DM13 | AN4 | BN13 | CN13 |
| DM14 | AN4 | BN13 | CN14 |
| DM15 | AN4 | BN13 | CN15 |
| DM16 | AN4 | BN13 | CN16 |
| DM17 | AN4 | BN13 | CN17 |
| DM18 | AN4 | BN13 | CN18 |
| DM19 | AN4 | BN13 | CN19 |
| DM20 | AN4 | BN13 | CN20 |
| DM21 | AN4 | BN13 | CN21 |
| DM22 | AN4 | BN13 | CN22 |
| DM23 | AN4 | BN13 | CN23 |
| DM24 | AN4 | BN13 | CN24 |
| DM25 | AN4 | BN13 | CN25 |
| DM26 | AN4 | BN13 | CN26 |
| DM27 | AN4 | BN13 | CN27 |
| DM28 | AN4 | BN13 | CN28 |
| DM29 | AN4 | BN13 | CN29 |
| DM30 | AN4 | BN13 | CN30 |
| DM31 | AN4 | BN13 | CN31 |
| DM32 | AN4 | BN13 | CN32 |
| DM33 | AN4 | BN13 | CN33 |
| DM34 | AN4 | BN13 | CN34 |
| DM35 | AN4 | BN13 | CN35 |
| DM36 | AN4 | BN13 | CN36 |
| DO1 | AN4 | BN14 | CN1 |
| DO2 | AN4 | BN14 | CN2 |
| DO3 | AN4 | BN14 | CN3 |
| DO4 | AN4 | BN14 | CN4 |
| DO5 | AN4 | BN14 | CN5 |
| DO6 | AN4 | BN14 | CN6 |
| DO7 | AN4 | BN14 | CN7 |
| DO8 | AN4 | BN14 | CN8 |
| DO9 | AN4 | BN14 | CN9 |
| DO10 | AN4 | BN14 | CN10 |
| DO11 | AN4 | BN14 | CN11 |
| DO12 | AN4 | BN14 | CN12 |
| DO13 | AN4 | BN14 | CN13 |
| DO14 | AN4 | BN14 | CN14 |
| DO15 | AN4 | BN14 | CN15 |
| DO16 | AN4 | BN14 | CN16 |
| DO17 | AN4 | BN14 | CN17 |
| DO18 | AN4 | BN14 | CN18 |
| DO19 | AN4 | BN14 | CN19 |
| DO20 | AN4 | BN14 | CN20 |
| DO21 | AN4 | BN14 | CN21 |
| DO22 | AN4 | BN14 | CN22 |
| DO23 | AN4 | BN14 | CN23 |
| DO24 | AN4 | BN14 | CN24 |
| DO25 | AN4 | BN14 | CN25 |
| DO26 | AN4 | BN14 | CN26 |
| DO27 | AN4 | BN14 | CN27 |
| DO28 | AN4 | BN14 | CN28 |
| DO29 | AN4 | BN14 | CN29 |
| DO30 | AN4 | BN14 | CN30 |
| DO31 | AN4 | BN14 | CN31 |
| DO32 | AN4 | BN14 | CN32 |
| DO33 | AN4 | BN14 | CN33 |
| DO34 | AN4 | BN14 | CN34 |
| DO35 | AN4 | BN14 | CN35 |
| DO36 | AN4 | BN14 | CN36 |
| DP1 | AN4 | BN15 | CN1 |
| DP2 | AN4 | BN15 | CN2 |
| DP3 | AN4 | BN15 | CN3 |
| DP4 | AN4 | BN15 | CN4 |
| DP5 | AN4 | BN15 | CN5 |
| DP6 | AN4 | BN15 | CN6 |
| DP7 | AN4 | BN15 | CN7 |
| DP8 | AN4 | BN15 | CN8 |
| DP9 | AN4 | BN15 | CN9 |
| DP10 | AN4 | BN15 | CN10 |
| DP11 | AN4 | BN15 | CN11 |
| DP12 | AN4 | BN15 | CN12 |
| DP13 | AN4 | BN15 | CN13 |
| DP14 | AN4 | BN15 | CN14 |
| DP15 | AN4 | BN15 | CN15 |
| DP16 | AN4 | BN15 | CN16 |
| DP17 | AN4 | BN15 | CN17 |
| DP18 | AN4 | BN15 | CN18 |
| DP19 | AN4 | BN15 | CN19 |
| DP20 | AN4 | BN15 | CN20 |
| DP21 | AN4 | BN15 | CN21 |
| DP22 | AN4 | BN15 | CN22 |
| DP23 | AN4 | BN15 | CN23 |
| DP24 | AN4 | BN15 | CN24 |
| DP25 | AN4 | BN15 | CN25 |
| DP26 | AN4 | BN15 | CN26 |
| DP27 | AN4 | BN15 | CN27 |
| DP28 | AN4 | BN15 | CN28 |
| DP29 | AN4 | BN15 | CN29 |
| DP30 | AN4 | BN15 | CN30 |
| DP31 | AN4 | BN15 | CN31 |
| DP32 | AN4 | BN15 | CN32 |
| DP33 | AN4 | BN15 | CN33 |
| DP34 | AN4 | BN15 | CN34 |
| DP35 | AN4 | BN15 | CN35 |
| DP36 | AN4 | BN15 | CN36 |
| DQ1 | AN4 | BN16 | CN1 |
| DQ2 | AN4 | BN16 | CN2 |
| DQ3 | AN4 | BN16 | CN3 |
| DQ4 | AN4 | BN16 | CN4 |
| DQ5 | AN4 | BN16 | CN5 |
| DQ6 | AN4 | BN16 | CN6 |
| DQ7 | AN4 | BN16 | CN7 |
| DQ8 | AN4 | BN16 | CN8 |
| DQ9 | AN4 | BN16 | CN9 |
| DQ10 | AN4 | BN16 | CN10 |
| DQ11 | AN4 | BN16 | CN11 |
| DQ12 | AN4 | BN16 | CN12 |
| DQ13 | AN4 | BN16 | CN13 |
| DQ14 | AN4 | BN16 | CN14 |
| DQ15 | AN4 | BN16 | CN15 |
| DQ16 | AN4 | BN16 | CN16 |
| DQ17 | AN4 | BN16 | CN17 |
| DQ18 | AN4 | BN16 | CN18 |
| DQ19 | AN4 | BN16 | CN19 |
| DQ20 | AN4 | BN16 | CN20 |
| DQ21 | AN4 | BN16 | CN21 |
| DQ22 | AN4 | BN16 | CN22 |
| DQ23 | AN4 | BN16 | CN23 |
| DQ24 | AN4 | BN16 | CN24 |
| DQ25 | AN4 | BN16 | CN25 |
| DQ26 | AN4 | BN16 | CN26 |
| DQ27 | AN4 | BN16 | CN27 |
| DQ28 | AN4 | BN16 | CN28 |
| DQ29 | AN4 | BN16 | CN29 |
| DQ30 | AN4 | BN16 | CN30 |
| DQ31 | AN4 | BN16 | CN31 |
| DQ32 | AN4 | BN16 | CN32 |
| DQ33 | AN4 | BN16 | CN33 |
| DQ34 | AN4 | BN16 | CN34 |
| DQ35 | AN4 | BN16 | CN35 |
| DQ36 | AN4 | BN16 | CN36 |
| DR1 | AN4 | BN17 | CN1 |
| DR2 | AN4 | BN17 | CN2 |
| DR3 | AN4 | BN17 | CN3 |
| DR4 | AN4 | BN17 | CN4 |
| DR5 | AN4 | BN17 | CN5 |
| DR6 | AN4 | BN17 | CN6 |
| DR7 | AN4 | BN17 | CN7 |
| DR8 | AN4 | BN17 | CN8 |
| DR9 | AN4 | BN17 | CN9 |
| DR10 | AN4 | BN17 | CN10 |
| DR11 | AN4 | BN17 | CN11 |
| DR12 | AN4 | BN17 | CN12 |
| DR13 | AN4 | BN17 | CN13 |
| DR14 | AN4 | BN17 | CN14 |
| DR15 | AN4 | BN17 | CN15 |
| DR16 | AN4 | BN17 | CN16 |
| DR17 | AN4 | BN17 | CN17 |
| DR18 | AN4 | BN17 | CN18 |
| DR19 | AN4 | BN17 | CN19 |
| DR20 | AN4 | BN17 | CN20 |
| DR21 | AN4 | BN17 | CN21 |
| DR22 | AN4 | BN17 | CN22 |
| DR23 | AN4 | BN17 | CN23 |
| DR24 | AN4 | BN17 | CN24 |
| DR25 | AN4 | BN17 | CN25 |
| DR26 | AN4 | BN17 | CN26 |
| DR27 | AN4 | BN17 | CN27 |
| DR28 | AN4 | BN17 | CN28 |
| DR29 | AN4 | BN17 | CN29 |
| DR30 | AN4 | BN17 | CN30 |
| DR31 | AN4 | BN17 | CN31 |
| DR32 | AN4 | BN17 | CN32 |
| DR33 | AN4 | BN17 | CN33 |
| DR34 | AN4 | BN17 | CN34 |
| DR35 | AN4 | BN17 | CN35 |
| DR36 | AN4 | BN17 | CN36 |
| DS1 | AN4 | BN18 | CN1 |
| DS2 | AN4 | BN18 | CN2 |
| DS3 | AN4 | BN18 | CN3 |
| DS4 | AN4 | BN18 | CN4 |
| DS5 | AN4 | BN18 | CN5 |
| DS6 | AN4 | BN18 | CN6 |
| DS7 | AN4 | BN18 | CN7 |
| DS8 | AN4 | BN18 | CN8 |
| DS9 | AN4 | BN18 | CN9 |
| DS10 | AN4 | BN18 | CN10 |
| DS11 | AN4 | BN18 | CN11 |
| DS12 | AN4 | BN18 | CN12 |
| DS13 | AN4 | BN18 | CN13 |
| DS14 | AN4 | BN18 | CN14 |
| DS15 | AN4 | BN18 | CN15 |
| DS16 | AN4 | BN18 | CN16 |
| DS17 | AN4 | BN18 | CN17 |
| DS18 | AN4 | BN18 | CN18 |
| DS19 | AN4 | BN18 | CN19 |
| DS20 | AN4 | BN18 | CN20 |
| DS21 | AN4 | BN18 | CN21 |
| DS22 | AN4 | BN18 | CN22 |
| DS23 | AN4 | BN18 | CN23 |
| DS24 | AN4 | BN18 | CN24 |
| DS25 | AN4 | BN18 | CN25 |
| DS26 | AN4 | BN18 | CN26 |
| DS27 | AN4 | BN18 | CN27 |
| DS28 | AN4 | BN18 | CN28 |
| DS29 | AN4 | BN18 | CN29 |
| DS30 | AN4 | BN18 | CN30 |
| DS31 | AN4 | BN18 | CN31 |
| DS32 | AN4 | BN18 | CN32 |
| DS33 | AN4 | BN18 | CN33 |
| DS34 | AN4 | BN18 | CN34 |
| DS35 | AN4 | BN18 | CN35 |
| DS36 | AN4 | BN18 | CN36 |
| DT1 | AN4 | BN19 | CN1 |
| DT2 | AN4 | BN19 | CN2 |
| DT3 | AN4 | BN19 | CN3 |
| DT4 | AN4 | BN19 | CN4 |
| DT5 | AN4 | BN19 | CN5 |
| DT6 | AN4 | BN19 | CN6 |
| DT7 | AN4 | BN19 | CN7 |
| DT8 | AN4 | BN19 | CN8 |
| DT9 | AN4 | BN19 | CN9 |
| DT10 | AN4 | BN19 | CN10 |
| DT11 | AN4 | BN19 | CN11 |
| DT12 | AN4 | BN19 | CN12 |
| DT13 | AN4 | BN19 | CN13 |
| DT14 | AN4 | BN19 | CN14 |
| DT15 | AN4 | BN19 | CN15 |
| DT16 | AN4 | BN19 | CN16 |
| DT17 | AN4 | BN19 | CN17 |
| DT18 | AN4 | BN19 | CN18 |
| DT19 | AN4 | BN19 | CN19 |
| DT20 | AN4 | BN19 | CN20 |
| DT21 | AN4 | BN19 | CN21 |
| DT22 | AN4 | BN19 | CN22 |
| DT23 | AN4 | BN19 | CN23 |
| DT24 | AN4 | BN19 | CN24 |
| DT25 | AN4 | BN19 | CN25 |
| DT26 | AN4 | BN19 | CN26 |
| DT27 | AN4 | BN19 | CN27 |
| DT28 | AN4 | BN19 | CN28 |
| DT29 | AN4 | BN19 | CN29 |
| DT30 | AN4 | BN19 | CN30 |
| DT31 | AN4 | BN19 | CN31 |
| DT32 | AN4 | BN19 | CN32 |
| DT33 | AN4 | BN19 | CN33 |
| DT34 | AN4 | BN19 | CN34 |
| DT35 | AN4 | BN19 | CN35 |
| DT36 | AN4 | BN19 | CN36 |
| DU1 | AN4 | BN20 | CN1 |
| DU2 | AN4 | BN20 | CN2 |
| DU3 | AN4 | BN20 | CN3 |
| DU4 | AN4 | BN20 | CN4 |
| DU5 | AN4 | BN20 | CN5 |
| DU6 | AN4 | BN20 | CN6 |
| DU7 | AN4 | BN20 | CN7 |
| DU8 | AN4 | BN20 | CN8 |
| DU9 | AN4 | BN20 | CN9 |
| DU10 | AN4 | BN20 | CN10 |
| DU11 | AN4 | BN20 | CN11 |
| DU12 | AN4 | BN20 | CN12 |
| DU13 | AN4 | BN20 | CN13 |
| DU14 | AN4 | BN20 | CN14 |
| DU15 | AN4 | BN20 | CN15 |
| DU16 | AN4 | BN20 | CN16 |
| DU17 | AN4 | BN20 | CN17 |
| DU18 | AN4 | BN20 | CN18 |
| DU19 | AN4 | BN20 | CN19 |
| DU20 | AN4 | BN20 | CN20 |
| DU21 | AN4 | BN20 | CN21 |
| DU22 | AN4 | BN20 | CN22 |
| DU23 | AN4 | BN20 | CN23 |
| DU24 | AN4 | BN20 | CN24 |
| DU25 | AN4 | BN20 | CN25 |
| DU26 | AN4 | BN20 | CN26 |
| DU27 | AN4 | BN20 | CN27 |
| DU28 | AN4 | BN20 | CN28 |
| DU29 | AN4 | BN20 | CN29 |
| DU30 | AN4 | BN20 | CN30 |
| DU31 | AN4 | BN20 | CN31 |
| DU32 | AN4 | BN20 | CN32 |
| DU33 | AN4 | BN20 | CN33 |
| DU34 | AN4 | BN20 | CN34 |
| DU35 | AN4 | BN20 | CN35 |
| DU36 | AN4 | BN20 | CN36 |
| DV1 | AN4 | BN21 | CN1 |
| DV2 | AN4 | BN21 | CN2 |
| DV3 | AN4 | BN21 | CN3 |
| DV4 | AN4 | BN21 | CN4 |
| DV5 | AN4 | BN21 | CN5 |
| DV6 | AN4 | BN21 | CN6 |
| DV7 | AN4 | BN21 | CN7 |
| DV8 | AN4 | BN21 | CN8 |
| DV9 | AN4 | BN21 | CN9 |
| DV10 | AN4 | BN21 | CN10 |
| DV11 | AN4 | BN21 | CN11 |
| DV12 | AN4 | BN21 | CN12 |
| DV13 | AN4 | BN21 | CN13 |
| DV14 | AN4 | BN21 | CN14 |
| DV15 | AN4 | BN21 | CN15 |
| DV16 | AN4 | BN21 | CN16 |
| DV17 | AN4 | BN21 | CN17 |
| DV18 | AN4 | BN21 | CN18 |
| DV19 | AN4 | BN21 | CN19 |
| DV20 | AN4 | BN21 | CN20 |
| DV21 | AN4 | BN21 | CN21 |
| DV22 | AN4 | BN21 | CN22 |
| DV23 | AN4 | BN21 | CN23 |
| DV24 | AN4 | BN21 | CN24 |
| DV25 | AN4 | BN21 | CN25 |
| DV26 | AN4 | BN21 | CN26 |
| DV27 | AN4 | BN21 | CN27 |
| DV28 | AN4 | BN21 | CN28 |
| DV29 | AN4 | BN21 | CN29 |
| DV30 | AN4 | BN21 | CN30 |
| DV31 | AN4 | BN21 | CN31 |
| DV32 | AN4 | BN21 | CN32 |
| DV33 | AN4 | BN21 | CN33 |
| DV34 | AN4 | BN21 | CN34 |
| DV35 | AN4 | BN21 | CN35 |
| DV36 | AN4 | BN21 | CN36 |
| DW1 | AN4 | BN22 | CN1 |
| DW2 | AN4 | BN22 | CN2 |
| DW3 | AN4 | BN22 | CN3 |
| DW4 | AN4 | BN22 | CN4 |
| DW5 | AN4 | BN22 | CN5 |
| DW6 | AN4 | BN22 | CN6 |
| DW7 | AN4 | BN22 | CN7 |
| DW8 | AN4 | BN22 | CN8 |
| DW9 | AN4 | BN22 | CN9 |
| DW10 | AN4 | BN22 | CN10 |
| DW11 | AN4 | BN22 | CN11 |
| DW12 | AN4 | BN22 | CN12 |
| DW13 | AN4 | BN22 | CN13 |
| DW14 | AN4 | BN22 | CN14 |
| DW15 | AN4 | BN22 | CN15 |
| DW16 | AN4 | BN22 | CN16 |
| DW17 | AN4 | BN22 | CN17 |
| DW18 | AN4 | BN22 | CN18 |
| DW19 | AN4 | BN22 | CN19 |
| DW20 | AN4 | BN22 | CN20 |
| DW21 | AN4 | BN22 | CN21 |
| DW22 | AN4 | BN22 | CN22 |
| DW23 | AN4 | BN22 | CN23 |
| DW24 | AN4 | BN22 | CN24 |
| DW25 | AN4 | BN22 | CN25 |
| DW26 | AN4 | BN22 | CN26 |
| DW27 | AN4 | BN22 | CN27 |
| DW28 | AN4 | BN22 | CN28 |
| DW29 | AN4 | BN22 | CN29 |
| DW30 | AN4 | BN22 | CN30 |
| DW31 | AN4 | BN22 | CN31 |
| DW32 | AN4 | BN22 | CN32 |
| DW33 | AN4 | BN22 | CN33 |
| DW34 | AN4 | BN22 | CN34 |
| DW35 | AN4 | BN22 | CN35 |
| DW36 | AN4 | BN22 | CN36 |
| EA1 | AN5 | BN1 | CN1 |
| EA2 | AN5 | BN1 | CN2 |
| EA3 | AN5 | BN1 | CN3 |
| EA4 | AN5 | BN1 | CN4 |
| EA5 | AN5 | BN1 | CN5 |
| EA6 | AN5 | BN1 | CN6 |
| EA7 | AN5 | BN1 | CN7 |
| EA8 | AN5 | BN1 | CN8 |
| EA9 | AN5 | BN1 | CN9 |
| EA10 | AN5 | BN1 | CN10 |
| EA11 | AN5 | BN1 | CN11 |
| EA12 | AN5 | BN1 | CN12 |
| EA13 | AN5 | BN1 | CN13 |
| EA14 | AN5 | BN1 | CN14 |
| EA15 | AN5 | BN1 | CN15 |
| EA16 | AN5 | BN1 | CN16 |
| EA17 | AN5 | BN1 | CN17 |
| EA18 | AN5 | BN1 | CN18 |
| EA19 | AN5 | BN1 | CN19 |
| EA20 | AN5 | BN1 | CN20 |
| EA21 | AN5 | BN1 | CN21 |
| EA22 | AN5 | BN1 | CN22 |
| EA23 | AN5 | BN1 | CN23 |
| EA24 | AN5 | BN1 | CN24 |
| EA25 | AN5 | BN1 | CN25 |
| EA26 | AN5 | BN1 | CN26 |
| EA27 | AN5 | BN1 | CN27 |
| EA28 | AN5 | BN1 | CN28 |
| EA29 | AN5 | BN1 | CN29 |
| EA30 | AN5 | BN1 | CN30 |
| EA31 | AN5 | BN1 | CN31 |
| EA32 | AN5 | BN1 | CN32 |
| EA33 | AN5 | BN1 | CN33 |
| EA34 | AN5 | BN1 | CN34 |
| EA35 | AN5 | BN1 | CN35 |
| EA36 | AN5 | BN1 | CN36 |
| EB1 | AN5 | BN2 | CN1 |
| EB2 | AN5 | BN2 | CN2 |
| EB3 | AN5 | BN2 | CN3 |
| EB4 | AN5 | BN2 | CN4 |
| EB5 | AN5 | BN2 | CN5 |
| EB6 | AN5 | BN2 | CN6 |
| EB7 | AN5 | BN2 | CN7 |
| EB8 | AN5 | BN2 | CN8 |
| EB9 | AN5 | BN2 | CN9 |
| EB10 | AN5 | BN2 | CN10 |
| EB11 | AN5 | BN2 | CN11 |
| EB12 | AN5 | BN2 | CN12 |
| EB13 | AN5 | BN2 | CN13 |
| EB14 | AN5 | BN2 | CN14 |
| EB15 | AN5 | BN2 | CN15 |
| EB16 | AN5 | BN2 | CN16 |
| EB17 | AN5 | BN2 | CN17 |
| EB18 | AN5 | BN2 | CN18 |
| EB19 | AN5 | BN2 | CN19 |
| EB20 | AN5 | BN2 | CN20 |
| EB21 | AN5 | BN2 | CN21 |
| EB22 | AN5 | BN2 | CN22 |
| EB23 | AN5 | BN2 | CN23 |
| EB24 | AN5 | BN2 | CN24 |
| EB25 | AN5 | BN2 | CN25 |
| EB26 | AN5 | BN2 | CN26 |
| EB27 | AN5 | BN2 | CN27 |
| EB28 | AN5 | BN2 | CN28 |
| EB29 | AN5 | BN2 | CN29 |
| EB30 | AN5 | BN2 | CN30 |
| EB31 | AN5 | BN2 | CN31 |
| EB32 | AN5 | BN2 | CN32 |
| EB33 | AN5 | BN2 | CN33 |
| EB34 | AN5 | BN2 | CN34 |
| EB35 | AN5 | BN2 | CN35 |
| EB36 | AN5 | BN2 | CN36 |
| EC1 | AN5 | BN3 | CN1 |
| EC2 | AN5 | BN3 | CN2 |
| EC3 | AN5 | BN3 | CN3 |
| EC4 | AN5 | BN3 | CN4 |
| EC5 | AN5 | BN3 | CN5 |
| EC6 | AN5 | BN3 | CN6 |
| EC7 | AN5 | BN3 | CN7 |
| EC8 | AN5 | BN3 | CN8 |
| EC9 | AN5 | BN3 | CN9 |
| EC10 | AN5 | BN3 | CN10 |
| EC11 | AN5 | BN3 | CN11 |
| EC12 | AN5 | BN3 | CN12 |
| EC13 | AN5 | BN3 | CN13 |
| EC14 | AN5 | BN3 | CN14 |
| EC15 | AN5 | BN3 | CN15 |
| EC16 | AN5 | BN3 | CN16 |
| EC17 | AN5 | BN3 | CN17 |
| EC18 | AN5 | BN3 | CN18 |
| EC19 | AN5 | BN3 | CN19 |
| EC20 | AN5 | BN3 | CN20 |
| EC21 | AN5 | BN3 | CN21 |
| EC22 | AN5 | BN3 | CN22 |
| EC23 | AN5 | BN3 | CN23 |
| EC24 | AN5 | BN3 | CN24 |
| EC25 | AN5 | BN3 | CN25 |
| EC26 | AN5 | BN3 | CN26 |
| EC27 | AN5 | BN3 | CN27 |
| EC28 | AN5 | BN3 | CN28 |
| EC29 | AN5 | BN3 | CN29 |
| EC30 | AN5 | BN3 | CN30 |
| EC31 | AN5 | BN3 | CN31 |
| EC32 | AN5 | BN3 | CN32 |
| EC33 | AN5 | BN3 | CN33 |
| EC34 | AN5 | BN3 | CN34 |
| EC35 | AN5 | BN3 | CN35 |
| EC36 | AN5 | BN3 | CN36 |
| ED1 | AN5 | BN4 | CN1 |
| ED2 | AN5 | BN4 | CN2 |
| ED3 | AN5 | BN4 | CN3 |
| ED4 | AN5 | BN4 | CN4 |
| ED5 | AN5 | BN4 | CN5 |
| ED6 | AN5 | BN4 | CN6 |
| ED7 | AN5 | BN4 | CN7 |
| ED8 | AN5 | BN4 | CN8 |
| ED9 | AN5 | BN4 | CN9 |
| ED10 | AN5 | BN4 | CN10 |
| ED11 | AN5 | BN4 | CN11 |
| ED12 | AN5 | BN4 | CN12 |
| ED13 | AN5 | BN4 | CN13 |
| ED14 | AN5 | BN4 | CN14 |
| ED15 | AN5 | BN4 | CN15 |
| ED16 | AN5 | BN4 | CN16 |
| ED17 | AN5 | BN4 | CN17 |
| ED18 | AN5 | BN4 | CN18 |
| ED19 | AN5 | BN4 | CN19 |
| ED20 | AN5 | BN4 | CN20 |
| ED21 | AN5 | BN4 | CN21 |
| ED22 | AN5 | BN4 | CN22 |
| ED23 | AN5 | BN4 | CN23 |
| ED24 | AN5 | BN4 | CN24 |
| ED25 | AN5 | BN4 | CN25 |
| ED26 | AN5 | BN4 | CN26 |
| ED27 | AN5 | BN4 | CN27 |
| ED28 | AN5 | BN4 | CN28 |
| ED29 | AN5 | BN4 | CN29 |
| ED30 | AN5 | BN4 | CN30 |
| ED31 | AN5 | BN4 | CN31 |
| ED32 | AN5 | BN4 | CN32 |
| ED33 | AN5 | BN4 | CN33 |
| ED34 | AN5 | BN4 | CN34 |
| ED35 | AN5 | BN4 | CN35 |
| ED36 | AN5 | BN4 | CN36 |
| EE1 | AN5 | BN5 | CN1 |
| EE2 | AN5 | BN5 | CN2 |
| EE3 | AN5 | BN5 | CN3 |
| EE4 | AN5 | BN5 | CN4 |
| EE5 | AN5 | BN5 | CN5 |
| EE6 | AN5 | BN5 | CN6 |
| EE7 | AN5 | BN5 | CN7 |
| EE8 | AN5 | BN5 | CN8 |
| EE9 | AN5 | BN5 | CN9 |
| EE10 | AN5 | BN5 | CN10 |
| EE11 | AN5 | BN5 | CN11 |
| EE12 | AN5 | BN5 | CN12 |
| EE13 | AN5 | BN5 | CN13 |
| EE14 | AN5 | BN5 | CN14 |
| EE15 | AN5 | BN5 | CN15 |
| EE16 | AN5 | BN5 | CN16 |
| EE17 | AN5 | BN5 | CN17 |
| EE18 | AN5 | BN5 | CN18 |
| EE19 | AN5 | BN5 | CN19 |
| EE20 | AN5 | BN5 | CN20 |
| EE21 | AN5 | BN5 | CN21 |
| EE22 | AN5 | BN5 | CN22 |
| EE23 | AN5 | BN5 | CN23 |
| EE24 | AN5 | BN5 | CN24 |
| EE25 | AN5 | BN5 | CN25 |
| EE26 | AN5 | BN5 | CN26 |
| EE27 | AN5 | BN5 | CN27 |
| EE28 | AN5 | BN5 | CN28 |
| EE29 | AN5 | BN5 | CN29 |
| EE30 | AN5 | BN5 | CN30 |
| EE31 | AN5 | BN5 | CN31 |
| EE32 | AN5 | BN5 | CN32 |
| EE33 | AN5 | BN5 | CN33 |
| EE34 | AN5 | BN5 | CN34 |
| EE35 | AN5 | BN5 | CN35 |
| EE36 | AN5 | BN5 | CN36 |
| EF1 | AN5 | BN6 | CN1 |
| EF2 | AN5 | BN6 | CN2 |
| EF3 | AN5 | BN6 | CN3 |
| EF4 | AN5 | BN6 | CN4 |
| EF5 | AN5 | BN6 | CN5 |
| EF6 | AN5 | BN6 | CN6 |
| EF7 | AN5 | BN6 | CN7 |
| EF8 | AN5 | BN6 | CN8 |
| EF9 | AN5 | BN6 | CN9 |
| EF10 | AN5 | BN6 | CN10 |
| EF11 | AN5 | BN6 | CN11 |
| EF12 | AN5 | BN6 | CN12 |
| EF13 | AN5 | BN6 | CN13 |
| EF14 | AN5 | BN6 | CN14 |
| EF15 | AN5 | BN6 | CN15 |
| EF16 | AN5 | BN6 | CN16 |
| EF17 | AN5 | BN6 | CN17 |
| EF18 | AN5 | BN6 | CN18 |
| EF19 | AN5 | BN6 | CN19 |
| EF20 | AN5 | BN6 | CN20 |
| EF21 | AN5 | BN6 | CN21 |
| EF22 | AN5 | BN6 | CN22 |
| EF23 | AN5 | BN6 | CN23 |
| EF24 | AN5 | BN6 | CN24 |
| EF25 | AN5 | BN6 | CN25 |
| EF26 | AN5 | BN6 | CN26 |
| EF27 | AN5 | BN6 | CN27 |
| EF28 | AN5 | BN6 | CN28 |
| EF29 | AN5 | BN6 | CN29 |
| EF30 | AN5 | BN6 | CN30 |
| EF31 | AN5 | BN6 | CN31 |
| EF32 | AN5 | BN6 | CN32 |
| EF33 | AN5 | BN6 | CN33 |
| EF34 | AN5 | BN6 | CN34 |
| EF35 | AN5 | BN6 | CN35 |
| EF36 | AN5 | BN6 | CN36 |
| EG1 | AN5 | BN7 | CN1 |
| EG2 | AN5 | BN7 | CN2 |
| EG3 | AN5 | BN7 | CN3 |
| EG4 | AN5 | BN7 | CN4 |
| EG5 | AN5 | BN7 | CN5 |
| EG6 | AN5 | BN7 | CN6 |
| EG7 | AN5 | BN7 | CN7 |
| EG8 | AN5 | BN7 | CN8 |
| EG9 | AN5 | BN7 | CN9 |
| EG10 | AN5 | BN7 | CN10 |
| EG11 | AN5 | BN7 | CN11 |
| EG12 | AN5 | BN7 | CN12 |
| EG13 | AN5 | BN7 | CN13 |
| EG14 | AN5 | BN7 | CN14 |
| EG15 | AN5 | BN7 | CN15 |
| EG16 | AN5 | BN7 | CN16 |
| EG17 | AN5 | BN7 | CN17 |
| EG18 | AN5 | BN7 | CN18 |
| EG19 | AN5 | BN7 | CN19 |
| EG20 | AN5 | BN7 | CN20 |
| EG21 | AN5 | BN7 | CN21 |
| EG22 | AN5 | BN7 | CN22 |
| EG23 | AN5 | BN7 | CN23 |
| EG24 | AN5 | BN7 | CN24 |
| EG25 | AN5 | BN7 | CN25 |
| EG26 | AN5 | BN7 | CN26 |
| EG27 | AN5 | BN7 | CN27 |
| EG28 | AN5 | BN7 | CN28 |
| EG29 | AN5 | BN7 | CN29 |
| EG30 | AN5 | BN7 | CN30 |
| EG31 | AN5 | BN7 | CN31 |
| EG32 | AN5 | BN7 | CN32 |
| EG33 | AN5 | BN7 | CN33 |
| EG34 | AN5 | BN7 | CN34 |
| EG35 | AN5 | BN7 | CN35 |
| EG36 | AN5 | BN7 | CN36 |
| EH1 | AN5 | BN8 | CN1 |
| EH2 | AN5 | BN8 | CN2 |
| EH3 | AN5 | BN8 | CN3 |
| EH4 | AN5 | BN8 | CN4 |
| EH5 | AN5 | BN8 | CN5 |
| EH6 | AN5 | BN8 | CN6 |
| EH7 | AN5 | BN8 | CN7 |
| EH8 | AN5 | BN8 | CN8 |
| EH9 | AN5 | BN8 | CN9 |
| EH10 | AN5 | BN8 | CN10 |
| EH11 | AN5 | BN8 | CN11 |
| EH12 | AN5 | BN8 | CN12 |
| EH13 | AN5 | BN8 | CN13 |
| EH14 | AN5 | BN8 | CN14 |
| EH15 | AN5 | BN8 | CN15 |
| EH16 | AN5 | BN8 | CN16 |
| EH17 | AN5 | BN8 | CN17 |
| EH18 | AN5 | BN8 | CN18 |
| EH19 | AN5 | BN8 | CN19 |
| EH20 | AN5 | BN8 | CN20 |
| EH21 | AN5 | BN8 | CN21 |
| EH22 | AN5 | BN8 | CN22 |
| EH23 | AN5 | BN8 | CN23 |
| EH24 | AN5 | BN8 | CN24 |
| EH25 | AN5 | BN8 | CN25 |
| EH26 | AN5 | BN8 | CN26 |
| EH27 | AN5 | BN8 | CN27 |
| EH28 | AN5 | BN8 | CN28 |
| EH29 | AN5 | BN8 | CN29 |
| EH30 | AN5 | BN8 | CN30 |
| EH31 | AN5 | BN8 | CN31 |
| EH32 | AN5 | BN8 | CN32 |
| EH33 | AN5 | BN8 | CN33 |
| EH34 | AN5 | BN8 | CN34 |
| EH35 | AN5 | BN8 | CN35 |
| EH36 | AN5 | BN8 | CN36 |
| EI1 | AN5 | BN9 | CN1 |
| EI2 | AN5 | BN9 | CN2 |
| EI3 | AN5 | BN9 | CN3 |
| EI4 | AN5 | BN9 | CN4 |
| EI5 | AN5 | BN9 | CN5 |
| EI6 | AN5 | BN9 | CN6 |
| EI7 | AN5 | BN9 | CN7 |
| EI8 | AN5 | BN9 | CN8 |
| EI9 | AN5 | BN9 | CN9 |
| EI10 | AN5 | BN9 | CN10 |
| EI11 | AN5 | BN9 | CN11 |
| EI12 | AN5 | BN9 | CN12 |
| EI13 | AN5 | BN9 | CN13 |
| EI14 | AN5 | BN9 | CN14 |
| EI15 | AN5 | BN9 | CN15 |
| EI16 | AN5 | BN9 | CN16 |
| EI17 | AN5 | BN9 | CN17 |
| EI18 | AN5 | BN9 | CN18 |
| EI19 | AN5 | BN9 | CN19 |
| EI20 | AN5 | BN9 | CN20 |
| EI21 | AN5 | BN9 | CN21 |
| EI22 | AN5 | BN9 | CN22 |
| EI23 | AN5 | BN9 | CN23 |
| EI24 | AN5 | BN9 | CN24 |
| EI25 | AN5 | BN9 | CN25 |
| EI26 | AN5 | BN9 | CN26 |
| EI27 | AN5 | BN9 | CN27 |
| EI28 | AN5 | BN9 | CN28 |
| EI29 | AN5 | BN9 | CN29 |
| EI30 | AN5 | BN9 | CN30 |
| EI31 | AN5 | BN9 | CN31 |
| EI32 | AN5 | BN9 | CN32 |
| EI33 | AN5 | BN9 | CN33 |
| EI34 | AN5 | BN9 | CN34 |
| EI35 | AN5 | BN9 | CN35 |
| EI36 | AN5 | BN9 | CN36 |
| EJ1 | AN5 | BN10 | CN1 |
| EJ2 | AN5 | BN10 | CN2 |
| EJ3 | AN5 | BN10 | CN3 |
| EJ4 | AN5 | BN10 | CN4 |
| EJ5 | AN5 | BN10 | CN5 |
| EJ6 | AN5 | BN10 | CN6 |
| EJ7 | AN5 | BN10 | CN7 |
| EJ8 | AN5 | BN10 | CN8 |
| EJ9 | AN5 | BN10 | CN9 |
| EJ10 | AN5 | BN10 | CN10 |
| EJ11 | AN5 | BN10 | CN11 |
| EJ12 | AN5 | BN10 | CN12 |
| EJ13 | AN5 | BN10 | CN13 |
| EJ14 | AN5 | BN10 | CN14 |
| EJ15 | AN5 | BN10 | CN15 |
| EJ16 | AN5 | BN10 | CN16 |
| EJ17 | AN5 | BN10 | CN17 |
| EJ18 | AN5 | BN10 | CN18 |
| EJ19 | AN5 | BN10 | CN19 |
| EJ20 | AN5 | BN10 | CN20 |
| EJ21 | AN5 | BN10 | CN21 |
| EJ22 | AN5 | BN10 | CN22 |
| EJ23 | AN5 | BN10 | CN23 |
| EJ24 | AN5 | BN10 | CN24 |
| EJ25 | AN5 | BN10 | CN25 |
| EJ26 | AN5 | BN10 | CN26 |
| EJ27 | AN5 | BN10 | CN27 |
| EJ28 | AN5 | BN10 | CN28 |
| EJ29 | AN5 | BN10 | CN29 |
| EJ30 | AN5 | BN10 | CN30 |
| EJ31 | AN5 | BN10 | CN31 |
| EJ32 | AN5 | BN10 | CN32 |
| EJ33 | AN5 | BN10 | CN33 |
| EJ34 | AN5 | BN10 | CN34 |
| EJ35 | AN5 | BN10 | CN35 |
| EJ36 | AN5 | BN10 | CN36 |
| EK1 | AN5 | BN11 | CN1 |
| EK2 | AN5 | BN11 | CN2 |
| EK3 | AN5 | BN11 | CN3 |
| EK4 | AN5 | BN11 | CN4 |
| EK5 | AN5 | BN11 | CN5 |
| EK6 | AN5 | BN11 | CN6 |
| EK7 | AN5 | BN11 | CN7 |
| EK8 | AN5 | BN11 | CN8 |
| EK9 | AN5 | BN11 | CN9 |
| EK10 | AN5 | BN11 | CN10 |
| EK11 | AN5 | BN11 | CN11 |
| EK12 | AN5 | BN11 | CN12 |
| EK13 | AN5 | BN11 | CN13 |
| EK14 | AN5 | BN11 | CN14 |
| EK15 | AN5 | BN11 | CN15 |
| EK16 | AN5 | BN11 | CN16 |
| EK17 | AN5 | BN11 | CN17 |
| EK18 | AN5 | BN11 | CN18 |
| EK19 | AN5 | BN11 | CN19 |
| EK20 | AN5 | BN11 | CN20 |
| EK21 | AN5 | BN11 | CN21 |
| EK22 | AN5 | BN11 | CN22 |
| EK23 | AN5 | BN11 | CN23 |
| EK24 | AN5 | BN11 | CN24 |
| EK25 | AN5 | BN11 | CN25 |
| EK26 | AN5 | BN11 | CN26 |
| EK27 | AN5 | BN11 | CN27 |
| EK28 | AN5 | BN11 | CN28 |
| EK29 | AN5 | BN11 | CN29 |
| EK30 | AN5 | BN11 | CN30 |
| EK31 | AN5 | BN11 | CN31 |
| EK32 | AN5 | BN11 | CN32 |
| EK33 | AN5 | BN11 | CN33 |
| EK34 | AN5 | BN11 | CN34 |
| EK35 | AN5 | BN11 | CN35 |
| EK36 | AN5 | BN11 | CN36 |
| EL1 | AN5 | BN12 | CN1 |
| EL2 | AN5 | BN12 | CN2 |
| EL3 | AN5 | BN12 | CN3 |
| EL4 | AN5 | BN12 | CN4 |
| EL5 | AN5 | BN12 | CN5 |
| EL6 | AN5 | BN12 | CN6 |
| EL7 | AN5 | BN12 | CN7 |
| EL8 | AN5 | BN12 | CN8 |
| EL9 | AN5 | BN12 | CN9 |
| EL10 | AN5 | BN12 | CN10 |
| EL11 | AN5 | BN12 | CN11 |
| EL12 | AN5 | BN12 | CN12 |
| EL13 | AN5 | BN12 | CN13 |
| EL14 | AN5 | BN12 | CN14 |
| EL15 | AN5 | BN12 | CN15 |
| EL16 | AN5 | BN12 | CN16 |
| EL17 | AN5 | BN12 | CN17 |
| EL18 | AN5 | BN12 | CN18 |
| EL19 | AN5 | BN12 | CN19 |
| EL20 | AN5 | BN12 | CN20 |
| EL21 | AN5 | BN12 | CN21 |
| EL22 | AN5 | BN12 | CN22 |
| EL23 | AN5 | BN12 | CN23 |
| EL24 | AN5 | BN12 | CN24 |
| EL25 | AN5 | BN12 | CN25 |
| EL26 | AN5 | BN12 | CN26 |
| EL27 | AN5 | BN12 | CN27 |
| EL28 | AN5 | BN12 | CN28 |
| EL29 | AN5 | BN12 | CN29 |
| EL30 | AN5 | BN12 | CN30 |
| EL31 | AN5 | BN12 | CN31 |
| EL32 | AN5 | BN12 | CN32 |
| EL33 | AN5 | BN12 | CN33 |
| EL34 | AN5 | BN12 | CN34 |
| EL35 | AN5 | BN12 | CN35 |
| EL36 | AN5 | BN12 | CN36 |
| EM1 | AN5 | BN13 | CN1 |
| EM2 | AN5 | BN13 | CN2 |
| EM3 | AN5 | BN13 | CN3 |
| EM4 | AN5 | BN13 | CN4 |
| EM5 | AN5 | BN13 | CN5 |
| EM6 | AN5 | BN13 | CN6 |
| EM7 | AN5 | BN13 | CN7 |
| EM8 | AN5 | BN13 | CN8 |
| EM9 | AN5 | BN13 | CN9 |
| EM10 | AN5 | BN13 | CN10 |
| EM11 | AN5 | BN13 | CN11 |
| EM12 | AN5 | BN13 | CN12 |
| EM13 | AN5 | BN13 | CN13 |
| EM14 | AN5 | BN13 | CN14 |
| EM15 | AN5 | BN13 | CN15 |
| EM16 | AN5 | BN13 | CN16 |
| EM17 | AN5 | BN13 | CN17 |
| EM18 | AN5 | BN13 | CN18 |
| EM19 | AN5 | BN13 | CN19 |
| EM20 | AN5 | BN13 | CN20 |
| EM21 | AN5 | BN13 | CN21 |
| EM22 | AN5 | BN13 | CN22 |
| EM23 | AN5 | BN13 | CN23 |
| EM24 | AN5 | BN13 | CN24 |
| EM25 | AN5 | BN13 | CN25 |
| EM26 | AN5 | BN13 | CN26 |
| EM27 | AN5 | BN13 | CN27 |
| EM28 | AN5 | BN13 | CN28 |
| EM29 | AN5 | BN13 | CN29 |
| EM30 | AN5 | BN13 | CN30 |
| EM31 | AN5 | BN13 | CN31 |
| EM32 | AN5 | BN13 | CN32 |
| EM33 | AN5 | BN13 | CN33 |
| EM34 | AN5 | BN13 | CN34 |
| EM35 | AN5 | BN13 | CN35 |
| EM36 | AN5 | BN13 | CN36 |
| EO1 | AN5 | BN14 | CN1 |
| EO2 | AN5 | BN14 | CN2 |
| EO3 | AN5 | BN14 | CN3 |
| EO4 | AN5 | BN14 | CN4 |
| EO5 | AN5 | BN14 | CN5 |
| EO6 | AN5 | BN14 | CN6 |
| EO7 | AN5 | BN14 | CN7 |
| EO8 | AN5 | BN14 | CN8 |
| EO9 | AN5 | BN14 | CN9 |
| EO10 | AN5 | BN14 | CN10 |
| EO11 | AN5 | BN14 | CN11 |
| EO12 | AN5 | BN14 | CN12 |
| EO13 | AN5 | BN14 | CN13 |
| EO14 | AN5 | BN14 | CN14 |
| EO15 | AN5 | BN14 | CN15 |
| EO16 | AN5 | BN14 | CN16 |
| EO17 | AN5 | BN14 | CN17 |
| EO18 | AN5 | BN14 | CN18 |
| EO19 | AN5 | BN14 | CN19 |
| EO20 | AN5 | BN14 | CN20 |
| EO21 | AN5 | BN14 | CN21 |
| EO22 | AN5 | BN14 | CN22 |
| EO23 | AN5 | BN14 | CN23 |
| EO24 | AN5 | BN14 | CN24 |
| EO25 | AN5 | BN14 | CN25 |
| EO26 | AN5 | BN14 | CN26 |
| EO27 | AN5 | BN14 | CN27 |
| EO28 | AN5 | BN14 | CN28 |
| EO29 | AN5 | BN14 | CN29 |
| EO30 | AN5 | BN14 | CN30 |
| EO31 | AN5 | BN14 | CN31 |
| EO32 | AN5 | BN14 | CN32 |
| EO33 | AN5 | BN14 | CN33 |
| EO34 | AN5 | BN14 | CN34 |
| EO35 | AN5 | BN14 | CN35 |
| EO36 | AN5 | BN14 | CN36 |
| EP1 | AN5 | BN15 | CN1 |
| EP2 | AN5 | BN15 | CN2 |
| EP3 | AN5 | BN15 | CN3 |
| EP4 | AN5 | BN15 | CN4 |
| EP5 | AN5 | BN15 | CN5 |
| EP6 | AN5 | BN15 | CN6 |
| EP7 | AN5 | BN15 | CN7 |
| EP8 | AN5 | BN15 | CN8 |
| EP9 | AN5 | BN15 | CN9 |
| EP10 | AN5 | BN15 | CN10 |
| EP11 | AN5 | BN15 | CN11 |
| EP12 | AN5 | BN15 | CN12 |
| EP13 | AN5 | BN15 | CN13 |
| EP14 | AN5 | BN15 | CN14 |
| EP15 | AN5 | BN15 | CN15 |
| EP16 | AN5 | BN15 | CN16 |
| EP17 | AN5 | BN15 | CN17 |
| EP18 | AN5 | BN15 | CN18 |
| EP19 | AN5 | BN15 | CN19 |
| EP20 | AN5 | BN15 | CN20 |
| EP21 | AN5 | BN15 | CN21 |
| EP22 | AN5 | BN15 | CN22 |
| EP23 | AN5 | BN15 | CN23 |
| EP24 | AN5 | BN15 | CN24 |
| EP25 | AN5 | BN15 | CN25 |
| EP26 | AN5 | BN15 | CN26 |
| EP27 | AN5 | BN15 | CN27 |
| EP28 | AN5 | BN15 | CN28 |
| EP29 | AN5 | BN15 | CN29 |
| EP30 | AN5 | BN15 | CN30 |
| EP31 | AN5 | BN15 | CN31 |
| EP32 | AN5 | BN15 | CN32 |
| EP33 | AN5 | BN15 | CN33 |
| EP34 | AN5 | BN15 | CN34 |
| EP35 | AN5 | BN15 | CN35 |
| EP36 | AN5 | BN15 | CN36 |
| EQ1 | AN5 | BN16 | CN1 |
| EQ2 | AN5 | BN16 | CN2 |
| EQ3 | AN5 | BN16 | CN3 |
| EQ4 | AN5 | BN16 | CN4 |
| EQ5 | AN5 | BN16 | CN5 |
| EQ6 | AN5 | BN16 | CN6 |
| EQ7 | AN5 | BN16 | CN7 |
| EQ8 | AN5 | BN16 | CN8 |
| EQ9 | AN5 | BN16 | CN9 |
| EQ10 | AN5 | BN16 | CN10 |
| EQ11 | AN5 | BN16 | CN11 |
| EQ12 | AN5 | BN16 | CN12 |
| EQ13 | AN5 | BN16 | CN13 |
| EQ14 | AN5 | BN16 | CN14 |
| EQ15 | AN5 | BN16 | CN15 |
| EQ16 | AN5 | BN16 | CN16 |
| EQ17 | AN5 | BN16 | CN17 |
| EQ18 | AN5 | BN16 | CN18 |
| EQ19 | AN5 | BN16 | CN19 |
| EQ20 | AN5 | BN16 | CN20 |
| EQ21 | AN5 | BN16 | CN21 |
| EQ22 | AN5 | BN16 | CN22 |
| EQ23 | AN5 | BN16 | CN23 |
| EQ24 | AN5 | BN16 | CN24 |
| EQ25 | AN5 | BN16 | CN25 |
| EQ26 | AN5 | BN16 | CN26 |
| EQ27 | AN5 | BN16 | CN27 |
| EQ28 | AN5 | BN16 | CN28 |
| EQ29 | AN5 | BN16 | CN29 |
| EQ30 | AN5 | BN16 | CN30 |
| EQ31 | AN5 | BN16 | CN31 |
| EQ32 | AN5 | BN16 | CN32 |
| EQ33 | AN5 | BN16 | CN33 |
| EQ34 | AN5 | BN16 | CN34 |
| EQ35 | AN5 | BN16 | CN35 |
| EQ36 | AN5 | BN16 | CN36 |
| ER1 | AN5 | BN17 | CN1 |
| ER2 | AN5 | BN17 | CN2 |
| ER3 | AN5 | BN17 | CN3 |
| ER4 | AN5 | BN17 | CN4 |
| ER5 | AN5 | BN17 | CN5 |
| ER6 | AN5 | BN17 | CN6 |
| ER7 | AN5 | BN17 | CN7 |
| ER8 | AN5 | BN17 | CN8 |
| ER9 | AN5 | BN17 | CN9 |
| ER10 | AN5 | BN17 | CN10 |
| ER11 | AN5 | BN17 | CN11 |
| ER12 | AN5 | BN17 | CN12 |
| ER13 | AN5 | BN17 | CN13 |
| ER14 | AN5 | BN17 | CN14 |
| ER15 | AN5 | BN17 | CN15 |
| ER16 | AN5 | BN17 | CN16 |
| ER17 | AN5 | BN17 | CN17 |
| ER18 | AN5 | BN17 | CN18 |
| ER19 | AN5 | BN17 | CN19 |
| ER20 | AN5 | BN17 | CN20 |
| ER21 | AN5 | BN17 | CN21 |
| ER22 | AN5 | BN17 | CN22 |
| ER23 | AN5 | BN17 | CN23 |
| ER24 | AN5 | BN17 | CN24 |
| ER25 | AN5 | BN17 | CN25 |
| ER26 | AN5 | BN17 | CN26 |
| ER27 | AN5 | BN17 | CN27 |
| ER28 | AN5 | BN17 | CN28 |
| ER29 | AN5 | BN17 | CN29 |
| ER30 | AN5 | BN17 | CN30 |
| ER31 | AN5 | BN17 | CN31 |
| ER32 | AN5 | BN17 | CN32 |
| ER33 | AN5 | BN17 | CN33 |
| ER34 | AN5 | BN17 | CN34 |
| ER35 | AN5 | BN17 | CN35 |
| ER36 | AN5 | BN17 | CN36 |
| ES1 | AN5 | BN18 | CN1 |
| ES2 | AN5 | BN18 | CN2 |
| ES3 | AN5 | BN18 | CN3 |
| ES4 | AN5 | BN18 | CN4 |
| ES5 | AN5 | BN18 | CN5 |
| ES6 | AN5 | BN18 | CN6 |
| ES7 | AN5 | BN18 | CN7 |
| ES8 | AN5 | BN18 | CN8 |
| ES9 | AN5 | BN18 | CN9 |
| ES10 | AN5 | BN18 | CN10 |
| ES11 | AN5 | BN18 | CN11 |
| ES12 | AN5 | BN18 | CN12 |
| ES13 | AN5 | BN18 | CN13 |
| ES14 | AN5 | BN18 | CN14 |
| ES15 | AN5 | BN18 | CN15 |
| ES16 | AN5 | BN18 | CN16 |
| ES17 | AN5 | BN18 | CN17 |
| ES18 | AN5 | BN18 | CN18 |
| ES19 | AN5 | BN18 | CN19 |
| ES20 | AN5 | BN18 | CN20 |
| ES21 | AN5 | BN18 | CN21 |
| ES22 | AN5 | BN18 | CN22 |
| ES23 | AN5 | BN18 | CN23 |
| ES24 | AN5 | BN18 | CN24 |
| ES25 | AN5 | BN18 | CN25 |
| ES26 | AN5 | BN18 | CN26 |
| ES27 | AN5 | BN18 | CN27 |
| ES28 | AN5 | BN18 | CN28 |
| ES29 | AN5 | BN18 | CN29 |
| ES30 | AN5 | BN18 | CN30 |
| ES31 | AN5 | BN18 | CN31 |
| ES32 | AN5 | BN18 | CN32 |
| ES33 | AN5 | BN18 | CN33 |
| ES34 | AN5 | BN18 | CN34 |
| ES35 | AN5 | BN18 | CN35 |
| ES36 | AN5 | BN18 | CN36 |
| ET1 | AN5 | BN19 | CN1 |
| ET2 | AN5 | BN19 | CN2 |
| ET3 | AN5 | BN19 | CN3 |
| ET4 | AN5 | BN19 | CN4 |
| ET5 | AN5 | BN19 | CN5 |
| ET6 | AN5 | BN19 | CN6 |
| ET7 | AN5 | BN19 | CN7 |
| ET8 | AN5 | BN19 | CN8 |
| ET9 | AN5 | BN19 | CN9 |
| ET10 | AN5 | BN19 | CN10 |
| ET11 | AN5 | BN19 | CN11 |
| ET12 | AN5 | BN19 | CN12 |
| ET13 | AN5 | BN19 | CN13 |
| ET14 | AN5 | BN19 | CN14 |
| ET15 | AN5 | BN19 | CN15 |
| ET16 | AN5 | BN19 | CN16 |
| ET17 | AN5 | BN19 | CN17 |
| ET18 | AN5 | BN19 | CN18 |
| ET19 | AN5 | BN19 | CN19 |
| ET20 | AN5 | BN19 | CN20 |
| ET21 | AN5 | BN19 | CN21 |
| ET22 | AN5 | BN19 | CN22 |
| ET23 | AN5 | BN19 | CN23 |
| ET24 | AN5 | BN19 | CN24 |
| ET25 | AN5 | BN19 | CN25 |
| ET26 | AN5 | BN19 | CN26 |
| ET27 | AN5 | BN19 | CN27 |
| ET28 | AN5 | BN19 | CN28 |
| ET29 | AN5 | BN19 | CN29 |
| ET30 | AN5 | BN19 | CN30 |
| ET31 | AN5 | BN19 | CN31 |
| ET32 | AN5 | BN19 | CN32 |
| ET33 | AN5 | BN19 | CN33 |
| ET34 | AN5 | BN19 | CN34 |
| ET35 | AN5 | BN19 | CN35 |
| ET36 | AN5 | BN19 | CN36 |
| EU1 | AN5 | BN20 | CN1 |
| EU2 | AN5 | BN20 | CN2 |
| EU3 | AN5 | BN20 | CN3 |
| EU4 | AN5 | BN20 | CN4 |
| EU5 | AN5 | BN20 | CN5 |
| EU6 | AN5 | BN20 | CN6 |
| EU7 | AN5 | BN20 | CN7 |
| EU8 | AN5 | BN20 | CN8 |
| EU9 | AN5 | BN20 | CN9 |
| EU10 | AN5 | BN20 | CN10 |
| EU11 | AN5 | BN20 | CN11 |
| EU12 | AN5 | BN20 | CN12 |
| EU13 | AN5 | BN20 | CN13 |
| EU14 | AN5 | BN20 | CN14 |
| EU15 | AN5 | BN20 | CN15 |
| EU16 | AN5 | BN20 | CN16 |
| EU17 | AN5 | BN20 | CN17 |
| EU18 | AN5 | BN20 | CN18 |
| EU19 | AN5 | BN20 | CN19 |
| EU20 | AN5 | BN20 | CN20 |
| EU21 | AN5 | BN20 | CN21 |
| EU22 | AN5 | BN20 | CN22 |
| EU23 | AN5 | BN20 | CN23 |
| EU24 | AN5 | BN20 | CN24 |
| EU25 | AN5 | BN20 | CN25 |
| EU26 | AN5 | BN20 | CN26 |
| EU27 | AN5 | BN20 | CN27 |
| EU28 | AN5 | BN20 | CN28 |
| EU29 | AN5 | BN20 | CN29 |
| EU30 | AN5 | BN20 | CN30 |
| EU31 | AN5 | BN20 | CN31 |
| EU32 | AN5 | BN20 | CN32 |
| EU33 | AN5 | BN20 | CN33 |
| EU34 | AN5 | BN20 | CN34 |
| EU35 | AN5 | BN20 | CN35 |
| EU36 | AN5 | BN20 | CN36 |
| EV1 | AN5 | BN21 | CN1 |
| EV2 | AN5 | BN21 | CN2 |
| EV3 | AN5 | BN21 | CN3 |
| EV4 | AN5 | BN21 | CN4 |
| EV5 | AN5 | BN21 | CN5 |
| EV6 | AN5 | BN21 | CN6 |
| EV7 | AN5 | BN21 | CN7 |
| EV8 | AN5 | BN21 | CN8 |
| EV9 | AN5 | BN21 | CN9 |
| EV10 | AN5 | BN21 | CN10 |
| EV11 | AN5 | BN21 | CN11 |
| EV12 | AN5 | BN21 | CN12 |
| EV13 | AN5 | BN21 | CN13 |
| EV14 | AN5 | BN21 | CN14 |
| EV15 | AN5 | BN21 | CN15 |
| EV16 | AN5 | BN21 | CN16 |
| EV17 | AN5 | BN21 | CN17 |
| EV18 | AN5 | BN21 | CN18 |
| EV19 | AN5 | BN21 | CN19 |
| EV20 | AN5 | BN21 | CN20 |
| EV21 | AN5 | BN21 | CN21 |
| EV22 | AN5 | BN21 | CN22 |
| EV23 | AN5 | BN21 | CN23 |
| EV24 | AN5 | BN21 | CN24 |
| EV25 | AN5 | BN21 | CN25 |
| EV26 | AN5 | BN21 | CN26 |
| EV27 | AN5 | BN21 | CN27 |
| EV28 | AN5 | BN21 | CN28 |
| EV29 | AN5 | BN21 | CN29 |
| EV30 | AN5 | BN21 | CN30 |
| EV31 | AN5 | BN21 | CN31 |
| EV32 | AN5 | BN21 | CN32 |
| EV33 | AN5 | BN21 | CN33 |
| EV34 | AN5 | BN21 | CN34 |
| EV35 | AN5 | BN21 | CN35 |
| EV36 | AN5 | BN21 | CN36 |
| EW1 | AN5 | BN22 | CN1 |
| EW2 | AN5 | BN22 | CN2 |
| EW3 | AN5 | BN22 | CN3 |
| EW4 | AN5 | BN22 | CN4 |
| EW5 | AN5 | BN22 | CN5 |
| EW6 | AN5 | BN22 | CN6 |
| EW7 | AN5 | BN22 | CN7 |
| EW8 | AN5 | BN22 | CN8 |
| EW9 | AN5 | BN22 | CN9 |
| EW10 | AN5 | BN22 | CN10 |
| EW11 | AN5 | BN22 | CN11 |
| EW12 | AN5 | BN22 | CN12 |
| EW13 | AN5 | BN22 | CN13 |
| EW14 | AN5 | BN22 | CN14 |
| EW15 | AN5 | BN22 | CN15 |
| EW16 | AN5 | BN22 | CN16 |
| EW17 | AN5 | BN22 | CN17 |
| EW18 | AN5 | BN22 | CN18 |
| EW19 | AN5 | BN22 | CN19 |
| EW20 | AN5 | BN22 | CN20 |
| EW21 | AN5 | BN22 | CN21 |
| EW22 | AN5 | BN22 | CN22 |
| EW23 | AN5 | BN22 | CN23 |
| EW24 | AN5 | BN22 | CN24 |
| EW25 | AN5 | BN22 | CN25 |
| EW26 | AN5 | BN22 | CN26 |
| EW27 | AN5 | BN22 | CN27 |
| EW28 | AN5 | BN22 | CN28 |
| EW29 | AN5 | BN22 | CN29 |
| EW30 | AN5 | BN22 | CN30 |
| EW31 | AN5 | BN22 | CN31 |
| EW32 | AN5 | BN22 | CN32 |
| EW33 | AN5 | BN22 | CN33 |
| EW34 | AN5 | BN22 | CN34 |
| EW35 | AN5 | BN22 | CN35 |
| EW36 | AN5 | BN22 | CN36. |

The light emitting element ED of an embodiment may include at least one selected from among the compounds illustrated in Compound Combination Table 1 in the hole transport region HTR. The amine compound of an embodiment includes the first substituent, the second substituent, and the third substituent, which are directly or indirectly connected with the nitrogen atom of the amine, and may accomplish the long lifetime of the light emitting element.

For example, in one or more embodiments, the amine compound of an embodiment essentially includes the second substituent including an α-naphthyl moiety in which an aryl group is connected at position 2 of the α-naphthyl moiety, and the third substituent including a β-naphthyl moiety. The amine compound of an embodiment has improved orientation through the intermolecular interaction between two types (kinds) of naphthyl groups at different connection positions with the amine moiety, and may show excellent or suitable electrical stability and high charge transport capacity. Accordingly, when the amine compound of an embodiment is applied to a light emitting element, element lifetime may be improved.

In the light emitting element ED of one or more embodiments, the hole transport region HTR may further include a compound represented by Formula H-1.

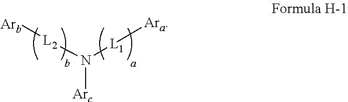

In Formula H-1, L₁ and L₂ may each independently be a direct linkage, a substituted or unsubstituted arylene group having 6 to 30 ring-forming carbon atoms, or a substituted or unsubstituted heteroarylene group having 2 to 30 ring-forming carbon atoms. a and b may each independently be an integer of 0 to 10. In some embodiments, when a or b is an integer of 2 or greater, a plurality of L₁'s and L₂'s may each independently be a substituted or unsubstituted arylene group having 6 to 30 ring-forming carbon atoms, or a substituted or unsubstituted heteroarylene group having 2 to 30 ring-forming carbon atoms.

In Formula H-1, Arₐ and Ar_{b} may each independently be a substituted or unsubstituted aryl group having 6 to 30 ring-forming carbon atoms, or a substituted or unsubstituted heteroaryl group having 2 to 30 ring-forming carbon atoms. In some embodiments, in Formula H-1, Ar_{c} may be a substituted or unsubstituted aryl group having 6 to 30 ring-forming carbon atoms, or a substituted or unsubstituted heteroaryl group having 2 to 30 ring-forming carbon atoms.

The compound represented by Formula H-1 may be a monoamine compound. In some embodiments, the compound represented by Formula H-2 may be a diamine compound in which at least one selected from among Arₐ to Ar_{c} includes an amine group as a substituent. In some embodiments, the compound represented by Formula H-1 may be a carbazole-based compound including a substituted or unsubstituted carbazole group in at least one of Arₐ or Ar_{b}, or a fluorene-based compound including a substituted or unsubstituted fluorene group in at least one of Arₐ or Ar_{b}.

The compound represented by Formula H-1 may be represented by any one selected from among compounds in Compound Group H. However, the compounds listed in Compound Group H are mere examples, and the compounds represented by Formula H-1 are not limited to those represented by Compound Group H:

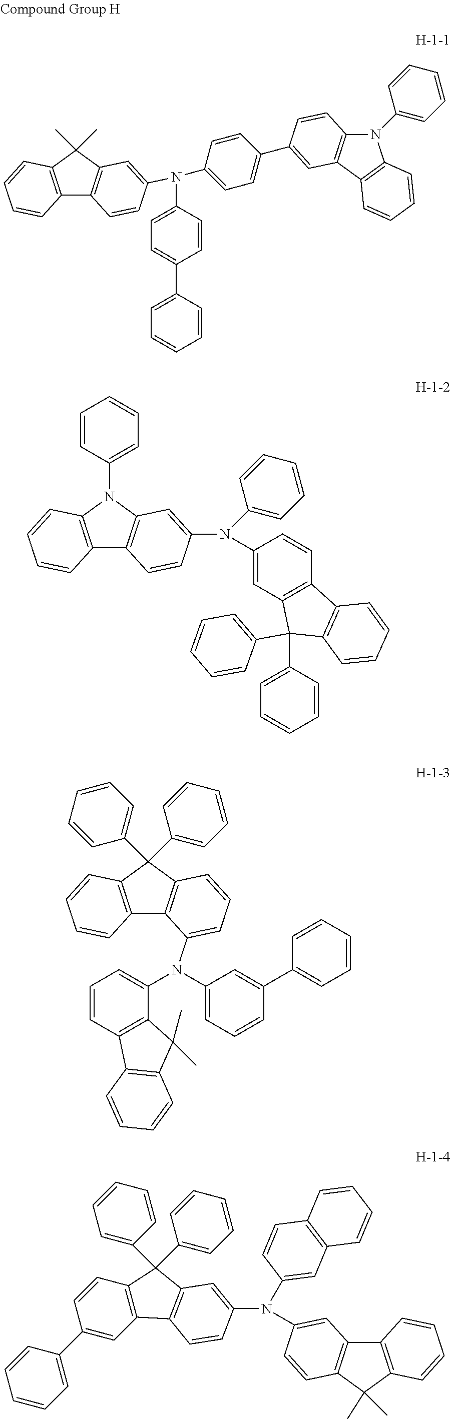 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21 ; CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21]

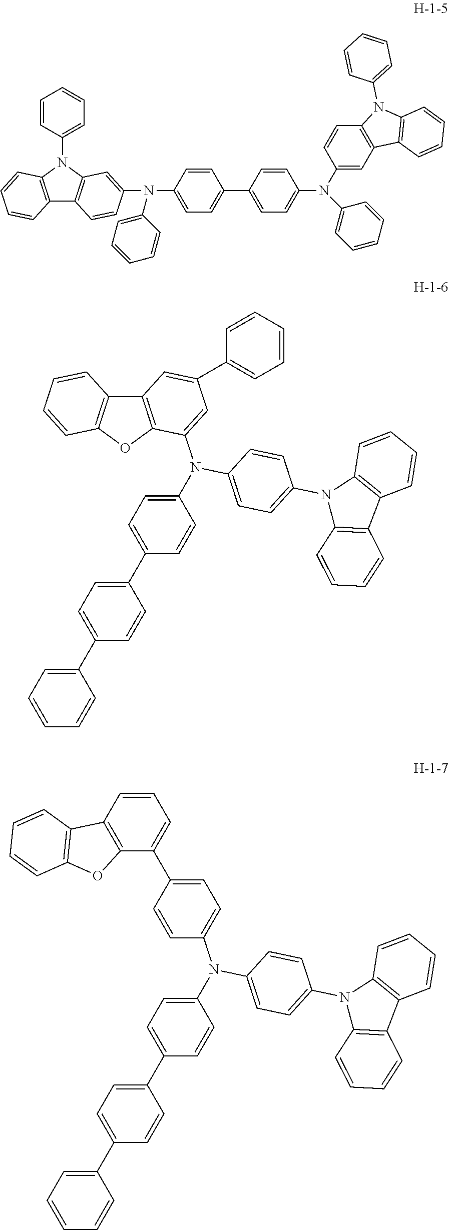 [SMILES: c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1]

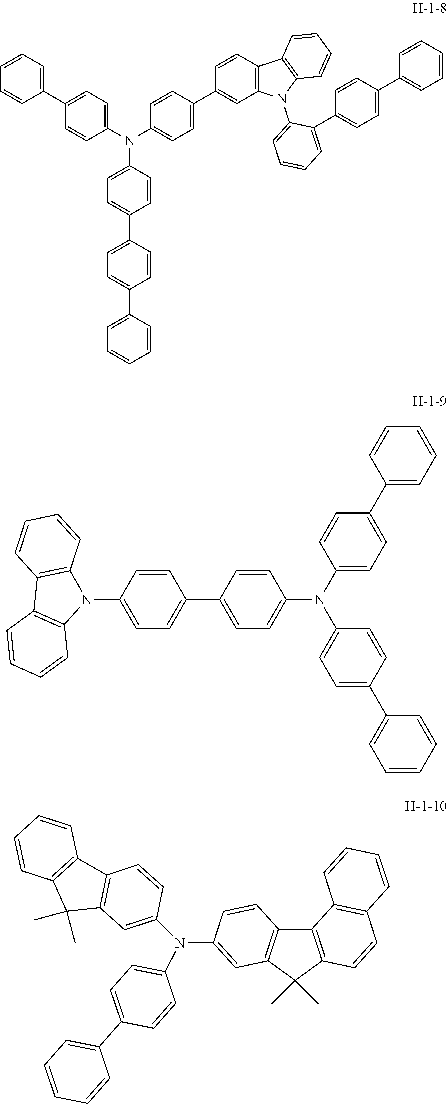 [SMILES: c1ccc(-c2ccc(-c3ccc(N(c4ccc(-c5ccccc5)cc4)c4ccc(-c5ccc6c7ccccc7n(-c7ccccc7-c7ccc(-c8ccccc8)cc7)c6c5)cc4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2ccccc2-c2ccc(N(c3ccc(-c4ccccc4)cc3)c3ccc4c(c3)C(C)(C)c3ccc5ccccc5c3-4)cc21]

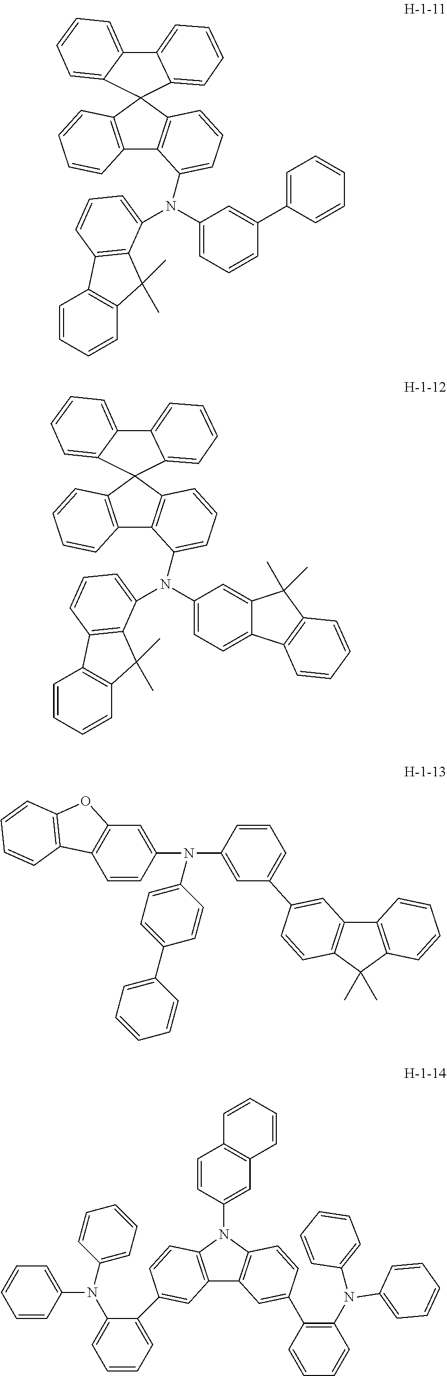

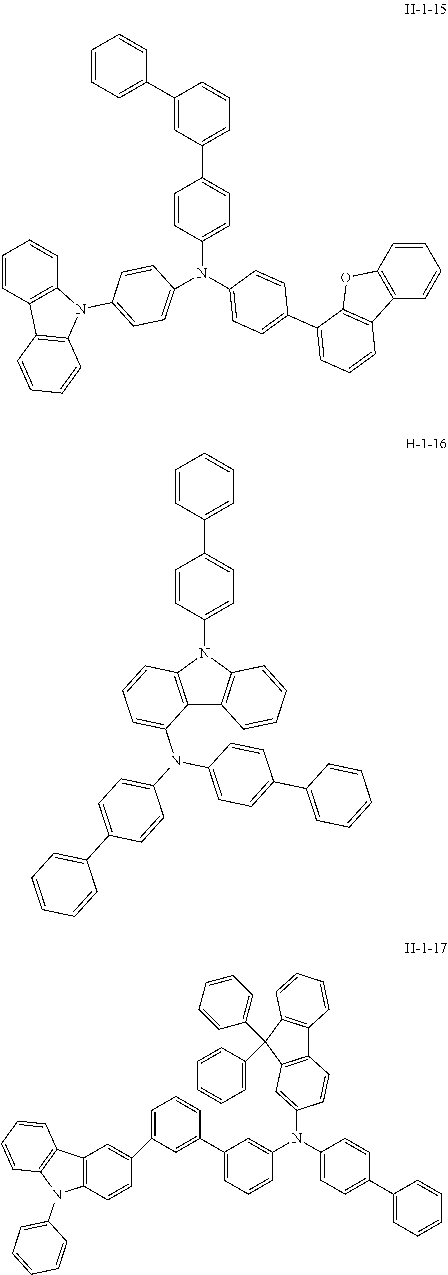 [SMILES: c1ccc(-c2cccc(-c3ccc(N(c4ccc(-c5cccc6c5oc5ccccc56)cc4)c4ccc(-n5c6ccccc6c6ccccc65)cc4)cc3)c2)cc1 ; c1ccc(-c2ccc(N(c3ccc(-c4ccccc4)cc3)c3cccc4c3c3ccccc3n4-c3ccc(-c4ccccc4)cc3)cc2)cc1 ; c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1]

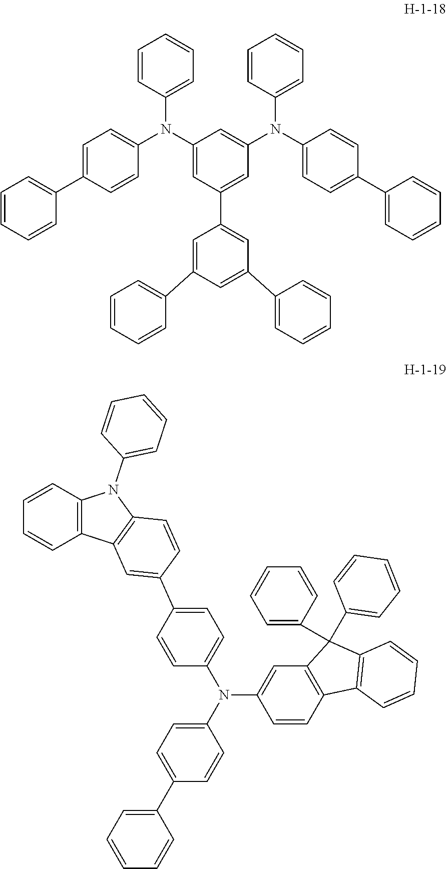 [SMILES: c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

In one or more embodiments, the hole transport region HTR may include a phthalocyanine compound such as copper phthalocyanine; N¹,N^{1′}-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino] triphenylamine (m-MTDATA), 4,4′,4″-tris(N,N-diphenylamino)triphenylamine (TDATA), 4,4′,4″-tris[N-(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(naphthalen-1-yl)-N,N′-diphenyl-benzidine (NPB), triphenylamine-containing polyetherketone (TPAPEK), 4-isopropyl-4′-methyldiphenyliodonium [tetrakis(pentafluorophenyl)borate], dipyrazino[2,3-f: 2′,3′-h]quinoxaline-2,3,6,7,10,11-hexacarbonitrile (HATCN), etc.

In one or more embodiments, the hole transport region HTR may include a carbazole-based derivative such as N-phenyl carbazole or polyvinyl carbazole, a fluorene-based derivative, a triphenylamine-based derivative such as N, N′-bis(3-methylphenyl)-N,N′-diphenyl-[1,1-biphenyl]-4,4′-diamine (TPD) or 4,4′,4″-tris(N-carbazolyl)triphenylamine (TCTA), N,N′-di(naphthalen-1-yl)-N, N′-diphenyl-benzidine (NPB), 4,4′-cyclohexylidene bis[N,N-bis(4-methylphenyl]benzenamine] (TAPC), 4,4′-bis[N, N′-(3-tolyl)amino]-3,3′-dimethylbiphenyl (HMTPD), 1,3-bis(N-carbazolyl)benzene (mCP), etc.

In some embodiments, the hole transport region HTR may include 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), etc.

The hole transport region HTR may include at least one selected from the above-described compounds of the hole transport region in at least one of the hole injection layer HIL, the hole transport layer HTL, or the electron blocking layer EBL.

A thickness of the hole transport region HTR may be from about 100 Å to about 10,000 Å, for example, from about 100 Å to about 5,000 Å. When the hole transport region HTR includes a hole injection layer HIL, the hole injection layer HIL may have, for example, a thickness of about 30 Å to about 1,000 Å. When the hole transport region HTR includes a hole transport layer HTL, the hole transport layer HTL may have a thickness of about 250 Å to about 1,000 Å. For example, when the hole transport region HTR includes an electron blocking layer EBL, the electron blocking layer EBL may have a thickness of about 10 Å to about 1,000 Å. When the thicknesses of the hole transport region HTR, the hole injection layer HIL, the hole transport layer HTL, and the electron blocking layer EBL satisfy the above-described ranges, satisfactory hole transport properties may be achieved without a substantial increase in driving voltage.

The hole transport region HTR may further include a charge generating material to increase conductivity in addition to the above-described materials. The charge generating material may be dispersed substantially uniformly or non-uniformly in the hole transport region HTR. The charge generating material may be, for example, a p-dopant. The p-dopant may include at least one of a halogenated metal compound, a quinone derivative, a metal oxide, or a cyano group-containing compound, but embodiments of the present disclosure are not limited thereto. For example, in one or more embodiments, the p-dopant may include a metal halide compound such as CuI or RbI, a quinone derivative such as tetracyanoquinodimethane (TCNQ) or 2,3,5,6-tetrafluoro-7,7,8,8-tetracyanoquinodimethane (F4-TCNQ), a metal oxide such as tungsten oxide or molybdenum oxide, a cyano group-containing compound such as dipyrazino[2,3-f: 2′,3′-h] quinoxaline-2,3,6,7,10,11-hexacarbonitrile (HATCN) or 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), etc., but embodiments of the present disclosure are not limited thereto.

As described above, in some embodiments, the hole transport region HTR may further include at least one of a buffer layer or an electron blocking layer EBL in addition to the hole injection layer HIL and the hole transport layer HTL. The buffer layer may compensate for a resonance distance according to the wavelength of light emitted from the emission layer EML and may thus increase light emission efficiency. A material that may be included in the hole transport region HTR may be utilized as a material to be included in the buffer layer. The electron blocking layer EBL may be a layer that serves to prevent or reduce the electron injection from the electron transport region ETR to the hole transport region HTR.

The emission layer EML may be provided on the hole transport region HTR. The emission layer EML may have a thickness of, for example, about 100 Å to about 1,000 Å or about 100 Å to about 300 Å. The emission layer EML may have a single layer formed of a single material, a single layer formed of a plurality of different materials, or a multilayer structure having a plurality of layers formed of a plurality of different materials.

In the light emitting element ED of an embodiment, the emission layer EML may be to emit blue light. The light emitting element ED of an embodiment may include the amine compound of one or more embodiments in the hole transport region HTR and may show high efficiency and long-life characteristics in a blue emission region. However, embodiments of the present disclosure are not limited thereto.

In one or more embodiments, in the light emitting element ED of an embodiment, the emission layer EML may include an anthracene derivative, a pyrene derivative, a fluoranthene derivative, a chrysene derivative, a dehydrobenzanthracene derivative, and/or a triphenylene derivative. For example, in some embodiments, the emission layer EML may include the anthracene derivative or the pyrene derivative.

In each light emitting element ED of one or more embodiments illustrated in FIGS. 3 to 6, the emission layer EML may further include a suitable host and dopant. For example, in some embodiments, the emission layer EML may include a compound represented by Formula E-1. The compound represented by Formula E-1 may be utilized as a fluorescent host material.

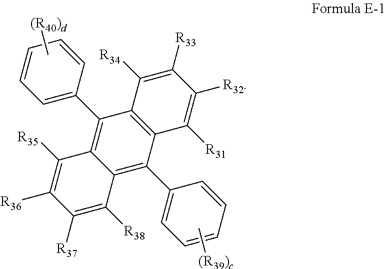

In Formula E-1, R₃₁ to R₄ may each independently be hydrogen, deuterium, a halogen, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, and/or may be bonded to an adjacent group to form a ring. In some embodiments, one or more selected from among R₃₁ to R₄₀ may be bonded to an adjacent group to form a saturated hydrocarbon ring or an unsaturated hydrocarbon ring, a saturated heterocycle, or an unsaturated heterocycle.

In Formula E-1, c and d may each independently be an integer of 0 to 5.

The compound represented by Formula E-1 may be any one selected from among Compound E1 to Compound E19:

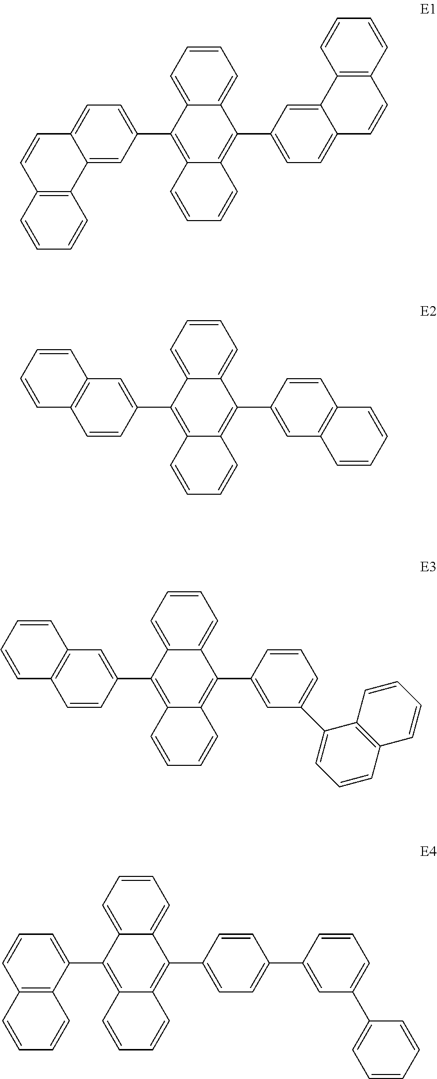

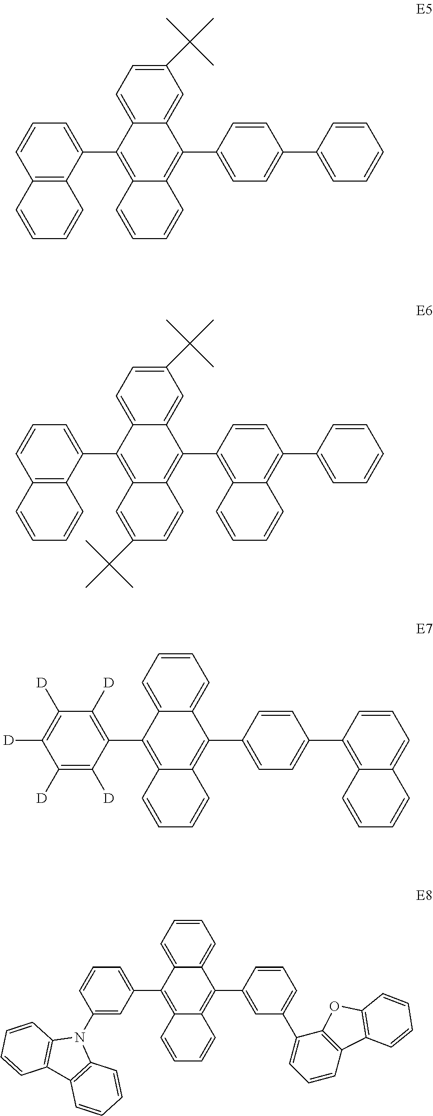

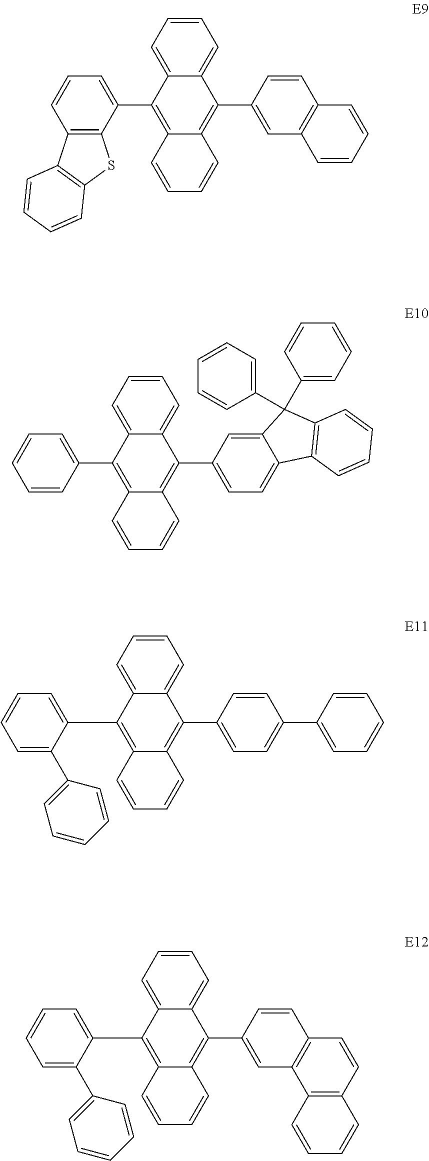

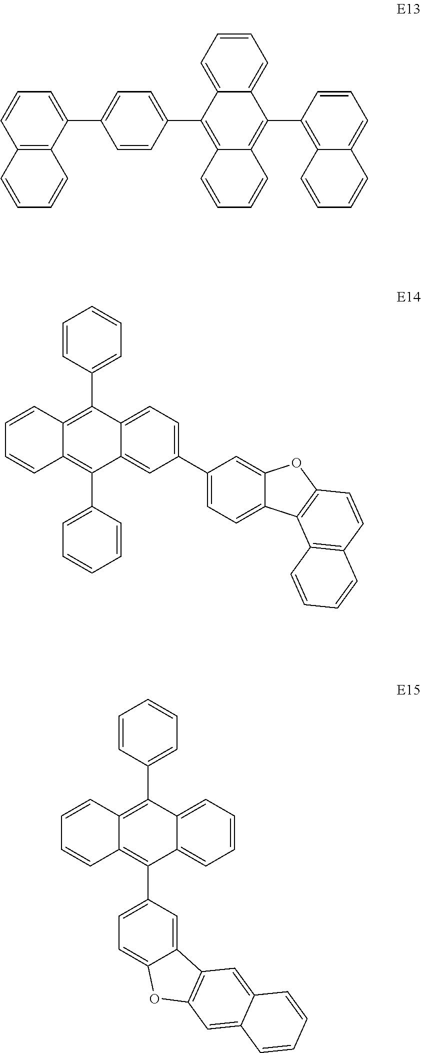

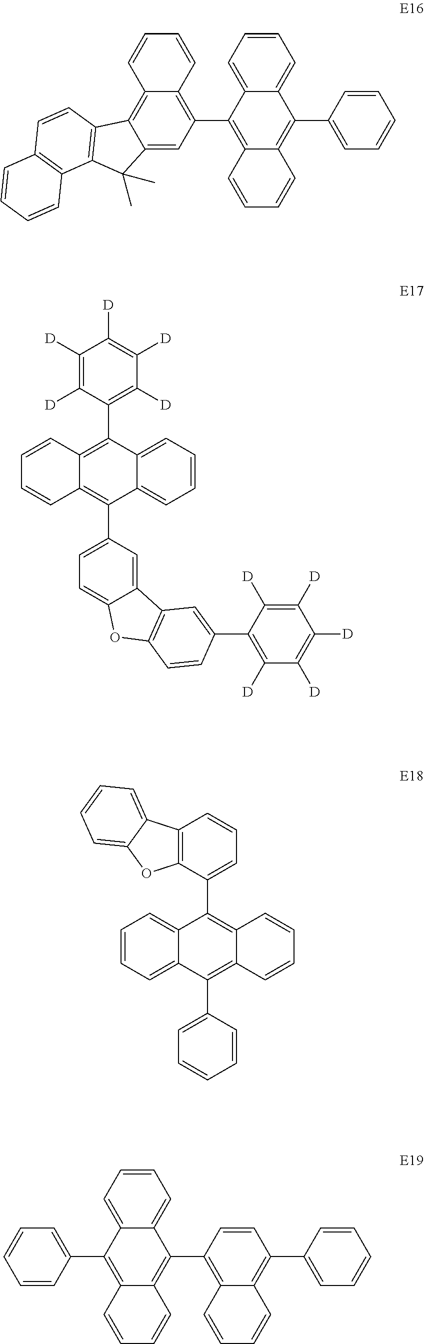

In one or more embodiments, the emission layer EML may include a compound represented by Formula E-2a or Formula E-2b. The compound represented by Formula E-2a or Formula E-2b may be utilized as a phosphorescent host material.

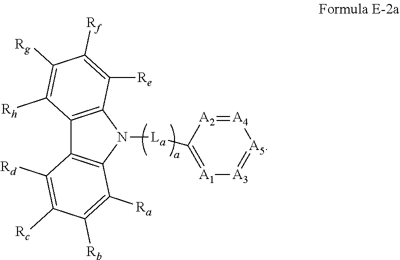

In Formula E-2a, a may be an integer of 0 to 10, and La may be a direct linkage, a substituted or unsubstituted arylene group having 6 to 30 ring-forming carbon atoms, or a substituted or unsubstituted heteroarylene group having 2 to 30 ring-forming carbon atoms. In some embodiments, when a is an integer of 2 or greater, a plurality of La's may each independently be a substituted or unsubstituted arylene group having 6 to 30 ring-forming carbon atoms, or a substituted or unsubstituted heteroarylene group having 2 to 30 ring-forming carbon atoms.

In some embodiments, in Formula E-2a, A₁ to A₅ may each independently be N or CRᵢ. Rₐ to Rᵢ may each independently be hydrogen, deuterium, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or may be bonded to an adjacent group to form a ring. In some embodiments, one or more selected from among Rₐ to Rᵢ may be bonded to an adjacent group to form a hydrocarbon ring or a heterocycle containing N, O, S, etc., as a ring-forming atom.

In some embodiments, in Formula E-2a, two or three selected from among A₁ to A₅ may be N, and the rest may be CRᵢ.

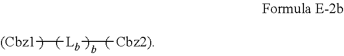

In Formula E-2b, Cbz1 and Cbz2 may each independently be an unsubstituted carbazole group, or a carbazole group substituted with an aryl group having 6 to 30 ring-forming carbon atoms. L_{b} is a direct linkage, a substituted or unsubstituted arylene group having 6 to 30 ring-forming carbon atoms, or a substituted or unsubstituted heteroarylene group having 2 to 30 ring-forming carbon atoms. Here, b is an integer of 0 to 10, and when b is an integer of 2 or more, a plurality of L_{b}'s may each independently be a substituted or unsubstituted arylene group having 6 to 30 ring-forming carbon atoms, or a substituted or unsubstituted heteroarylene group having 2 to 30 ring-forming carbon atoms.

The compound represented by Formula E-2a or Formula E-2b may be any one selected from among the compounds of Compound Group E-2. However, the compounds listed in Compound Group E-2 are mere examples, and the compound represented by Formula E-2a or Formula E-2b is not limited to those represented in Compound Group E-2.

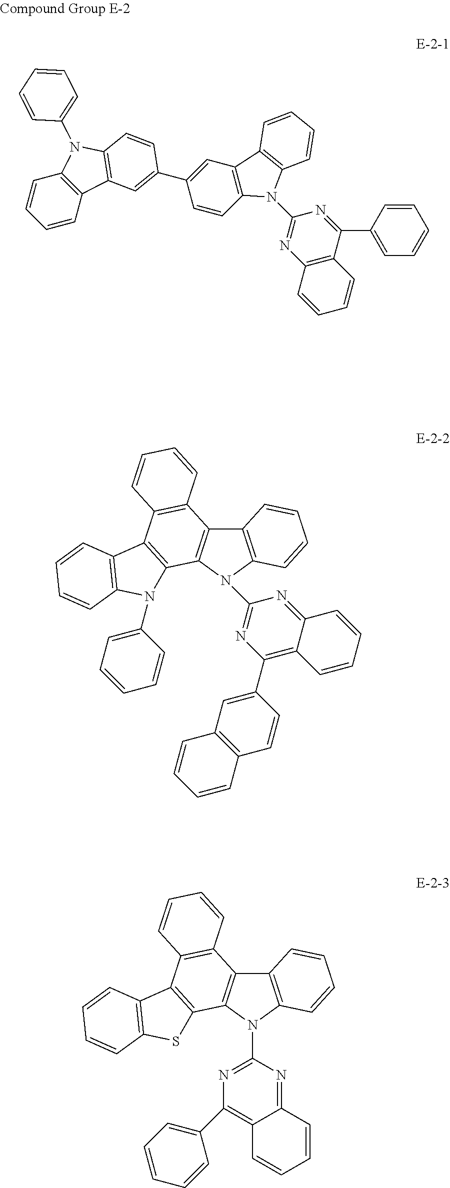 [SMILES: c1ccc(-c2nc(-n3c4ccccc4c4cc(-c5ccc6c(c5)c5ccccc5n6-c5ccccc5)ccc43)nc3ccccc23)cc1 ; c1ccc(-n2c3ccccc3c3c4ccccc4c4c5ccccc5n(-c5nc(-c6ccc7ccccc7c6)c6ccccc6n5)c4c32)cc1 ; c1ccc(-c2nc(-n3c4ccccc4c4c5ccccc5c5c6ccccc6sc5c43)nc3ccccc23)cc1]

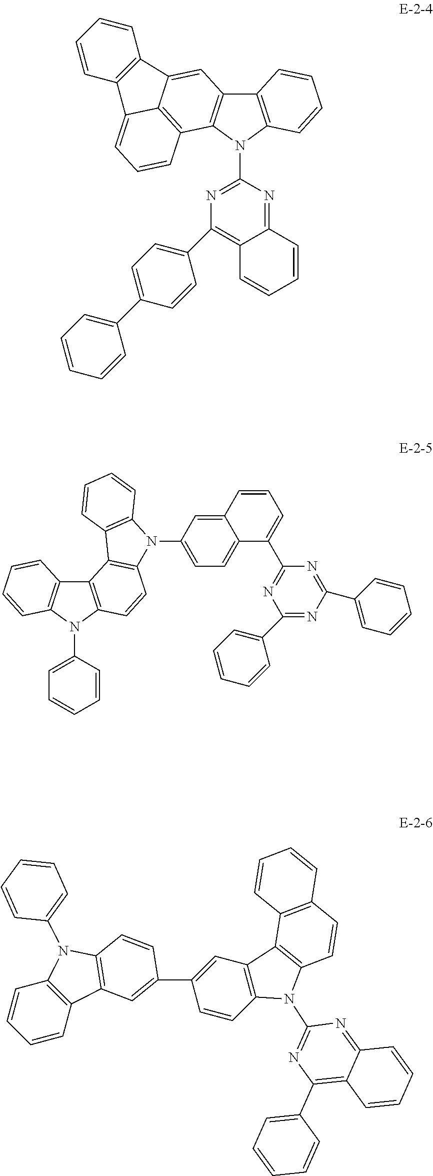 [SMILES: c1ccc(-c2ccc(-c3nc(-n4c5ccccc5c5cc6c7c(cccc7c54)-c4ccccc4-6)nc4ccccc34)cc2)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1]

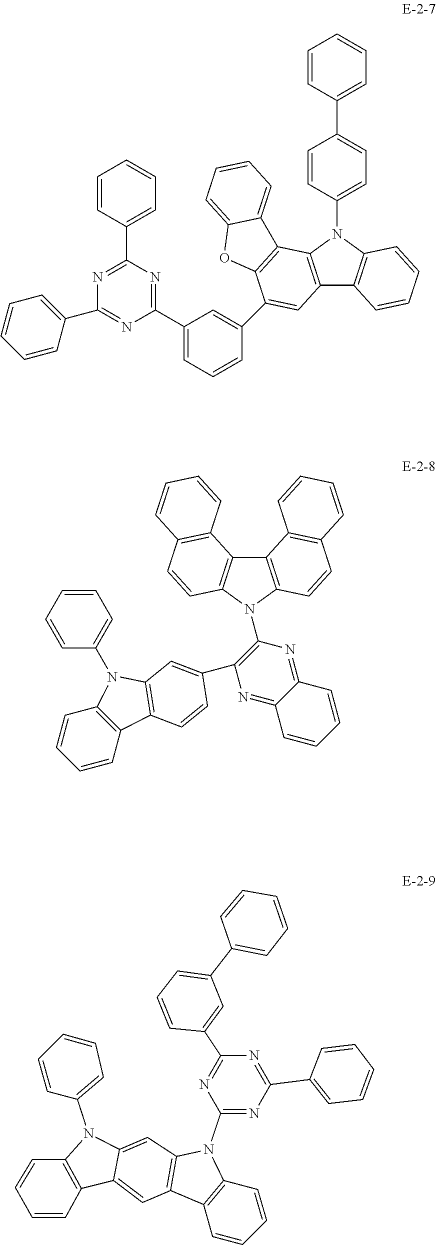 [SMILES: c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-n2c3ccccc3c3ccc(-c4nc5ccccc5nc4-n4c5ccc6ccccc6c5c5c6ccccc6ccc54)cc32)cc1 ; c1ccc(-c2cccc(-c3nc(-c4ccccc4)nc(-n4c5ccccc5c5cc6c7ccccc7n(-c7ccccc7)c6cc54)n3)c2)cc1]

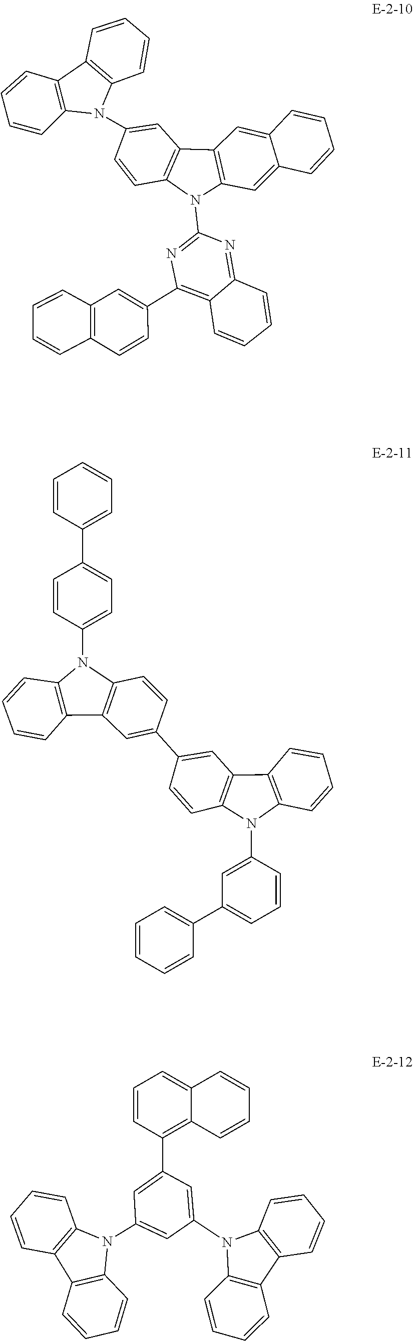 [SMILES: c1ccc2cc(-c3nc(-n4c5ccc(-n6c7ccccc7c7ccccc76)cc5c5cc6ccccc6cc54)nc4ccccc34)ccc2c1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc2c(-c3cc(-n4c5ccccc5c5ccccc54)cc(-n4c5ccccc5c5ccccc54)c3)cccc2c1]

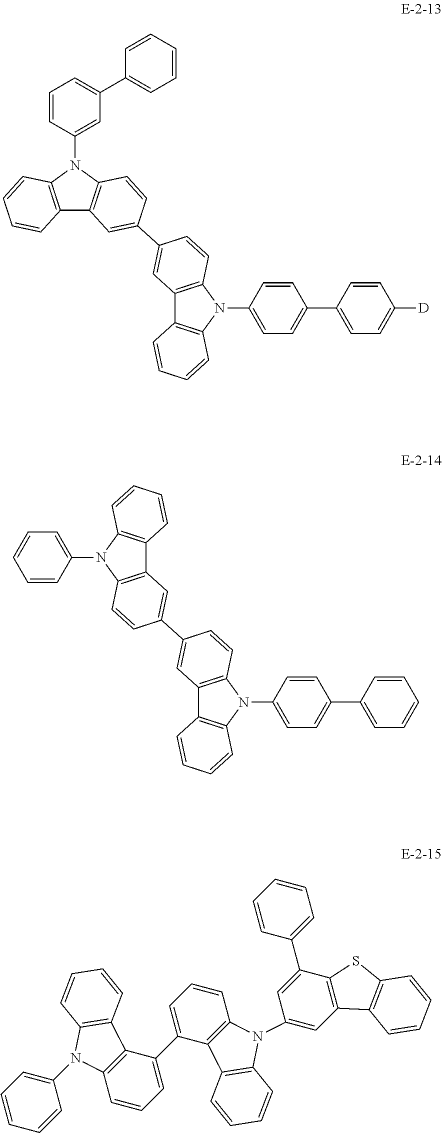 [SMILES: [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

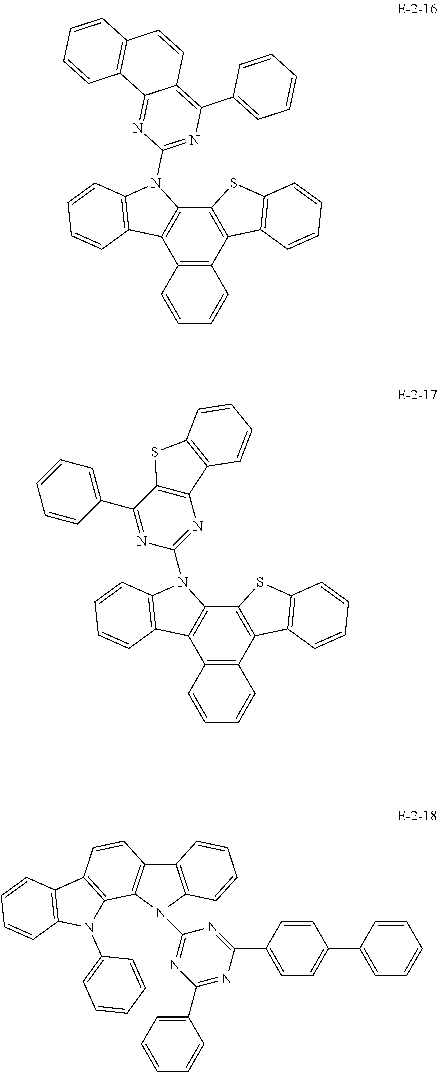 [SMILES: c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1 ; c1ccc(-c2ccc(-c3nc(-c4ccccc4)nc(-n4c5ccccc5c5ccc6c7ccccc7n(-c7ccccc7)c6c54)n3)cc2)cc1]

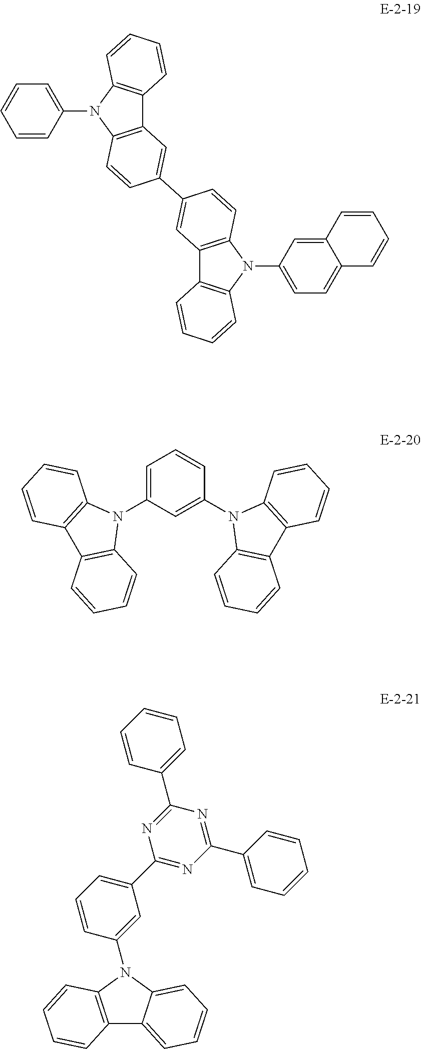 [SMILES: c1ccc(-n2c3ccccc3c3cc(-c4ccc5c(c4)c4ccccc4n5-c4ccc5ccccc5c4)ccc32)cc1 ; c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1]

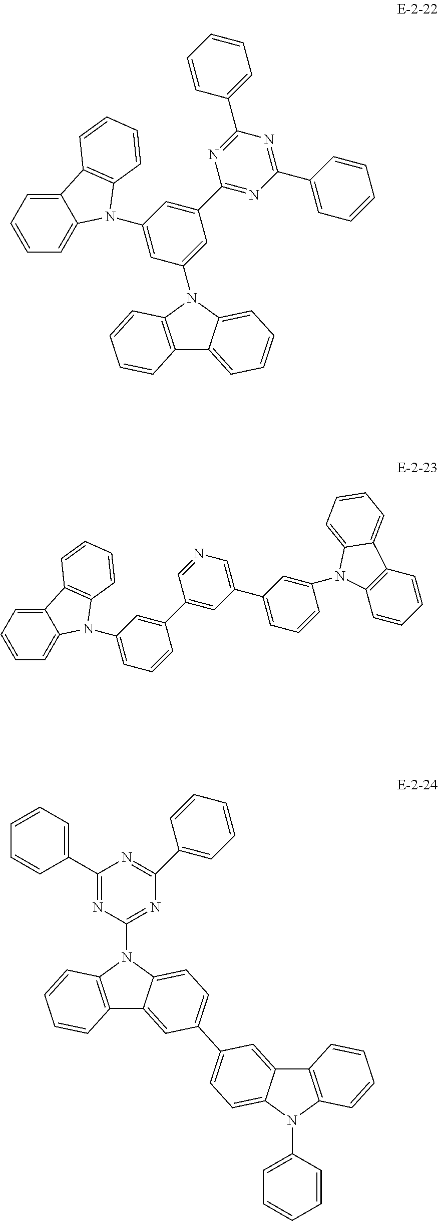 [SMILES: c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-c2nc(-c3ccccc3)nc(-n3c4ccccc4c4cc(-c5ccc6c(c5)c5ccccc5n6-c5ccccc5)ccc43)n2)cc1]

In one or more embodiments, the emission layer EML may further include a material suitable in the art as a host material. For example, the emission layer EML may include, as a host material, at least one of bis(4-(9H-carbazol-9-yl)phenyl)diphenylsilane (BCPDS), (4-(1-(4-(diphenylamino)phenyl)cyclohexyl)phenyl)diphenyl-phosphine oxide (POPCPA), bis[2-(diphenylphosphino)phenyl]ether oxide (DPEPO), 4,4′-bis(N-carbazolyl)-1,1′-biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), or 1,3,5-tris(1-phenyl-1H-benzo[d]imidazole-2-yl)benzene (TPBi). However, embodiments of the present disclosure are not limited thereto, for example, tris(8-hydroxyquinolinato)aluminum (Alq₃), 9,10-di(naphthalen-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetrasiloxane (DPSiO₄), etc. may be utilized as a host material.

In one or more embodiments, the emission layer EML may include a compound represented by Formula M-a. The compound represented by Formula M-a may be utilized as a phosphorescent dopant material.

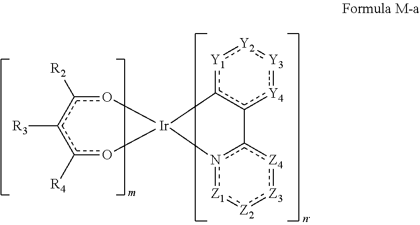

In Formula M-a, Y₁ to Y₄ and Z₁ to Z₄ may each independently be CR₁ or N, R₁ to R₄ may each independently be hydrogen, deuterium, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or may be bonded to an adjacent group to form a ring. In Formula M-a, m is 0 or 1, and n is 2 or 3. In Formula M-a, when m is 0, n is 3, and when m is 1, n is 2.

The compound represented by Formula M-a may be utilized as a phosphorescent dopant.

The compound represented by Formula M-a may be any one selected from among Compound M-a1 to Compound M-a25. However, Compounds M-a1 to M-a25 are mere examples, and the compound represented by Formula M-a is not limited to those represented by Compounds M-a1 to M-a25.

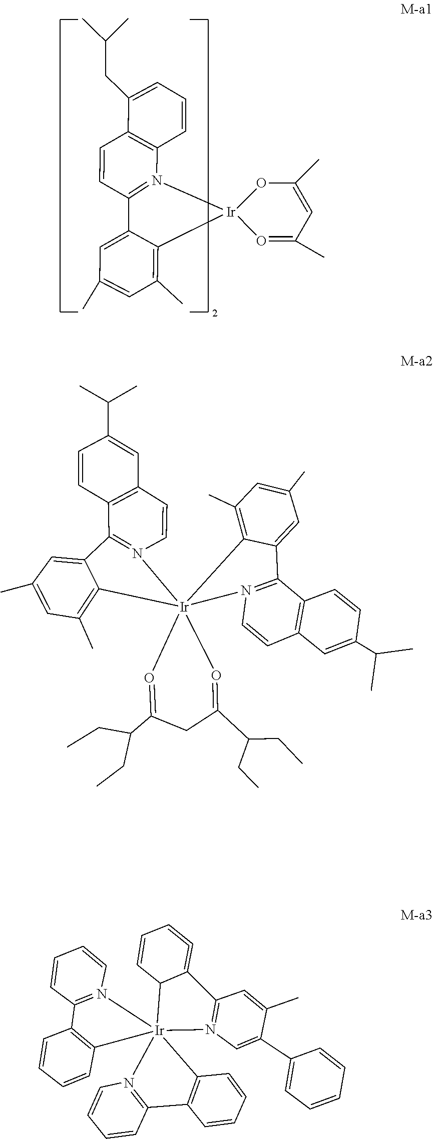

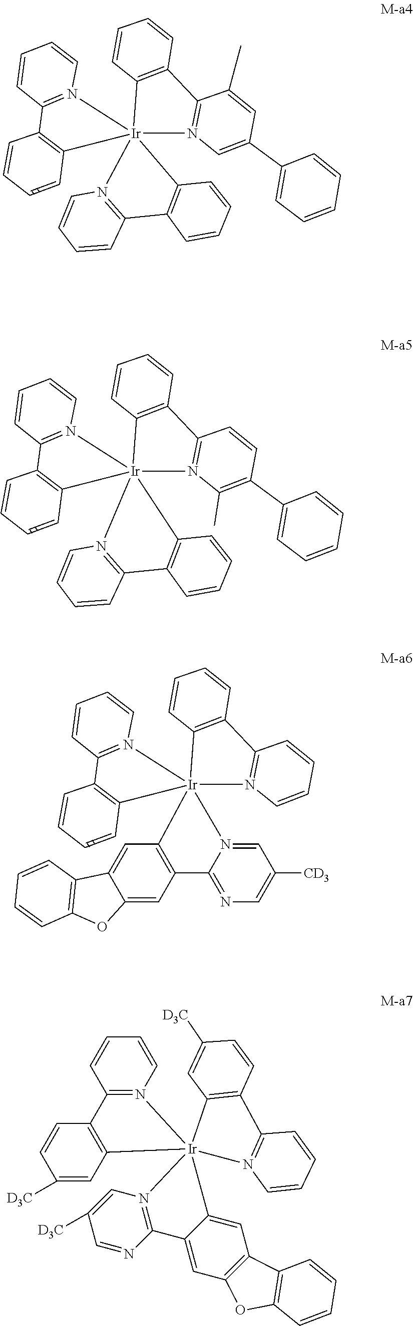

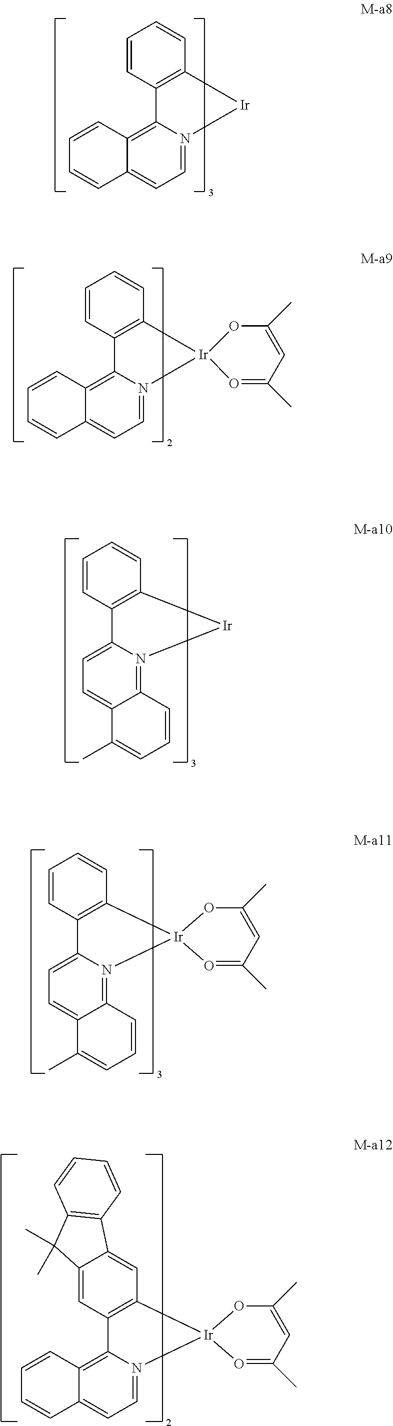

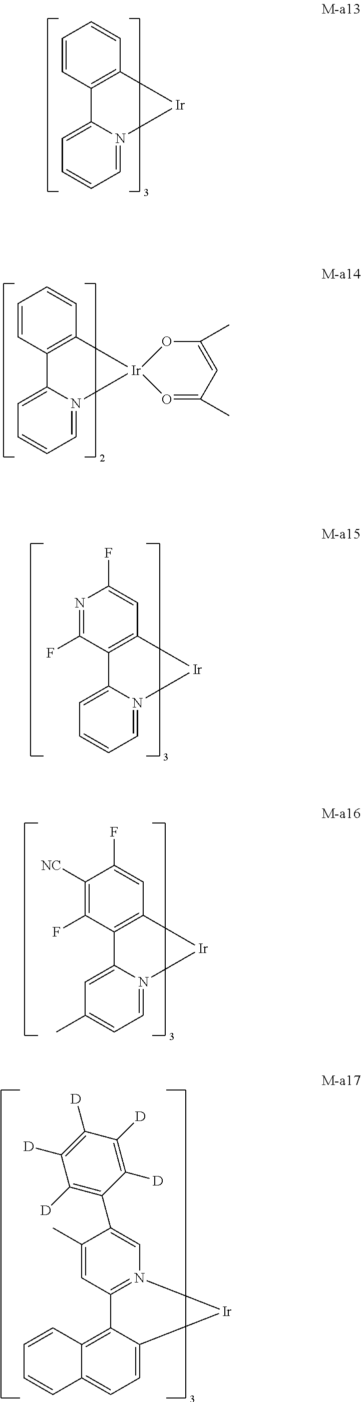

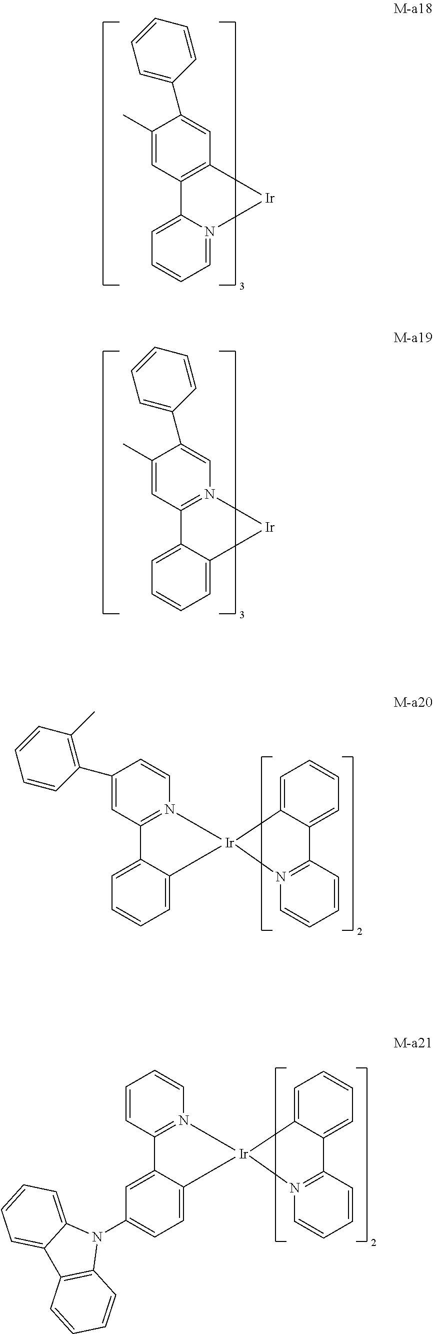

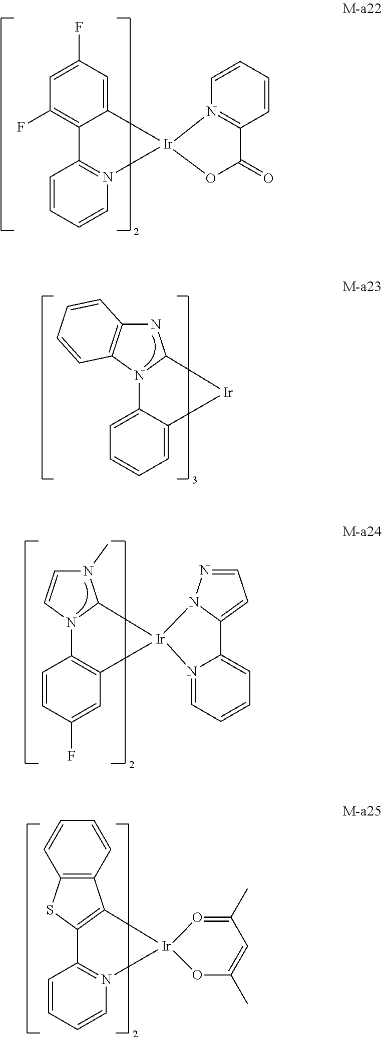

In one or more embodiments, the compound M-a1 and the compound M-a2 may be utilized as a red dopant material, and the compound M-a3 to the compound M-a7 may be utilized as a green dopant material.

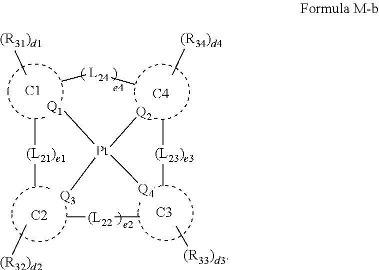

In Formula M-b, Q₁ to Q₄ may each independently be C or N, and C1 to C4 may each independently be a substituted or unsubstituted hydrocarbon ring having 5 to 30 ring-forming carbon atoms, or a substituted or unsubstituted hetero ring having 2 to 30 ring-forming carbon atoms. L₂₁ to L₂₄ may each independently be a direct linkage,

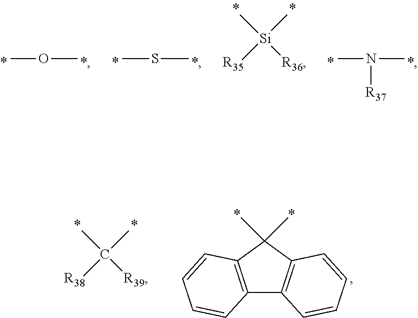

a substituted or unsubstituted alkylene group having 1 to 20 carbons, a substituted or unsubstituted arylene group having 6 to 30 ring-forming carbon atoms, or a substituted or unsubstituted heteroarylene group having 2 to 30 ring-forming carbon atoms, and e1 to e4 may each independently be 0 or 1. R₃₁ to R₃₉ may each independently be hydrogen, deuterium, a halogen, a cyano group, a substituted or unsubstituted amine group, a substituted or unsubstituted alkyl group having 1 to 20 carbons, a substituted or unsubstituted aryl group having 6 to 30 ring-forming carbon atoms, or a substituted or unsubstituted heteroaryl group having 2 to 30 ring-forming carbon atoms, and/or forms a ring by being coupled to an adjacent group, and d1 to d4 may each independently be an integer of 0 to 4.

The compound represented by Formula M-b may be utilized as a blue phosphorescent dopant or a green phosphorescent dopant. In some embodiments, the compound represented by Formula M-b may further be included in the emission layer EML as an auxiliary dopant in one or more embodiments.

The compound represented by Formula M-b may be any one selected from among compound M-b-1 to compound M-b-11. However, the compounds are only mere examples, and the compound represented by Formula M-b is not limited to the compound M-b-1 to the compound M-b-11.

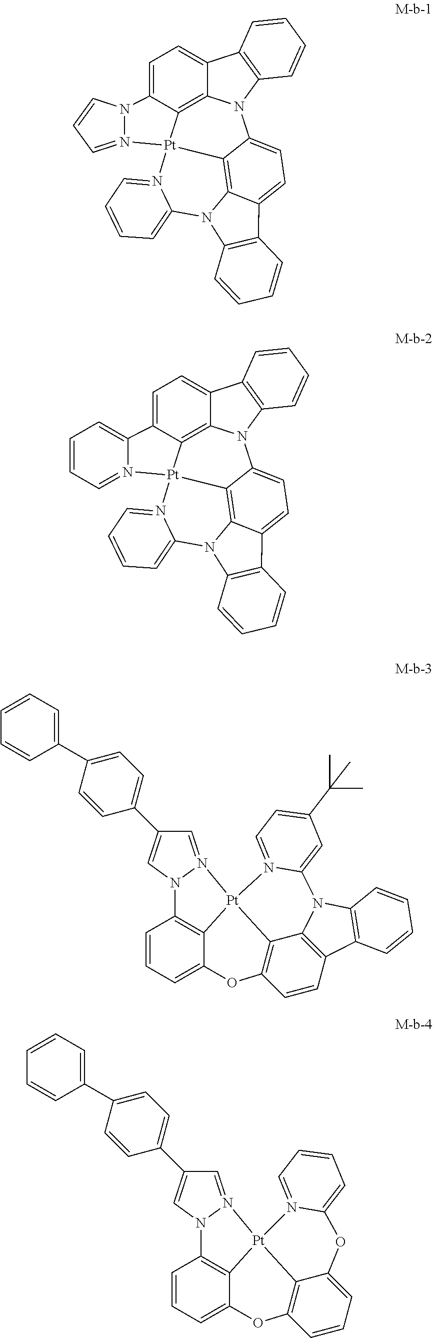

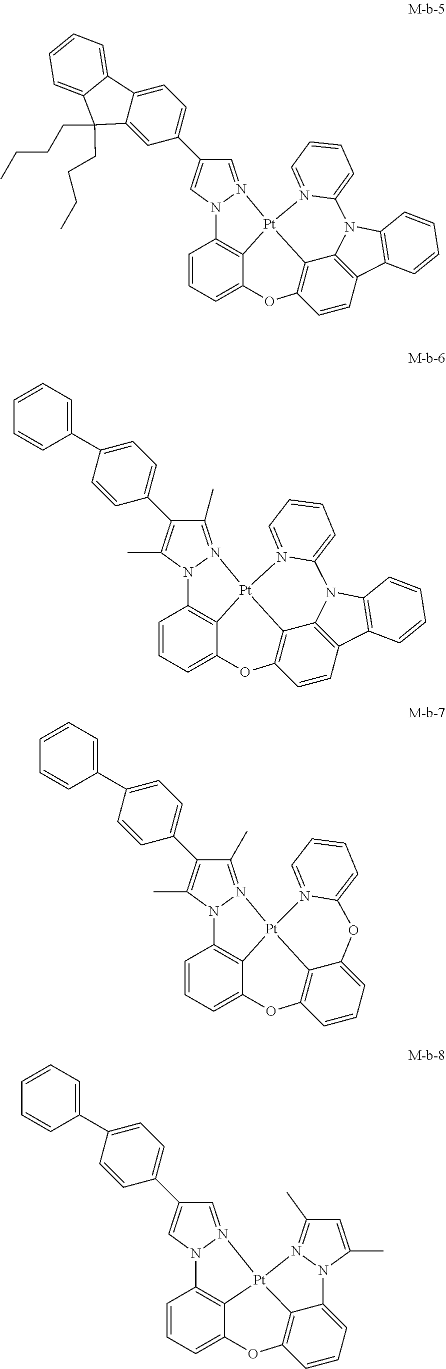

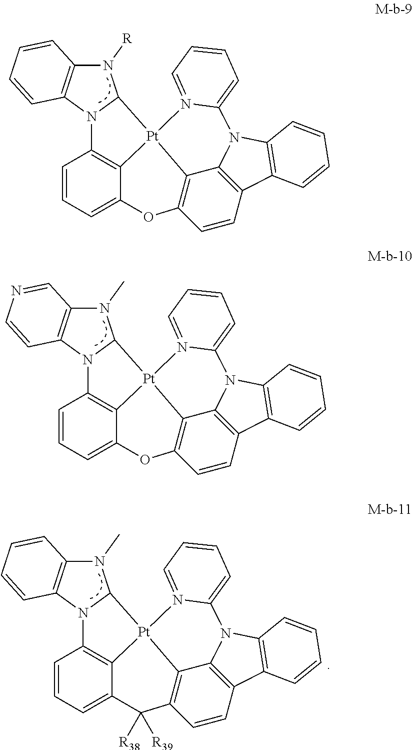

In the compounds above, R, R₃₈, and R₃₉ may each independently be hydrogen, deuterium, a halogen, a cyano group, a substituted or unsubstituted amine group, a substituted or unsubstituted alkyl group having 1 to 20 carbons, a substituted or unsubstituted aryl group having 6 to 30 ring-forming carbon atoms, or a substituted or unsubstituted heteroaryl group having 2 to 30 ring-forming carbon atoms.

In one or more embodiments, the emission layer EML may include a compound represented by any one selected from among Formula F-a to Formula F-c. The compound represented by Formula F-a to Formula F-c may be utilized as a fluorescence dopant material.

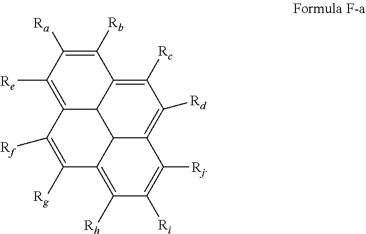

In Formula F-a, two selected from among Rₐ to Rⱼ may each independently be substituted with *—NAr₁Ar₂. The others (the remainder), which are not substituted with *—NAr₁Ar₂, among Rₐ to Rⱼ may each independently be hydrogen, deuterium, a halogen, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In *—NAr₁Ar₂, Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. For example, in some embodiments, at least one of Ar₁ or Ar₂ may be a heteroaryl group containing O or S as a ring-forming atom.

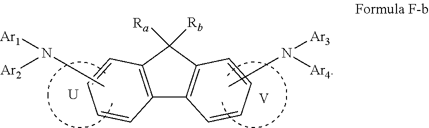

In Formula F-b above, Rₐ and Rb may each independently be hydrogen, deuterium, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or may be bonded to an adjacent group to form a ring. Ar₁ to Ar₄ may each independently be a substituted or unsubstituted aryl group having 6 to 30 ring-forming carbon atoms, or a substituted or unsubstituted heteroaryl group having 2 to 30 ring-forming carbon atoms.

In Formula F-b, U and V may each independently be a substituted or unsubstituted hydrocarbon ring having 5 to 30 ring-forming carbon atoms, or a substituted or unsubstituted heterocycle having 2 to 30 ring-forming carbon atoms. At least one selected from among Ar₁ to Ar₄ may be a heteroaryl group containing O or S as a ring-forming atom.

In Formula F-b, the number of rings represented by U and V may each independently be 0 or 1. For example, in Formula F-b, it refers to that when the number of U or V is 1, one ring constitutes a fused ring at a portion indicated by U or V, and when the number of U or V is 0, a ring indicated by U or V does not exist. For example, when the number of U is 0 and the number of V is 1, or when the number of U is 1 and the number of V is 0, the fused ring having a fluorene core in Formula F-b may be a cyclic compound having four rings. In some embodiments, when each number of U and V is 0, the fused ring in Formula F-b may be a cyclic compound having three rings. In some embodiments, when each number of U and Vis 1, the fused ring having a fluorene core in Formula F-b may be a cyclic compound having five rings.

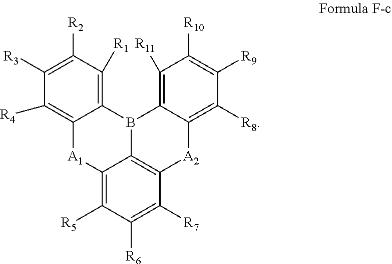

In Formula F-c, A₁ and A₂ may each independently be O, S, Se, or NRₘ, and Rₘ may be hydrogen, deuterium, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. R₁ to R₁₁ may each independently be hydrogen, deuterium, a halogen, a cyano group, a substituted or unsubstituted amine group, a substituted or unsubstituted boryl group, a substituted or unsubstituted oxy group, a substituted or unsubstituted thio group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or may be bonded to an adjacent group to form a ring.

In Formula F-c, A₁ and A₂ may each independently be bonded to substituents of an adjacent ring to form a fused ring. For example, when A₁ and A₂ may each independently be NRₘ, A₁ may be bonded to R₄ or R₅ to form a ring. In some embodiments, A₂ may be bonded to R₇ or R₈ to form a ring.

In one or more embodiments, the emission layer EML may further include, as a suitable dopant material, a styryl derivative (e.g., 1,4-bis[2-(3-N-ethylcarbazolyl)vinyl]benzene (BCzVB), 4-(di-p-tolylamino)-4′-[(di-p-tolylamino)styryl]stilbene (DPAVB), and N-(4-((E)-2-(6-((E)-4-(diphenylamino)styryl)naphthalen-2-yl)vinyl)phenyl)-N-phenylbenzenamine (N-BDAVBi), 4,4′-bis[2-(4-(N,N-diphenylamino)phenyl)vinyl]biphenyl (DPAVBi)), perylene and/or a derivative thereof (e.g., 2,5,8,11-tetra-t-butylperylene (TBP)), pyrene and/or a derivative thereof (e.g., 1,1-dipyrene, 1,4-dipyrenylbenzene, 1,4-bis(N,N-diphenylamino)pyrene), etc.

In one or more embodiments, the emission layer EML may further include a suitable phosphorescence dopant material. For example, a metal complex containing iridium (Ir), platinum (Pt), osmium (Os), aurum (Au), titanium (Ti), zirconium (Zr), hafnium (Hf), europium (Eu), terbium (Tb), or thulium (Tm) may be utilized as a phosphorescent dopant. For example, in some embodiments, iridium(III) bis(4,6-difluorophenylpyridinato-N, C2) (FIrpic), bis(2,4-difluorophenylpyridinato)-tetrakis(1-pyrazolyl)borate iridium(III) (Fir6), and/or platinum octaethyl porphyrin (PtOEP) may be utilized as a phosphorescent dopant. However, embodiments of the present disclosure are not limited thereto.

In some embodiments, the emission layer EML may include a hole transport host and an electron transport host. In some embodiments, the emission layer EML may include an auxiliary dopant and a light emitting dopant. In some embodiments, as the auxiliary dopant, a phosphorescence dopant material or a thermally activated delayed fluorescence dopant material may be included. For example, in an embodiment, the emission layer EML may include a hole transport host, an electron transport host, an auxiliary dopant, and a light emitting dopant.

In some embodiments, in the emission layer EML, exciplex may be formed by the hole transport host and the electron transport host. In these embodiments, the triplet energy of the exciplex formed by the hole transport host and the electron transport host may correspond to the energy gap T1 between the lowest unoccupied molecular orbital (LUMO) energy level of the electron transport host and the highest occupied molecular orbital (HOMO) energy level of the hole transport host.

In one or more embodiments, the triplet energy (T1) of the exciplex formed by the hole transport host and the electron transport host may be about 2.4 eV to about 3.0 eV. In some embodiments, the triplet energy of the exciplex may be a smaller value than the energy gap of each host material. Accordingly, the exciplex may have the triplet energy of about 3.0 eV or less, which is the energy gap between the hole transport host and the electron transport host.

In one or more embodiments, the emission layer EML may include a quantum dot material. In some embodiments, the quantum dot material may have a core-shell structure. The core of the quantum dot may be selected from a Group II-VI compound, a Group III-VI compound, a Group I-III-IV compound, a Group III-V compound, a Group III-II-V compound, a Group IV-VI compound, a Group IV element, a Group IV compound, and combinations thereof.

The Group II-VI compound may be selected from the group consisting of a binary compound selected from the group consisting of CdSe, CdTe, CdS, ZnS, ZnSe, ZnTe, ZnO, HgS, HgSe, HgTe, MgSe, MgS, and mixtures thereof, a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and mixtures thereof, and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, and mixtures thereof.

The Group III-VI compound may include a binary compound such as In₂S₃ or In₂Se₃, a ternary compound such as InGaS₃ or InGaSe₃, or any combination thereof.

The Group I-III-VI compound may be selected from a ternary compound selected from the group consisting of AgInS, AgInS₂, CuInS, CuInS₂, AgGaS₂, CuGaS₂ CuGaO₂, AgGaO₂, AgAlO₂, and mixtures thereof, and a quaternary compound such as AgInGaS₂ and/or CuInGaS₂.

The Group III-V compound may be selected from the group consisting of a binary compound selected from the group consisting of GaN, GaP, GaAs, GaSb, AlN, AlP, AlAs, AlSb, InN, InP, InAs, InSb, and mixtures thereof, a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and mixtures thereof, and a quaternary compound selected from the group consisting of GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and mixtures thereof. In some embodiments, the Group III-V compound may further include a Group II metal. For example, InZnP, etc., may be selected as a Group III-II-V compound.

The Group IV-VI compound may be selected from the group consisting of a binary compound selected from the group consisting of SnS, SnSe, SnTe, PbS, PbSe, PbTe, and mixtures thereof, a ternary compound selected from the group consisting of SnSeS, SnSeTe, SnSTe, PbSeS, PbSeTe, PbSTe, SnPbS, SnPbSe, SnPbTe, and mixtures thereof, and a quaternary compound selected from the group consisting of SnPbSSe, SnPbSeTe, SnPbSTe, and mixtures thereof. The Group IV element may be selected from the group consisting of Si, Ge, and a mixture thereof. The Group IV compound may be a binary compound selected from the group consisting of SiC, SiGe, and a mixture thereof.

Each element included in the binary compound, the ternary compound, or the quaternary compound may be present in substantially uniform concentrations in particle(s), or may be divided into partially different forms of concentration distributions and present in a same particle of the particles. In some embodiments, a core/shell structure in which one quantum dot surrounds another quantum dot may be present. In some embodiments, in the core/shell structure, concentration gradient by which the concentration of an element present in the shell decreases toward the core, may be shown.

In some embodiments, the quantum dot may have the above-described core/shell structure including a core containing nanocrystals and a shell around (e.g., surrounding) the core. The shell of the quantum dot may serve as a protection layer to prevent or reduce the chemical deformation of the core to maintain semiconductor properties, and/or a charging layer to impart electrophoresis properties to the quantum dot. The shell may be a single layer or multiple layers. An example of the shell of the quantum dots may include a metal or non-metal oxide, a semiconductor compound, or a combination thereof.

For example, in the shell, the metal or non-metal oxide may be a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄, and/or NiO, or a ternary compound such as MgAl₂O₄, CoFe₂O₄, NiFe₂O₄, and/or CoMn₂O₄, but embodiments of the present disclosure are not limited thereto.

Non-limiting examples of the semiconductor compound suitable as a shell may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but embodiments of the present disclosure are not limited thereto.

Each element included in a polynary compound such as the binary compound, or the ternary compound may be present in a particle with a substantially uniform or non-substantially uniform concentration distribution. For example, the formulae presented above may only refer to the types (kinds) of elements included in the compounds, and the elemental ratio in the compound may be different.

The quantum dot may have a full width of half maximum (FWHM) of an emission spectrum of about 45 nm or less, about 40 nm or less, or about 30 nm or less, and color purity or color reproducibility of the light emitting element may be improved within the above range of FWHM. In some embodiments, light emitted through such quantum dots is emitted in all directions so that a wide viewing angle may be improved.

In some embodiments, although the form of the quantum dot is not particularly limited as long as it is a form commonly utilized in the art, in some embodiments, the quantum dot in the form of spherical, pyramidal, multi-arm, or cubic nanoparticles, nanotubes, nanowires, nanofibers, nanoplate particles, etc. may be utilized.

As the size of the quantum dot is adjusted or the elemental ratio in the quantum dot compound is adjusted, it is possible to control the energy band gap, and thus light in one or more suitable wavelength ranges may be obtained in a quantum dot emission layer. Therefore, in some embodiments, the quantum dot as described above (utilizing different sizes of quantum dots or different elemental ratios in the quantum dot compound) may be utilized, and thus the light emitting element, which emits light in one or more suitable wavelengths, may be implemented. For example, the adjustment of the size of the quantum dot or the elemental ratio in the quantum dot compound may be selected to emit red, green, and/or blue light. In some embodiments, the quantum dots may be configured to emit white light by combining one or more suitable colors of light.

The quantum dot may control the color of light according to the particle size, and accordingly, the quantum dot may have one or more suitable emission colors such as blue, red, or green.

In the light emitting elements ED of one or more embodiments, shown in FIG. 3 to FIG. 6, the electron transport region ETR may be provided on the emission layer EML. The electron transport region ETR may include at least one of a hole blocking layer HBL, an electron transport layer ETL, or an electron injection layer EIL, but embodiments of the present disclosure are not limited thereto.

The electron transport region ETR may have a single layer formed of a single material, a single layer formed of a plurality of different materials, or a multilayer structure including a plurality of layers formed of a plurality of different materials.

For example, in some embodiments, the electron transport region ETR may have a single layer structure of an electron injection layer EIL or an electron transport layer ETL, and may have a single layer structure formed of an electron injection material and an electron transport material. In some embodiments, the electron transport region ETR may have a single layer structure formed of a plurality of different materials, or may have a structure in which an electron transport layer ETL/electron injection layer EIL, a hole blocking layer HBL/electron transport layer ETL/electron injection layer EIL are stacked in order (in each stated order) from the emission layer EML, but embodiments of the present disclosure are not limited thereto. The electron transport region ETR may have a thickness, for example, from about 1,000 Å to about 1,500 Å.

The electron transport region ETR may be formed utilizing one or more suitable methods such as a vacuum deposition method, a spin coating method, a casting method, a Langmuir-Blodgett (LB) method, an inkjet printing method, a laser printing method, and/or a laser induced thermal imaging (LITI) method.

In one or more embodiments, the electron transport region ETR may include a compound represented by Formula ET-1:

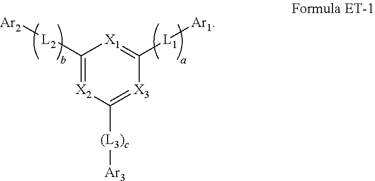

In Formula ET-1, at least one selected from among X₁ to X₃ is N, and the rest are CRₐ. Rₐ may be hydrogen, deuterium, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. Ar₁ to Ar₃ may each independently be hydrogen, deuterium, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In Formula ET-1, a to c may each independently be an integer of 0 to 10. In Formula ET-1, L₁ to L₃ may each independently be a direct linkage, a substituted or unsubstituted arylene group having 6 to 30 ring-forming carbon atoms, or a substituted or unsubstituted heteroarylene group having 2 to 30 ring-forming carbon atoms. In some embodiments, when a to c may each independently be an integer of 2 or more, L₁ to L₃ may each independently be a substituted or unsubstituted arylene group having 6 to 30 ring-forming carbon atoms, or a substituted or unsubstituted heteroarylene group having 2 to 30 ring-forming carbon atoms.

In one or more embodiments, the electron transport region ETR may include an anthracene-based compound. However, embodiments of the present disclosure are not limited thereto, and, in some embodiments, the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzimidazol-1-yl)phenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(biphenyl-4-yl)-4-phenyl-5-tert-butylphenyl-1,2,4-triazole (TAZ), 4-(naphthalen-1-yl)-3,5-diphenyl-4H-1,2,4-triazole (NTAZ), 2-(biphenyl-4-yl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), beryllium bis(benzoquinolin-10-olate) (Bebq₂), 9,10-di(naphthalene-2-yl)anthracene (ADN), 1,3-bis[3,5-di(pyridin-3-yl)phenyl]benzene (BmPyPhB), or a mixture thereof.

In one or more embodiments, the electron transport region ETR may include at least one selected from among Compound ET1 to Compound ET36:

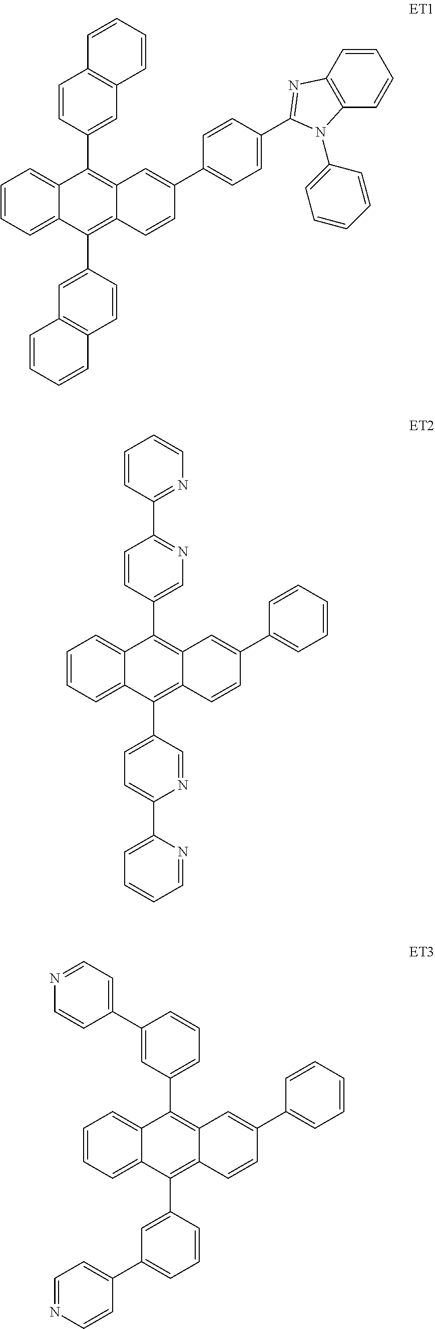 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

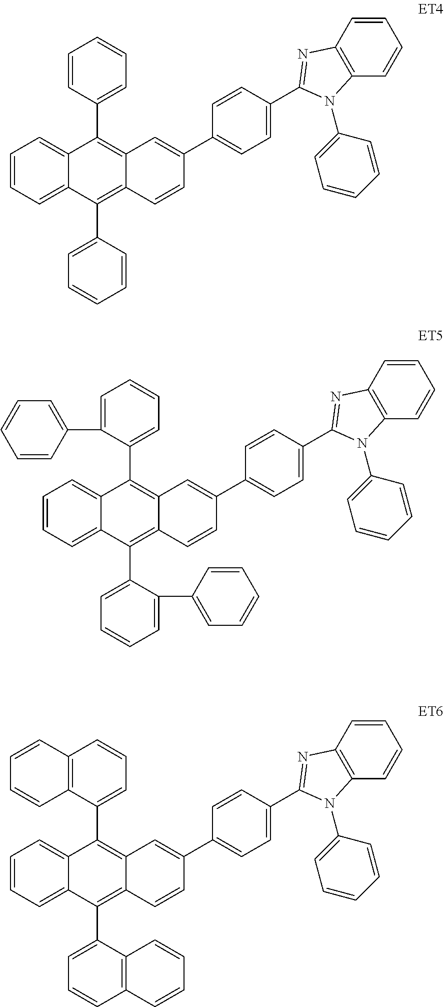 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

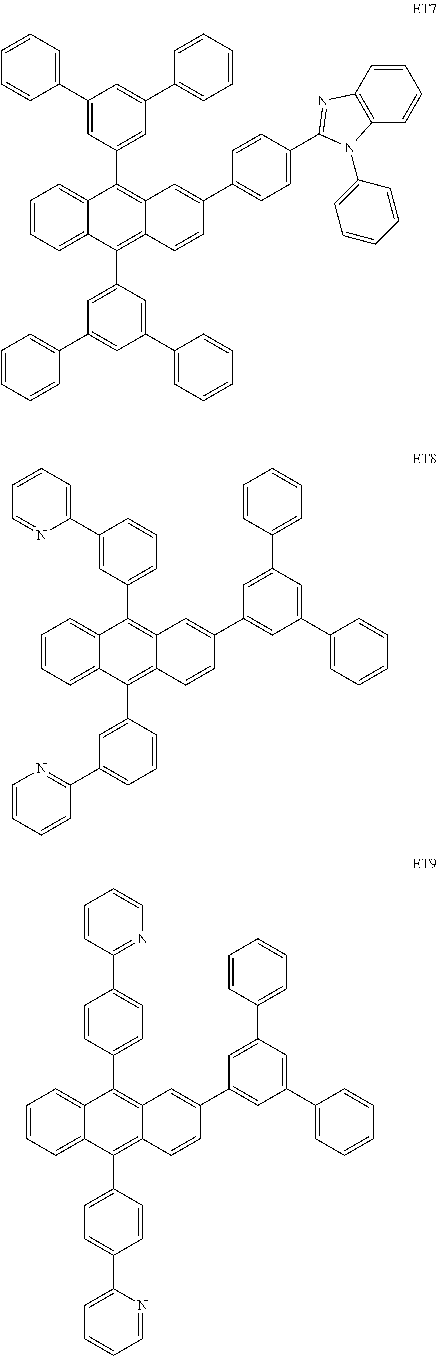 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

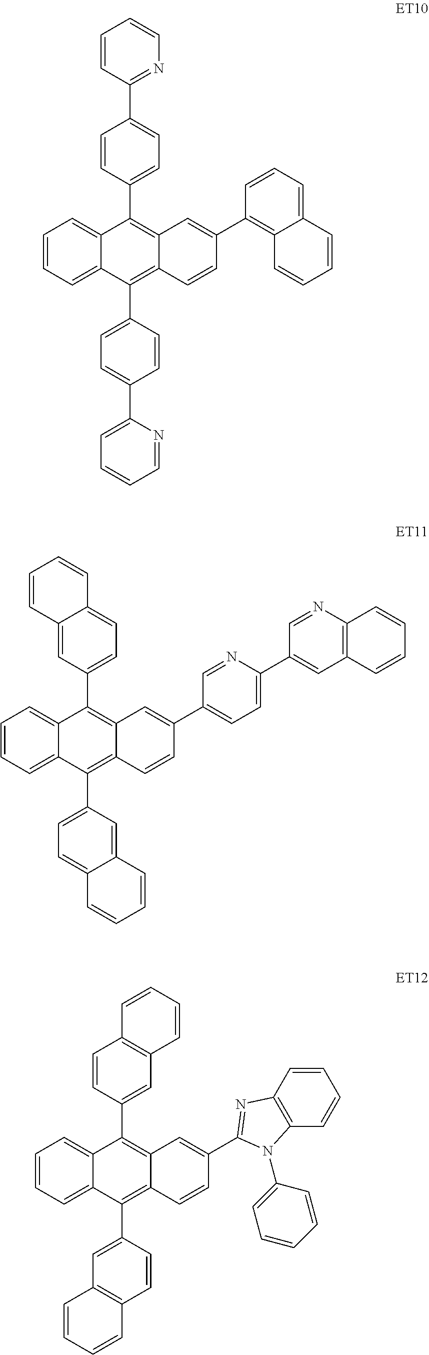 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

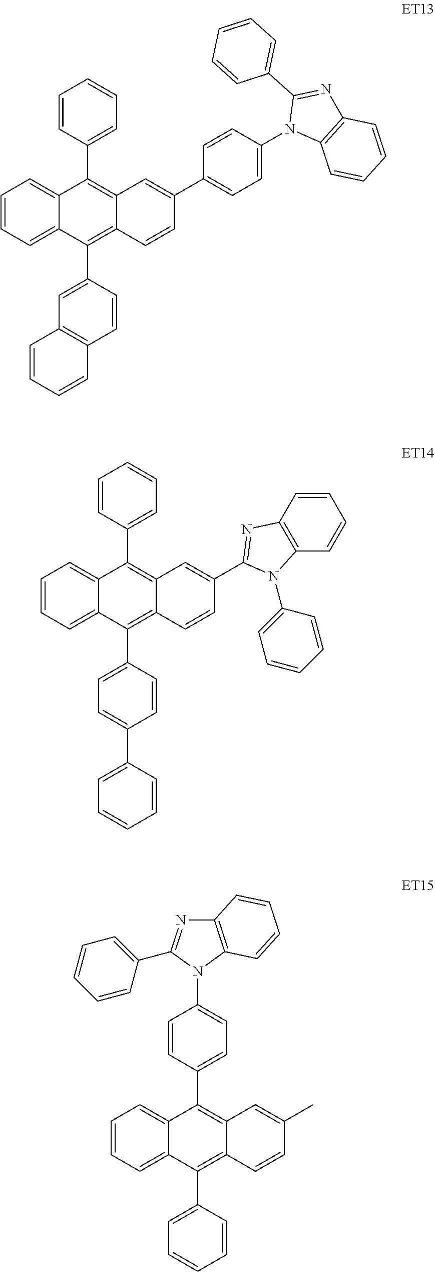 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

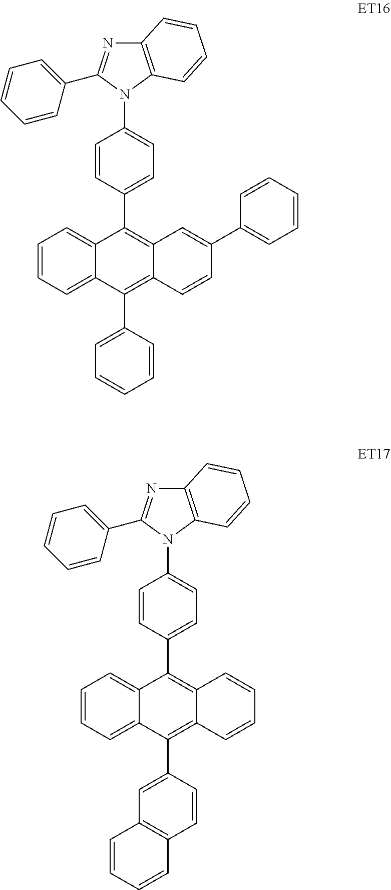 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1]

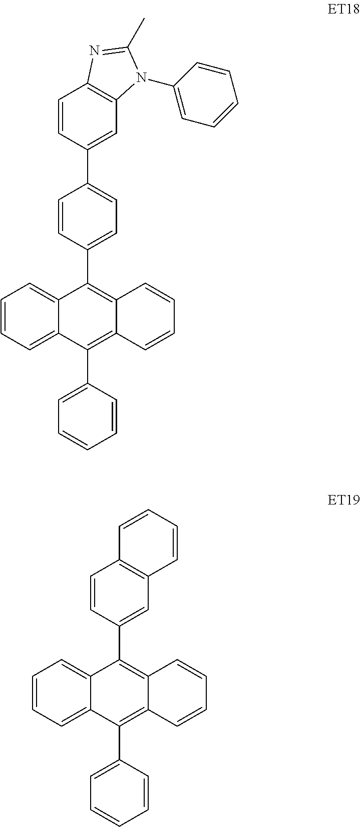

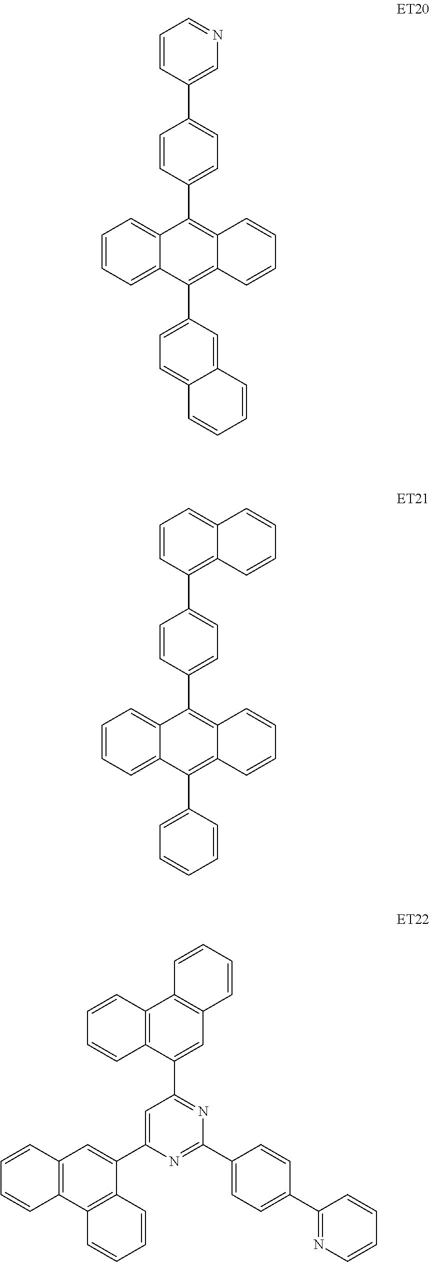

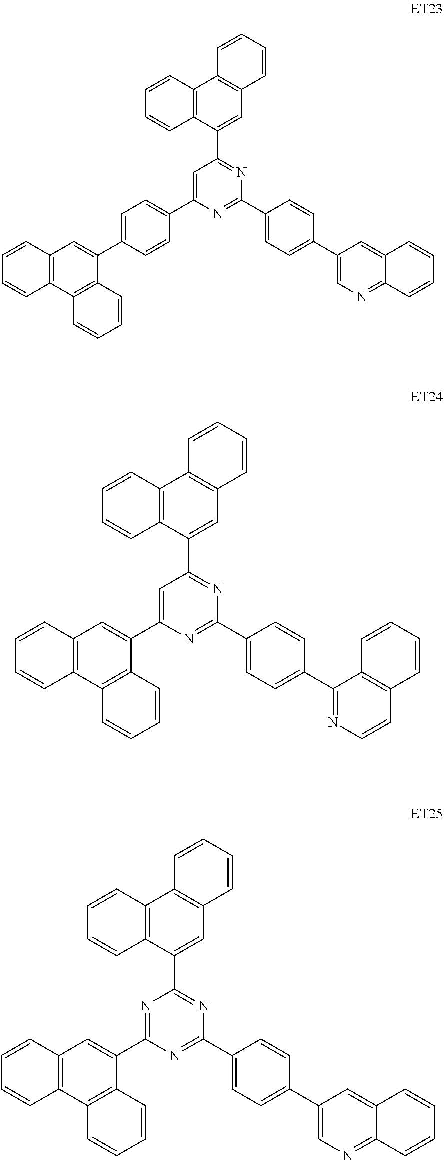 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1]

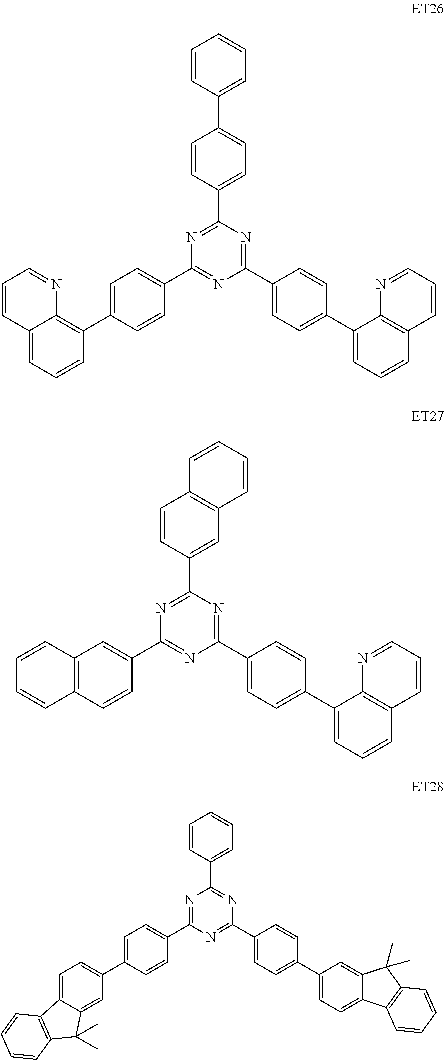 [SMILES: c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21]

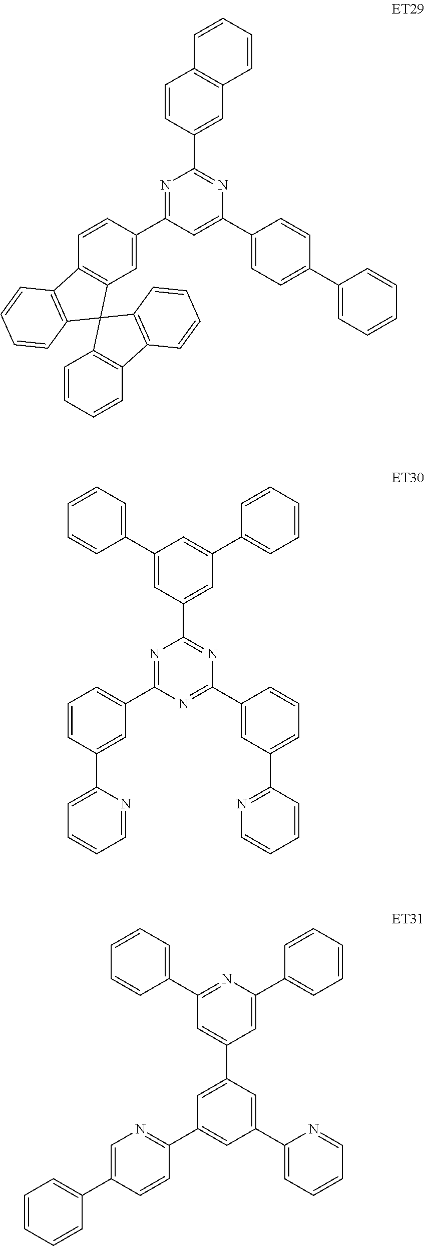 [SMILES: c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1 ; c1ccc(-c2ccc(-c3cc(-c4cc(-c5ccccc5)nc(-c5ccccc5)c4)cc(-c4ccccn4)c3)nc2)cc1]

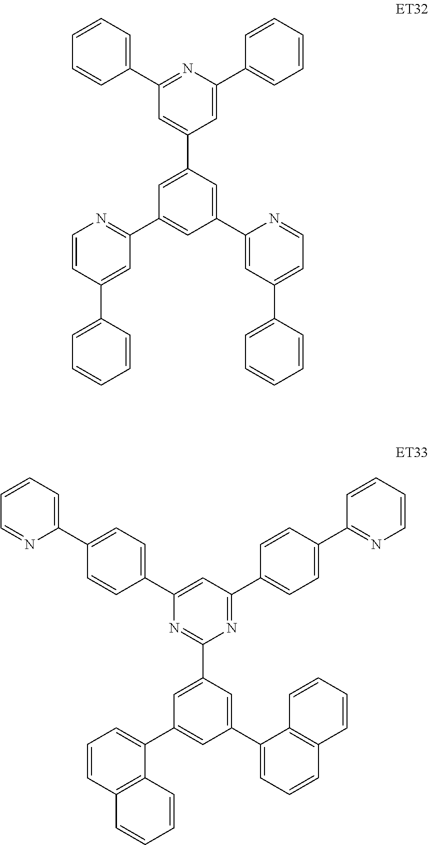 [SMILES: c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

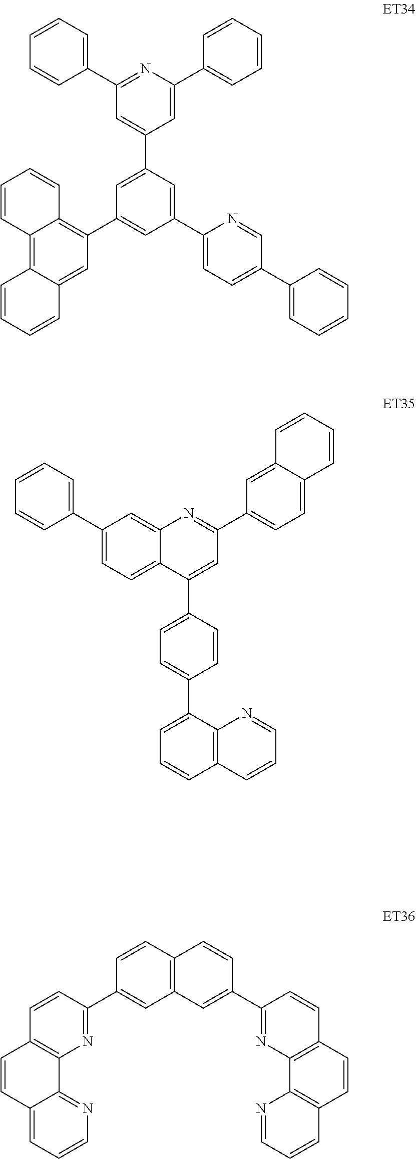 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

In some embodiments, the electron transport region ETR may include a metal halide such as LiF, NaCl, CsF, RbCl, RbI, CuI, and KI, a lanthanide metal such as Yb, or a co-deposited material of the metal halide and the lanthanide metal. For example, in some embodiments, the electron transport region ETR may include KI:Yb, RbI:Yb, LiF:Yb, etc., as a co-deposited material. In some embodiments, the electron transport region ETR may be formed utilizing a metal oxide such as Li₂O or BaO, or 8-hydroxyl-lithium quinolate (Liq), etc., but embodiments of the present disclosure are not limited thereto. In some embodiments, the electron transport region ETR may also be formed of a mixture material of an electron transport material and an insulating organometallic salt. The organometallic salt may be a material having an energy band gap of about 4 eV or more. For example, the organometallic salt may include, for example, a metal acetate, a metal benzoate, a metal acetoacetate, a metal acetylacetonate, and/or a metal stearate.

In one or more embodiments, the electron transport region ETR may further include at least one of 2,9-dimethyl-4,7-diphenyl-1,10-phenanthroline (BCP), diphenyl(4-(triphenylsilyl)phenyl)phosphine oxide (TSPO1), or 4,7-diphenyl-1,10-phenanthroline (Bphen) in addition to the above-described materials, but embodiments of the present disclosure are not limited thereto.

The electron transport region ETR may include the above-described compounds of the electron transport region in at least one of the electron injection layer EIL, the electron transport layer ETL, or the hole blocking layer HBL.

When the electron transport region ETR includes an electron transport layer ETL, the electron transport layer ETL may have a thickness of about 100 Å to about 1,000 Å, for example, about 150 Å to about 500 Å. When the thickness of the electron transport layer ETL satisfies the aforementioned range, satisfactory electron transport characteristics may be obtained without a substantial increase in driving voltage. When the electron transport region ETR includes an electron injection layer EIL, the electron injection layer EIL may have a thickness of about 1 Å to about 100 Å, for example, about 3 Å to about 90 Å. When the thickness of the electron injection layer EIL satisfies the above-described range, satisfactory electron injection characteristics may be obtained without a substantial increase in driving voltage.

The second electrode EL2 may be provided on the electron transport region ETR. The second electrode EL2 may be a common electrode. The second electrode EL2 may be a cathode or an anode, but embodiments of the present disclosure are not limited thereto. For example, when the first electrode EL1 is an anode, the second electrode EL2 may be a cathode, and when the first electrode EL1 is a cathode, the second electrode EL2 may be an anode.

The second electrode EL2 may be a transmissive electrode, a transflective electrode, or a reflective electrode. When the second electrode EL2 is a transmissive electrode, the second electrode EL2 may be formed of a transparent metal oxide, for example, indium tin oxide (ITO), indium zinc oxide (IZO), zinc oxide (ZnO), indium tin zinc oxide (ITZO), etc.

When the second electrode EL2 is a transflective electrode or a reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, W, or a compound or mixture thereof (e.g., AgMg, AgYb, or MgYb). In some embodiments, the second electrode EL2 may have a multilayer structure including a reflective film or a transflective film formed of one or more selected from the above-described materials, and a transparent conductive film formed of ITO, IZO, ZnO, ITZO, etc. For example, the second electrode EL2 may include the above-described metal materials, combinations of at least two metal materials of the above-described metal materials, oxides of the above-described metal materials, and/or the like.

In some embodiments, the second electrode EL2 may be connected with an auxiliary electrode. When the second electrode EL2 is connected with the auxiliary electrode, the resistance of the second electrode EL2 may be decreased.

In some embodiments, a capping layer CPL may further be disposed on the second electrode EL2 of the light emitting element ED. The capping layer CPL may include a multilayer or a single layer.

In one or more embodiments, the capping layer CPL may be an organic layer or an inorganic layer. For example, when the capping layer CPL contains an inorganic material, the inorganic material may include an alkali metal compound (e.g., LiF), an alkaline earth metal compound (e.g., MgF₂), SiON, SiNₓ, SiO_{y}, etc.

For example, in some embodiments, when the capping layer CPL includes an organic material, the organic material may include α-NPD, NPB, TPD, m-MTDATA, Alq₃, CuPc, N4,N4, N4′, N4′-tetra(biphenyl-4-yl)biphenyl-4,4′-diamine (TPD15), 4,4′,4″-tris(carbazol-9-yl)triphenylamine (TCTA), etc., an epoxy resin, and/or an acrylate such as methacrylate. However, embodiments of the present disclosure are not limited thereto, for example, in some embodiments, the capping layer CPL may include at least one selected from among Compounds P1 to P5:

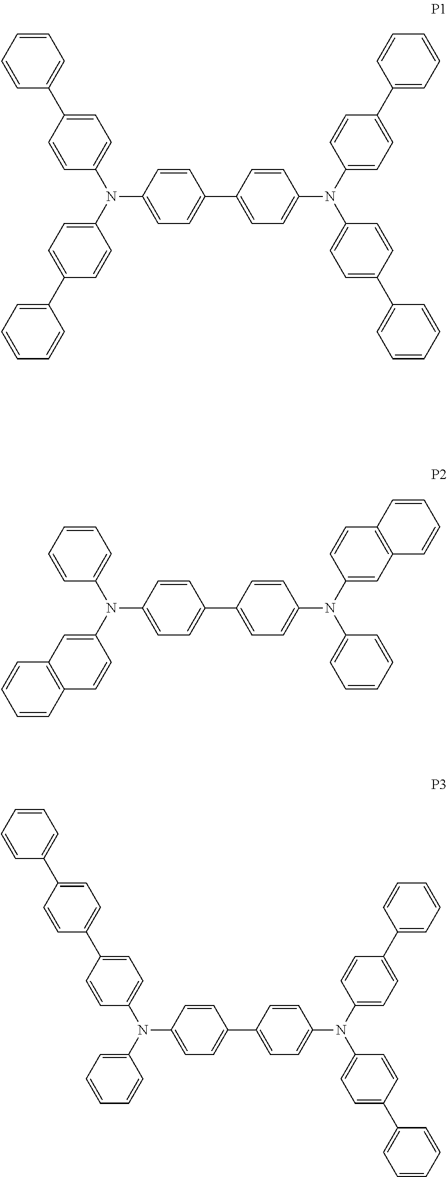 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

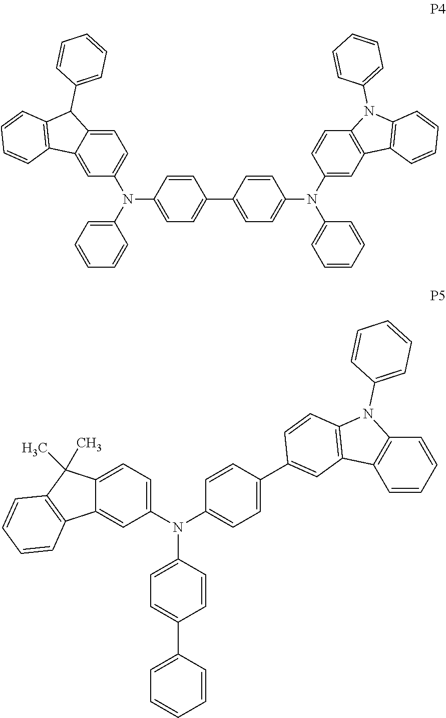 [SMILES: c1ccc(C2c3ccccc3-c3cc(N(c4ccccc4)c4ccc(-c5ccc(N(c6ccccc6)c6ccc7c(c6)c6ccccc6n7-c6ccccc6)cc5)cc4)ccc32)cc1 ; CC1(C)c2ccccc2-c2cc(N(c3ccc(-c4ccccc4)cc3)c3ccc(-c4ccc5c(c4)c4ccccc4n5-c4ccccc4)cc3)ccc21]

In some embodiments, the refractive index of the capping layer CPL may be about 1.6 or more. For example, the refractive index of the capping layer CPL may be about 1.6 or more with respect to light in a wavelength range of about 550 nm to about 660 nm.

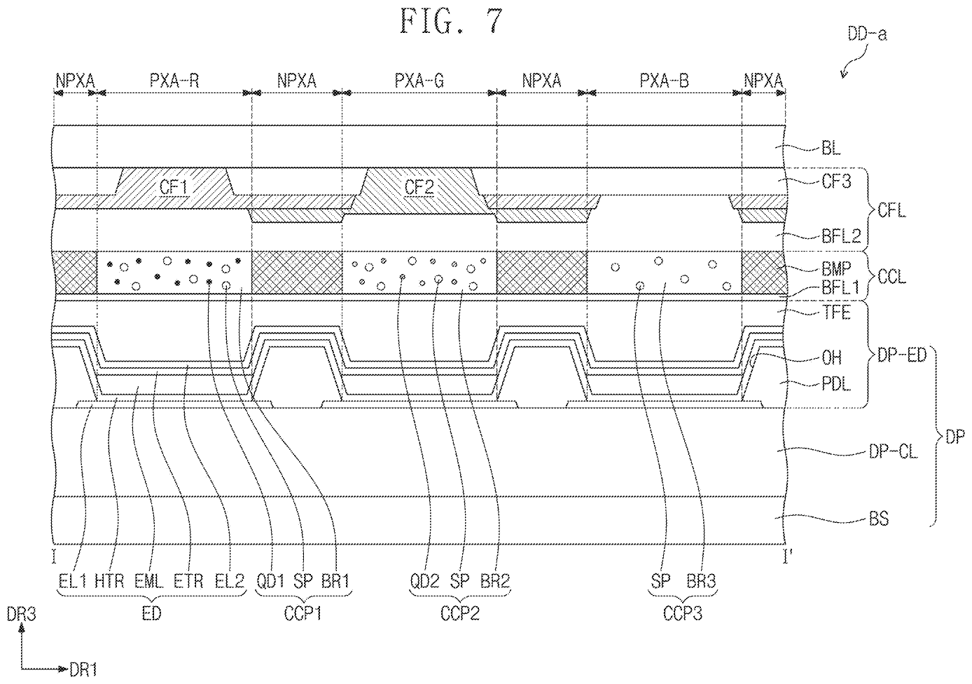

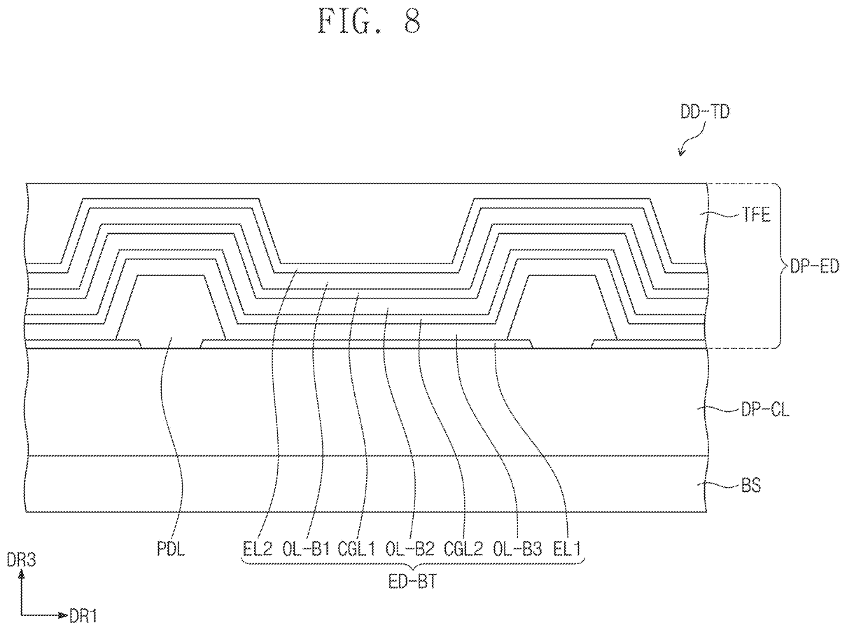

Each of FIGS. 7 to 10 is a cross-sectional view of a display apparatus according to one or more embodiments of the present disclosure. Hereinafter, in describing the display apparatuses of one or more embodiments with reference to FIGS. 7 to 10, the duplicated features which have been described in FIGS. 1 to 6 are not described again for conciseness, but their differences will be mainly described.

Referring to FIG. 7, a display apparatus DD-a according to one or more embodiments may include a display panel DP including a display device layer DP-ED, a light control layer CCL disposed on the display panel DP, and a color filter layer CFL.

In an embodiment illustrated in FIG. 7, the display panel DP may include a base layer BS, a circuit layer DP-CL provided on the base layer BS, and the display device layer DP-ED, and the display device layer DP-ED may include a light emitting element ED.

The light emitting element ED may include a first electrode EL1, a hole transport region HTR disposed on the first electrode EL1, an emission layer EML disposed on the hole transport region HTR, an electron transport region ETR disposed on the emission layer EML, and a second electrode EL2 disposed on the electron transport region ETR. In one or more embodiments, the structures of the light emitting elements of FIGS. 3 to 6 as described above may each be equally applied to the structure of the light emitting element ED illustrated in FIG. 7.

The hole transport region HTR of the light emitting element ED included in the display device DD-a according to an embodiment, may include the amine compound of one or more embodiments of the present disclosure.

Referring to FIG. 7, the emission layer EML may be disposed in an opening OH defined in a pixel defining film PDL. For example, the emission layer EML which is divided by the pixel defining film PDL and provided corresponding to each of light emitting regions PXA-R, PXA-G, and PXA-B may be to emit light in substantially the same wavelength range. In the display apparatus DD-a of an embodiment, the emission layer EML may be to emit blue light. In some embodiments, the emission layer EML may be provided as a common layer in the entire light emitting regions PXA-R, PXA-G, and PXA-B.

The light control layer CCL may be disposed on the display panel DP. The light control layer CCL may include a light conversion body. The light conversion body may be a quantum dot, a phosphor, and/or the like. The light conversion body may be to emit light by wavelength-converting provided light. For example, the light control layer CCL may a layer containing the quantum dot or a layer containing the phosphor.

The light control layer CCL may include a plurality of light control parts CCP1, CCP2, and CCP3. The light control parts CCP1, CCP2, and CCP3 may be spaced apart from each other.

Referring to FIG. 7, divided patterns BMP may be disposed between the light control parts CCP1, CCP2, and CCP3 which are spaced apart from each other, but embodiments of the present disclosure are not limited thereto. FIG. 7 illustrates that the divided patterns BMP do not overlap the light control parts CCP1, CCP2, and CCP3, but, in some embodiments, at least a portion of the edges of the light control parts CCP1, CCP2, and CCP3 may overlap at least a portion of the divided patterns BMP.

In one or more embodiments, the light control layer CCL may include a first light control part CCP1 containing a first quantum dot QD1 which converts first color light provided from the light emitting element ED into second color light, a second light control part CCP2 containing a second quantum dot QD2 which converts the first color light into third color light, and a third light control part CCP3 which transmits the first color light.

In one or more embodiments, the first light control part CCP1 may provide red light that is the second color light, and the second light control part CCP2 may provide green light that is the third color light. The third light control part CCP3 may provide blue light by transmitting the blue light that is the first color light provided from the light emitting element ED. For example, in some embodiments, the first quantum dot QD1 may be a red quantum dot to emit red light, and the second quantum dot QD2 may be a green quantum dot to emit green light. The same as described above regarding the quantum dot may be applied with respect to the quantum dots QD1 and QD2.

In some embodiments, the light control layer CCL may further include a scatterer SP. The first light control part CCP1 may include the first quantum dot QD1 and the scatterer SP, the second light control part CCP2 may include the second quantum dot QD2 and the scatterer SP, and the third light control part CCP3 may not include (e.g., may exclude) any quantum dot but include the scatterer SP.

The scatterer SP may be inorganic particles. For example, the scatterer SP may include at least one of TiO₂, ZnO, Al₂O₃, SiO₂, or hollow sphere silica. In one or more embodiments, the scatterer SP may include any one selected from among TiO₂, ZnO, Al₂O₃, SiO₂, and hollow sphere silica, or may be a mixture of at least two materials selected from among TiO₂, ZnO, Al₂O₃, SiO₂, and hollow sphere silica.

The first light control part CCP1, the second light control part CCP2, and the third light control part CCP3 may respectively include base resins BR1, BR2, and BR3 in which the quantum dots QD1 and QD2 and the scatterer SP are dispersed. In an embodiment, the first light control part CCP1 may include the first quantum dot QD1 and the scatterer SP dispersed in a first base resin BR1, the second light control part CCP2 may include the second quantum dot QD2 and the scatterer SP dispersed in a second base resin BR2, and the third light control part CCP3 may include the scatterer SP dispersed in a third base resin BR3.

The base resins BR1, BR2, and BR3 are media in which the quantum dots QD1 and QD2 and the scatterer SP are dispersed, and may be formed of one or more suitable resin compositions, which may be generally referred to as a binder. For example, the base resins BR1, BR2, and BR3 may each independently be acrylic-based resins, urethane-based resins, silicone-based resins, epoxy-based resins, etc. The base resins BR1, BR2, and BR3 may be transparent resins. In one or more embodiments, the first base resin BR1, the second base resin BR2, and the third base resin BR3 may be the same as or different from each other.

In one or more embodiments, the light control layer CCL may include a barrier layer BFL1. The barrier layer BFL1 may serve to prevent or reduce the penetration of moisture and/or oxygen (hereinafter, referred to as ‘moisture/oxygen’). The barrier layer BFL1 may block or reduce the light control parts CCP1, CCP2, and CCP3 from being exposed to moisture/oxygen. In some embodiments, the barrier layer BFL1 may cover the light control parts CCP1, CCP2, and CCP3. In some embodiments, a barrier layer BFL2 may be provided between the light control parts CCP1, CCP2, and CCP3 and the color filter layer CFL.

The barrier layers BFL1 and BFL2 may include at least one inorganic layer. For example, in some embodiments, the barrier layers BFL1 and BFL2 may include an inorganic material. For example, the barrier layers BFL1 and BFL2 may include a silicon nitride, an aluminum nitride, a zirconium nitride, a titanium nitride, a hafnium nitride, a tantalum nitride, a silicon oxide, an aluminum oxide, a titanium oxide, a tin oxide, a cerium oxide, a silicon oxynitride, a thin metal film which secures a transmittance, etc. In some embodiments, the barrier layers BFL1 and BFL2 may further include an organic film. The barrier layers BFL1 and BFL2 may each be formed of a single layer or a plurality of layers.

In the display apparatus DD-a of an embodiment, the color filter layer CFL may be disposed on the light control layer CCL. For example, in some embodiments, the color filter layer CFL may be directly disposed on the light control layer CCL. In these embodiments, the barrier layer BFL2 may not be provided.

The color filter layer CFL may include color filters CF1, CF2, and CF3. The color filter layer CFL may include a first filter CF1 configured to transmit the second color light, a second filter CF2 configured to transmit the third color light, and a third filter CF3 configured to transmit the first color light. For example, in some embodiments, the first filter CF1 may be a red filter, the second filter CF2 may be a green filter, and the third filter CF3 may be a blue filter. The filters CF1, CF2, and CF3 each may include a polymeric photosensitive resin and a pigment and/or dye. The first filter CF1 may include a red pigment and/or dye, the second filter CF2 may include a green pigment and/or dye, and the third filter CF3 may include a blue pigment and/or dye.

However, embodiments of the present disclosure are not limited thereto, for example, in some embodiments, the third filter CF3 may not include (e.g., may exclude) a pigment and/or dye (e.g., exclude any pigment or dye). The third filter CF3 may include a polymeric photosensitive resin and may not include (e.g., may exclude) a pigment and/or dye (e.g., exclude any pigment or dye). The third filter CF3 may be transparent. The third filter CF3 may be formed of a transparent photosensitive resin.

Furthermore, in an embodiment, the first filter CF1 and the second filter CF2 may be a yellow filter. The first filter CF1 and the second filter CF2 may not be separated but be provided as one filter. The first to third filters CF1, CF2, and CF3 may be disposed corresponding to the red light emitting region PXA-R, the green light emitting region PXA-G, and the blue light emitting region PXA-B, respectively.

In some embodiments, the color filter layer CFL may include a light blocking part. The color filter layer CFL may include a light blocking part disposed to overlap with the boundaries of neighboring filters CF1, CF2, and CF3. The light blocking part may be a black matrix. The light blocking part may be formed by including an organic light blocking material and/or an inorganic light blocking material, including a black pigment and/or a black dye. The light blocking part may divide the boundaries between adjacent filters CF1, CF2, and CF3. In some embodiments, the light blocking part may be formed as a blue filter.

A base substrate BL may be disposed on the color filter layer CFL. The base substrate BL may be a member which provides a base surface on which the color filter layer CFL, the light control layer CCL, and/or the like are disposed. The base substrate BL may be a glass substrate, a metal substrate, a plastic substrate, etc. However, embodiments of the present disclosure are not limited thereto, and the base substrate BL may be an inorganic layer, an organic layer, or a composite material layer. In some embodiments, the base substrate BL may not be provided.

FIG. 8 is a cross-sectional view illustrating a portion of a display apparatus according to one or more embodiments of the present disclosure. In a display apparatus DD-TD of an embodiment, a light emitting element ED-BT may include a plurality of light emitting structures OL-B1, OL-B2, and OL-B3. The light emitting element ED-BT may include a first electrode EL1 and a second electrode EL2 which face each other, and the plurality of light emitting structures OL-B1, OL-B2, and OL-B3 sequentially stacked in a thickness direction between the first electrode EL1 and the second electrode EL2. The light emitting structures OL-B1, OL-B2, and OL-B3 each may include an emission layer EML (FIG. 7) and a hole transport region HTR and an electron transport region ETR disposed with the emission layer EML (FIG. 7) located therebetween.

For example, in some embodiments, the light emitting element ED-BT included in the display apparatus DD-TD may be a light emitting element having a tandem structure and including a plurality of emission layers.

In one or more embodiments illustrated in FIG. 8, all light beams respectively emitted from the light emitting structures OL-B1, OL-B2, and OL-B3 may be blue light. However, embodiments of the present disclosure are not limited thereto, and the light beams respectively emitted from the light emitting structures OL-B1, OL-B2, and OL-B3 may have wavelength ranges different from each other. For example, in some embodiments, the light emitting element ED-BT including the plurality of light emitting structures OL-B1, OL-B2, and OL-B3 which emit light beams having wavelength ranges different from each other may be to emit white light (e.g., combined white light).

Charge generation layers CGL1 and CGL2 may be respectively disposed between two of the neighboring light emitting structures OL-B1, OL-B2, and OL-B3. The charge generation layers CGL1 and CGL2 may include a p-type or kind charge (e.g., P-charge) generation layer and/or an n-type or kind charge (e.g., N-charge) generation layer.

At least one selected from among light emitting structures OL-B1, OL-B2 and OL-B3 included in the display device DD-TD of an embodiment, may include the amine compound of one or more embodiments of the present disclosure.

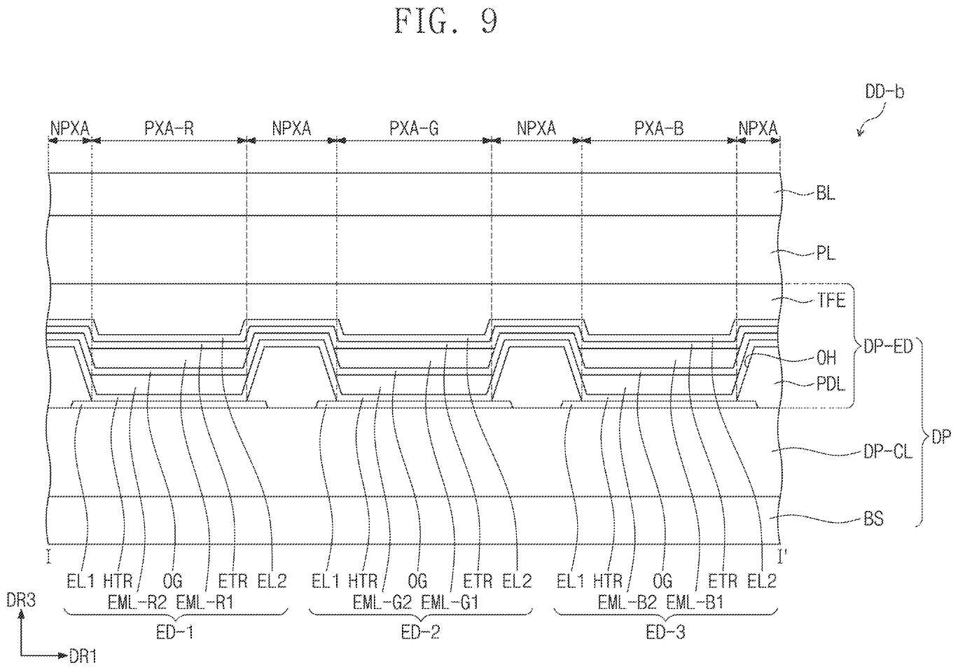

Referring to FIG. 9, a display apparatus DD-b according to an embodiment may include light emitting elements ED-1, ED-2, and ED-3 in each of which two emission layers are stacked. Compared with the display apparatus DD of an embodiment illustrated in FIG. 2, the embodiment illustrated in FIG. 9 has a difference in that the first to third light emitting elements ED-1, ED-2, and ED-3 each include two emission layers stacked in a thickness direction. In some embodiments, In each of the first to third light emitting elements ED-1, ED-2, and ED-3, the two emission layers may be to emit light in substantially the same wavelength region.

In one or more embodiments, the first light emitting element ED-1 may include a first red emission layer EML-R1 and a second red emission layer EML-R2. The second light emitting element ED-2 may include a first green emission layer EML-G1 and a second green emission layer EML-G2. In addition, the third light emitting element ED-3 may include a first blue emission layer EML-B1 and a second blue emission layer EML-B2. An emission auxiliary part OG may be disposed between the first red emission layer EML-R1 and the second red emission layer EML-R2, between the first green emission layer EML-G1 and the second green emission layer EML-G2, and between the first blue emission layer EML-B1 and the second blue emission layer EML-B2.

The emission auxiliary part OG may include a single layer or a multilayer. The emission auxiliary part OG may include a charge generation layer. In one or more embodiments, the emission auxiliary part OG may include an electron transport region, a charge generation layer, and a hole transport region that are sequentially stacked (e.g., in the stated order). The emission auxiliary part OG may be provided as a common layer throughout the first to third light emitting elements ED-1, ED-2, and ED-3. However, embodiments of the present disclosure are not limited thereto, and, in some embodiments, the emission auxiliary part OG may be provided by being patterned within openings OH defined in a pixel defining film PDL.

The first red emission layer EML-R1, the first green emission layer EML-G1, and the first blue emission layer EML-B1 may be disposed between the electron transport region ETR and the emission auxiliary part OG. The second red emission layer EML-R2, the second green emission layer EML-G2, and the second blue emission layer EML-B2 may be disposed between the emission auxiliary part OG and the hole transport region HTR.

For example, in one or more embodiments, the first light emitting element ED-1 may include a first electrode EL1, a hole transport region HTR, a second red emission layer EML-R2, an emission auxiliary part OG, a first red emission layer EML-R1, an electron transport region ETR, and a second electrode EL2, which are sequentially stacked. The second light emitting element ED-2 may include a first electrode EL1, a hole transport region HTR, a second green emission layer EML-G2, an emission auxiliary part OG, a first green emission layer EML-G1, an electron transport region ETR, and a second electrode EL2, which are sequentially stacked. The third light emitting element ED-3 may include a first electrode EL1, a hole transport region HTR, a second blue emission layer EML-B2, an emission auxiliary part OG, a first blue emission layer EML-B1, an electron transport region ETR, and a second electrode EL2, which are sequentially stacked.

In some embodiments, an optical auxiliary layer PL may be disposed on the display device layer DP-ED. The optical auxiliary layer PL may include a polarizing layer. The optical auxiliary layer PL may be disposed on the display panel DP to control reflected light in the display panel DP due to external light. In some embodiments, the optical auxiliary layer PL in the display apparatus may not be provided.

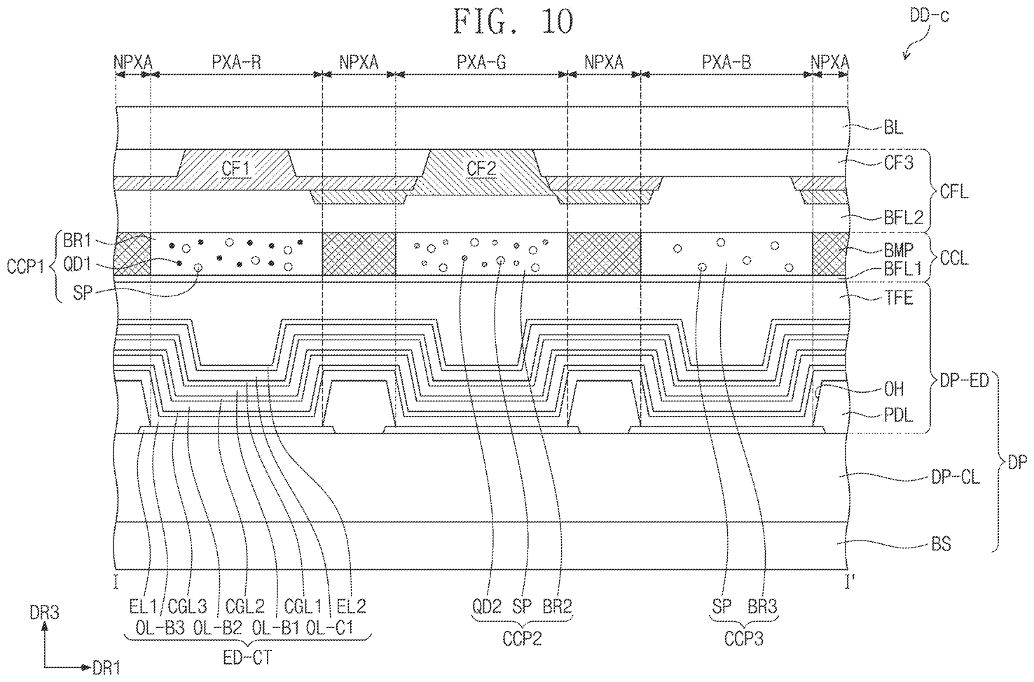

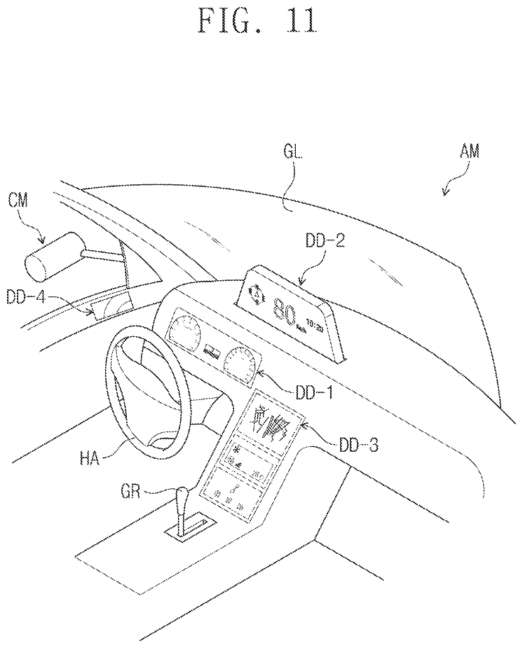

Unlike FIG. 8 and FIG. 9, FIG. 10 illustrates that a display apparatus DD-c includes four light emitting structures OL-B1, OL-B2, OL-B3, and OL-C1. A light emitting element ED-CT may include a first electrode EL1 and a second electrode EL2 which face each other, and first to fourth light emitting structures OL-B1, OL-B2, OL-B3, and OL-C1 that are stacked in a thickness direction between the first electrode EL1 and the second electrode EL2. Charge generation layers CGL1, CGL2, and CGL3 may be disposed between the first to fourth light emitting structures OL-B1, OL-B2, OL-B3, and OL-C1. In one or more embodiments, among the four light emitting structures, the first to third light emitting structures OL-B1, OL-B2, and OL-B3 may be to emit blue light, and the fourth light emitting structure OL-C1 may be to emit green light. However, embodiments of the present disclosure are not limited thereto, and the first to fourth light emitting structures OL-B1, OL-B2, OL-B3, and OL-C1 may be to emit light beams in different wavelength regions.

The charge generation layers CGL1, CGL2, and CGL3 disposed between adjacent light emitting structures OL-B1, OL-B2, OL-B3, and OL-C1 may include a p-type or kind charge (e.g., P-charge) generation layer and/or an n-type or kind charge (N-charge) generation layer.

In at least one selected from among the light emitting structures OL-B1, OL-B2, OL-B3 and OL-C1, included in the display device DD-c of an embodiment, the amine compound of one or more embodiments may be included.

The light emitting element ED according to one or more embodiments of the present disclosure may include the amine compound of one or more embodiments in at least one functional layer disposed between the first electrode EL1 and the second electrode EL2 to show improved emission efficiency and improved life characteristics. The light emitting element ED according to one or more embodiments may include the amine compound of one or more embodiments in at least one selected from among a hole transport region HTR, an emission layer EML, and an electron transport region ETR, disposed between the first electrode EL1 and the second electrode EL2, or in a capping layer CPL. For example, in one or more embodiments, the amine compound according to an embodiment may be included in the hole transport region HTR of the light emitting element ED, and the light emitting element may show high efficiency and long-life characteristics.

The amine compound of one or more embodiments includes a first core, and second and third substituents and may improve the stability of a material and improve hole transport properties. Accordingly, the lifetime and efficiency of the light emitting element including the amine compound of one or more embodiments may be improved. In some embodiments, the light emitting element may include the amine compound according to one or more embodiments in a hole transport layer of the light emitting element to show improved efficiency and lifetime characteristics.

FIG. 11 is a view illustrating a vehicle AM in which first to fourth display apparatuses DD-1, DD-2, DD-3, and DD-4 are disposed. At least one selected from among the first to fourth display apparatuses DD-1, DD-2, DD-3, and DD-4 may include the same configuration as one of the display apparatuses DD, DD-TD, DD-a, DD-b, and DD-c as described with reference to FIGS. 1, and 2, and 7 to 10.

FIG. 11 illustrates a vehicle AM, but this is a mere example, and the first to fourth display apparatuses DD-1, DD-2, DD-3, and DD-4 may be disposed in another transportation means such as bicycles, motorcycles, trains, ships, or airplanes. In some embodiments, at least one selected from among the first to fourth display apparatuses DD-1, DD-2, DD-3, and DD-4 including the same configuration as one of the display apparatuses DD, DD-TD, DD-a, DD-b, and DD-c of an embodiment may be employed in a personal computer, a laptop computer, a personal digital terminal, a game console, a portable electronic device, a television, a monitor, an outdoor billboard, and/or the like. These are merely provided as embodiments, and thus may be employed in other electronic apparatuses unless departing from the present disclosure.

At least one selected from among the first to fourth display apparatuses DD-1, DD-2, DD-3, and DD-4 may include the light emitting element ED of an embodiment as described with reference to FIGS. 3 to 6. The light emitting element ED of an embodiment may include the amine compound of one or more embodiments. At least one selected from among the first to fourth display devices DD-1, DD-2, DD-3, and DD-4 may include the light emitting element ED including the amine compound of one or more embodiments and may show improved display lifetime.

Referring to FIG. 11, a vehicle AM may include a steering wheel HA and a gear GR for the operation of the vehicle AM. In addition, the vehicle AM may include a front window GL disposed to face a driver.

The first display apparatus DD-1 may be disposed in a first region overlapping the steering wheel HA. For example, the first display apparatus DD-1 may be a digital cluster which displays first information of the vehicle AM. The first information may include a first scale which indicates a driving speed of the vehicle AM, a second scale which indicates an engine speed (that is, revolutions per minute (RPM)), an image which indicates a fuel state, etc. A first scale and a second scale may be indicated as a digital image.

The second display apparatus DD-2 may be disposed in a second region facing a driver seat and overlapping the front window GL. The driver seat may be a seat which the steering wheel HA faces. For example, the second display apparatus DD-2 may be a head up display (HUD) which displays second information of the vehicle AM. The second display apparatus DD-2 may be optically transparent. The second information may include digital numbers which indicate a driving speed, and may further include information such as the current time. Different from the drawing, the second information of the second display device DD-2 may be projected and displayed on the front window GL.

The third display apparatus DD-3 may be disposed in a third region adjacent to the gear GR. For example, the third display apparatus DD-3 may be disposed between the driver seat and a passenger seat and may be a center information display (CID) for the vehicle for displaying third information. The passenger seat may be a seat spaced apart from the driver seat with the gear GR disposed therebetween. The third information may include information about traffic (e.g., navigation information), playing music or radio or a video (or an image), temperatures inside the vehicle AM, etc.

The fourth display apparatus DD-4 may be spaced apart from the steering wheel HA and the gear GR, and may be disposed in a fourth region adjacent to a side of the vehicle AM. For example, the fourth display apparatus DD-4 may be a digital side-view mirror which displays fourth information. The fourth display apparatus DD-4 may display an image outside the vehicle AM taken by a camera module CM disposed outside the vehicle AM. The fourth information may include an image outside the vehicle AM.

The above-described first to fourth information may be mere examples, and the first to fourth display apparatuses DD-1, DD-2, DD-3, and DD-4 may further display information about the inside and outside of the vehicle AM. The first to fourth information may include different information. However, embodiments of the present disclosure are not limited thereto, and a part of the first to fourth information may include the same information as one another.

Hereinafter, referring to Examples and Comparative Examples, the amine compound according to one or more embodiments and the light emitting element according to one or more embodiments of the present disclosure will be explained in more detail. In addition, the Examples are illustrations to assist the understanding of the present disclosure, but the scope of the present disclosure is not limited thereto.

### Examples

### 1. Synthesis of Amine Compounds

First, the synthetic methods of the amine compounds according to one or more embodiments will be explained in more detail by illustrating the synthetic methods of Compound DS21, Compound DJ29, Compound DJ34, Compound DS5, Compound DL5, Compound AJ16, Compound DW5, and Compound DW21. In addition, the synthetic methods of the amine compounds explained hereinafter are examples, and the synthetic method of the amine compound according to one or more embodiments of the present disclosure is not limited to these examples.

### (1) Synthesis of Compound DS21

Compound DS21 according to one or more embodiments may be synthesized according to, for example, Reaction 1-1 and Reaction 1-2.

### 1) Synthesis of Compound X3

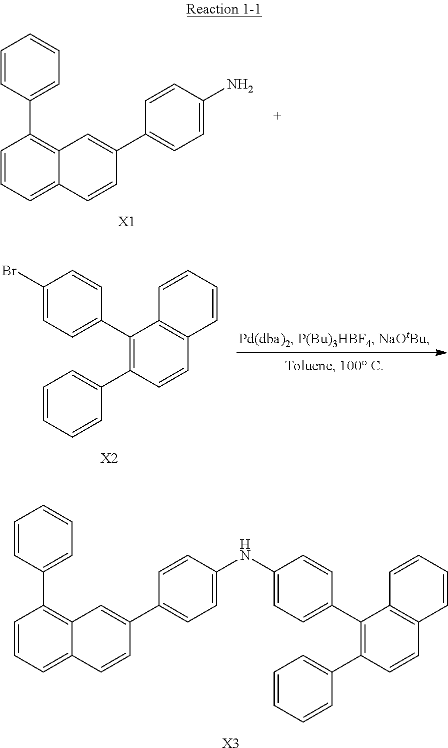

To Compound X1 (10 mmol), Compound X2 (10 mmol), NaOtBu (10 mmol), and P(Bu)₃HBF₄ (1 mmol), toluene (200 mL) was added, and then the gas (e.g., air) was removed therefrom. Under an argon atmosphere, bis(dibenzylideneacetone)palladium (Pd(dba)₂) (0.5 mmol) was added thereto, followed by heating and stirring at about 100° C. for about 6 hours. The reaction solution was cooled to room temperature, extracted with toluene, washed with H₂O and brine, and dried over Na₂SO₄. The solution thus obtained was concentrated and purified by column chromatography to obtain Compound X3 (6.7 mmol, 67%, MS 573.25).

### 2) Synthesis of Compound DS21

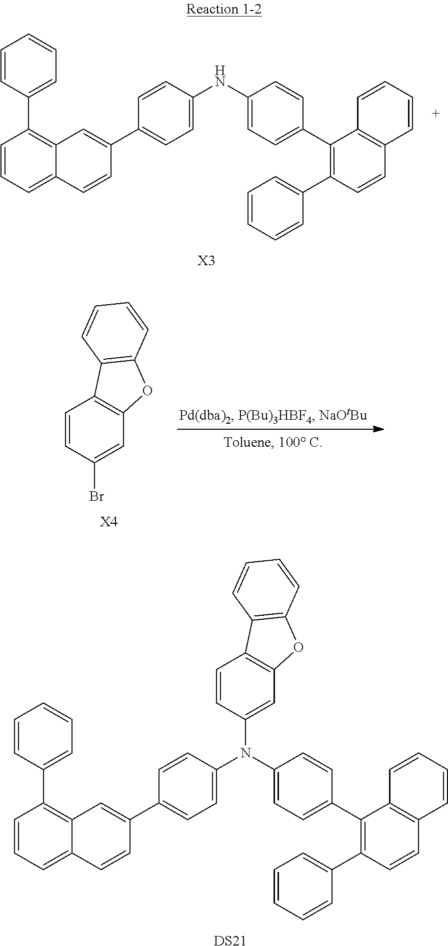

To Compound X3 (10 mmol), Compound X4 (10 mmol), NaOtBu (10 mmol), and P(Bu)₃HBF₄ (1 mmol), toluene (200 mL) was added, and then the gas (e.g., air) was removed therefrom. Under an argon atmosphere, bis(dibenzylideneacetone)palladium (0.5 mmol) was added thereto, followed by heating and stirring at about 100° C. for about 6 hours. The reaction solution was cooled to room temperature, extracted with toluene, washed with H₂O and brine, and dried over Na₂SO₄. The solution thus obtained was concentrated and purified by column chromatography to obtain Compound DS21 (8.2 mmol, 82%, MS 739.29).

### (2) Synthesis of Compound DJ29

Compound DJ29 according to one or more embodiments may be synthesized according to, for example, Reaction 2-1 and Reaction 2-2.

### 1) Synthesis of Compound X6

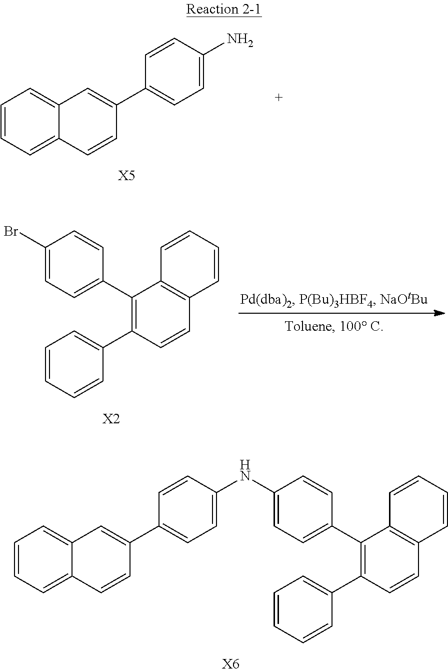

To Compound X5 (10 mmol), Compound X2 (10 mmol), NaOtBu (10 mmol), and P(Bu)₃HBF₄ (1 mmol), toluene (200 mL) was added, and then the gas (e.g., air) was removed therefrom. Under an argon atmosphere, bis(dibenzylideneacetone)palladium (0.5 mmol) was added thereto, followed by heating and stirring at about 100° C. for about 6 hours. The reaction solution was cooled to room temperature, extracted with toluene, washed with H₂O and brine, and dried over Na₂SO₄. The solution thus obtained was concentrated and purified by column chromatography to obtain Compound X6 (7.3 mmol, 73%, MS 497.21).

### 2) Synthesis of Compound DJ29

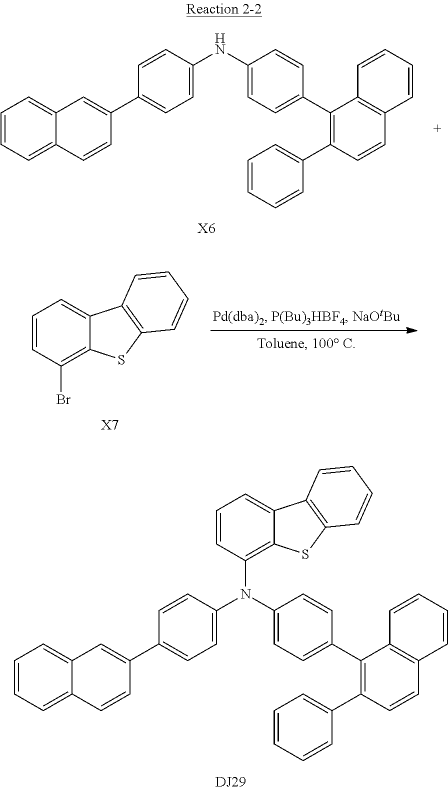 [SMILES: c1ccc(-c2ccc3ccccc3c2-c2ccc(Nc3ccc(-c4ccc5ccccc5c4)cc3)cc2)cc1 ; "X6" ; Brc1cccc2c1sc1ccccc12 ; "X7" ; c1ccc(-c2ccc3ccccc3c2-c2ccc(N(c3ccc(-c4ccc5ccccc5c4)cc3)c3cccc4c3sc3ccccc34)cc2)cc1 ; "DJ29"]

To Compound X6 (10 mmol), Compound X7 (10 mmol), NaOtBu (10 mmol), and P(Bu)₃HBF₄ (1 mmol), toluene (200 mL) was added, and then the gas (e.g., air) was removed therefrom. Under an argon atmosphere, bis(dibenzylideneacetone)palladium (0.5 mmol) was added thereto, followed by heating and stirring at about 100° C. for about 6 hours. The reaction solution was cooled to room temperature, extracted with toluene, washed with H₂O and brine, and dried over Na₂SO₄. The solution thus obtained was concentrated and purified by column chromatography to obtain Compound DJ29 (8.6 mmol, 86%, MS 679.23).

### (3) Synthesis of Compound DJ34

Compound DJ34 according to one or more embodiments may be synthesized according to, for example, Reaction 3.

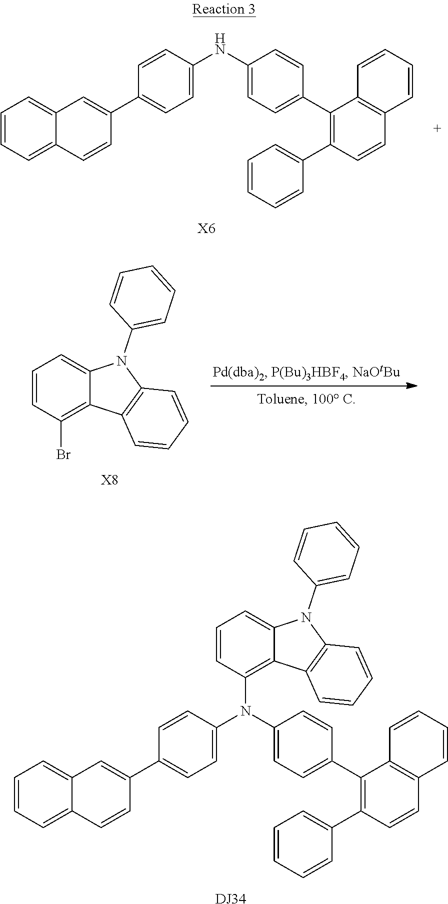

To Compound X6 (10 mmol), Compound X8 (10 mmol), NaOtBu (10 mmol), and P(Bu)₃HBF₄ (1 mmol), toluene (200 mL) was added, and then the gas (e.g., air) was removed therefrom. Under an argon atmosphere, bis(dibenzylideneacetone)palladium (0.5 mmol) was added thereto, followed by heating and stirring at about 100° C. for about 6 hours. The reaction solution was cooled to room temperature, extracted with toluene, washed with H₂O and brine, and dried over Na₂SO₄. The solution thus obtained was concentrated and purified by column chromatography to obtain Compound DJ34 (8.1 mmol, 81%, MS 638.30).

### (4) Synthesis of Compound DS5

Compound DS5 according to one or more embodiments may be synthesized according to, for example, Reaction 4

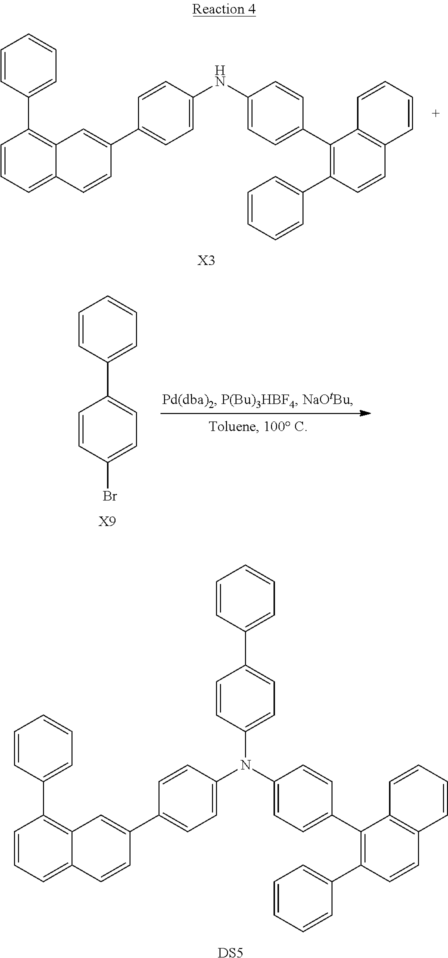

To Compound X3 (10 mmol), Compound X9 (10 mmol), NaOtBu (10 mmol), and P(Bu)₃HBF₄ (1 mmol), toluene (200 mL) was added, and then the gas (e.g., air) was removed therefrom. Under an argon atmosphere, bis(dibenzylideneacetone)palladium (0.5 mmol) was added thereto, followed by heating and stirring at about 100° C. for about 6 hours. The reaction solution was cooled to room temperature, extracted with toluene, washed with H₂O and brine, and dried over Na₂SO₄. The solution thus obtained was concentrated and purified by column chromatography to obtain Compound DS5 (7.9 mmol, 79%, MS 725.31).

### (5) Synthesis of Compound DL5

Compound DL5 according to one or more embodiments may be synthesized according to, for example, Reaction 5-1 and Reaction 5-2.

### 1) Synthesis of Compound X11

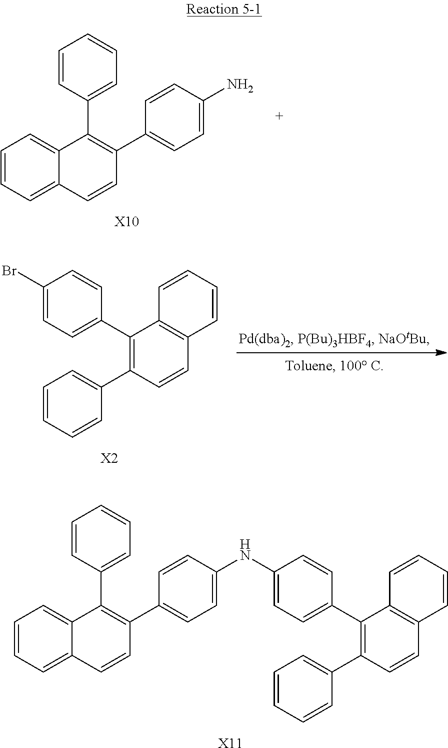

To Compound X10 (10 mmol), Compound X2 (10 mmol), NaOtBu (10 mmol), and P(Bu)₃HBF₄ (1 mmol), toluene (200 mL) was added, and then the gas (e.g., air) was removed therefrom. Under an argon atmosphere, bis(dibenzylideneacetone)palladium (0.5 mmol) was added thereto, followed by heating and stirring at about 100° C. for about 6 hours. The reaction solution was cooled to room temperature, extracted with toluene, washed with H₂O and brine, and dried over Na₂SO₄. The solution thus obtained was concentrated and purified by column chromatography to obtain Compound X11 (6.1 mmol, 61%, MS 573.25).

### 2) Synthesis of Compound DL5

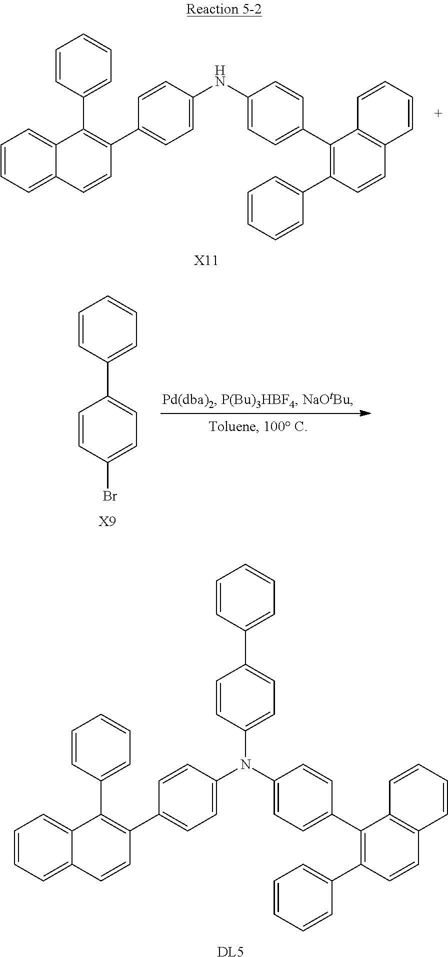

To Compound X11 (10 mmol), Compound X9 (10 mmol), NaOtBu (10 mmol), and P(Bu)₃HBF₄ (1 mmol), toluene (200 mL) was added, and then the gas (e.g., air) was removed therefrom. Under an argon atmosphere, bis(dibenzylideneacetone)palladium (0.5 mmol) was added thereto, followed by heating and stirring at about 100° C. for about 6 hours. The reaction solution was cooled to room temperature, extracted with toluene, washed with H₂O and brine, and dried over Na₂SO₄. The solution thus obtained was concentrated and purified by column chromatography to obtain Compound DL5 (7.8 mmol, 78%, MS 725.31).

### (6) Synthesis of Compound AJ16

Compound AJ16 according to one or more embodiments may be synthesized according to, for example, Reaction 6

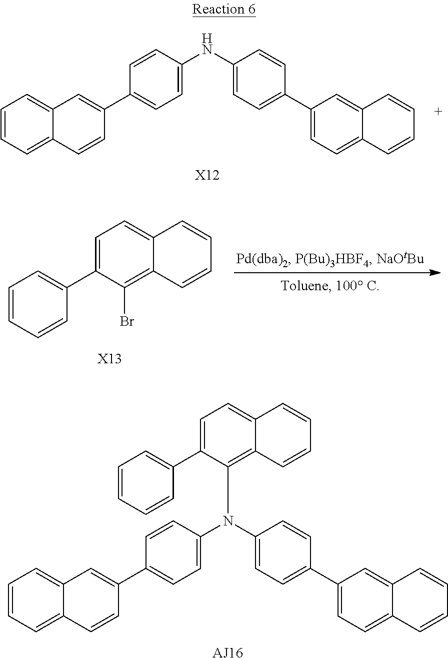

To Compound X12 (10 mmol), Compound X13 (10 mmol), NaOtBu (10 mmol), and P(Bu)₃HBF₄ (1 mmol), toluene (200 mL) was added, and then the gas (e.g., air) was removed therefrom. Under an argon atmosphere, bis(dibenzylideneacetone)palladium (0.5 mmol) was added thereto, followed by heating and stirring at about 100° C. for about 6 hours. The reaction solution was cooled to room temperature, extracted with toluene, washed with H₂O and brine, and dried over Na₂SO₄. The solution thus obtained was concentrated and purified by column chromatography to obtain Compound AJ16 (8.8 mmol, 88%, MS 623.26).

### (7) Synthesis of Compound DW5

Compound DW5 according to one or more embodiments may be synthesized according to, for example, Reaction 7-1 and Reaction 7-2.

### 1) Synthesis of Compound X15

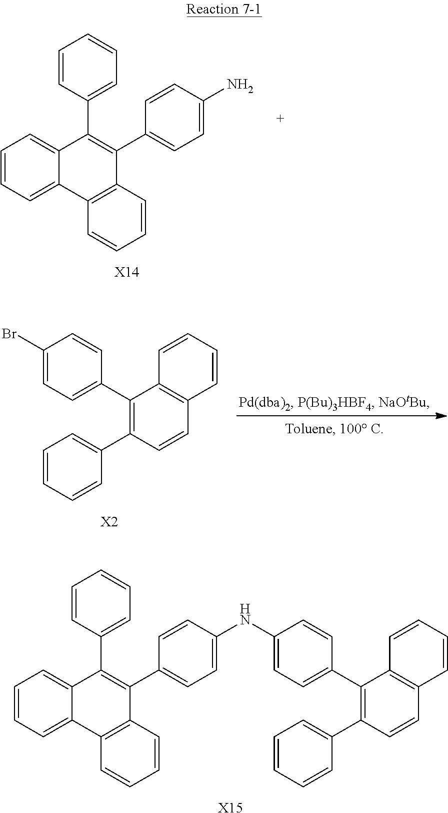

To Compound X14 (10 mmol), Compound X2 (10 mmol), NaOtBu (10 mmol), and P(Bu)₃HBF₄ (1 mmol), toluene (200 mL) was added, and then the gas (e.g., air) was removed therefrom. Under an argon atmosphere, bis(dibenzylideneacetone)palladium (0.5 mmol) was added thereto, followed by heating and stirring at about 100° C. for about 6 hours. The reaction solution was cooled to room temperature, extracted with toluene, washed with H₂O and brine, and dried over Na₂SO₄. The solution thus obtained was concentrated and purified by column chromatography to obtain Compound X15 (5.9 mmol, 59%, MS 623.26).

### 2) Synthesis of Compound DW5

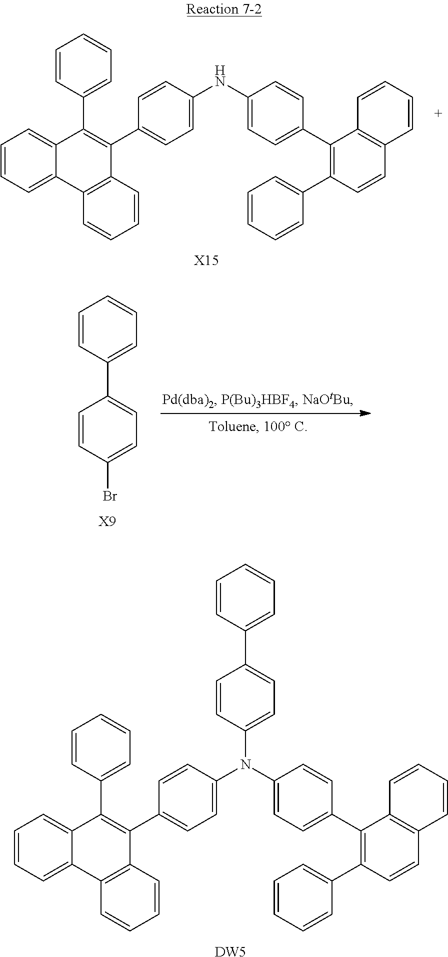

To Compound X15 (10 mmol), Compound X9 (10 mmol), NaOtBu (10 mmol), and P(Bu)₃HBF₄ (1 mmol), toluene (200 mL) was added, and then the gas (e.g., air) was removed therefrom. Under an argon atmosphere, bis(dibenzylideneacetone)palladium (0.5 mmol) was added thereto, followed by heating and stirring at about 100° C. for about 6 hours. The reaction solution was cooled to room temperature, extracted with toluene, washed with H₂O and brine, and dried over Na₂SO₄. The solution thus obtained was concentrated and purified by column chromatography to obtain Compound DW5 (9.1 mmol, 91%, MS 775.32).

### (8) Synthesis of Compound DW21

Compound DW21 according to one or more embodiments may be synthesized according to, for example, Reaction 8

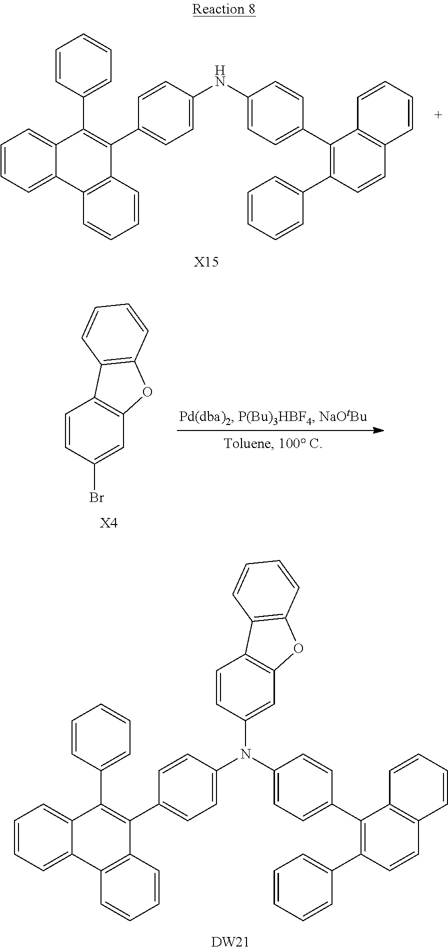

To Compound X15 (10 mmol), Compound X4 (10 mmol), NaOtBu (10 mmol), and P(Bu)₃HBF₄ (1 mmol), toluene (200 mL) was added, and then the gas (e.g., air) was removed therefrom. Under an argon atmosphere, bis(dibenzylideneacetone)palladium (0.5 mmol) was added thereto, followed by heating and stirring at about 100° C. for about 6 hours. The reaction solution was cooled to room temperature, extracted with toluene, washed with H₂O and brine, and dried over Na₂SO₄. The solution thus obtained was concentrated and purified by column chromatography to obtain Compound DW21 (8.7 mmol, 87%, MS 789.30).

### 2. Manufacture and Evaluation of Light Emitting Elements

A light emitting element of an embodiment, including the amine compound of an embodiment in a hole transport layer of the light emitting element was manufactured by a method described herein. Light emitting elements of Example 1 to Example 8 were manufactured utilizing the amine compounds of Compound DS21, Compound DJ29, Compound DJ34, Compound DS5, Compound DL5, Compound AJ16, Compound DW5, and Compound DW21, respectively, which are the above-explained Example Compounds as a hole transport material. Comparative Example 1 to Comparative Example 10 correspond to light emitting elements manufactured utilizing Comparative Compounds R1 to R10, respectively, as a hole transport material.

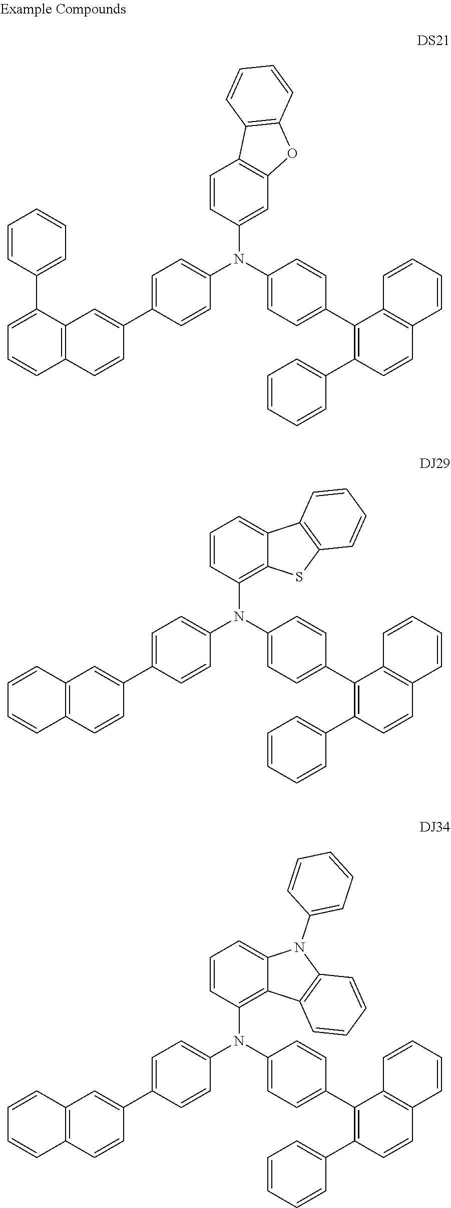 [SMILES: c1ccc(-c2ccc3ccccc3c2-c2ccc(N(c3ccc(-c4ccc5cccc(-c6ccccc6)c5c4)cc3)c3ccc4c(c3)oc3ccccc34)cc2)cc1 ; c1ccc(-c2ccc3ccccc3c2-c2ccc(N(c3ccc(-c4ccc5ccccc5c4)cc3)c3cccc4c3sc3ccccc34)cc2)cc1 ; c1ccc(-c2ccc3ccccc3c2-c2ccc(N(c3ccc(-c4ccc5ccccc5c4)cc3)c3cccc4c3c3ccccc3n4-c3ccccc3)cc2)cc1]

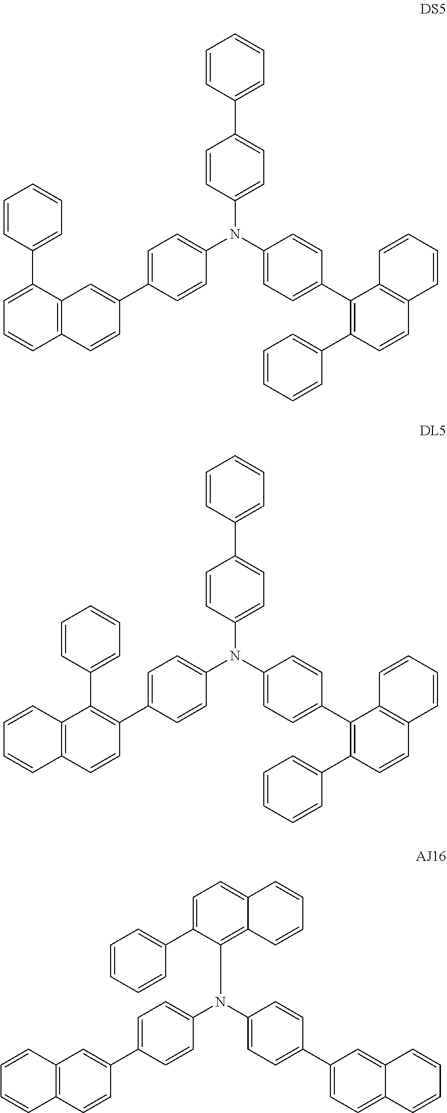 [SMILES: c1ccc(-c2ccc(N(c3ccc(-c4ccc5cccc(-c6ccccc6)c5c4)cc3)c3ccc(-c4c(-c5ccccc5)ccc5ccccc45)cc3)cc2)cc1 ; c1ccc(-c2ccc(N(c3ccc(-c4ccc5ccccc5c4-c4ccccc4)cc3)c3ccc(-c4c(-c5ccccc5)ccc5ccccc45)cc3)cc2)cc1 ; c1ccc(-c2ccc3ccccc3c2N(c2ccc(-c3ccc4ccccc4c3)cc2)c2ccc(-c3ccc4ccccc4c3)cc2)cc1]

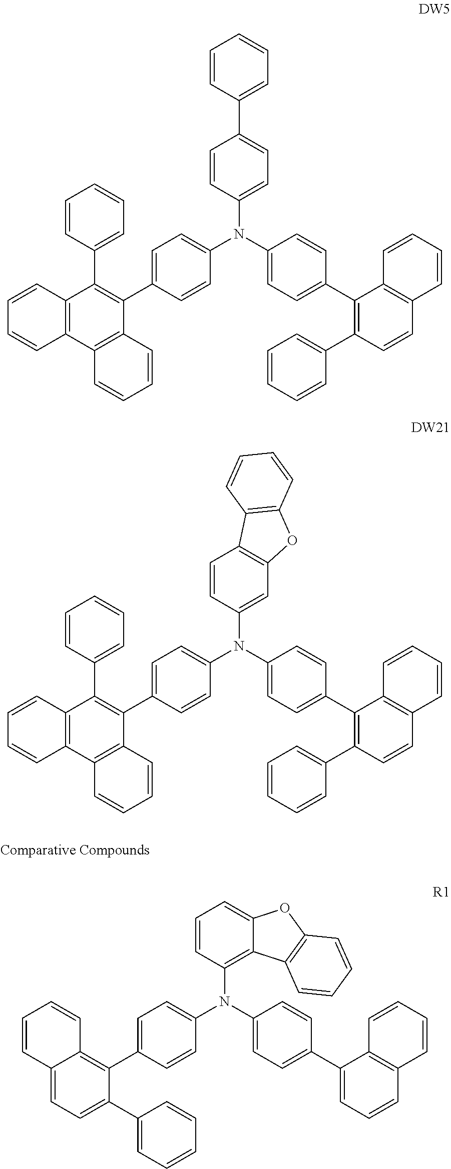 [SMILES: c1ccc(-c2ccc(N(c3ccc(-c4c(-c5ccccc5)ccc5ccccc45)cc3)c3ccc(-c4c(-c5ccccc5)c5ccccc5c5ccccc45)cc3)cc2)cc1 ; c1ccc(-c2ccc3ccccc3c2-c2ccc(N(c3ccc(-c4c(-c5ccccc5)c5ccccc5c5ccccc45)cc3)c3ccc4c(c3)oc3ccccc34)cc2)cc1 ; c1ccc(-c2ccc3ccccc3c2-c2ccc(N(c3ccc(-c4cccc5ccccc45)cc3)c3cccc4oc5ccccc5c34)cc2)cc1]

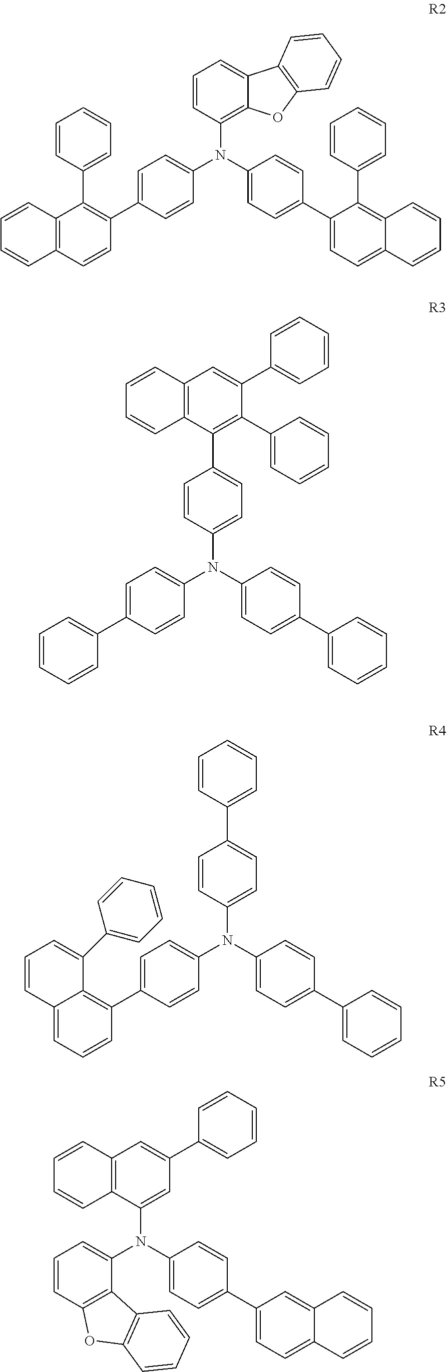 [SMILES: c1ccc(-c2c(-c3ccc(N(c4ccc(-c5ccc6ccccc6c5-c5ccccc5)cc4)c4cccc5c4oc4ccccc45)cc3)ccc3ccccc23)cc1 ; c1ccc(-c2ccc(N(c3ccc(-c4ccccc4)cc3)c3ccc(-c4c(-c5ccccc5)c(-c5ccccc5)cc5ccccc45)cc3)cc2)cc1 ; c1ccc(-c2ccc(N(c3ccc(-c4ccccc4)cc3)c3ccc(-c4cccc5cccc(-c6ccccc6)c45)cc3)cc2)cc1 ; c1ccc(-c2cc(N(c3ccc(-c4ccc5ccccc5c4)cc3)c3cccc4oc5ccccc5c34)c3ccccc3c2)cc1]

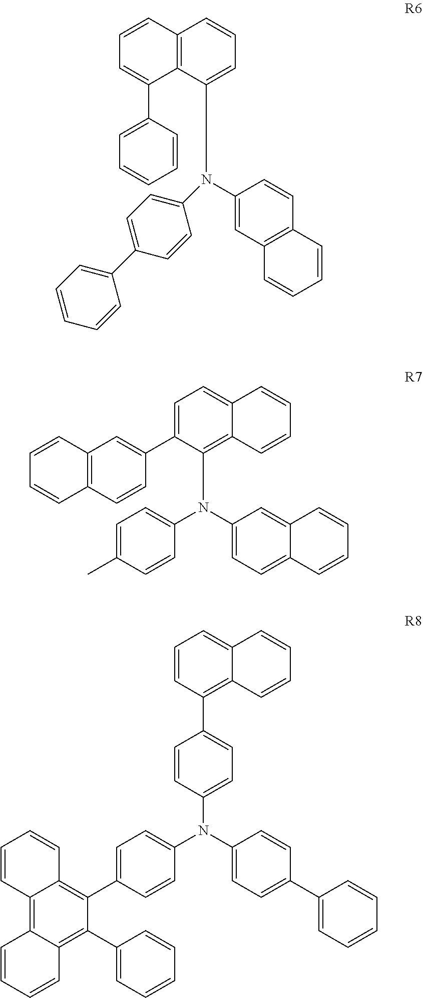 [SMILES: c1ccc(-c2ccc(N(c3ccc4ccccc4c3)c3cccc4cccc(-c5ccccc5)c34)cc2)cc1 ; Cc1ccc(N(c2ccc3ccccc3c2)c2c(-c3ccc4ccccc4c3)ccc3ccccc23)cc1 ; c1ccc(-c2ccc(N(c3ccc(-c4cccc5ccccc45)cc3)c3ccc(-c4c(-c5ccccc5)c5ccccc5c5ccccc45)cc3)cc2)cc1]

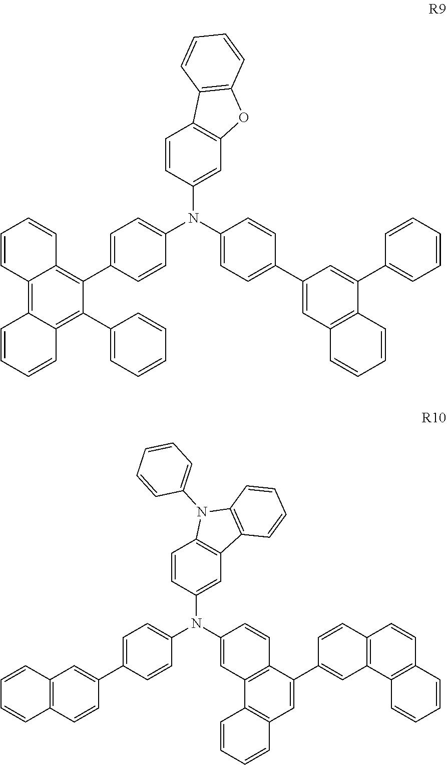 [SMILES: c1ccc(-c2cc(-c3ccc(N(c4ccc(-c5c(-c6ccccc6)c6ccccc6c6ccccc56)cc4)c4ccc5c(c4)oc4ccccc45)cc3)cc3ccccc23)cc1 ; c1ccc(-n2c3ccccc3c3cc(N(c4ccc(-c5ccc6ccccc6c5)cc4)c4ccc5c(-c6ccc7ccc8ccccc8c7c6)cc6ccccc6c5c4)ccc32)cc1]

### Manufacture of Light Emitting Elements

An ITO glass substrate with about 15 Ω/cm² (about 150 nm ITO in a thickness) of Corning Co. was cut into a size of 50 mm×50 mm×0.7 mm, washed with isopropyl alcohol and ultrapure water, cleansed utilizing ultrasonic waves for about 5 minutes, exposed to UV for about 30 minutes and treated with ozone. Then, 4,4′,4″-tris{N-(2-naphthyl)-N-phenylamino}-triphenylamine (2-TNATA) was vacuum deposited to a thickness of about 60 nm to form a hole injection layer. After that, the Example Compound or Comparative Compound was vacuum deposited to a thickness of about 30 nm to form a hole transport layer.

On the hole transport layer, a blue fluorescence host of 9,10-di(naphthalen-2-yl)anthracene (ADN) and a fluorescence dopant of 2,5,8,11-tetra-t-butylperylene (TBP) were co-deposited in a ratio (e.g., a vol %) of about 97:3 to form an emission layer with a thickness of about 25 nm.

On the emission layer, an electron transport layer was formed with a thickness of about 250 nm utilizing tris(8-hydroxyquinolinato)aluminum (Alq₃), and then, an electron injection layer was formed with a thickness of about 1 nm by depositing LiF. On the electron injection layer, a second electrode was formed with a thickness of about 100 nm utilizing aluminum (Al).

In addition, the compounds of the functional layers utilized for the manufacture of the light emitting elements are as follows.

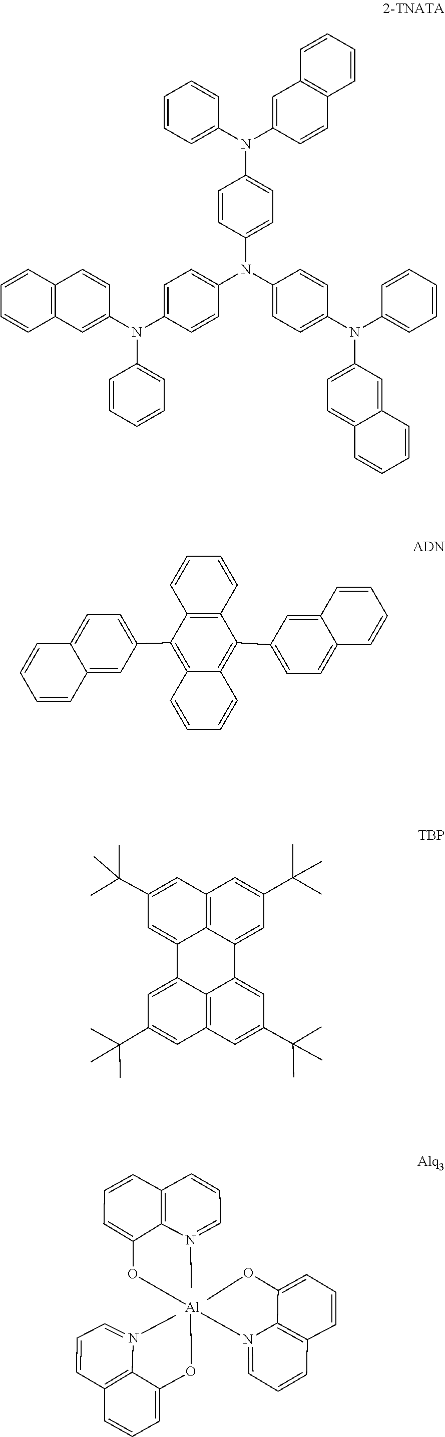

### Evaluation of Light Emitting Elements

Table 1 shows evaluation results on the light emitting elements of Examples to 8, and Comparative Examples 1 to 10. In Table 1, the evaluation results of the lifetime of the light emitting elements manufactured are shown.

In the evaluation results on the properties of the Examples and Comparative Examples, shown in Table 1, the element lifetime (LT95, %) represents element lifetime (LT95) taken for reducing an initial luminance of about 1000 cd/m2 to a 95% level. The element lifetime (LT95) was measured by continuously driving at a current density of about 10 mA/cm². A relative lifetime LT95, %, of each Example and each Comparative Example was calculated and presented with respect to the element lifetime (LT95) of Comparative Example 1.

TABLE 1

| | | Relative |
|---|---|---|
| Element | Hole transport | Lifetime |
| example | layer | LT95, % |
| Example 1 | Example | 123 |
| | Compound | |
| | DS21 | |
| Example 2 | Example | 116 |
| | Compound | |
| | DJ29 | |
| Example 3 | Example | 119 |
| | Compound | |
| | DJ34 | |
| Example 4 | Example | 121 |
| | Compound | |
| | DS5 | |
| Example 5 | Example | 118 |
| | Compound | |
| | DL5 | |
| Example 6 | Example | 113 |
| | Compound | |
| | AJ16 | |
| Example 7 | Example | 113 |
| | Compound | |
| | DW5 | |
| Example 8 | Example | 117 |
| | Compound | |
| | DW21 | |
| Comparative | Comparative | 100% |
| Example 1 | Compound R1 | |
| Comparative | Comparative | 103 |
| Example 2 | Compound R2 | |
| Comparative | Comparative | 99 |
| Example 3 | Compound R3 | |
| Comparative | Comparative | 102 |
| Example 4 | Compound R4 | |
| Comparative | Comparative | 99 |
| Example 5 | Compound R5 | |
| Comparative | Comparative | 96 |
| Example 6 | Compound R6 | |
| Comparative | Comparative | 95 |
| Example 7 | Compound R7 | |
| Comparative | Comparative | 101 |
| Example 8 | Compound R8 | |
| Comparative | Comparative | 101 |
| Example 9 | Compound R9 | |
| Comparative | Comparative | 97% |
| Example 10 | Compound R10 | |

Referring to the results of Table 1, it could be found that the light emitting elements of the Examples, utilizing the amine compounds according to embodiments of the present disclosure as the hole transport layer materials, each showed relatively long element lifetime when compared to the Comparative Examples.

The amine compound of one or more embodiments according to the present disclosure includes a first substituent, an α-naphthyl group in which an aryl group is substituted at position 2 (hereinafter, a second substituent), and a ß-naphthyl group (hereinafter, a third substituent), which are directly or indirectly connected with the nitrogen atom of the amine. The amine compounds included in Example 1 to Example 8 include second substituents and third substituents having different substitution positions, and specific long lifetime was confirmed in cases of including any aryl group or heteroaryl group as the first substituent. According to the present disclosure, without wishing to be bound by any theory, the increase of the lifetime of the light emitting element is thought as effects mainly due to the combination of naphthyl groups with different substitution positions, and the same level of effects are shown for an aryl group, a dibenzofuran group, a dibenzothiophene group, and a carbazole group.

Example 1 to Example 3 showed increased element lifetime and improved results of element properties when compared to Comparative Example 1 and Comparative Example 2. Without wishing to be bound by any theory, the effects are thought due to the inclusion of the second substituent and the third substituent in the compounds of Example 1 to Example 3. The intermolecular interaction of naphthyl groups having different substitution positions induces the improvement of orientation, to be a factor of the increase of the lifetime, which is position selective between the naphthyl groups at different substitution positions.

Example 4 and Example 5 showed markedly improved results of element lifetime when compared to Comparative Example 3 and Comparative Example 4. Without wishing to be bound by any theory, the effects are thought due to the combination of two different naphthyl groups (an α-naphthyl group and a ß-naphthyl group) in the compounds included in Example 4 and Example 5, separately. The intermolecular interaction of the naphthyl groups having different substitution positions induces the improvement of orientation, to be a factor of the increase of the lifetime, which is position selective between the naphthyl groups at different substitution positions.

When comparing Example 1 and Example 4 to Example 6, with Comparative Example 5 and Comparative Example 6, the Examples showed increased element lifetime and improved element properties over the Comparative Examples. The compounds included in Example 1 and Example 4 to Example 6 may show lifetime improving effects accompanied with the improvement of the intermolecular interaction of the naphthyl groups and the intermolecular orientation, and the excellent or suitable lifetime in contrast to Comparative Example 5 and Comparative Example 6 is considered to be effects by the position selectivity of the aryl groups substituted at the naphthyl groups. For example, as in the compounds included in Comparative Example 5 and Comparative Example 6, when aryl groups are substituted at position 3 and position 8 of the α-naphthyl group, different intermolecular orientation is shown. As in the compounds of the Examples according to the present disclosure, when the aryl group is substituted at position 2 of the α-naphthyl group, the increase of the lifetime is confirmed.

When Example 6 and Comparative Examples 5 and 6 are compared, the amine compound included in Example 6 includes both (e.g., simultaneously) the second substituent and the third substituent, and when the second substituent is directly connected with the nitrogen atom of the amine, long-life characteristics are shown.

When Example 6 and Comparative Example 7 are compared, in the amine compound included in Comparative Example 7, a β-naphthyl group is substituted at position 2 of an α-naphthyl group, and it is considered that intermolecular interaction of the α-naphthyl group with different two types (kinds) of the ß-naphthyl group substituted at the α-naphthyl group and the β-naphthyl group combined with the nitrogen atom of the amine occurred, and different intermolecular orientation was shown. Accordingly, it is thought that only when an aryl group excluding a naphthyl group is substituted at position 2 of the α-naphthyl group, the selective increase of the element lifetime is achieved.

In cases of the amine compounds included in Example 7 and Example 8, it is found that even in case of having a partial skeleton of the β-naphthyl group like a phenanthrene group, the improving effects of substitution selective orientation with an α-naphthyl group are shown. In addition, in case of including a phenanthrene group, the same level of effects of the improvement of the element lifetime is thought be achieved irrespective of the first substituent, the aryl group or the heteroaryl group.

When Example 8 and Comparative Example 8 are compared, the amine compound of an embodiment, included in Example 8, includes second and third substituents and shows increased element lifetime. For example, as in the amine compound included in Example 8, even though a partial skeleton of the ß-naphthyl group such as a phenanthrene group is included, intermolecular interaction may occur between different two types (kinds) of naphthyl groups and the increase of the lifetime accompanied with the improvement of orientation could be achieved.

When Example 8, and Comparative Examples 9 and 10 are compared, Example 8, including the amine compounds of embodiments showed increased element lifetime. The effects are due to the combination of the second substituent and the third substituent of the amine compounds included in Example 8. According to the present disclosure, when the α-naphthyl group and the partial skeleton of the ß-naphthyl group such as a phenanthrene group are included, like the amine compounds included in Example 8, it is thought that the improvement of element lifetime accompanied with the improvement of orientation may be achieved.

In one or more embodiments, the light emitting element includes the amine compound of one or more embodiments and may show long-life characteristics.

When the amine compound of one or more embodiments is applied to a light emitting element, long-life characteristics may be shown.

As utilized herein, the terms “substantially,” “about,” or similar terms are used as terms of approximation and not as terms of degree, and are intended to account for the inherent deviations in measured or calculated values that would be recognized by those of ordinary skill in the art. “About” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, ±20%, ±10%, ±5% of the stated value.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this disclosure, including the claims, to expressly recite any sub-range subsumed within the ranges expressly recited herein.

In the present disclosure, when particles are spherical, “size” indicates a particle diameter or an average particle diameter, and when the particles are non-spherical, the “size” indicates a major axis length or an average major axis length. The diameter (or size) of the particles may be measured utilizing a scanning electron microscope or a particle size analyzer. As the particle size analyzer, for example, HORIBA, LA-950 laser particle size analyzer, may be utilized. When the size of the particles is measured utilizing a particle size analyzer, the average particle diameter (or size) is referred to as D50. D50 refers to the average diameter (or size) of particles whose cumulative volume corresponds to 50 vol % in the particle size distribution (e.g., cumulative distribution), and refers to the value of the particle size corresponding to 50% from the smallest particle when the total number of particles is 100% in the distribution curve accumulated in the order of the smallest particle size to the largest particle size.

The light-emitting element, the display device/apparatus, or any other relevant apparatuses/devices or components according to embodiments of the present disclosure described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

Although the embodiments of the present disclosure have been described, it is understood that the present disclosure should not be limited to these embodiments, but one or more suitable changes and modifications can be made by one ordinary skilled in the art within the spirit and scope of the present disclosure as hereinafter claimed and equivalents thereof.

What is claimed is:

1. A light emitting element, comprising:
a first electrode;
a second electrode on the first electrode; and
at least one functional layer between the first electrode and the second electrode, wherein
the at least one functional layer comprises an amine compound represented by Formula 1: 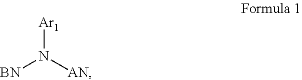
in Formula 1,
Ar₁ is a substituted or unsubstituted aryl group of 6 to 50 ring-forming carbon atoms, or a substituted or unsubstituted heteroaryl group of 5 to 50 ring-forming carbon atoms,
any one selected from among AN and BN is represented by Formula A, and the remainder is represented by Formula B: 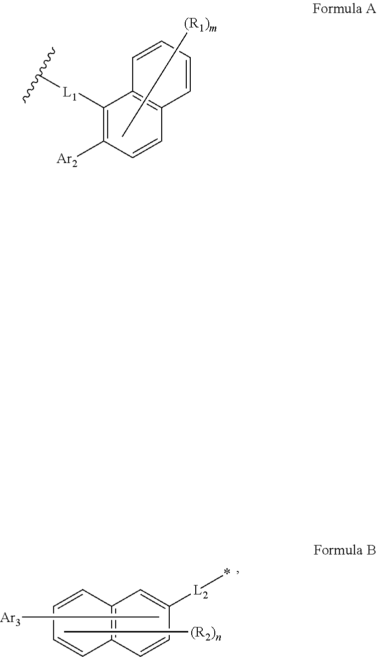
in Formula A and Formula B,
L₁ and L₂ are each independently a direct linkage, or a substituted or unsubstituted arylene group of 6 to 50 ring-forming carbon atoms,
R₁ is hydrogen, deuterium, a cyano group, or a substituted or unsubstituted alkyl group of 1 to 30 carbon atoms,
R₂ is hydrogen, deuterium, a cyano group, a substituted or unsubstituted alkyl group of 1 to 30 carbon atoms, or a substituted or unsubstituted aryl group of 6 to 30 ring-forming carbon atoms, and/or combined with an adjacent group to form a ring,
Ar₂ is a substituted or unsubstituted aryl group of 6 to 50 ring-forming carbon atoms,
Ar₃ is hydrogen, deuterium, or a substituted or unsubstituted aryl group of 6 to 50 ring-forming carbon atoms,
“n” and “m” are each independently an integer of 0 to 6,
provided that Ar₂ is not a substituted or unsubstituted naphthyl group, and
“ [symbol] ” and “—*” are positions connected with Formula 1.

2. The light emitting element of claim 1, wherein
Formula A is represented by Formula A1 or Formula A2, and
Formula B is represented by Formula B1 or Formula B2: 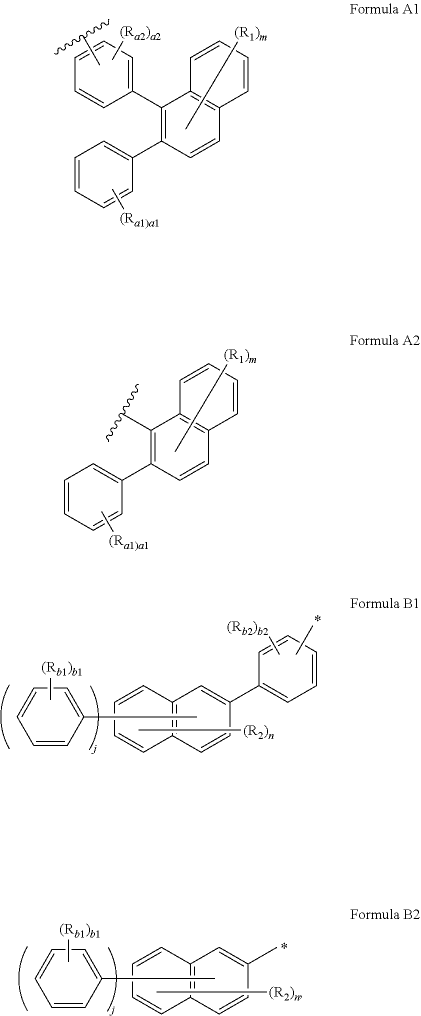
and
wherein, in Formula A1 and Formula A2,
Rₐ₁ is hydrogen, deuterium, or a substituted or unsubstituted aryl group of 6 to 15 ring-forming carbon atoms,
Rₐ₂ is hydrogen or deuterium,
a1 is an integer of 0 to 5, a2 is an integer of 0 to 4,
R₁ and “m” are each independently the same as defined in Formula A, and
in Formula B1 and Formula B2,
R_{b1} and R_{b2} are each independently hydrogen or deuterium,
b1 is an integer of 0 to 5, b2 is an integer of 0 to 4,
“j” is 0 or 1, and
R₂ and “n” are each independently the same as defined in Formula B.

3. The light emitting element of claim 1, wherein the amine compound represented by Formula 1 is represented by Formula 2: 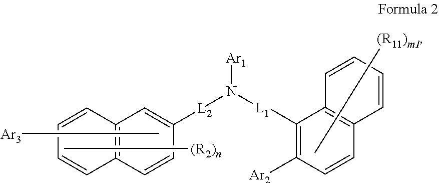
and
wherein, in Formula 2,
R₁₁ is hydrogen or deuterium,
m1 is an integer of 0 to 6, and
Ar₁ to Ar₃, L₁, L₂, R₂, and “n” are each independently the same as defined in Formula 1.

4. The light emitting element of claim 1, wherein the amine compound represented by Formula 1 is represented by Formula 3-1 or Formula 3-2: 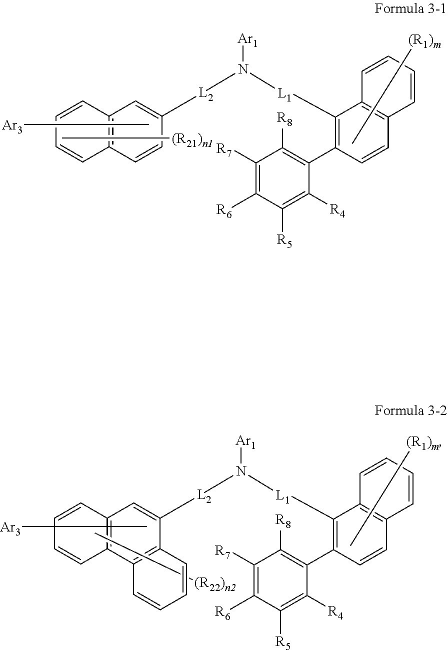
and
wherein, in Formula 3-1 and Formula 3-2,
R₄ to Re are each independently hydrogen, deuterium, or a substituted or unsubstituted aryl group of 6 to 15 ring-forming carbon atoms,
R₂₁ and R₂₂ are each independently hydrogen or deuterium,
n1 is an integer of 0 to 6,
n2 is an integer of 0 to 8, and
Ar₁, Ar₃, L₁, L₂, R₁, and “m” are each independently the same as defined in Formula 1.

5. The light emitting element of claim 1, wherein the amine compound represented by Formula 1 is represented by Formula 4-1 or Formula 4-2: 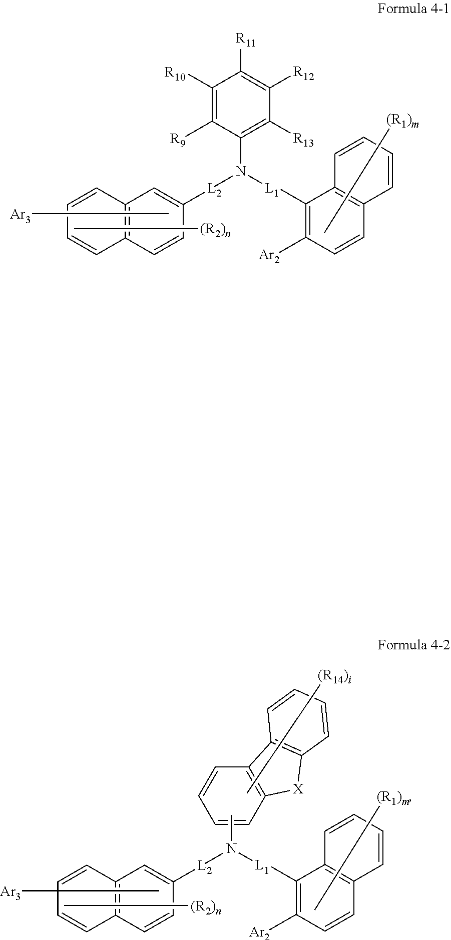
and
wherein, in Formula 4-1,
R₉ to R₁₃ are each independently hydrogen, deuterium, or a substituted or unsubstituted aryl group of 6 to 30 ring-forming carbon atoms, and/or substituents in at least one pair selected from among adjacent R₉ and R₁₀, R₁₀ and R₁₁, R₁₁ and R₁₂, and R₁₂ and R₁₃ are combined with each other to form an aromatic hydrocarbon ring,
in Formula 4-2,
X is O, S, NRₓ₁, or CRₓ₂Rₓ₃,
Rₓ₁ is a substituted or unsubstituted aryl group of 6 to 15 ring-forming carbon atoms,
Rₓ₂ and Rₓ₃ are each independently a substituted or unsubstituted alkyl group of to 10 carbon atoms, or a substituted or unsubstituted aryl group of 6 to 15 ring-forming carbon atoms, or Rₓ₂ and Rₓ₃ are combined with each other to form a ring,
R₁₄ is hydrogen, deuterium, or a substituted or unsubstituted aryl group of 6 to 15 ring-forming carbon atoms, and/or combined with an adjacent group to form a ring, and
“i” is an integer of 0 to 7, and
in Formula 4-1 and Formula 4-2,
Ar₂, Ar₃, L₁, L₂, R₁, R₂, “m” and “n” are each independently the same as defined in Formula 1.

6. The light emitting element of claim 1, wherein the amine compound represented by Formula 1 is represented by any one selected from among Formula 5-1 to Formula 5-4: 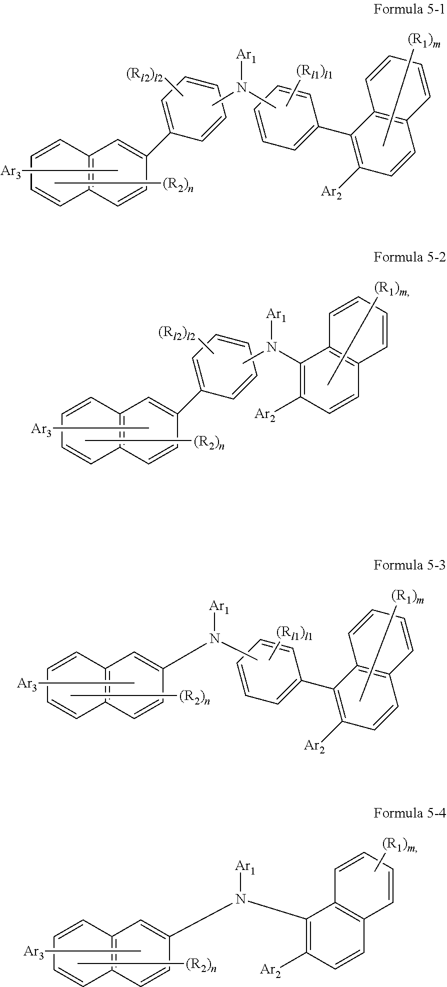
and
wherein, in Formula 5-1 to Formula 5-4,
Rₗ₁ and Rₗ₂ are each independently hydrogen or deuterium,
l1 and l2 are each independently an integer of 0 to 4, and
Ar₁ to Ar₃, R₁, R₂, “m” and “n” are each independently the same as defined in Formula 1.

7. The light emitting element of claim 1, wherein AN is represented by any one in Substituent Group A:
Substituent Group A

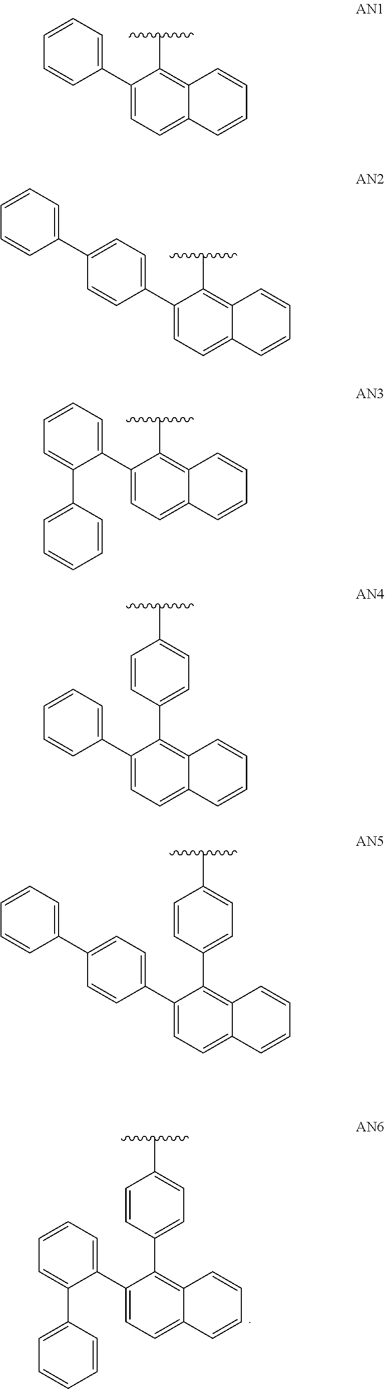 [SMILES: *c1c(-c2ccccc2)ccc2ccccc12 ; *c1c(-c2ccc(-c3ccccc3)cc2)ccc2ccccc12 ; *c1c(-c2ccccc2-c2ccccc2)ccc2ccccc12 ; *c1ccc(-c2c(-c3ccccc3)ccc3ccccc23)cc1 ; *c1ccc(-c2c(-c3ccc(-c4ccccc4)cc3)ccc3ccccc23)cc1 ; *c1ccc(-c2c(-c3ccccc3-c3ccccc3)ccc3ccccc23)cc1]

8. The light emitting element of claim 1, wherein BN is represented by any one in Substituent Group B:

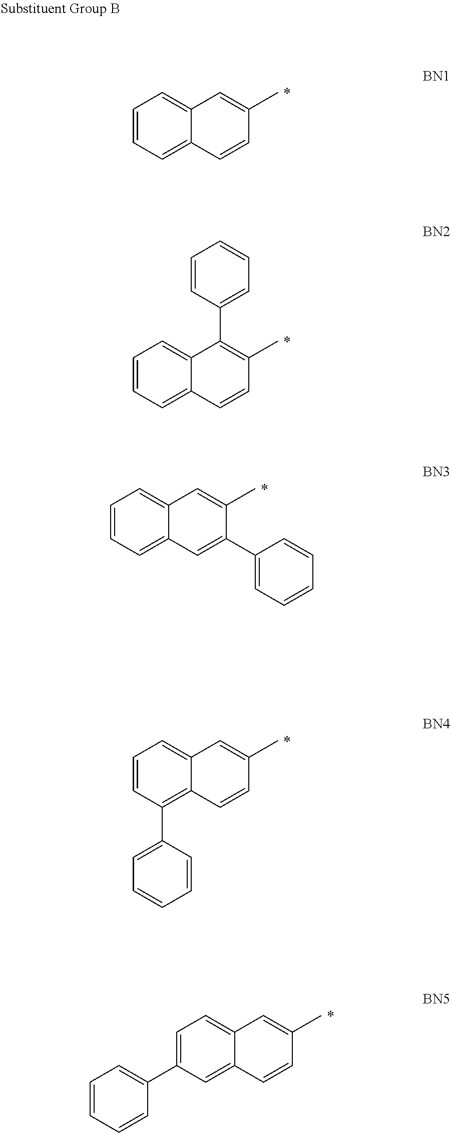

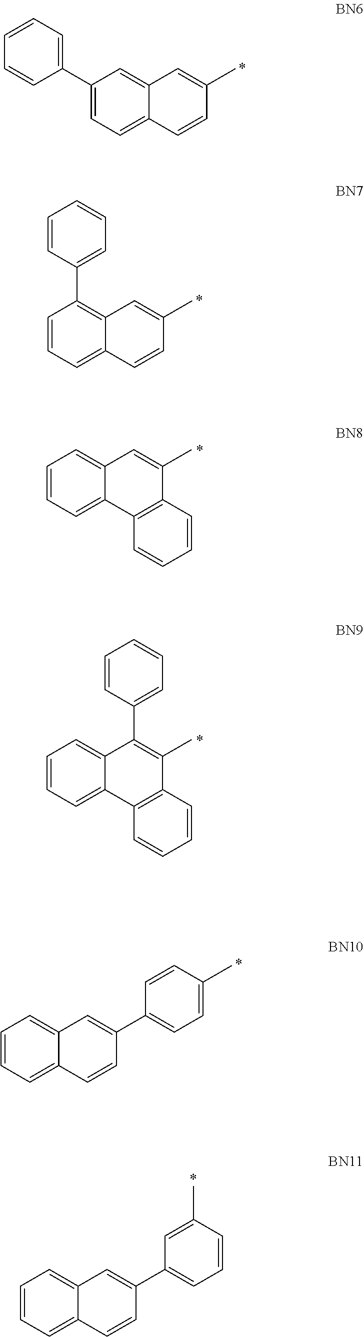

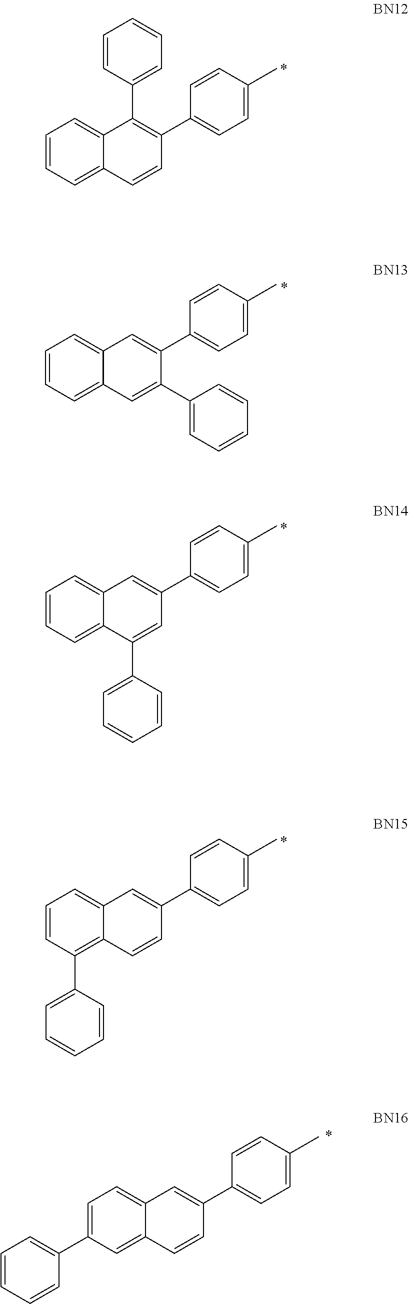

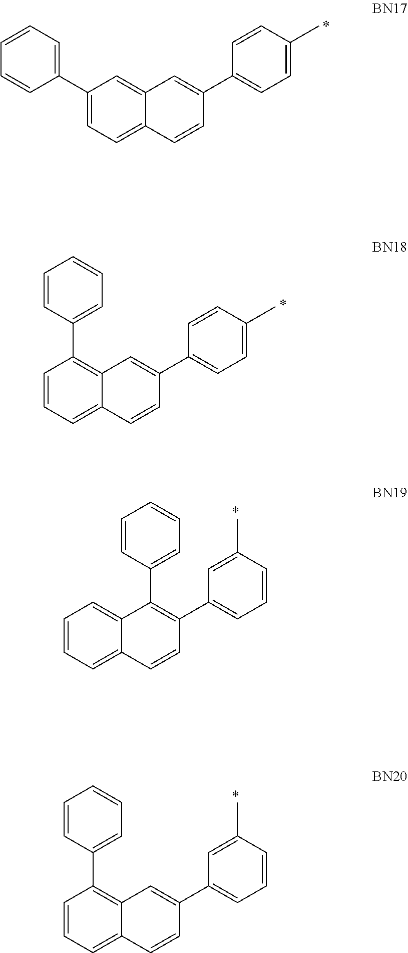

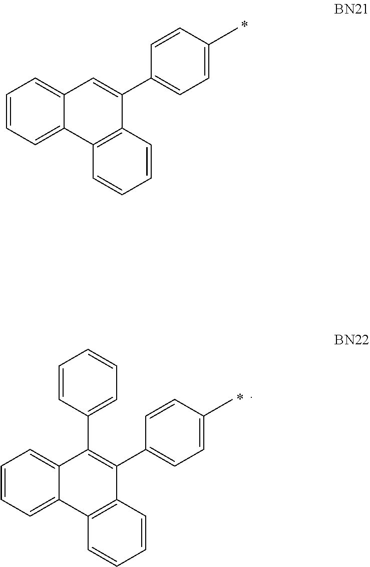

9. The light emitting element of claim 1, wherein Ar₁ is represented by any one in Substituent Group AR:

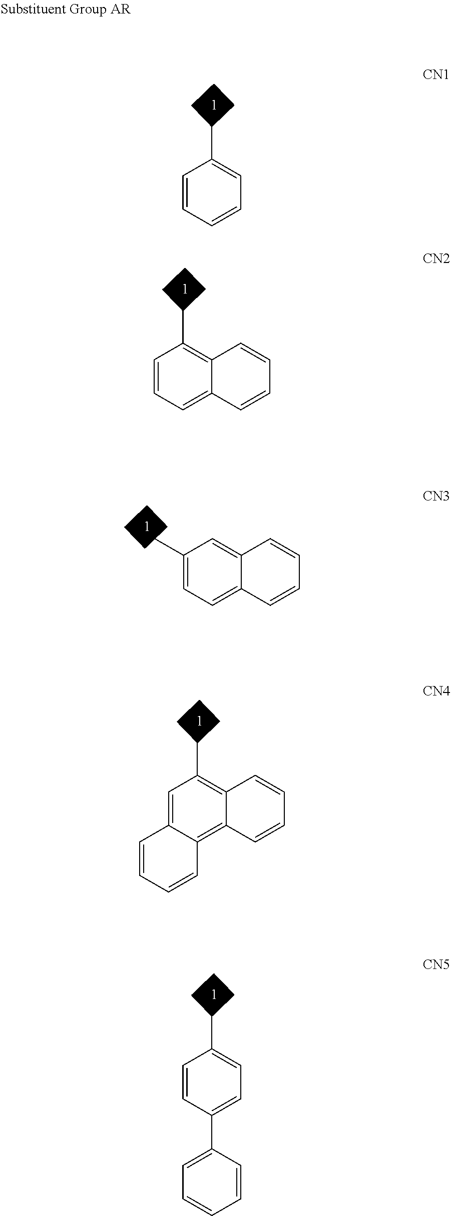

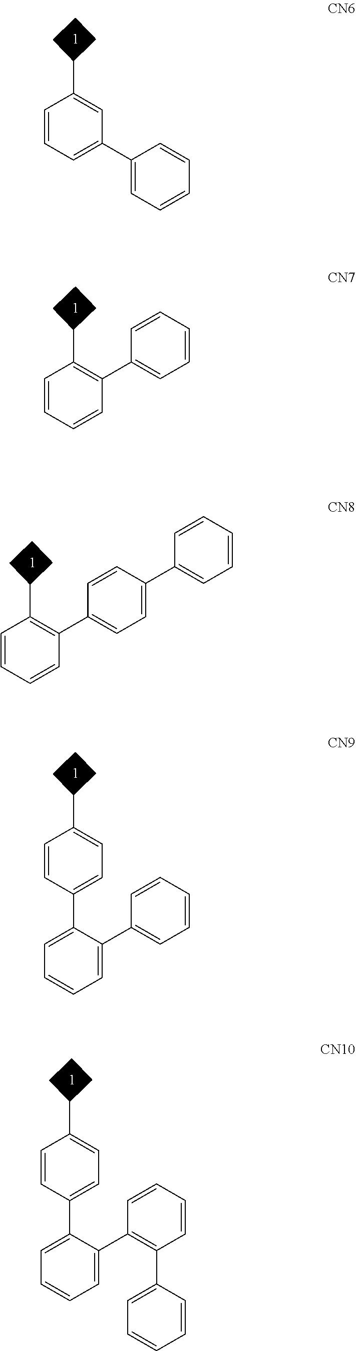

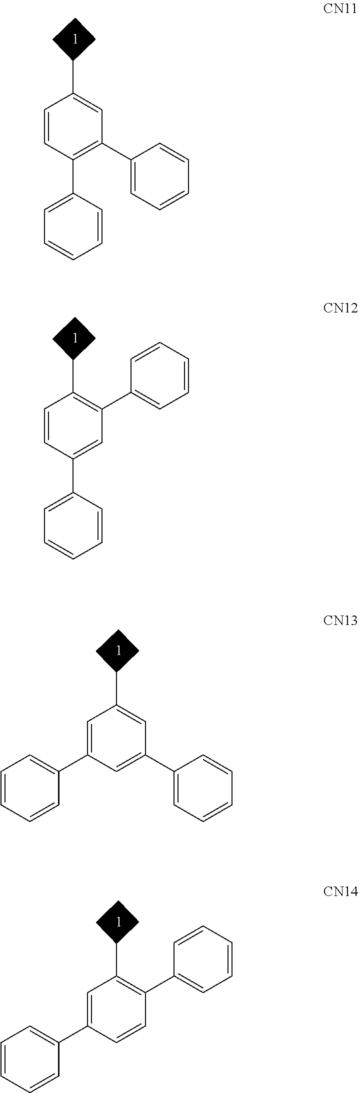

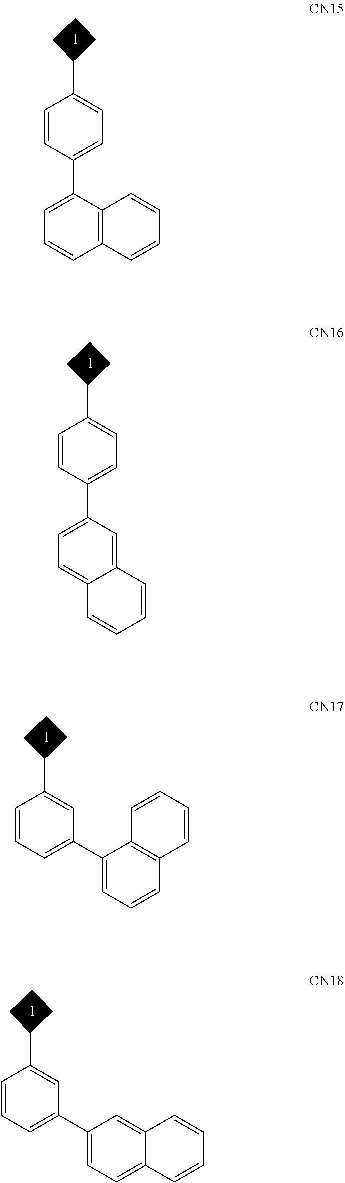

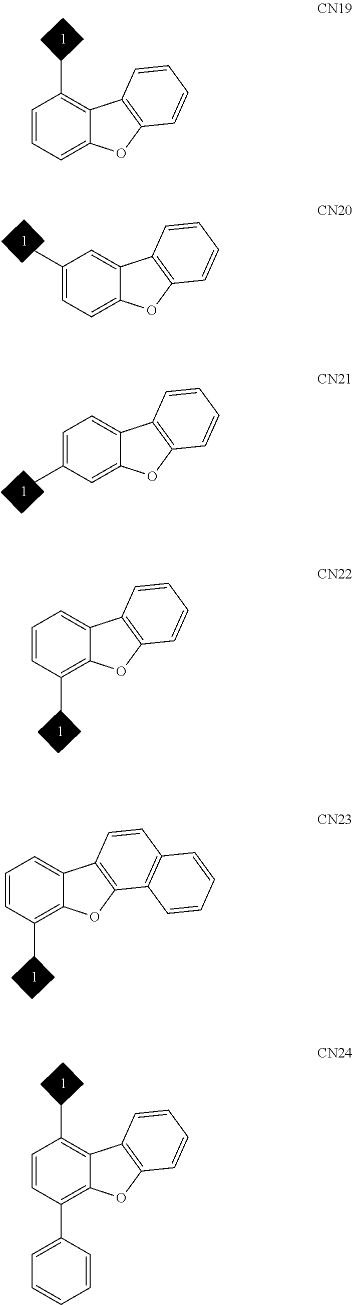 [SMILES: Cc1cccc2oc3ccccc3c12 ; Cc1ccc2oc3ccccc3c2c1 ; Cc1ccc2c(c1)oc1ccccc12 ; Cc1cccc2c1oc1ccccc12 ; Cc1cccc2c1oc1c3ccccc3ccc21 ; Cc1ccc(-c2ccccc2)c2oc3ccccc3c12]

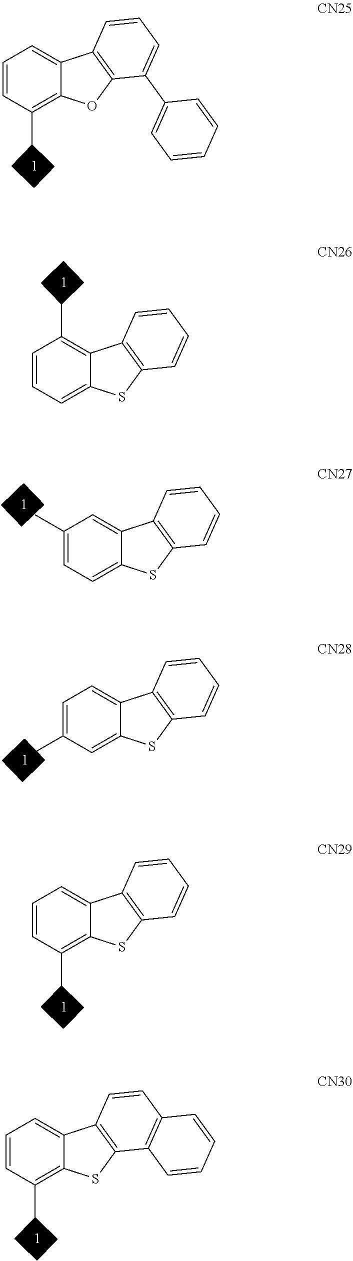 [SMILES: Cc1cccc2c1oc1c(-c3ccccc3)cccc12 ; Cc1cccc2sc3ccccc3c12 ; Cc1ccc2sc3ccccc3c2c1 ; Cc1ccc2c(c1)sc1ccccc12 ; Cc1cccc2c1sc1ccccc12 ; Cc1cccc2c1sc1c3ccccc3ccc21]

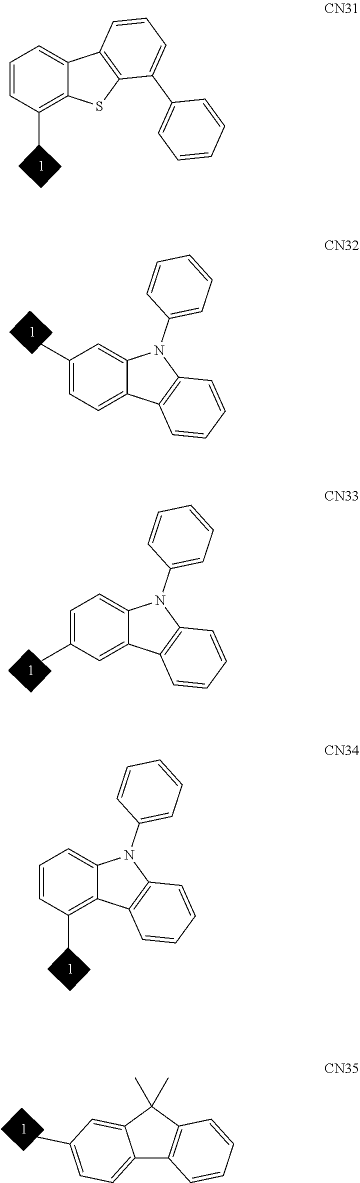

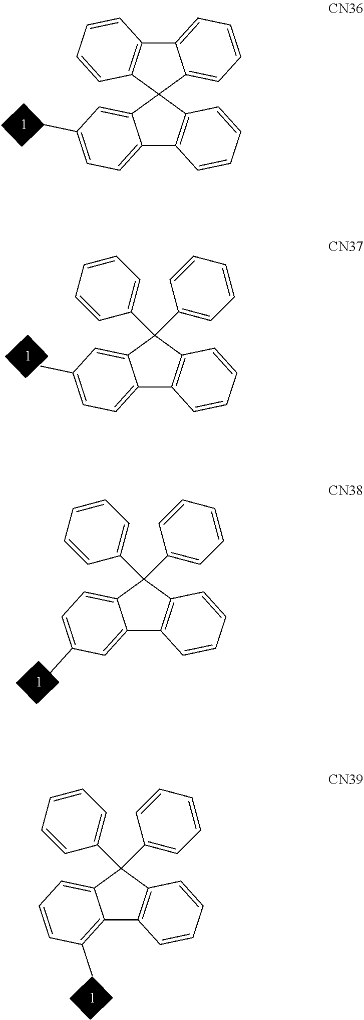

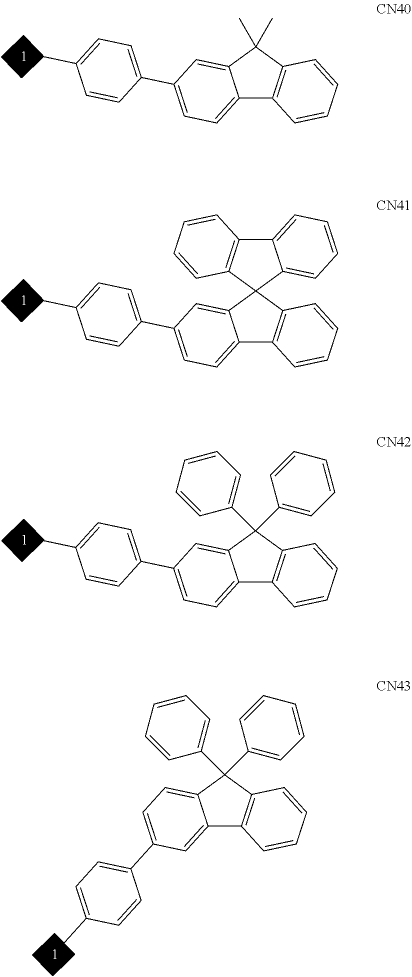

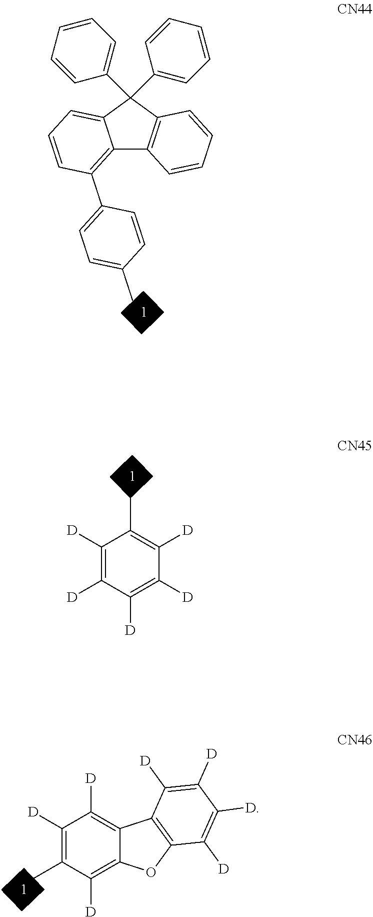

10. The light emitting element of claim 1, wherein the at least one functional layer comprises:
a hole transport region on the first electrode;
an emission layer on the hole transport region; and
an electron transport region on the emission layer, and
wherein the hole transport region comprises the amine compound.

11. The light emitting element of claim 10, wherein the hole transport region comprises:
a hole injection layer on the first electrode; and
a hole transport layer on the hole injection layer, and
wherein the hole transport layer comprises the amine compound.

12. An amine compound represented by Formula 1: 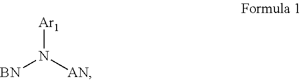
wherein, in Formula 1,
Ar₁ is a substituted or unsubstituted aryl group of 6 to 50 ring-forming carbon atoms, or a substituted or unsubstituted heteroaryl group of 3 to 50 ring-forming carbon atoms,
any one selected from among AN and BN is represented by Formula A, and the remainder is represented by Formula B: 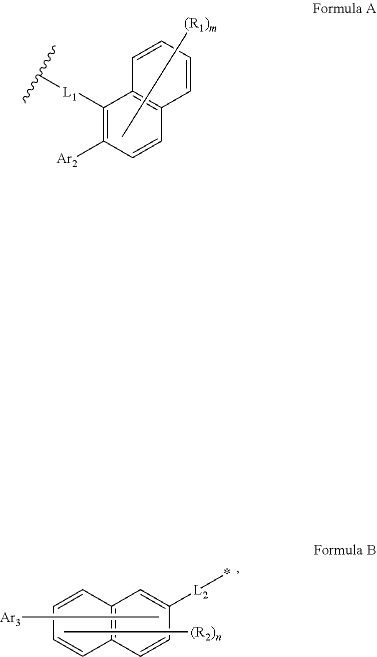
in Formula A and Formula B,
L₁ and L₂ are each independently a direct linkage, or a substituted or unsubstituted arylene group of 6 to 50 ring-forming carbon atoms,
R₁ is hydrogen, deuterium, a cyano group, or a substituted or unsubstituted alkyl group of 1 to 30 carbon atoms,
R₂ is hydrogen, deuterium, a cyano group, a substituted or unsubstituted alkyl group of 1 to 30 carbon atoms, or a substituted or unsubstituted aryl group of 6 to 30 ring-forming carbon atoms, and/or combined with an adjacent group to form a ring,
Ar₂ is a substituted or unsubstituted aryl group of 6 to 50 ring-forming carbon atoms,
Ar₃ is hydrogen, deuterium, or a substituted or unsubstituted aryl group of 6 to 50 ring-forming carbon atoms,
“n” and “m” are each independently an integer of 0 to 6,
provided that Ar₂ is not a substituted or unsubstituted naphthyl group, and
“ [symbol] ” and “—*” are positions connected with Formula 1.

13. The amine compound of claim 12, wherein Formula A is represented by Formula A1 or Formula A2: 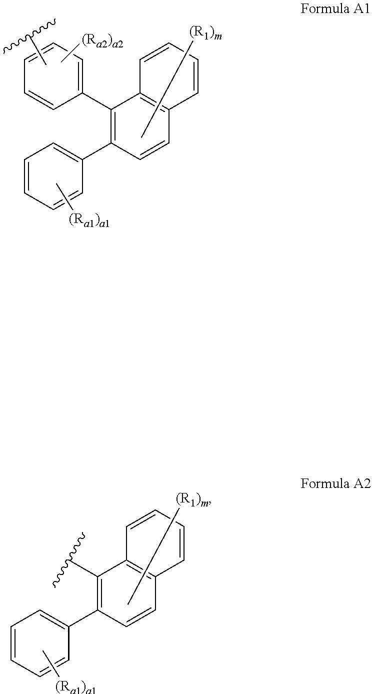
and
wherein, in Formula A1 and Formula A2,
Rₐ₁ is hydrogen, deuterium, or a substituted or unsubstituted aryl group of 6 to 15 ring-forming carbon atoms,
Rₐ₂ is hydrogen or deuterium,
a1 is an integer of 0 to 5, a2 is an integer of 0 to 4, and
R₁ and “m” are each independently the same as defined in Formula A.

14. The amine compound of claim 12, wherein Formula B is represented by Formula B1 or Formula B2: 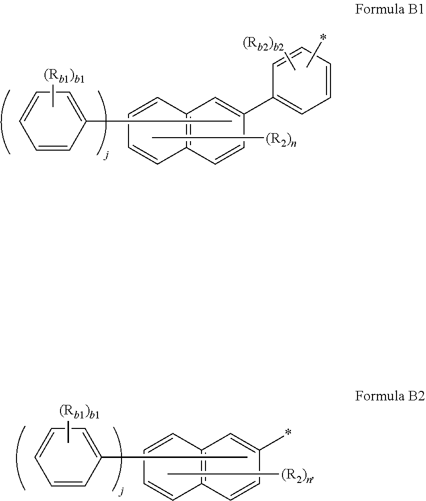
and
wherein, in Formula B1 and Formula B2,
R_{b1} and R_{b2} are each independently hydrogen or deuterium,
b1 is an integer of 0 to 5, b2 is an integer of 0 to 4,
“j” is 0 or 1, and
R₂ and “n” are each independently the same as defined in Formula B.

15. The amine compound of claim 14, wherein
Formula B1 is represented by Formula B1-1 or Formula B1-2, and
Formula B2 is represented by Formula B2-1 or Formula B2-2: 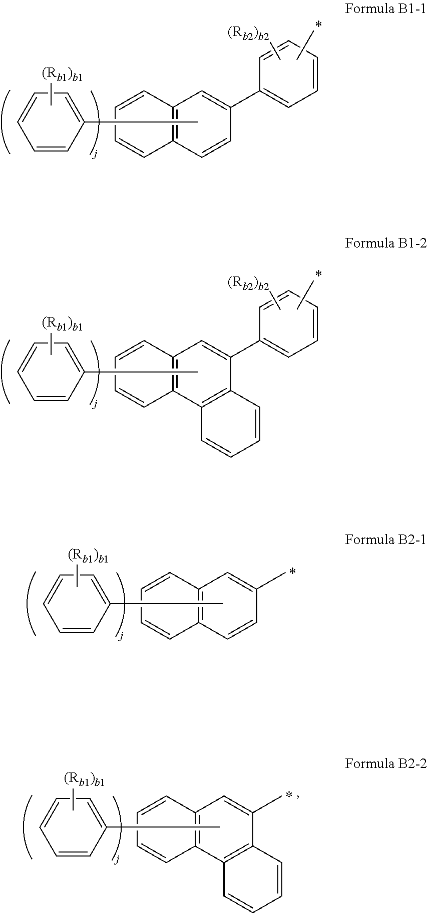
and
wherein, in Formula B1-1, Formula B1-2, Formula B2-1, and Formula B2-2,
R_{b1}, R_{b2}, b1, b2 and “j” are the same as defined in Formula B1 and Formula B2.

16. The amine compound of claim 12, wherein Formula 1 is represented by Formula 2: 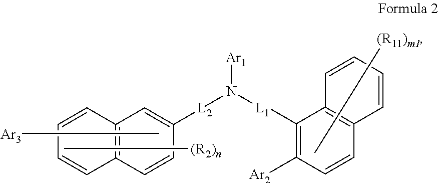
and
wherein, in Formula 2,
R₁₁ is hydrogen or deuterium,
m1 is an integer of 0 to 6, and
Ar₁ to Ar₃, L₁, L₂, R₂, and “n” are each independently the same as defined in Formula 1.

17. The amine compound of claim 12, wherein Formula 1 is represented by Formula 3-1 or Formula 3-2: 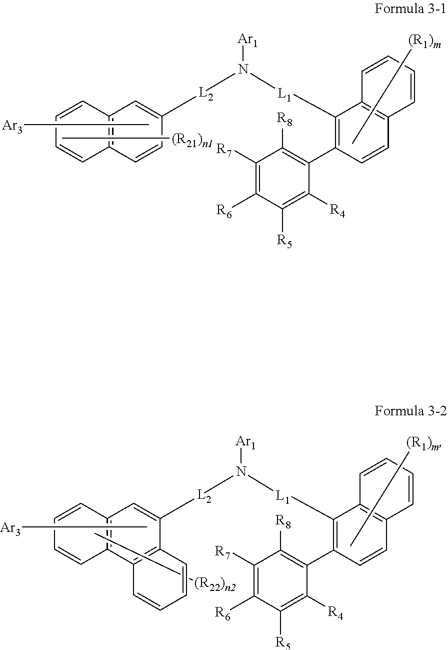
and
wherein, in Formula 3-1 and Formula 3-2,
R₄ to R₈ are each independently hydrogen, deuterium, or a substituted or unsubstituted aryl group of 6 to 15 ring-forming carbon atoms,
R₂₁ and R₂₂ are each independently hydrogen or deuterium,
n1 is an integer of 0 to 6,
n2 is an integer of 0 to 8, and
Ar₁, Ar₃, L₁, L₂, R₁, and “m” are each independently the same as defined in Formula 1.

18. The amine compound of claim 12, wherein Formula 1 is represented by Formula 4-1 or Formula 4-2: 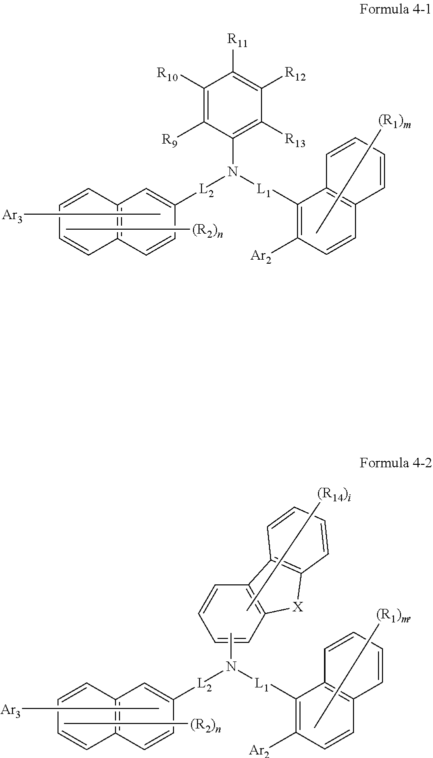
and
wherein, in Formula 4-1,
R₉ to R₁₃ are each independently hydrogen, deuterium, or a substituted or unsubstituted aryl group of 6 to 30 ring-forming carbon atoms, and/or substituents in at least one pair selected from among adjacent Ry and R₁₀, R₁₀ and R₁₁, R₁₁ and R₁₂, and R₁₂ and R₁₃ are combined with each other to form an aromatic hydrocarbon ring,
in Formula 4-2,
X is O, S, NRₓ₁, or CRₓ₂Rₓ₃,
Rₓ₁ is a substituted or unsubstituted aryl group of 6 to 15 ring-forming carbon atoms,
Rₓ₂ and Rₓ₃ are each independently a substituted or unsubstituted alkyl group of to 10 carbon atoms, or a substituted or unsubstituted aryl group of 6 to 15 ring-forming carbon atoms, or combined with an adjacent group to form a ring,
R₁₄ is hydrogen, deuterium, or a substituted or unsubstituted aryl group of 6 to 15 ring-forming carbon atoms, and/or combined with an adjacent group to form a ring, and
“i” is an integer of 0 to 7, and
in Formula 4-1 and Formula 4-2,
Ar₂, Ar₃, L₁, L₂, R₁, R₂, “m” and “n” are each independently the same as defined in Formula 1.

19. The amine compound of claim 12, wherein Formula 1 is represented by any one among Formula 5-1 to Formula 5-4: 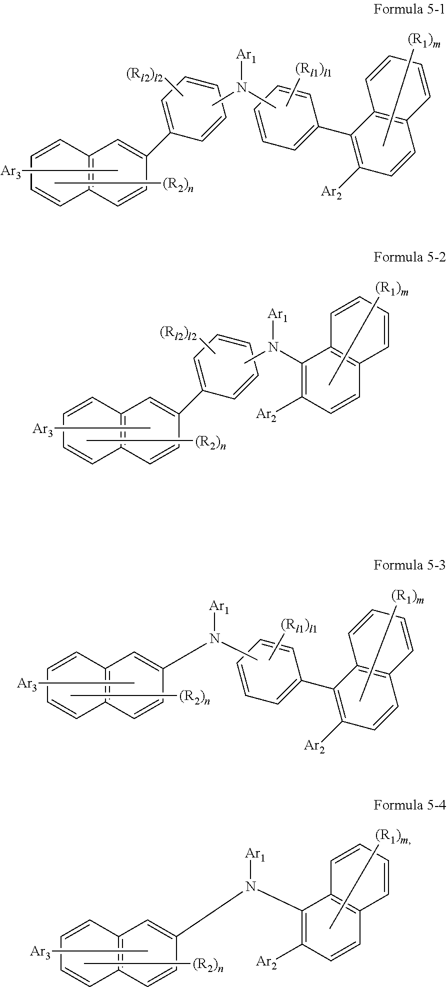
and
wherein, in Formula 5-1 to Formula 5-4,
Rₗ₁ and Rₗ₂ are each independently hydrogen or deuterium,
l1 and l2 are each independently an integer of 0 to 4, and
Ar₁ to Ar₃, R₁, R₂, “m” and “n” are each independently the same as defined in Formula 1.

20. The amine compound of claim 12, wherein AN is represented by any one in Substituent Group A:

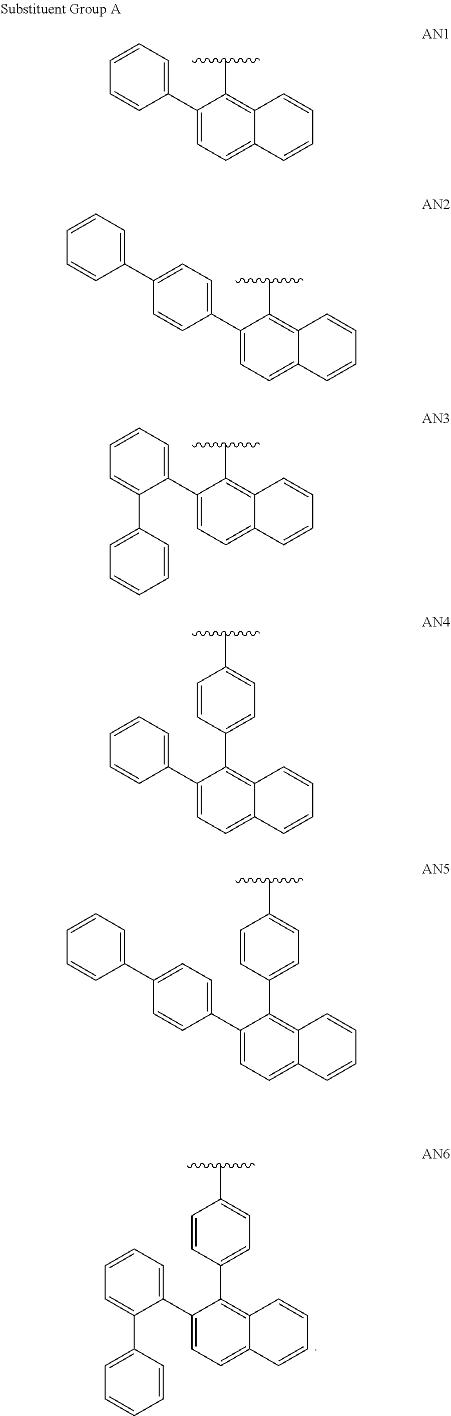 [SMILES: *c1c(-c2ccccc2)ccc2ccccc12 ; *c1c(-c2ccc(-c3ccccc3)cc2)ccc2ccccc12 ; *c1c(-c2ccccc2-c2ccccc2)ccc2ccccc12 ; *c1ccc(-c2c(-c3ccccc3)ccc3ccccc23)cc1 ; *c1ccc(-c2c(-c3ccc(-c4ccccc4)cc3)ccc3ccccc23)cc1 ; *c1ccc(-c2c(-c3ccccc3-c3ccccc3)ccc3ccccc23)cc1]

21. The amine compound of claim 12, wherein BN is represented by any one in Substituent Group B:

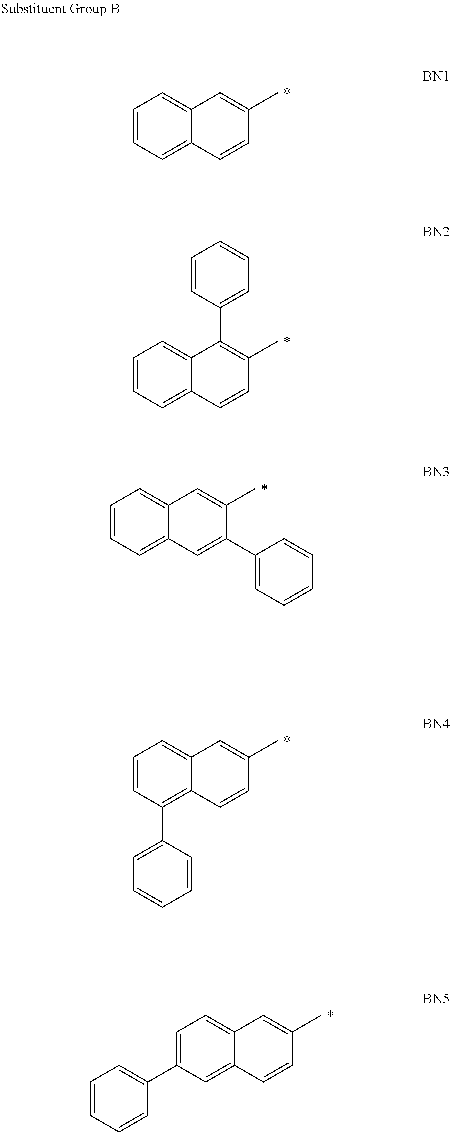

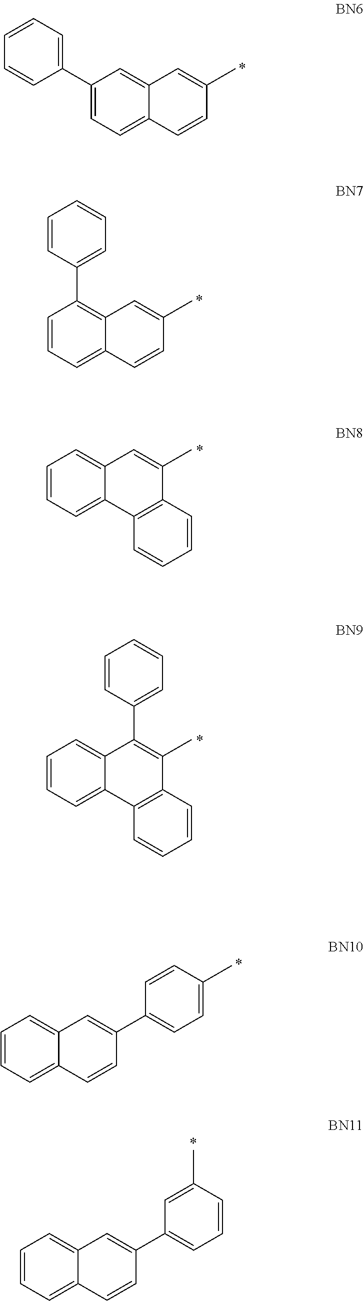

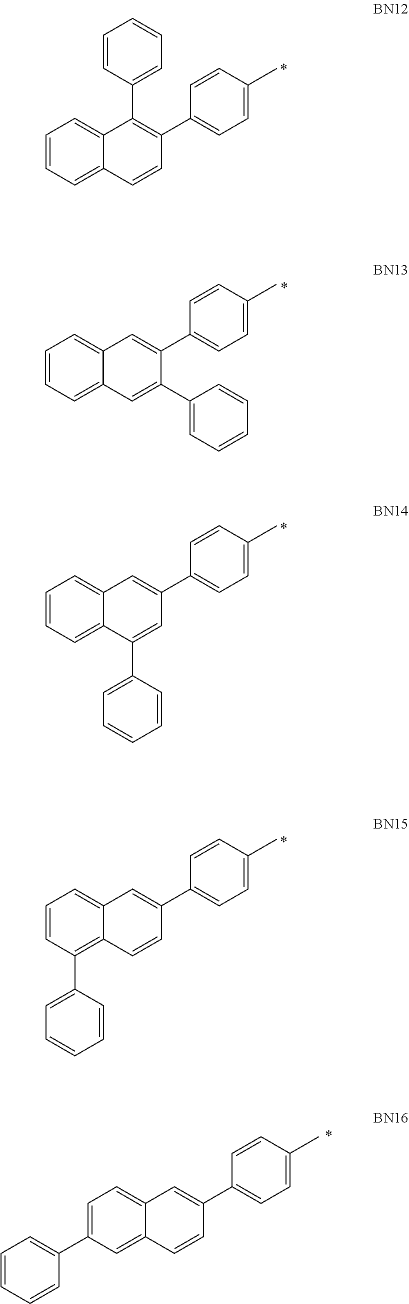

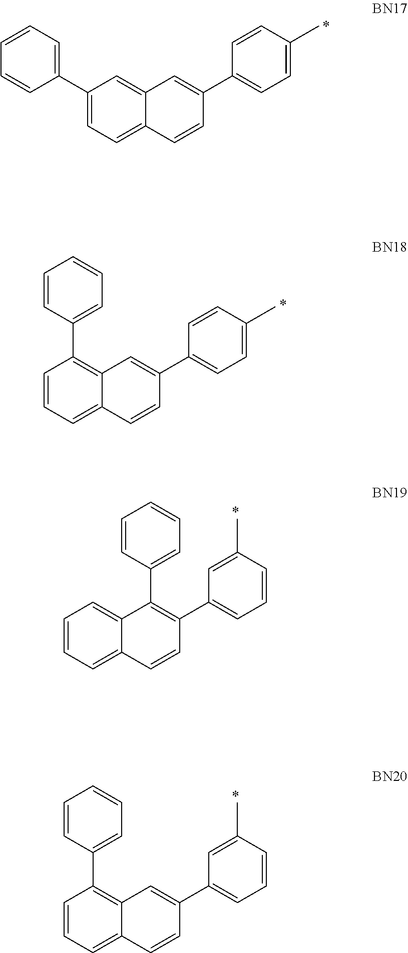

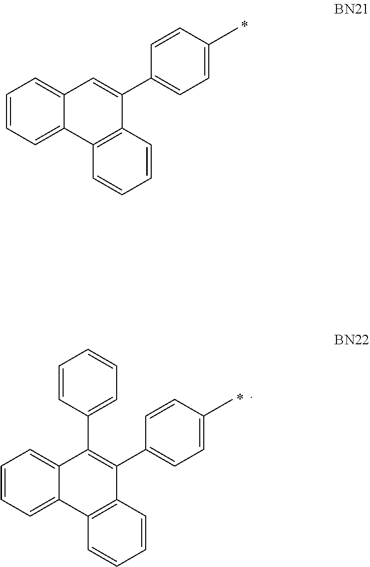

22. The amine compound of claim 12, wherein Ar₁ is represented by any one in Substituent Group AR:

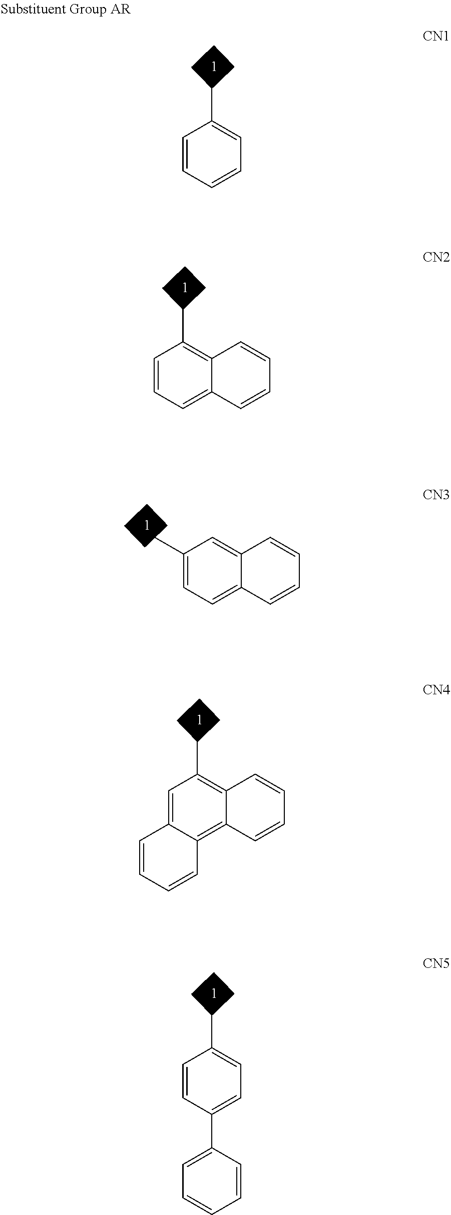

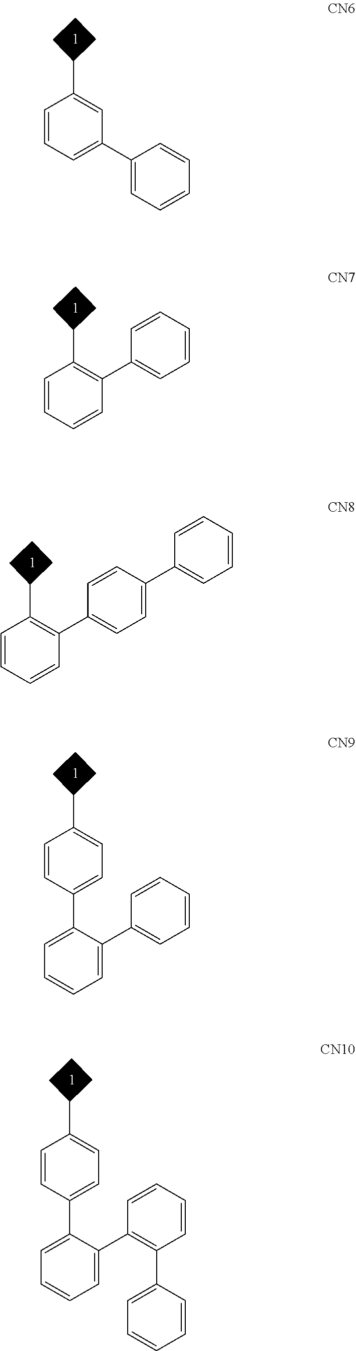

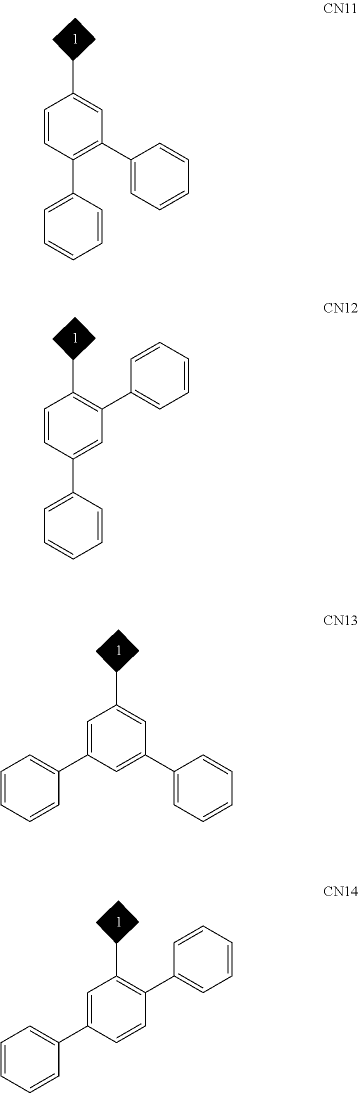

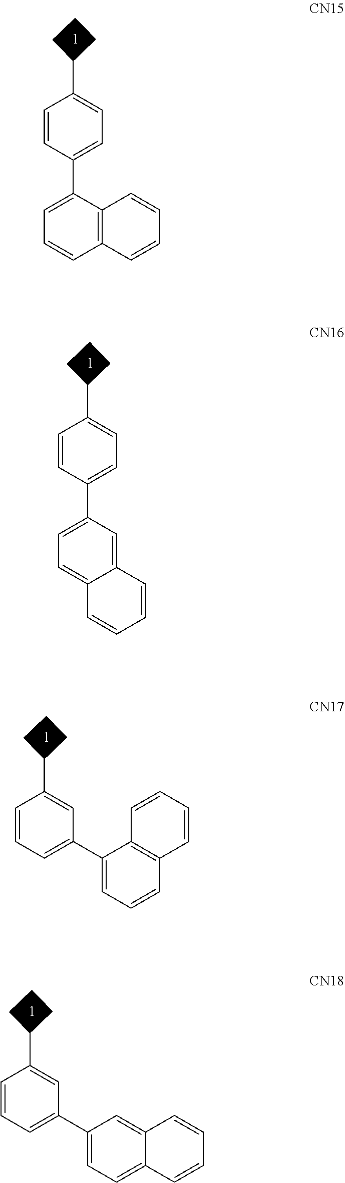

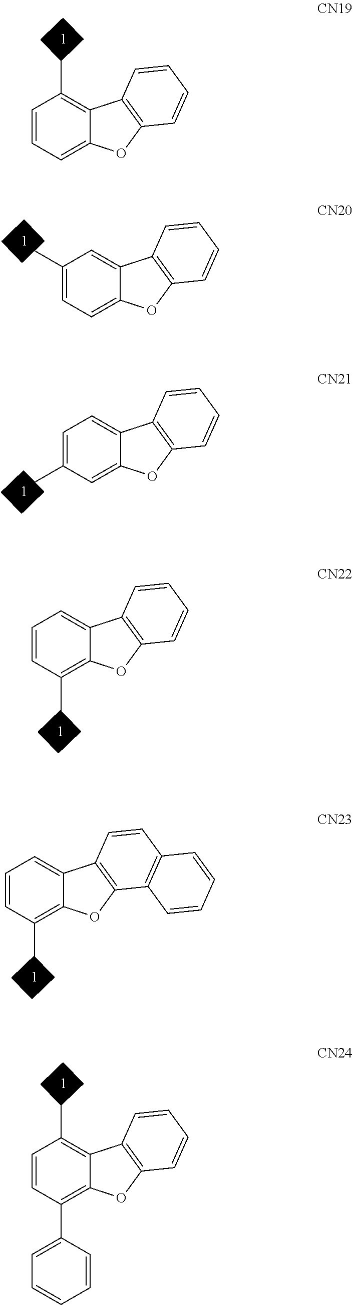 [SMILES: Cc1cccc2oc3ccccc3c12 ; Cc1ccc2oc3ccccc3c2c1 ; Cc1ccc2c(c1)oc1ccccc12 ; Cc1cccc2c1oc1ccccc12 ; Cc1cccc2c1oc1c3ccccc3ccc21 ; Cc1ccc(-c2ccccc2)c2oc3ccccc3c12]

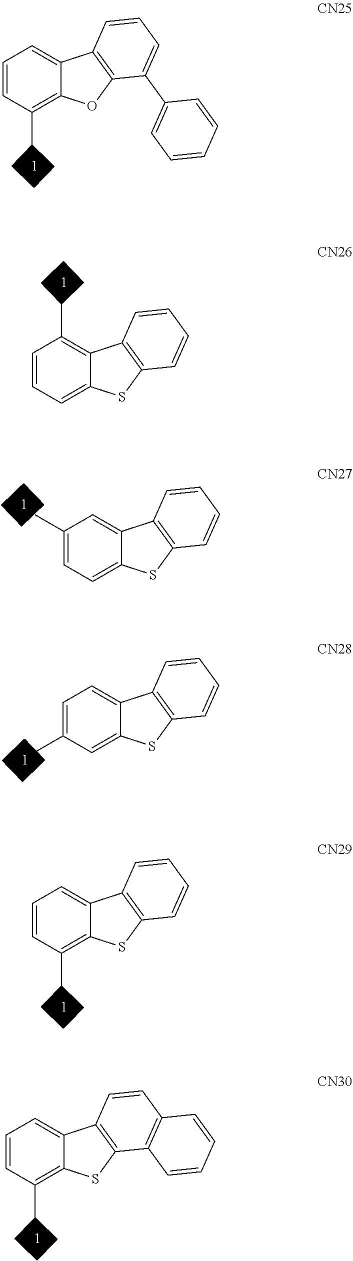 [SMILES: Cc1cccc2c1oc1c(-c3ccccc3)cccc12 ; Cc1cccc2sc3ccccc3c12 ; Cc1ccc2sc3ccccc3c2c1 ; Cc1ccc2c(c1)sc1ccccc12 ; Cc1cccc2c1sc1ccccc12 ; Cc1cccc2c1sc1c3ccccc3ccc21]

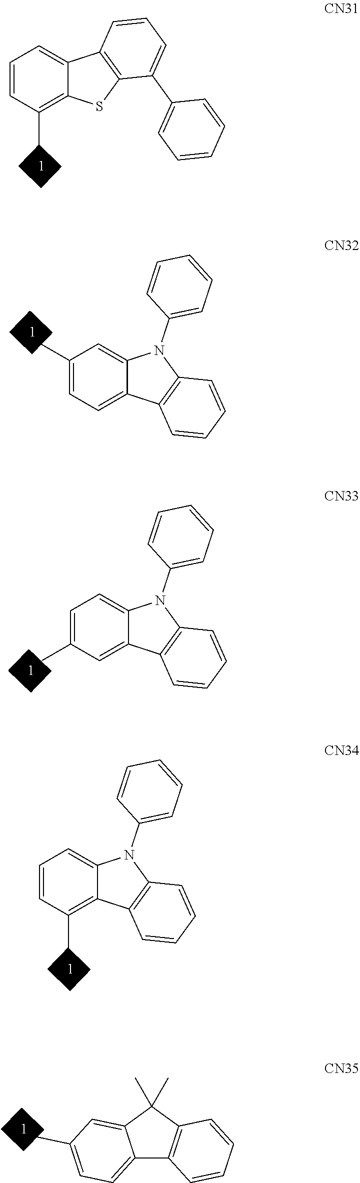

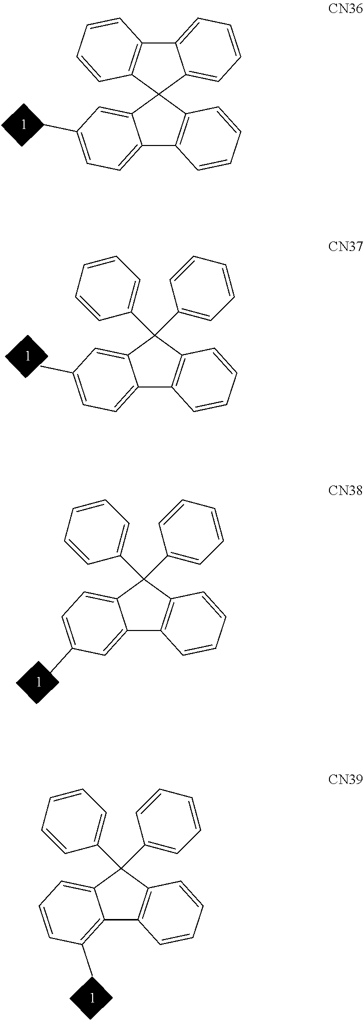

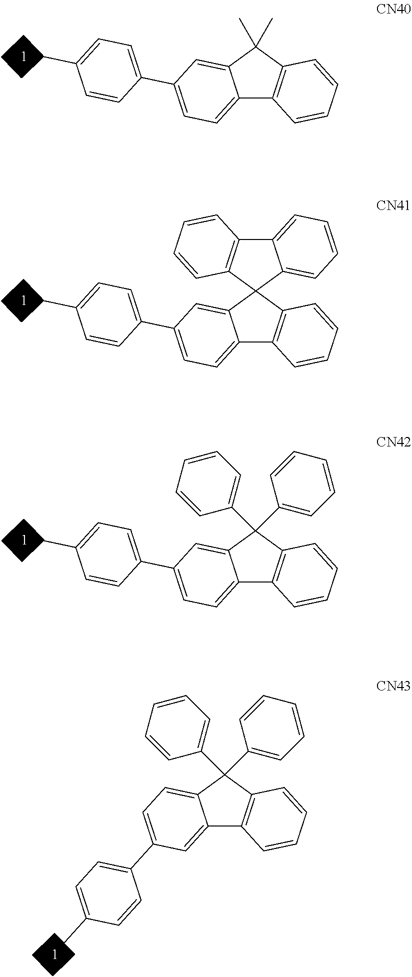

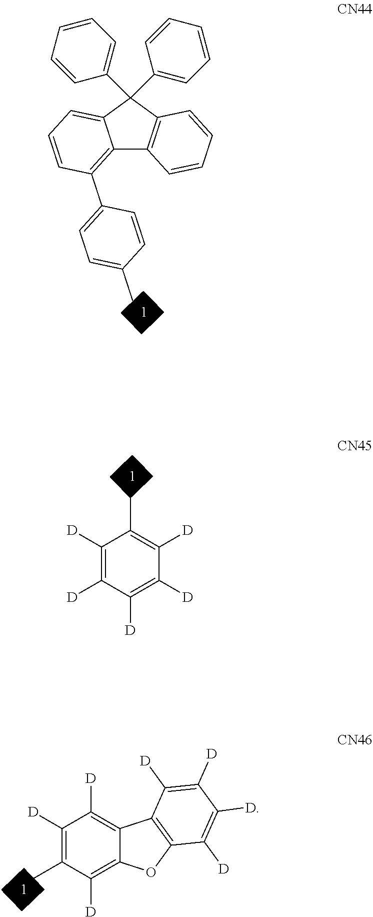

23. The amine compound of claim 12, wherein AN is represented by any one in Substituent Group A, BN is represented by any one in Substituent Group B, Ar₁ is represented by any one in Substituent Group AR, and Formula 1 satisfies any one selected from among substituent combinations represented in Compound Combination Table 1: 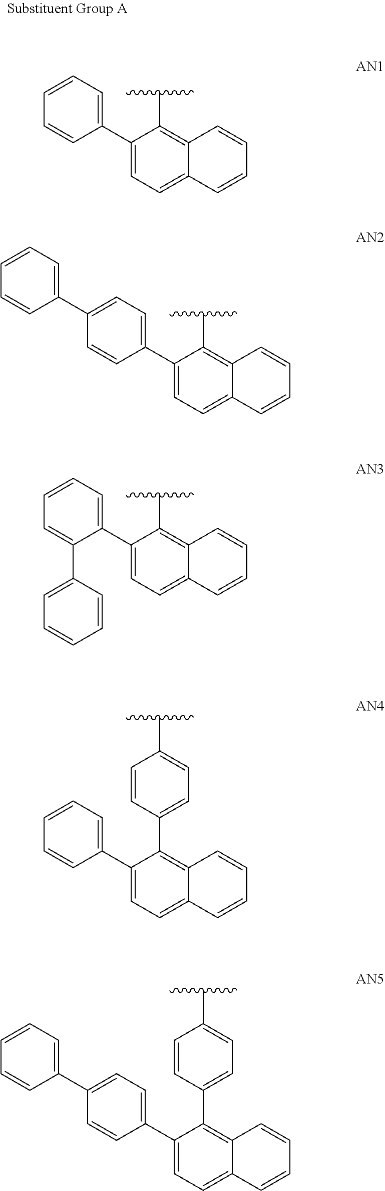 [SMILES: *c1c(-c2ccccc2)ccc2ccccc12 ; *c1c(-c2ccc(-c3ccccc3)cc2)ccc2ccccc12 ; *c1c(-c2ccccc2-c2ccccc2)ccc2ccccc12 ; *c1ccc(-c2c(-c3ccccc3)ccc3ccccc23)cc1 ; *c1ccc(-c2c(-c3ccc(-c4ccccc4)cc3)ccc3ccccc23)cc1] 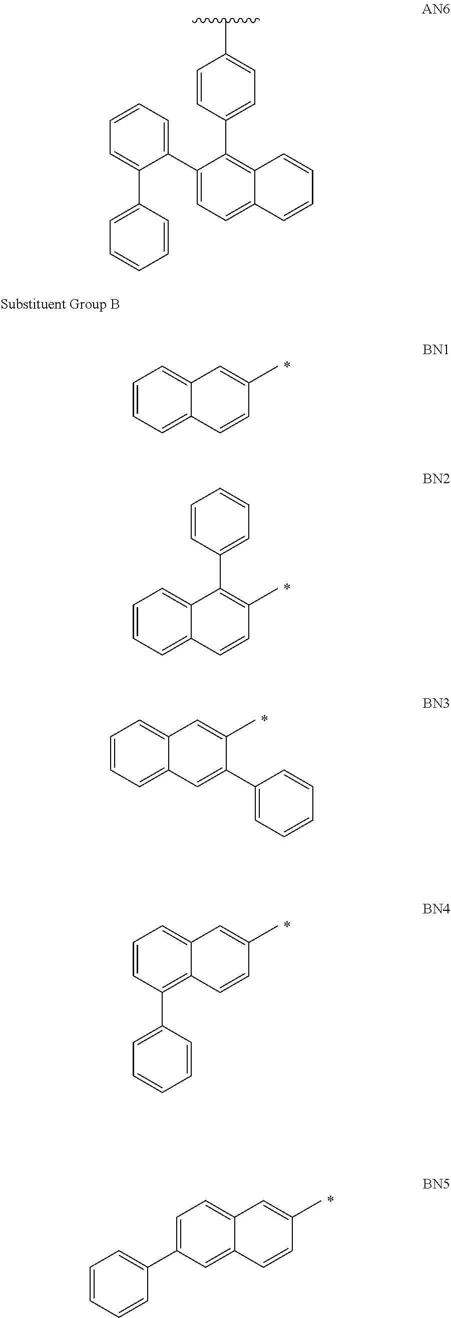 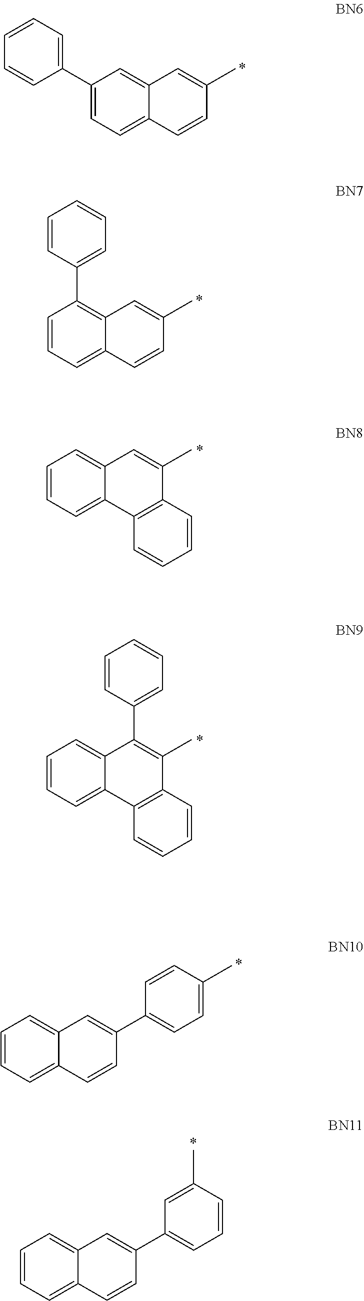 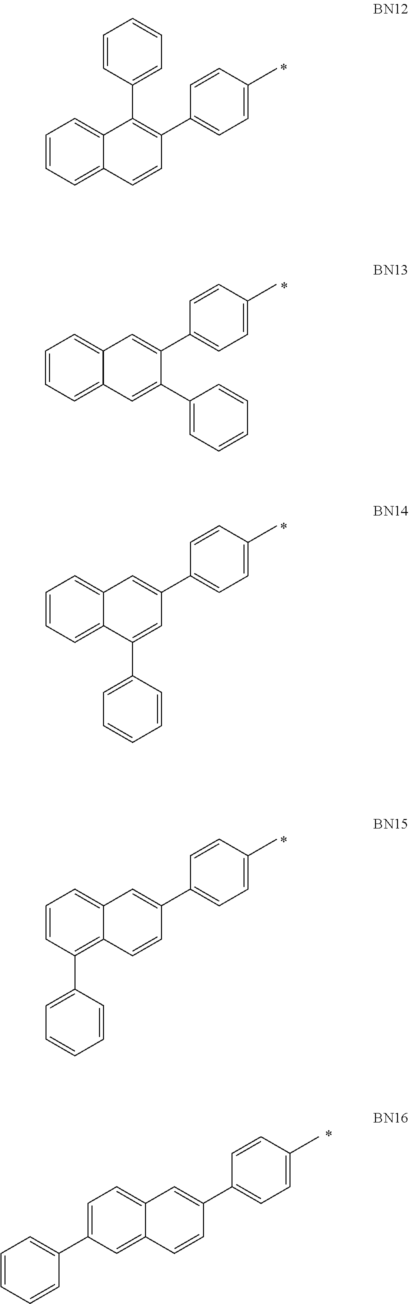 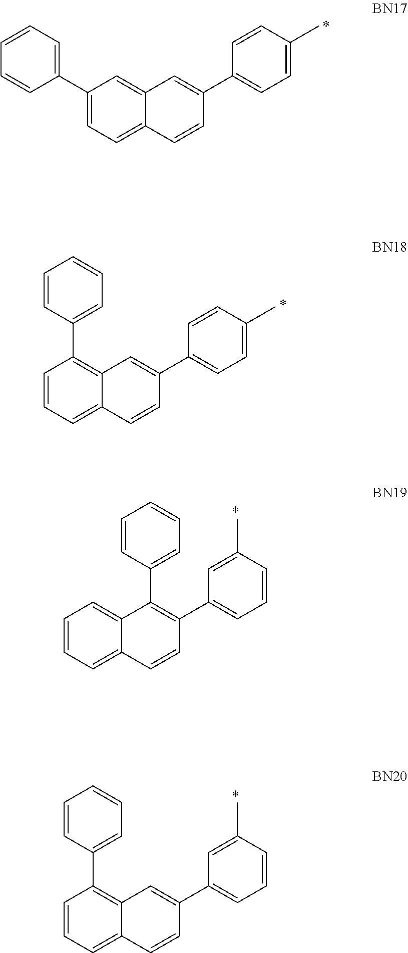 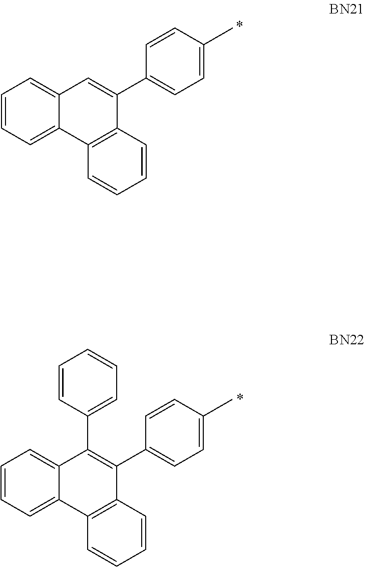 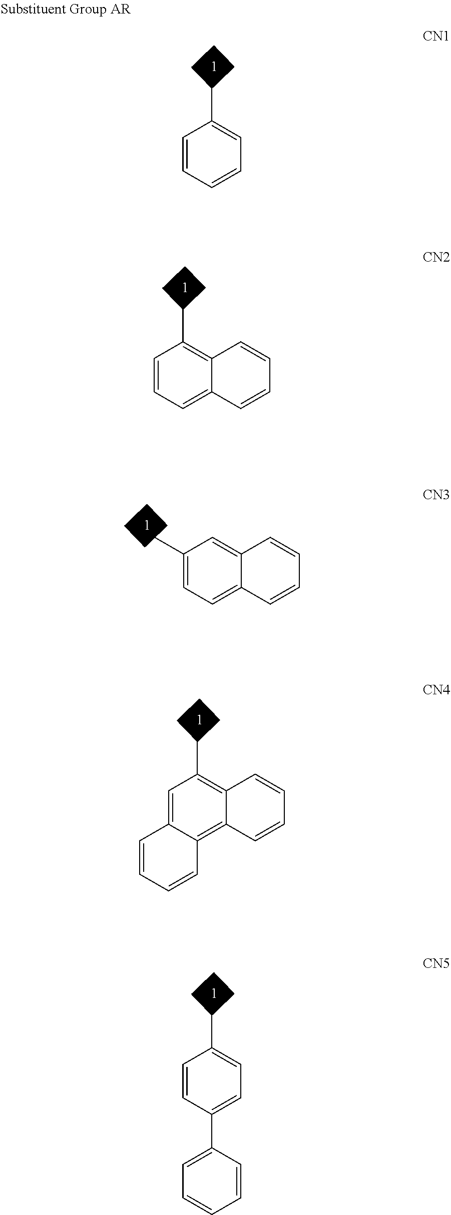 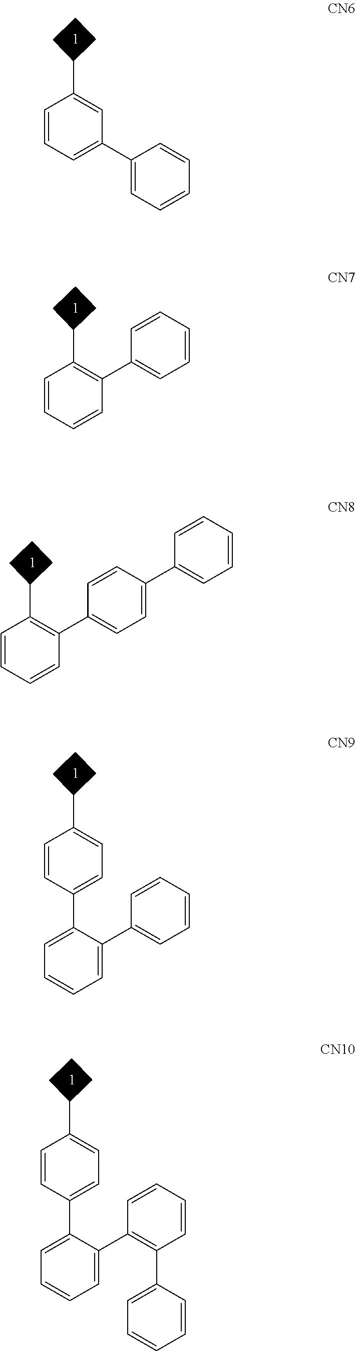 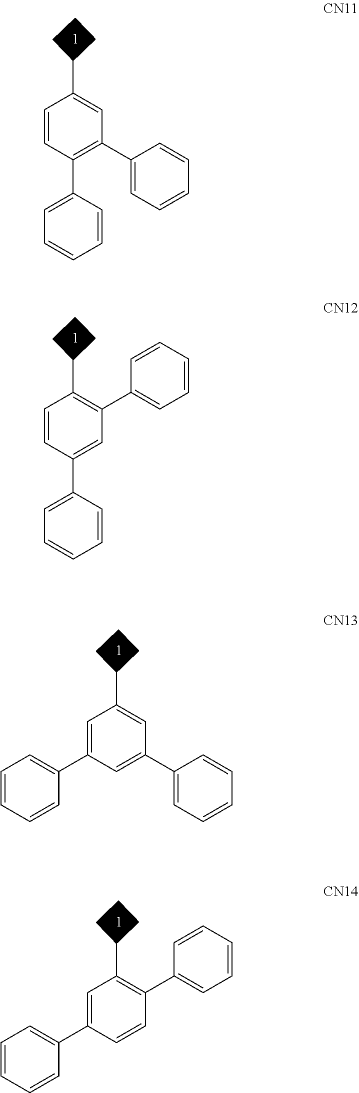 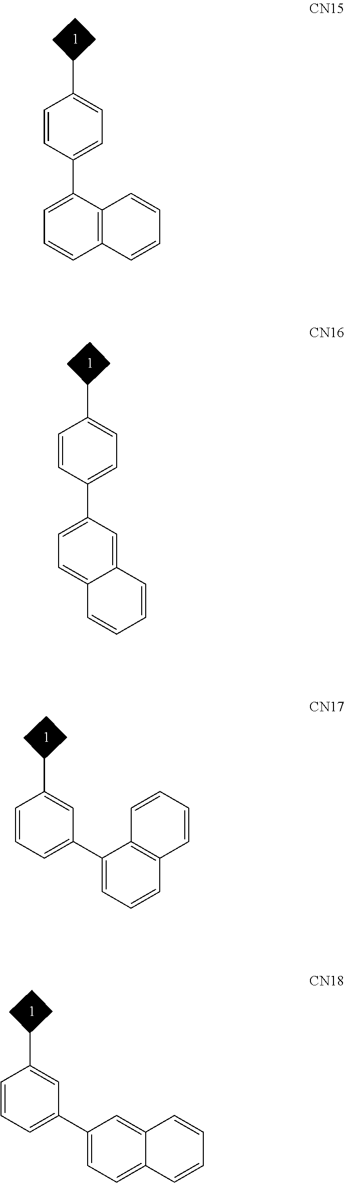 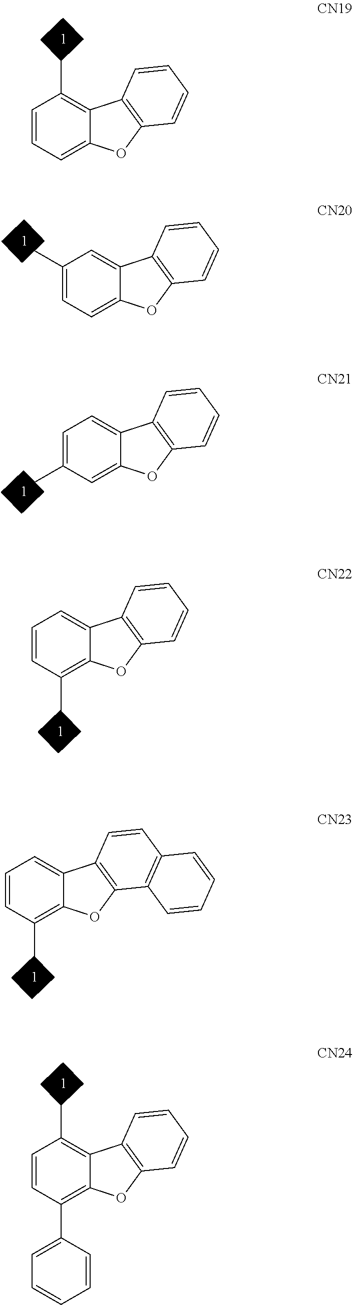 [SMILES: Cc1cccc2oc3ccccc3c12 ; Cc1ccc2oc3ccccc3c2c1 ; Cc1ccc2c(c1)oc1ccccc12 ; Cc1cccc2c1oc1ccccc12 ; Cc1cccc2c1oc1c3ccccc3ccc21 ; Cc1ccc(-c2ccccc2)c2oc3ccccc3c12] 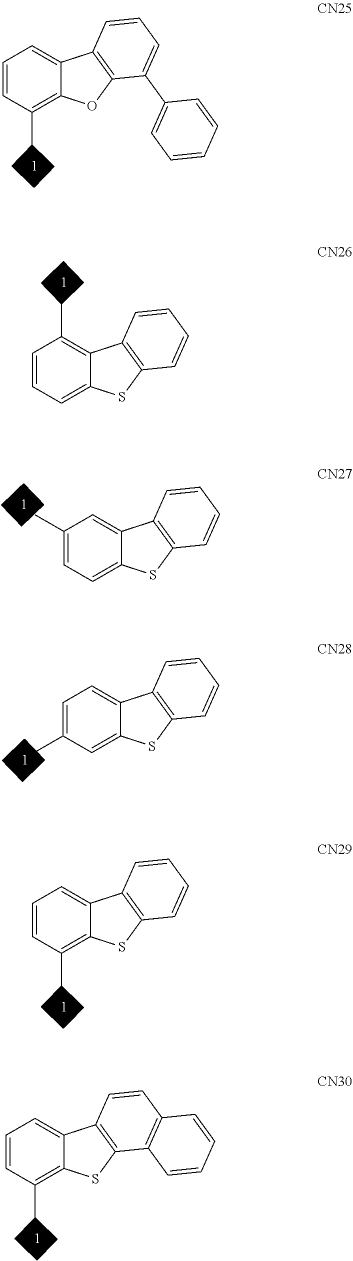 [SMILES: Cc1cccc2c1oc1c(-c3ccccc3)cccc12 ; Cc1cccc2sc3ccccc3c12 ; Cc1ccc2sc3ccccc3c2c1 ; Cc1ccc2c(c1)sc1ccccc12 ; Cc1cccc2c1sc1ccccc12 ; Cc1cccc2c1sc1c3ccccc3ccc21] 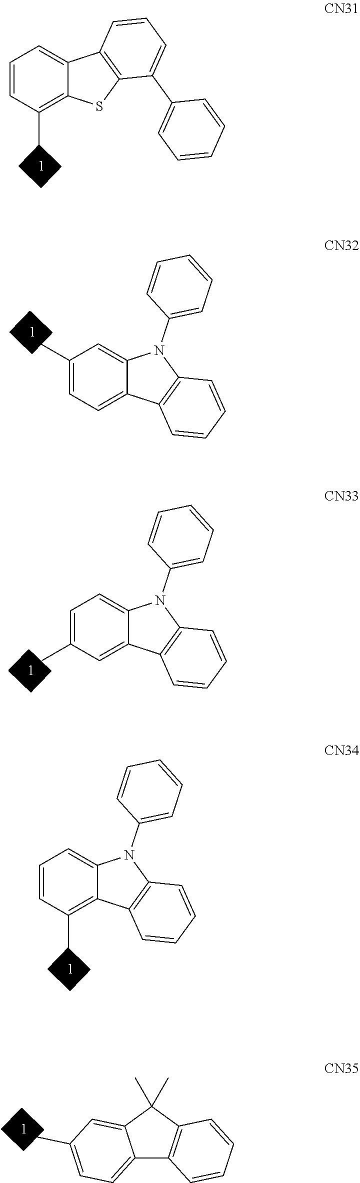 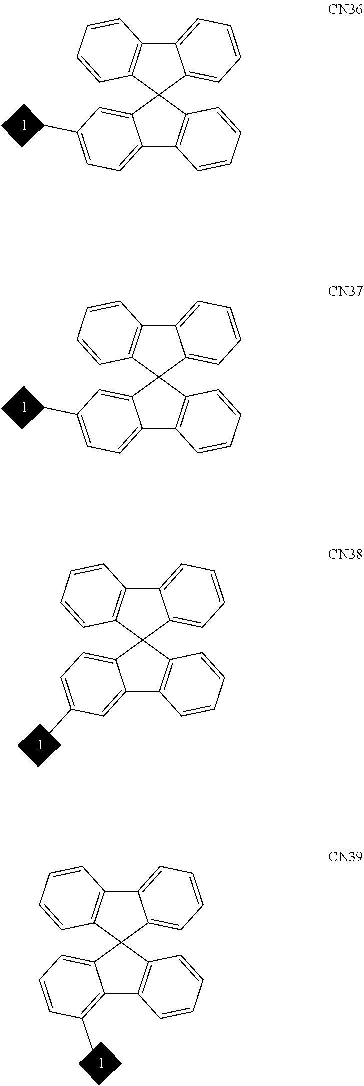 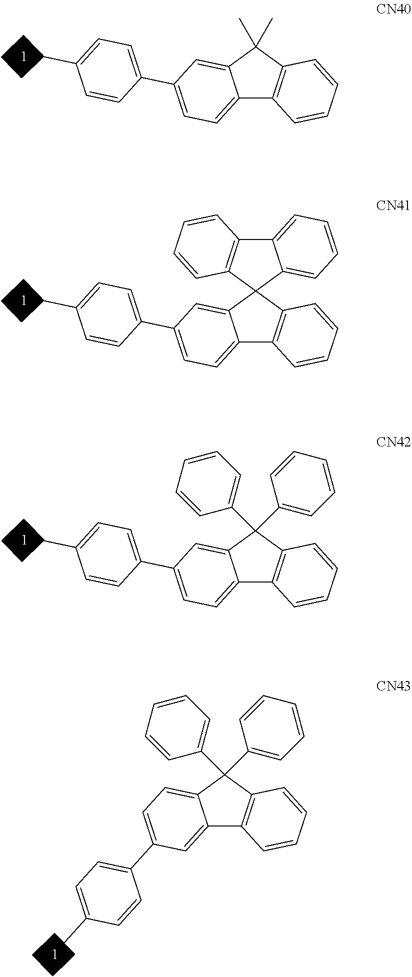 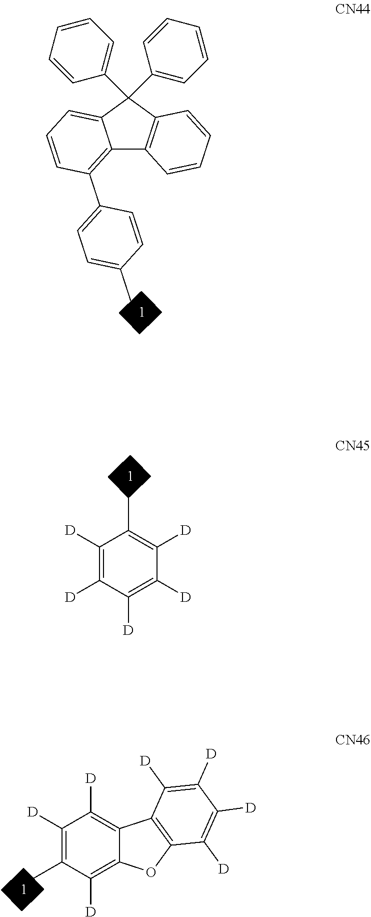
Compound Combination Table 1

| Compound | | | |
|---|---|---|---|
| NO. | AN | BN | AR₁ |
| AA1 | AN1 | BN1 | CN1 |
| AA2 | AN1 | BN1 | CN2 |
| AA3 | AN1 | BN1 | CN3 |
| AA4 | AN1 | BN1 | CN4 |
| AA5 | AN1 | BN1 | CN5 |
| AA6 | AN1 | BN1 | CN6 |
| AA7 | AN1 | BN1 | CN7 |
| AA8 | AN1 | BN1 | CN8 |
| AA9 | AN1 | BN1 | CN9 |
| AA10 | AN1 | BN1 | CN10 |
| AA11 | AN1 | BN1 | CN11 |
| AA12 | AN1 | BN1 | CN12 |
| AA13 | AN1 | BN1 | CN13 |
| AA14 | AN1 | BN1 | CN14 |
| AA15 | AN1 | BN1 | CN15 |
| AA16 | AN1 | BN1 | CN16 |
| AA17 | AN1 | BN1 | CN17 |
| AA18 | AN1 | BN1 | CN18 |
| AA19 | AN1 | BN1 | CN19 |
| AA20 | AN1 | BN1 | CN20 |
| AA21 | AN1 | BN1 | CN21 |
| AA22 | AN1 | BN1 | CN22 |
| AA23 | AN1 | BN1 | CN23 |
| AA24 | AN1 | BN1 | CN24 |
| AA25 | AN1 | BN1 | CN25 |
| AA26 | AN1 | BN1 | CN26 |
| AA27 | AN1 | BN1 | CN27 |
| AA28 | AN1 | BN1 | CN28 |
| AA29 | AN1 | BN1 | CN29 |
| AA30 | AN1 | BN1 | CN30 |
| AA31 | AN1 | BN1 | CN31 |
| AA32 | AN1 | BN1 | CN32 |
| AA33 | AN1 | BN1 | CN33 |
| AA34 | AN1 | BN1 | CN34 |
| AA35 | AN1 | BN1 | CN35 |
| AA36 | AN1 | BN1 | CN36 |
| AB1 | AN1 | BN2 | CN1 |
| AB2 | AN1 | BN2 | CN2 |
| AB3 | AN1 | BN2 | CN3 |
| AB4 | AN1 | BN2 | CN4 |
| AB5 | AN1 | BN2 | CN5 |
| AB6 | AN1 | BN2 | CN6 |
| AB7 | AN1 | BN2 | CN7 |
| AB8 | AN1 | BN2 | CN8 |
| AB9 | AN1 | BN2 | CN9 |
| AB10 | AN1 | BN2 | CN10 |
| AB11 | AN1 | BN2 | CN11 |
| AB12 | AN1 | BN2 | CN12 |
| AB13 | AN1 | BN2 | CN13 |
| AB14 | AN1 | BN2 | CN14 |
| AB15 | AN1 | BN2 | CN15 |
| AB16 | AN1 | BN2 | CN16 |
| AB17 | AN1 | BN2 | CN17 |
| AB18 | AN1 | BN2 | CN18 |
| AB19 | AN1 | BN2 | CN19 |
| AB20 | AN1 | BN2 | CN20 |
| AB21 | AN1 | BN2 | CN21 |
| AB22 | AN1 | BN2 | CN22 |
| AB23 | AN1 | BN2 | CN23 |
| AB24 | AN1 | BN2 | CN24 |
| AB25 | AN1 | BN2 | CN25 |
| AB26 | AN1 | BN2 | CN26 |
| AB27 | AN1 | BN2 | CN27 |
| AB28 | AN1 | BN2 | CN28 |
| AB29 | AN1 | BN2 | CN29 |
| AB30 | AN1 | BN2 | CN30 |
| AB31 | AN1 | BN2 | CN31 |
| AB32 | AN1 | BN2 | CN32 |
| AB33 | AN1 | BN2 | CN33 |
| AB34 | AN1 | BN2 | CN34 |
| AB35 | AN1 | BN2 | CN35 |
| AB36 | AN1 | BN2 | CN36 |
| AC1 | AN1 | BN3 | CN1 |
| AC2 | AN1 | BN3 | CN2 |
| AC3 | AN1 | BN3 | CN3 |
| AC4 | AN1 | BN3 | CN4 |
| AC5 | AN1 | BN3 | CN5 |
| AC6 | AN1 | BN3 | CN6 |
| AC7 | AN1 | BN3 | CN7 |
| AC8 | AN1 | BN3 | CN8 |
| AC9 | AN1 | BN3 | CN9 |
| AC10 | AN1 | BN3 | CN10 |
| AC11 | AN1 | BN3 | CN11 |
| AC12 | AN1 | BN3 | CN12 |
| AC13 | AN1 | BN3 | CN13 |
| AC14 | AN1 | BN3 | CN14 |
| AC15 | AN1 | BN3 | CN15 |
| AC16 | AN1 | BN3 | CN16 |
| AC17 | AN1 | BN3 | CN17 |
| AC18 | AN1 | BN3 | CN18 |
| AC19 | AN1 | BN3 | CN19 |
| AC20 | AN1 | BN3 | CN20 |
| AC21 | AN1 | BN3 | CN21 |
| AC22 | AN1 | BN3 | CN22 |
| AC23 | AN1 | BN3 | CN23 |
| AC24 | AN1 | BN3 | CN24 |
| AC25 | AN1 | BN3 | CN25 |
| AC26 | AN1 | BN3 | CN26 |
| AC27 | AN1 | BN3 | CN27 |
| AC28 | AN1 | BN3 | CN28 |
| AC29 | AN1 | BN3 | CN29 |
| AC30 | AN1 | BN3 | CN30 |
| AC31 | AN1 | BN3 | CN31 |
| AC32 | AN1 | BN3 | CN32 |
| AC33 | AN1 | BN3 | CN33 |
| AC34 | AN1 | BN3 | CN34 |
| AC35 | AN1 | BN3 | CN35 |
| AC36 | AN1 | BN3 | CN36 |
| AD1 | AN1 | BN4 | CN1 |
| AD2 | AN1 | BN4 | CN2 |
| AD3 | AN1 | BN4 | CN3 |
| AD4 | AN1 | BN4 | CN4 |
| AD5 | AN1 | BN4 | CN5 |
| AD6 | AN1 | BN4 | CN6 |
| AD7 | AN1 | BN4 | CN7 |
| AD8 | AN1 | BN4 | CN8 |
| AD9 | AN1 | BN4 | CN9 |
| AD10 | AN1 | BN4 | CN10 |
| AD11 | AN1 | BN4 | CN11 |
| AD12 | AN1 | BN4 | CN12 |
| AD13 | AN1 | BN4 | CN13 |
| AD14 | AN1 | BN4 | CN14 |
| AD15 | AN1 | BN4 | CN15 |
| AD16 | AN1 | BN4 | CN16 |
| AD17 | AN1 | BN4 | CN17 |
| AD18 | AN1 | BN4 | CN18 |
| AD19 | AN1 | BN4 | CN19 |
| AD20 | AN1 | BN4 | CN20 |
| AD21 | AN1 | BN4 | CN21 |
| AD22 | AN1 | BN4 | CN22 |
| AD23 | AN1 | BN4 | CN23 |
| AD24 | AN1 | BN4 | CN24 |
| AD25 | AN1 | BN4 | CN25 |
| AD26 | AN1 | BN4 | CN26 |
| AD27 | AN1 | BN4 | CN27 |
| AD28 | AN1 | BN4 | CN28 |
| AD29 | AN1 | BN4 | CN29 |
| AD30 | AN1 | BN4 | CN30 |
| AD31 | AN1 | BN4 | CN31 |
| AD32 | AN1 | BN4 | CN32 |
| AD33 | AN1 | BN4 | CN33 |
| AD34 | AN1 | BN4 | CN34 |
| AD35 | AN1 | BN4 | CN35 |
| AD36 | AN1 | BN4 | CN36 |
| AE1 | AN1 | BN5 | CN1 |
| AE2 | AN1 | BN5 | CN2 |
| AE3 | AN1 | BN5 | CN3 |
| AE4 | AN1 | BN5 | CN4 |
| AE5 | AN1 | BN5 | CN5 |
| AE6 | AN1 | BN5 | CN6 |
| AE7 | AN1 | BN5 | CN7 |
| AE8 | AN1 | BN5 | CN8 |
| AE9 | AN1 | BN5 | CN9 |
| AE10 | AN1 | BN5 | CN10 |
| AE11 | AN1 | BN5 | CN11 |
| AE12 | AN1 | BN5 | CN12 |
| AE13 | AN1 | BN5 | CN13 |
| AE14 | AN1 | BN5 | CN14 |
| AE15 | AN1 | BN5 | CN15 |
| AE16 | AN1 | BN5 | CN16 |
| AE17 | AN1 | BN5 | CN17 |
| AE18 | AN1 | BN5 | CN18 |
| AE19 | AN1 | BN5 | CN19 |
| AE20 | AN1 | BN5 | CN20 |
| AE21 | AN1 | BN5 | CN21 |
| AE22 | AN1 | BN5 | CN22 |
| AE23 | AN1 | BN5 | CN23 |
| AE24 | AN1 | BN5 | CN24 |
| AE25 | AN1 | BN5 | CN25 |
| AE26 | AN1 | BN5 | CN26 |
| AE27 | AN1 | BN5 | CN27 |
| AE28 | AN1 | BN5 | CN28 |
| AE29 | AN1 | BN5 | CN29 |
| AE30 | AN1 | BN5 | CN30 |
| AE31 | AN1 | BN5 | CN31 |
| AE32 | AN1 | BN5 | CN32 |
| AE33 | AN1 | BN5 | CN33 |
| AE34 | AN1 | BN5 | CN34 |
| AE35 | AN1 | BN5 | CN35 |
| AE36 | AN1 | BN5 | CN36 |
| AF1 | AN1 | BN6 | CN1 |
| AF2 | AN1 | BN6 | CN2 |
| AF3 | AN1 | BN6 | CN3 |
| AF4 | AN1 | BN6 | CN4 |
| AF5 | AN1 | BN6 | CN5 |
| AF6 | AN1 | BN6 | CN6 |
| AF7 | AN1 | BN6 | CN7 |
| AF8 | AN1 | BN6 | CN8 |
| AF9 | AN1 | BN6 | CN9 |
| AF10 | AN1 | BN6 | CN10 |
| AF11 | AN1 | BN6 | CN11 |
| AF12 | AN1 | BN6 | CN12 |
| AF13 | AN1 | BN6 | CN13 |
| AF14 | AN1 | BN6 | CN14 |
| AF15 | AN1 | BN6 | CN15 |
| AF16 | AN1 | BN6 | CN16 |
| AF17 | AN1 | BN6 | CN17 |
| AF18 | AN1 | BN6 | CN18 |
| AF19 | AN1 | BN6 | CN19 |
| AF20 | AN1 | BN6 | CN20 |
| AF21 | AN1 | BN6 | CN21 |
| AF22 | AN1 | BN6 | CN22 |
| AF23 | AN1 | BN6 | CN23 |
| AF24 | AN1 | BN6 | CN24 |
| AF25 | AN1 | BN6 | CN25 |
| AF26 | AN1 | BN6 | CN26 |
| AF27 | AN1 | BN6 | CN27 |
| AF28 | AN1 | BN6 | CN28 |
| AF29 | AN1 | BN6 | CN29 |
| AF30 | AN1 | BN6 | CN30 |
| AF31 | AN1 | BN6 | CN31 |
| AF32 | AN1 | BN6 | CN32 |
| AF33 | AN1 | BN6 | CN33 |
| AF34 | AN1 | BN6 | CN34 |
| AF35 | AN1 | BN6 | CN35 |
| AF36 | AN1 | BN6 | CN36 |
| AG1 | AN1 | BN7 | CN1 |
| AG2 | AN1 | BN7 | CN2 |
| AG3 | AN1 | BN7 | CN3 |
| AG4 | AN1 | BN7 | CN4 |
| AG5 | AN1 | BN7 | CN5 |
| AG6 | AN1 | BN7 | CN6 |
| AG7 | AN1 | BN7 | CN7 |
| AG8 | AN1 | BN7 | CN8 |
| AG9 | AN1 | BN7 | CN9 |
| AG10 | AN1 | BN7 | CN10 |
| AG11 | AN1 | BN7 | CN11 |
| AG12 | AN1 | BN7 | CN12 |
| AG13 | AN1 | BN7 | CN13 |
| AG14 | AN1 | BN7 | CN14 |
| AG15 | AN1 | BN7 | CN15 |
| AG16 | AN1 | BN7 | CN16 |
| AG17 | AN1 | BN7 | CN17 |
| AG18 | AN1 | BN7 | CN18 |
| AG19 | AN1 | BN7 | CN19 |
| AG20 | AN1 | BN7 | CN20 |
| AG21 | AN1 | BN7 | CN21 |
| AG22 | AN1 | BN7 | CN22 |
| AG23 | AN1 | BN7 | CN23 |
| AG24 | AN1 | BN7 | CN24 |
| AG25 | AN1 | BN7 | CN25 |
| AG26 | AN1 | BN7 | CN26 |
| AG27 | AN1 | BN7 | CN27 |
| AG28 | AN1 | BN7 | CN28 |
| AG29 | AN1 | BN7 | CN29 |
| AG30 | AN1 | BN7 | CN30 |
| AG31 | AN1 | BN7 | CN31 |
| AG32 | AN1 | BN7 | CN32 |
| AG33 | AN1 | BN7 | CN33 |
| AG34 | AN1 | BN7 | CN34 |
| AG35 | AN1 | BN7 | CN35 |
| AG36 | AN1 | BN7 | CN36 |
| AH1 | AN1 | BN8 | CN1 |
| AH2 | AN1 | BN8 | CN2 |
| AH3 | AN1 | BN8 | CN3 |
| AH4 | AN1 | BN8 | CN4 |
| AH5 | AN1 | BN8 | CN5 |
| AH6 | AN1 | BN8 | CN6 |
| AH7 | AN1 | BN8 | CN7 |
| AH8 | AN1 | BN8 | CN8 |
| AH9 | AN1 | BN8 | CN9 |
| AH10 | AN1 | BN8 | CN10 |
| AH11 | AN1 | BN8 | CN11 |
| AH12 | AN1 | BN8 | CN12 |
| AH13 | AN1 | BN8 | CN13 |
| AH14 | AN1 | BN8 | CN14 |
| AH15 | AN1 | BN8 | CN15 |
| AH16 | AN1 | BN8 | CN16 |
| AH17 | AN1 | BN8 | CN17 |
| AH18 | AN1 | BN8 | CN18 |
| AH19 | AN1 | BN8 | CN19 |
| AH20 | AN1 | BN8 | CN20 |
| AH21 | AN1 | BN8 | CN21 |
| AH22 | AN1 | BN8 | CN22 |
| AH23 | AN1 | BN8 | CN23 |
| AH24 | AN1 | BN8 | CN24 |
| AH25 | AN1 | BN8 | CN25 |
| AH26 | AN1 | BN8 | CN26 |
| AH27 | AN1 | BN8 | CN27 |
| AH28 | AN1 | BN8 | CN28 |
| AH29 | AN1 | BN8 | CN29 |
| AH30 | AN1 | BN8 | CN30 |
| AH31 | AN1 | BN8 | CN31 |
| AH32 | AN1 | BN8 | CN32 |
| AH33 | AN1 | BN8 | CN33 |
| AH34 | AN1 | BN8 | CN34 |
| AH35 | AN1 | BN8 | CN35 |
| AH36 | AN1 | BN8 | CN36 |
| AI1 | AN1 | BN9 | CN1 |
| AI2 | AN1 | BN9 | CN2 |
| AI3 | AN1 | BN9 | CN3 |
| AI4 | AN1 | BN9 | CN4 |
| AI5 | AN1 | BN9 | CN5 |
| AI6 | AN1 | BN9 | CN6 |
| AI7 | AN1 | BN9 | CN7 |
| AI8 | AN1 | BN9 | CN8 |
| AI9 | AN1 | BN9 | CN9 |
| AI10 | AN1 | BN9 | CN10 |
| AI11 | AN1 | BN9 | CN11 |
| AI12 | AN1 | BN9 | CN12 |
| AI13 | AN1 | BN9 | CN13 |
| AI14 | AN1 | BN9 | CN14 |
| AI15 | AN1 | BN9 | CN15 |
| AI16 | AN1 | BN9 | CN16 |
| AI17 | AN1 | BN9 | CN17 |
| AI18 | AN1 | BN9 | CN18 |
| AI19 | AN1 | BN9 | CN19 |
| AI20 | AN1 | BN9 | CN20 |
| AI21 | AN1 | BN9 | CN21 |
| AI22 | AN1 | BN9 | CN22 |
| AI23 | AN1 | BN9 | CN23 |
| AI24 | AN1 | BN9 | CN24 |
| AI25 | AN1 | BN9 | CN25 |
| AI26 | AN1 | BN9 | CN26 |
| AI27 | AN1 | BN9 | CN27 |
| AI28 | AN1 | BN9 | CN28 |
| AI29 | AN1 | BN9 | CN29 |
| AI30 | AN1 | BN9 | CN30 |
| AI31 | AN1 | BN9 | CN31 |
| AI32 | AN1 | BN9 | CN32 |
| AI33 | AN1 | BN9 | CN33 |
| AI34 | AN1 | BN9 | CN34 |
| AI35 | AN1 | BN9 | CN35 |
| AI36 | AN1 | BN9 | CN36 |
| AJ1 | AN1 | BN10 | CN1 |
| AJ2 | AN1 | BN10 | CN2 |
| AJ3 | AN1 | BN10 | CN3 |
| AJ4 | AN1 | BN10 | CN4 |
| AJ5 | AN1 | BN10 | CN5 |
| AJ6 | AN1 | BN10 | CN6 |
| AJ7 | AN1 | BN10 | CN7 |
| AJ8 | AN1 | BN10 | CN8 |
| AJ9 | AN1 | BN10 | CN9 |
| AJ10 | AN1 | BN10 | CN10 |
| AJ11 | AN1 | BN10 | CN11 |
| AJ12 | AN1 | BN10 | CN12 |
| AJ13 | AN1 | BN10 | CN13 |
| AJ14 | AN1 | BN10 | CN14 |
| AJ15 | AN1 | BN10 | CN15 |
| AJ16 | AN1 | BN10 | CN16 |
| AJ17 | AN1 | BN10 | CN17 |
| AJ18 | AN1 | BN10 | CN18 |
| AJ19 | AN1 | BN10 | CN19 |
| AJ20 | AN1 | BN10 | CN20 |
| AJ21 | AN1 | BN10 | CN21 |
| AJ22 | AN1 | BN10 | CN22 |
| AJ23 | AN1 | BN10 | CN23 |
| AJ24 | AN1 | BN10 | CN24 |
| AJ25 | AN1 | BN10 | CN25 |
| AJ26 | AN1 | BN10 | CN26 |
| AJ27 | AN1 | BN10 | CN27 |
| AJ28 | AN1 | BN10 | CN28 |
| AJ29 | AN1 | BN10 | CN29 |
| AJ30 | AN1 | BN10 | CN30 |
| AJ31 | AN1 | BN10 | CN31 |
| AJ32 | AN1 | BN10 | CN32 |
| AJ33 | AN1 | BN10 | CN33 |
| AJ34 | AN1 | BN10 | CN34 |
| AJ35 | AN1 | BN10 | CN35 |
| AJ36 | AN1 | BN10 | CN36 |
| AK1 | AN1 | BN11 | CN1 |
| AK2 | AN1 | BN11 | CN2 |
| AK3 | AN1 | BN11 | CN3 |
| AK4 | AN1 | BN11 | CN4 |
| AK5 | AN1 | BN11 | CN5 |
| AK6 | AN1 | BN11 | CN6 |
| AK7 | AN1 | BN11 | CN7 |
| AK8 | AN1 | BN11 | CN8 |
| AK9 | AN1 | BN11 | CN9 |
| AK10 | AN1 | BN11 | CN10 |
| AK11 | AN1 | BN11 | CN11 |
| AK12 | AN1 | BN11 | CN12 |
| AK13 | AN1 | BN11 | CN13 |
| AK14 | AN1 | BN11 | CN14 |
| AK15 | AN1 | BN11 | CN15 |
| AK16 | AN1 | BN11 | CN16 |
| AK17 | AN1 | BN11 | CN17 |
| AK18 | AN1 | BN11 | CN18 |
| AK19 | AN1 | BN11 | CN19 |
| AK20 | AN1 | BN11 | CN20 |
| AK21 | AN1 | BN11 | CN21 |
| AK22 | AN1 | BN11 | CN22 |
| AK23 | AN1 | BN11 | CN23 |
| AK24 | AN1 | BN11 | CN24 |
| AK25 | AN1 | BN11 | CN25 |
| AK26 | AN1 | BN11 | CN26 |
| AK27 | AN1 | BN11 | CN27 |
| AK28 | AN1 | BN11 | CN28 |
| AK29 | AN1 | BN11 | CN29 |
| AK30 | AN1 | BN11 | CN30 |
| AK31 | AN1 | BN11 | CN31 |
| AK32 | AN1 | BN11 | CN32 |
| AK33 | AN1 | BN11 | CN33 |
| AK34 | AN1 | BN11 | CN34 |
| AK35 | AN1 | BN11 | CN35 |
| AK36 | AN1 | BN11 | CN36 |
| AL1 | AN1 | BN12 | CN1 |
| AL2 | AN1 | BN12 | CN2 |
| AL3 | AN1 | BN12 | CN3 |
| AL4 | AN1 | BN12 | CN4 |
| AL5 | AN1 | BN12 | CN5 |
| AL6 | AN1 | BN12 | CN6 |
| AL7 | AN1 | BN12 | CN7 |
| AL8 | AN1 | BN12 | CN8 |
| AL9 | AN1 | BN12 | CN9 |
| AL10 | AN1 | BN12 | CN10 |
| AL11 | AN1 | BN12 | CN11 |
| AL12 | AN1 | BN12 | CN12 |
| AL13 | AN1 | BN12 | CN13 |
| AL14 | AN1 | BN12 | CN14 |
| AL15 | AN1 | BN12 | CN15 |
| AL16 | AN1 | BN12 | CN16 |
| AL17 | AN1 | BN12 | CN17 |
| AL18 | AN1 | BN12 | CN18 |
| AL19 | AN1 | BN12 | CN19 |
| AL20 | AN1 | BN12 | CN20 |
| AL21 | AN1 | BN12 | CN21 |
| AL22 | AN1 | BN12 | CN22 |
| AL23 | AN1 | BN12 | CN23 |
| AL24 | AN1 | BN12 | CN24 |
| AL25 | AN1 | BN12 | CN25 |
| AL26 | AN1 | BN12 | CN26 |
| AL27 | AN1 | BN12 | CN27 |
| AL28 | AN1 | BN12 | CN28 |
| AL29 | AN1 | BN12 | CN29 |
| AL30 | AN1 | BN12 | CN30 |
| AL31 | AN1 | BN12 | CN31 |
| AL32 | AN1 | BN12 | CN32 |
| AL33 | AN1 | BN12 | CN33 |
| AL34 | AN1 | BN12 | CN34 |
| AL35 | AN1 | BN12 | CN35 |
| AL36 | AN1 | BN12 | CN36 |
| AM1 | AN1 | BN13 | CN1 |
| AM2 | AN1 | BN13 | CN2 |
| AM3 | AN1 | BN13 | CN3 |
| AM4 | AN1 | BN13 | CN4 |
| AM5 | AN1 | BN13 | CN5 |
| AM6 | AN1 | BN13 | CN6 |
| AM7 | AN1 | BN13 | CN7 |
| AM8 | AN1 | BN13 | CN8 |
| AM9 | AN1 | BN13 | CN9 |
| AM10 | AN1 | BN13 | CN10 |
| AM11 | AN1 | BN13 | CN11 |
| AM12 | AN1 | BN13 | CN12 |
| AM13 | AN1 | BN13 | CN13 |
| AM14 | AN1 | BN13 | CN14 |
| AM15 | AN1 | BN13 | CN15 |
| AM16 | AN1 | BN13 | CN16 |
| AM17 | AN1 | BN13 | CN17 |
| AM18 | AN1 | BN13 | CN18 |
| AM19 | AN1 | BN13 | CN19 |
| AM20 | AN1 | BN13 | CN20 |
| AM21 | AN1 | BN13 | CN21 |
| AM22 | AN1 | BN13 | CN22 |
| AM23 | AN1 | BN13 | CN23 |
| AM24 | AN1 | BN13 | CN24 |
| AM25 | AN1 | BN13 | CN25 |
| AM26 | AN1 | BN13 | CN26 |
| AM27 | AN1 | BN13 | CN27 |
| AM28 | AN1 | BN13 | CN28 |
| AM29 | AN1 | BN13 | CN29 |
| AM30 | AN1 | BN13 | CN30 |
| AM31 | AN1 | BN13 | CN31 |
| AM32 | AN1 | BN13 | CN32 |
| AM33 | AN1 | BN13 | CN33 |
| AM34 | AN1 | BN13 | CN34 |
| AM35 | AN1 | BN13 | CN35 |
| AM36 | AN1 | BN13 | CN36 |
| AO1 | AN1 | BN14 | CN1 |
| AO2 | AN1 | BN14 | CN2 |
| AO3 | AN1 | BN14 | CN3 |
| AO4 | AN1 | BN14 | CN4 |
| AO5 | AN1 | BN14 | CN5 |
| AO6 | AN1 | BN14 | CN6 |
| AO7 | AN1 | BN14 | CN7 |
| AO8 | AN1 | BN14 | CN8 |
| AO9 | AN1 | BN14 | CN9 |
| AO10 | AN1 | BN14 | CN10 |
| AO11 | AN1 | BN14 | CN11 |
| AO12 | AN1 | BN14 | CN12 |
| AO13 | AN1 | BN14 | CN13 |
| AO14 | AN1 | BN14 | CN14 |
| AO15 | AN1 | BN14 | CN15 |
| AO16 | AN1 | BN14 | CN16 |
| AO17 | AN1 | BN14 | CN17 |
| AO18 | AN1 | BN14 | CN18 |
| AO19 | AN1 | BN14 | CN19 |
| AO20 | AN1 | BN14 | CN20 |
| AO21 | AN1 | BN14 | CN21 |
| AO22 | AN1 | BN14 | CN22 |
| AO23 | AN1 | BN14 | CN23 |
| AO24 | AN1 | BN14 | CN24 |
| AO25 | AN1 | BN14 | CN25 |
| AO26 | AN1 | BN14 | CN26 |
| AO27 | AN1 | BN14 | CN27 |
| AO28 | AN1 | BN14 | CN28 |
| AO29 | AN1 | BN14 | CN29 |
| AO30 | AN1 | BN14 | CN30 |
| AO31 | AN1 | BN14 | CN31 |
| AO32 | AN1 | BN14 | CN32 |
| AO33 | AN1 | BN14 | CN33 |
| AO34 | AN1 | BN14 | CN34 |
| AO35 | AN1 | BN14 | CN35 |
| AO36 | AN1 | BN14 | CN36 |
| AP1 | AN1 | BN15 | CN1 |
| AP2 | AN1 | BN15 | CN2 |
| AP3 | AN1 | BN15 | CN3 |
| AP4 | AN1 | BN15 | CN4 |
| AP5 | AN1 | BN15 | CN5 |
| AP6 | AN1 | BN15 | CN6 |
| AP7 | AN1 | BN15 | CN7 |
| AP8 | AN1 | BN15 | CN8 |
| AP9 | AN1 | BN15 | CN9 |
| AP10 | AN1 | BN15 | CN10 |
| AP11 | AN1 | BN15 | CN11 |
| AP12 | AN1 | BN15 | CN12 |
| AP13 | AN1 | BN15 | CN13 |
| AP14 | AN1 | BN15 | CN14 |
| AP15 | AN1 | BN15 | CN15 |
| AP16 | AN1 | BN15 | CN16 |
| AP17 | AN1 | BN15 | CN17 |
| AP18 | AN1 | BN15 | CN18 |
| AP19 | AN1 | BN15 | CN19 |
| AP20 | AN1 | BN15 | CN20 |
| AP21 | AN1 | BN15 | CN21 |
| AP22 | AN1 | BN15 | CN22 |
| AP23 | AN1 | BN15 | CN23 |
| AP24 | AN1 | BN15 | CN24 |
| AP25 | AN1 | BN15 | CN25 |
| AP26 | AN1 | BN15 | CN26 |
| AP27 | AN1 | BN15 | CN27 |
| AP28 | AN1 | BN15 | CN28 |
| AP29 | AN1 | BN15 | CN29 |
| AP30 | AN1 | BN15 | CN30 |
| AP31 | AN1 | BN15 | CN31 |
| AP32 | AN1 | BN15 | CN32 |
| AP33 | AN1 | BN15 | CN33 |
| AP34 | AN1 | BN15 | CN34 |
| AP35 | AN1 | BN15 | CN35 |
| AP36 | AN1 | BN15 | CN36 |
| AQ1 | AN1 | BN16 | CN1 |
| AQ2 | AN1 | BN16 | CN2 |
| AQ3 | AN1 | BN16 | CN3 |
| AQ4 | AN1 | BN16 | CN4 |
| AQ5 | AN1 | BN16 | CN5 |
| AQ6 | AN1 | BN16 | CN6 |
| AQ7 | AN1 | BN16 | CN7 |
| AQ8 | AN1 | BN16 | CN8 |
| AQ9 | AN1 | BN16 | CN9 |
| AQ10 | AN1 | BN16 | CN10 |
| AQ11 | AN1 | BN16 | CN11 |
| AQ12 | AN1 | BN16 | CN12 |
| AQ13 | AN1 | BN16 | CN13 |
| AQ14 | AN1 | BN16 | CN14 |
| AQ15 | AN1 | BN16 | CN15 |
| AQ16 | AN1 | BN16 | CN16 |
| AQ17 | AN1 | BN16 | CN17 |
| AQ18 | AN1 | BN16 | CN18 |
| AQ19 | AN1 | BN16 | CN19 |
| AQ20 | AN1 | BN16 | CN20 |
| AQ21 | AN1 | BN16 | CN21 |
| AQ22 | AN1 | BN16 | CN22 |
| AQ23 | AN1 | BN16 | CN23 |
| AQ24 | AN1 | BN16 | CN24 |
| AQ25 | AN1 | BN16 | CN25 |
| AQ26 | AN1 | BN16 | CN26 |
| AQ27 | AN1 | BN16 | CN27 |
| AQ28 | AN1 | BN16 | CN28 |
| AQ29 | AN1 | BN16 | CN29 |
| AQ30 | AN1 | BN16 | CN30 |
| AQ31 | AN1 | BN16 | CN31 |
| AQ32 | AN1 | BN16 | CN32 |
| AQ33 | AN1 | BN16 | CN33 |
| AQ34 | AN1 | BN16 | CN34 |
| AQ35 | AN1 | BN16 | CN35 |
| AQ36 | AN1 | BN16 | CN36 |
| AR1 | AN1 | BN17 | CN1 |
| AR2 | AN1 | BN17 | CN2 |
| AR3 | AN1 | BN17 | CN3 |
| AR4 | AN1 | BN17 | CN4 |
| AR5 | AN1 | BN17 | CN5 |
| AR6 | AN1 | BN17 | CN6 |
| AR7 | AN1 | BN17 | CN7 |
| AR8 | AN1 | BN17 | CN8 |
| AR9 | AN1 | BN17 | CN9 |
| AR10 | AN1 | BN17 | CN10 |
| AR11 | AN1 | BN17 | CN11 |
| AR12 | AN1 | BN17 | CN12 |
| AR13 | AN1 | BN17 | CN13 |
| AR14 | AN1 | BN17 | CN14 |
| AR15 | AN1 | BN17 | CN15 |
| AR16 | AN1 | BN17 | CN16 |
| AR17 | AN1 | BN17 | CN17 |
| AR18 | AN1 | BN17 | CN18 |
| AR19 | AN1 | BN17 | CN19 |
| AR20 | AN1 | BN17 | CN20 |
| AR21 | AN1 | BN17 | CN21 |
| AR22 | AN1 | BN17 | CN22 |
| AR23 | AN1 | BN17 | CN23 |
| AR24 | AN1 | BN17 | CN24 |
| AR25 | AN1 | BN17 | CN25 |
| AR26 | AN1 | BN17 | CN26 |
| AR27 | AN1 | BN17 | CN27 |
| AR28 | AN1 | BN17 | CN28 |
| AR29 | AN1 | BN17 | CN29 |
| AR30 | AN1 | BN17 | CN30 |
| AR31 | AN1 | BN17 | CN31 |
| AR32 | AN1 | BN17 | CN32 |
| AR33 | AN1 | BN17 | CN33 |
| AR34 | AN1 | BN17 | CN34 |
| AR35 | AN1 | BN17 | CN35 |
| AR36 | AN1 | BN17 | CN36 |
| AS1 | AN1 | BN18 | CN1 |
| AS2 | AN1 | BN18 | CN2 |
| AS3 | AN1 | BN18 | CN3 |
| AS4 | AN1 | BN18 | CN4 |
| AS5 | AN1 | BN18 | CN5 |
| AS6 | AN1 | BN18 | CN6 |
| AS7 | AN1 | BN18 | CN7 |
| AS8 | AN1 | BN18 | CN8 |
| AS9 | AN1 | BN18 | CN9 |
| AS10 | AN1 | BN18 | CN10 |
| AS11 | AN1 | BN18 | CN11 |
| AS12 | AN1 | BN18 | CN12 |
| AS13 | AN1 | BN18 | CN13 |
| AS14 | AN1 | BN18 | CN14 |
| AS15 | AN1 | BN18 | CN15 |
| AS16 | AN1 | BN18 | CN16 |
| AS17 | AN1 | BN18 | CN17 |
| AS18 | AN1 | BN18 | CN18 |
| AS19 | AN1 | BN18 | CN19 |
| AS20 | AN1 | BN18 | CN20 |
| AS21 | AN1 | BN18 | CN21 |
| AS22 | AN1 | BN18 | CN22 |
| AS23 | AN1 | BN18 | CN23 |
| AS24 | AN1 | BN18 | CN24 |
| AS25 | AN1 | BN18 | CN25 |
| AS26 | AN1 | BN18 | CN26 |
| AS27 | AN1 | BN18 | CN27 |
| AS28 | AN1 | BN18 | CN28 |
| AS29 | AN1 | BN18 | CN29 |
| AS30 | AN1 | BN18 | CN30 |
| AS31 | AN1 | BN18 | CN31 |
| AS32 | AN1 | BN18 | CN32 |
| AS33 | AN1 | BN18 | CN33 |
| AS34 | AN1 | BN18 | CN34 |
| AS35 | AN1 | BN18 | CN35 |
| AS36 | AN1 | BN18 | CN36 |
| AT1 | AN1 | BN19 | CN1 |
| AT2 | AN1 | BN19 | CN2 |
| AT3 | AN1 | BN19 | CN3 |
| AT4 | AN1 | BN19 | CN4 |
| AT5 | AN1 | BN19 | CN5 |
| AT6 | AN1 | BN19 | CN6 |
| AT7 | AN1 | BN19 | CN7 |
| AT8 | AN1 | BN19 | CN8 |
| AT9 | AN1 | BN19 | CN9 |
| AT10 | AN1 | BN19 | CN10 |
| AT11 | AN1 | BN19 | CN11 |
| AT12 | AN1 | BN19 | CN12 |
| AT13 | AN1 | BN19 | CN13 |
| AT14 | AN1 | BN19 | CN14 |
| AT15 | AN1 | BN19 | CN15 |
| AT16 | AN1 | BN19 | CN16 |
| AT17 | AN1 | BN19 | CN17 |
| AT18 | AN1 | BN19 | CN18 |
| AT19 | AN1 | BN19 | CN19 |
| AT20 | AN1 | BN19 | CN20 |
| AT21 | AN1 | BN19 | CN21 |
| AT22 | AN1 | BN19 | CN22 |
| AT23 | AN1 | BN19 | CN23 |
| AT24 | AN1 | BN19 | CN24 |
| AT25 | AN1 | BN19 | CN25 |
| AT26 | AN1 | BN19 | CN26 |
| AT27 | AN1 | BN19 | CN27 |
| AT28 | AN1 | BN19 | CN28 |
| AT29 | AN1 | BN19 | CN29 |
| AT30 | AN1 | BN19 | CN30 |
| AT31 | AN1 | BN19 | CN31 |
| AT32 | AN1 | BN19 | CN32 |
| AT33 | AN1 | BN19 | CN33 |
| AT34 | AN1 | BN19 | CN34 |
| AT35 | AN1 | BN19 | CN35 |
| AT36 | AN1 | BN19 | CN36 |
| AU1 | AN1 | BN20 | CN1 |
| AU2 | AN1 | BN20 | CN2 |
| AU3 | AN1 | BN20 | CN3 |
| AU4 | AN1 | BN20 | CN4 |
| AU5 | AN1 | BN20 | CN5 |
| AU6 | AN1 | BN20 | CN6 |
| AU7 | AN1 | BN20 | CN7 |
| AU8 | AN1 | BN20 | CN8 |
| AU9 | AN1 | BN20 | CN9 |
| AU10 | AN1 | BN20 | CN10 |
| AU11 | AN1 | BN20 | CN11 |
| AU12 | AN1 | BN20 | CN12 |
| AU13 | AN1 | BN20 | CN13 |
| AU14 | AN1 | BN20 | CN14 |
| AU15 | AN1 | BN20 | CN15 |
| AU16 | AN1 | BN20 | CN16 |
| AU17 | AN1 | BN20 | CN17 |
| AU18 | AN1 | BN20 | CN18 |
| AU19 | AN1 | BN20 | CN19 |
| AU20 | AN1 | BN20 | CN20 |
| AU21 | AN1 | BN20 | CN21 |
| AU22 | AN1 | BN20 | CN22 |
| AU23 | AN1 | BN20 | CN23 |
| AU24 | AN1 | BN20 | CN24 |
| AU25 | AN1 | BN20 | CN25 |
| AU26 | AN1 | BN20 | CN26 |
| AU27 | AN1 | BN20 | CN27 |
| AU28 | AN1 | BN20 | CN28 |
| AU29 | AN1 | BN20 | CN29 |
| AU30 | AN1 | BN20 | CN30 |
| AU31 | AN1 | BN20 | CN31 |
| AU32 | AN1 | BN20 | CN32 |
| AU33 | AN1 | BN20 | CN33 |
| AU34 | AN1 | BN20 | CN34 |
| AU35 | AN1 | BN20 | CN35 |
| AU36 | AN1 | BN20 | CN36 |
| AV1 | AN1 | BN21 | CN1 |
| AV2 | AN1 | BN21 | CN2 |
| AV3 | AN1 | BN21 | CN3 |
| AV4 | AN1 | BN21 | CN4 |
| AV5 | AN1 | BN21 | CN5 |
| AV6 | AN1 | BN21 | CN6 |
| AV7 | AN1 | BN21 | CN7 |
| AV8 | AN1 | BN21 | CN8 |
| AV9 | AN1 | BN21 | CN9 |
| AV10 | AN1 | BN21 | CN10 |
| AV11 | AN1 | BN21 | CN11 |
| AV12 | AN1 | BN21 | CN12 |
| AV13 | AN1 | BN21 | CN13 |
| AV14 | AN1 | BN21 | CN14 |
| AV15 | AN1 | BN21 | CN15 |
| AV16 | AN1 | BN21 | CN16 |
| AV17 | AN1 | BN21 | CN17 |
| AV18 | AN1 | BN21 | CN18 |
| AV19 | AN1 | BN21 | CN19 |
| AV20 | AN1 | BN21 | CN20 |
| AV21 | AN1 | BN21 | CN21 |
| AV22 | AN1 | BN21 | CN22 |
| AV23 | AN1 | BN21 | CN23 |
| AV24 | AN1 | BN21 | CN24 |
| AV25 | AN1 | BN21 | CN25 |
| AV26 | AN1 | BN21 | CN26 |
| AV27 | AN1 | BN21 | CN27 |
| AV28 | AN1 | BN21 | CN28 |
| AV29 | AN1 | BN21 | CN29 |
| AV30 | AN1 | BN21 | CN30 |
| AV31 | AN1 | BN21 | CN31 |
| AV32 | AN1 | BN21 | CN32 |
| AV33 | AN1 | BN21 | CN33 |
| AV34 | AN1 | BN21 | CN34 |
| AV35 | AN1 | BN21 | CN35 |
| AV36 | AN1 | BN21 | CN36 |
| AW1 | AN1 | BN22 | CN1 |
| AW2 | AN1 | BN22 | CN2 |
| AW3 | AN1 | BN22 | CN3 |
| AW4 | AN1 | BN22 | CN4 |
| AW5 | AN1 | BN22 | CN5 |
| AW6 | AN1 | BN22 | CN6 |
| AW7 | AN1 | BN22 | CN7 |
| AW8 | AN1 | BN22 | CN8 |
| AW9 | AN1 | BN22 | CN9 |
| AW10 | AN1 | BN22 | CN10 |
| AW11 | AN1 | BN22 | CN11 |
| AW12 | AN1 | BN22 | CN12 |
| AW13 | AN1 | BN22 | CN13 |
| AW14 | AN1 | BN22 | CN14 |
| AW15 | AN1 | BN22 | CN15 |
| AW16 | AN1 | BN22 | CN16 |
| AW17 | AN1 | BN22 | CN17 |
| AW18 | AN1 | BN22 | CN18 |
| AW19 | AN1 | BN22 | CN19 |
| AW20 | AN1 | BN22 | CN20 |
| AW21 | AN1 | BN22 | CN21 |
| AW22 | AN1 | BN22 | CN22 |
| AW23 | AN1 | BN22 | CN23 |
| AW24 | AN1 | BN22 | CN24 |
| AW25 | AN1 | BN22 | CN25 |
| AW26 | AN1 | BN22 | CN26 |
| AW27 | AN1 | BN22 | CN27 |
| AW28 | AN1 | BN22 | CN28 |
| AW29 | AN1 | BN22 | CN29 |
| AW30 | AN1 | BN22 | CN30 |
| AW31 | AN1 | BN22 | CN31 |
| AW32 | AN1 | BN22 | CN32 |
| AW33 | AN1 | BN22 | CN33 |
| AW34 | AN1 | BN22 | CN34 |
| AW35 | AN1 | BN22 | CN35 |
| AW36 | AN1 | BN22 | CN36 |
| BA1 | AN2 | BN1 | CN1 |
| BA2 | AN2 | BN1 | CN2 |
| BA3 | AN2 | BN1 | CN3 |
| BA4 | AN2 | BN1 | CN4 |
| BA5 | AN2 | BN1 | CN5 |
| BA6 | AN2 | BN1 | CN6 |
| BA7 | AN2 | BN1 | CN7 |
| BA8 | AN2 | BN1 | CN8 |
| BA9 | AN2 | BN1 | CN9 |
| BA10 | AN2 | BN1 | CN10 |
| BA11 | AN2 | BN1 | CN11 |
| BA12 | AN2 | BN1 | CN12 |
| BA13 | AN2 | BN1 | CN13 |
| BA14 | AN2 | BN1 | CN14 |
| BA15 | AN2 | BN1 | CN15 |
| BA16 | AN2 | BN1 | CN16 |
| BA17 | AN2 | BN1 | CN17 |
| BA18 | AN2 | BN1 | CN18 |
| BA19 | AN2 | BN1 | CN19 |
| BA20 | AN2 | BN1 | CN20 |
| BA21 | AN2 | BN1 | CN21 |
| BA22 | AN2 | BN1 | CN22 |
| BA23 | AN2 | BN1 | CN23 |
| BA24 | AN2 | BN1 | CN24 |
| BA25 | AN2 | BN1 | CN25 |
| BA26 | AN2 | BN1 | CN26 |
| BA27 | AN2 | BN1 | CN27 |
| BA28 | AN2 | BN1 | CN28 |
| BA29 | AN2 | BN1 | CN29 |
| BA30 | AN2 | BN1 | CN30 |
| BA31 | AN2 | BN1 | CN31 |
| BA32 | AN2 | BN1 | CN32 |
| BA33 | AN2 | BN1 | CN33 |
| BA34 | AN2 | BN1 | CN34 |
| BA35 | AN2 | BN1 | CN35 |
| BA36 | AN2 | BN1 | CN36 |
| BB1 | AN2 | BN2 | CN1 |
| BB2 | AN2 | BN2 | CN2 |
| BB3 | AN2 | BN2 | CN3 |
| BB4 | AN2 | BN2 | CN4 |
| BB5 | AN2 | BN2 | CN5 |
| BB6 | AN2 | BN2 | CN6 |
| BB7 | AN2 | BN2 | CN7 |
| BB8 | AN2 | BN2 | CN8 |
| BB9 | AN2 | BN2 | CN9 |
| BB10 | AN2 | BN2 | CN10 |
| BB11 | AN2 | BN2 | CN11 |
| BB12 | AN2 | BN2 | CN12 |
| BB13 | AN2 | BN2 | CN13 |
| BB14 | AN2 | BN2 | CN14 |
| BB15 | AN2 | BN2 | CN15 |
| BB16 | AN2 | BN2 | CN16 |
| BB17 | AN2 | BN2 | CN17 |
| BB18 | AN2 | BN2 | CN18 |
| BB19 | AN2 | BN2 | CN19 |
| BB20 | AN2 | BN2 | CN20 |
| BB21 | AN2 | BN2 | CN21 |
| BB22 | AN2 | BN2 | CN22 |
| BB23 | AN2 | BN2 | CN23 |
| BB24 | AN2 | BN2 | CN24 |
| BB25 | AN2 | BN2 | CN25 |
| BB26 | AN2 | BN2 | CN26 |
| BB27 | AN2 | BN2 | CN27 |
| BB28 | AN2 | BN2 | CN28 |
| BB29 | AN2 | BN2 | CN29 |
| BB30 | AN2 | BN2 | CN30 |
| BB31 | AN2 | BN2 | CN31 |
| BB32 | AN2 | BN2 | CN32 |
| BB33 | AN2 | BN2 | CN33 |
| BB34 | AN2 | BN2 | CN34 |
| BB35 | AN2 | BN2 | CN35 |
| BB36 | AN2 | BN2 | CN36 |
| BC1 | AN2 | BN3 | CN1 |
| BC2 | AN2 | BN3 | CN2 |
| BC3 | AN2 | BN3 | CN3 |
| BC4 | AN2 | BN3 | CN4 |
| BC5 | AN2 | BN3 | CN5 |
| BC6 | AN2 | BN3 | CN6 |
| BC7 | AN2 | BN3 | CN7 |
| BC8 | AN2 | BN3 | CN8 |
| BC9 | AN2 | BN3 | CN9 |
| BC10 | AN2 | BN3 | CN10 |
| BC11 | AN2 | BN3 | CN11 |
| BC12 | AN2 | BN3 | CN12 |
| BC13 | AN2 | BN3 | CN13 |
| BC14 | AN2 | BN3 | CN14 |
| BC15 | AN2 | BN3 | CN15 |
| BC16 | AN2 | BN3 | CN16 |
| BC17 | AN2 | BN3 | CN17 |
| BC18 | AN2 | BN3 | CN18 |
| BC19 | AN2 | BN3 | CN19 |
| BC20 | AN2 | BN3 | CN20 |
| BC21 | AN2 | BN3 | CN21 |
| BC22 | AN2 | BN3 | CN22 |
| BC23 | AN2 | BN3 | CN23 |
| BC24 | AN2 | BN3 | CN24 |
| BC25 | AN2 | BN3 | CN25 |
| BC26 | AN2 | BN3 | CN26 |
| BC27 | AN2 | BN3 | CN27 |
| BC28 | AN2 | BN3 | CN28 |
| BC29 | AN2 | BN3 | CN29 |
| BC30 | AN2 | BN3 | CN30 |
| BC31 | AN2 | BN3 | CN31 |
| BC32 | AN2 | BN3 | CN32 |
| BC33 | AN2 | BN3 | CN33 |
| BC34 | AN2 | BN3 | CN34 |
| BC35 | AN2 | BN3 | CN35 |
| BC36 | AN2 | BN3 | CN36 |
| BD1 | AN2 | BN4 | CN1 |
| BD2 | AN2 | BN4 | CN2 |
| BD3 | AN2 | BN4 | CN3 |
| BD4 | AN2 | BN4 | CN4 |
| BD5 | AN2 | BN4 | CN5 |
| BD6 | AN2 | BN4 | CN6 |
| BD7 | AN2 | BN4 | CN7 |
| BD8 | AN2 | BN4 | CN8 |
| BD9 | AN2 | BN4 | CN9 |
| BD10 | AN2 | BN4 | CN10 |
| BD11 | AN2 | BN4 | CN11 |
| BD12 | AN2 | BN4 | CN12 |
| BD13 | AN2 | BN4 | CN13 |
| BD14 | AN2 | BN4 | CN14 |
| BD15 | AN2 | BN4 | CN15 |
| BD16 | AN2 | BN4 | CN16 |
| BD17 | AN2 | BN4 | CN17 |
| BD18 | AN2 | BN4 | CN18 |
| BD19 | AN2 | BN4 | CN19 |
| BD20 | AN2 | BN4 | CN20 |
| BD21 | AN2 | BN4 | CN21 |
| BD22 | AN2 | BN4 | CN22 |
| BD23 | AN2 | BN4 | CN23 |
| BD24 | AN2 | BN4 | CN24 |
| BD25 | AN2 | BN4 | CN25 |
| BD26 | AN2 | BN4 | CN26 |
| BD27 | AN2 | BN4 | CN27 |
| BD28 | AN2 | BN4 | CN28 |
| BD29 | AN2 | BN4 | CN29 |
| BD30 | AN2 | BN4 | CN30 |
| BD31 | AN2 | BN4 | CN31 |
| BD32 | AN2 | BN4 | CN32 |
| BD33 | AN2 | BN4 | CN33 |
| BD34 | AN2 | BN4 | CN34 |
| BD35 | AN2 | BN4 | CN35 |
| BD36 | AN2 | BN4 | CN36 |
| BE1 | AN2 | BN5 | CN1 |
| BE2 | AN2 | BN5 | CN2 |
| BE3 | AN2 | BN5 | CN3 |
| BE4 | AN2 | BN5 | CN4 |
| BE5 | AN2 | BN5 | CN5 |
| BE6 | AN2 | BN5 | CN6 |
| BE7 | AN2 | BN5 | CN7 |
| BE8 | AN2 | BN5 | CN8 |
| BE9 | AN2 | BN5 | CN9 |
| BE10 | AN2 | BN5 | CN10 |
| BE11 | AN2 | BN5 | CN11 |
| BE12 | AN2 | BN5 | CN12 |
| BE13 | AN2 | BN5 | CN13 |
| BE14 | AN2 | BN5 | CN14 |
| BE15 | AN2 | BN5 | CN15 |
| BE16 | AN2 | BN5 | CN16 |
| BE17 | AN2 | BN5 | CN17 |
| BE18 | AN2 | BN5 | CN18 |
| BE19 | AN2 | BN5 | CN19 |
| BE20 | AN2 | BN5 | CN20 |
| BE21 | AN2 | BN5 | CN21 |
| BE22 | AN2 | BN5 | CN22 |
| BE23 | AN2 | BN5 | CN23 |
| BE24 | AN2 | BN5 | CN24 |
| BE25 | AN2 | BN5 | CN25 |
| BE26 | AN2 | BN5 | CN26 |
| BE27 | AN2 | BN5 | CN27 |
| BE28 | AN2 | BN5 | CN28 |
| BE29 | AN2 | BN5 | CN29 |
| BE30 | AN2 | BN5 | CN30 |
| BE31 | AN2 | BN5 | CN31 |
| BE32 | AN2 | BN5 | CN32 |
| BE33 | AN2 | BN5 | CN33 |
| BE34 | AN2 | BN5 | CN34 |
| BE35 | AN2 | BN5 | CN35 |
| BE36 | AN2 | BN5 | CN36 |
| BF1 | AN2 | BN6 | CN1 |
| BF2 | AN2 | BN6 | CN2 |
| BF3 | AN2 | BN6 | CN3 |
| BF4 | AN2 | BN6 | CN4 |
| BF5 | AN2 | BN6 | CN5 |
| BF6 | AN2 | BN6 | CN6 |
| BF7 | AN2 | BN6 | CN7 |
| BF8 | AN2 | BN6 | CN8 |
| BF9 | AN2 | BN6 | CN9 |
| BF10 | AN2 | BN6 | CN10 |
| BF11 | AN2 | BN6 | CN11 |
| BF12 | AN2 | BN6 | CN12 |
| BF13 | AN2 | BN6 | CN13 |
| BF14 | AN2 | BN6 | CN14 |
| BF15 | AN2 | BN6 | CN15 |
| BF16 | AN2 | BN6 | CN16 |
| BF17 | AN2 | BN6 | CN17 |
| BF18 | AN2 | BN6 | CN18 |
| BF19 | AN2 | BN6 | CN19 |
| BF20 | AN2 | BN6 | CN20 |
| BF21 | AN2 | BN6 | CN21 |
| BF22 | AN2 | BN6 | CN22 |
| BF23 | AN2 | BN6 | CN23 |
| BF24 | AN2 | BN6 | CN24 |
| BF25 | AN2 | BN6 | CN25 |
| BF26 | AN2 | BN6 | CN26 |
| BF27 | AN2 | BN6 | CN27 |
| BF28 | AN2 | BN6 | CN28 |
| BF29 | AN2 | BN6 | CN29 |
| BF30 | AN2 | BN6 | CN30 |
| BF31 | AN2 | BN6 | CN31 |
| BF32 | AN2 | BN6 | CN32 |
| BF33 | AN2 | BN6 | CN33 |
| BF34 | AN2 | BN6 | CN34 |
| BF35 | AN2 | BN6 | CN35 |
| BF36 | AN2 | BN6 | CN36 |
| BG1 | AN2 | BN7 | CN1 |
| BG2 | AN2 | BN7 | CN2 |
| BG3 | AN2 | BN7 | CN3 |
| BG4 | AN2 | BN7 | CN4 |
| BG5 | AN2 | BN7 | CN5 |
| BG6 | AN2 | BN7 | CN6 |
| BG7 | AN2 | BN7 | CN7 |
| BG8 | AN2 | BN7 | CN8 |
| BG9 | AN2 | BN7 | CN9 |
| BG10 | AN2 | BN7 | CN10 |
| BG11 | AN2 | BN7 | CN11 |
| BG12 | AN2 | BN7 | CN12 |
| BG13 | AN2 | BN7 | CN13 |
| BG14 | AN2 | BN7 | CN14 |
| BG15 | AN2 | BN7 | CN15 |
| BG16 | AN2 | BN7 | CN16 |
| BG17 | AN2 | BN7 | CN17 |
| BG18 | AN2 | BN7 | CN18 |
| BG19 | AN2 | BN7 | CN19 |
| BG20 | AN2 | BN7 | CN20 |
| BG21 | AN2 | BN7 | CN21 |
| BG22 | AN2 | BN7 | CN22 |
| BG23 | AN2 | BN7 | CN23 |
| BG24 | AN2 | BN7 | CN24 |
| BG25 | AN2 | BN7 | CN25 |
| BG26 | AN2 | BN7 | CN26 |
| BG27 | AN2 | BN7 | CN27 |
| BG28 | AN2 | BN7 | CN28 |
| BG29 | AN2 | BN7 | CN29 |
| BG30 | AN2 | BN7 | CN30 |
| BG31 | AN2 | BN7 | CN31 |
| BG32 | AN2 | BN7 | CN32 |
| BG33 | AN2 | BN7 | CN33 |
| BG34 | AN2 | BN7 | CN34 |
| BG35 | AN2 | BN7 | CN35 |
| BG36 | AN2 | BN7 | CN36 |
| BH1 | AN2 | BN8 | CN1 |
| BH2 | AN2 | BN8 | CN2 |
| BH3 | AN2 | BN8 | CN3 |
| BH4 | AN2 | BN8 | CN4 |
| BH5 | AN2 | BN8 | CN5 |
| BH6 | AN2 | BN8 | CN6 |
| BH7 | AN2 | BN8 | CN7 |
| BH8 | AN2 | BN8 | CN8 |
| BH9 | AN2 | BN8 | CN9 |
| BH10 | AN2 | BN8 | CN10 |
| BH11 | AN2 | BN8 | CN11 |
| BH12 | AN2 | BN8 | CN12 |
| BH13 | AN2 | BN8 | CN13 |
| BH14 | AN2 | BN8 | CN14 |
| BH15 | AN2 | BN8 | CN15 |
| BH16 | AN2 | BN8 | CN16 |
| BH17 | AN2 | BN8 | CN17 |
| BH18 | AN2 | BN8 | CN18 |
| BH19 | AN2 | BN8 | CN19 |
| BH20 | AN2 | BN8 | CN20 |
| BH21 | AN2 | BN8 | CN21 |
| BH22 | AN2 | BN8 | CN22 |
| BH23 | AN2 | BN8 | CN23 |
| BH24 | AN2 | BN8 | CN24 |
| BH25 | AN2 | BN8 | CN25 |
| BH26 | AN2 | BN8 | CN26 |
| BH27 | AN2 | BN8 | CN27 |
| BH28 | AN2 | BN8 | CN28 |
| BH29 | AN2 | BN8 | CN29 |
| BH30 | AN2 | BN8 | CN30 |
| BH31 | AN2 | BN8 | CN31 |
| BH32 | AN2 | BN8 | CN32 |
| BH33 | AN2 | BN8 | CN33 |
| BH34 | AN2 | BN8 | CN34 |
| BH35 | AN2 | BN8 | CN35 |
| BH36 | AN2 | BN8 | CN36 |
| BI1 | AN2 | BN9 | CN1 |
| BI2 | AN2 | BN9 | CN2 |
| BI3 | AN2 | BN9 | CN3 |
| BI4 | AN2 | BN9 | CN4 |
| BI5 | AN2 | BN9 | CN5 |
| BI6 | AN2 | BN9 | CN6 |
| BI7 | AN2 | BN9 | CN7 |
| BI8 | AN2 | BN9 | CN8 |
| BI9 | AN2 | BN9 | CN9 |
| BI10 | AN2 | BN9 | CN10 |
| BI11 | AN2 | BN9 | CN11 |
| BI12 | AN2 | BN9 | CN12 |
| BI13 | AN2 | BN9 | CN13 |
| BI14 | AN2 | BN9 | CN14 |
| BI15 | AN2 | BN9 | CN15 |
| BI16 | AN2 | BN9 | CN16 |
| BI17 | AN2 | BN9 | CN17 |
| BI18 | AN2 | BN9 | CN18 |
| BI19 | AN2 | BN9 | CN19 |
| BI20 | AN2 | BN9 | CN20 |
| BI21 | AN2 | BN9 | CN21 |
| BI22 | AN2 | BN9 | CN22 |
| BI23 | AN2 | BN9 | CN23 |
| BI24 | AN2 | BN9 | CN24 |
| BI25 | AN2 | BN9 | CN25 |
| BI26 | AN2 | BN9 | CN26 |
| BI27 | AN2 | BN9 | CN27 |
| BI28 | AN2 | BN9 | CN28 |
| BI29 | AN2 | BN9 | CN29 |
| BI30 | AN2 | BN9 | CN30 |
| BI31 | AN2 | BN9 | CN31 |
| BI32 | AN2 | BN9 | CN32 |
| BI33 | AN2 | BN9 | CN33 |
| BI34 | AN2 | BN9 | CN34 |
| BI35 | AN2 | BN9 | CN35 |
| BI36 | AN2 | BN9 | CN36 |
| BJ1 | AN2 | BN10 | CN1 |
| BJ2 | AN2 | BN10 | CN2 |
| BJ3 | AN2 | BN10 | CN3 |
| BJ4 | AN2 | BN10 | CN4 |
| BJ5 | AN2 | BN10 | CN5 |
| BJ6 | AN2 | BN10 | CN6 |
| BJ7 | AN2 | BN10 | CN7 |
| BJ8 | AN2 | BN10 | CN8 |
| BJ9 | AN2 | BN10 | CN9 |
| BJ10 | AN2 | BN10 | CN10 |
| BJ11 | AN2 | BN10 | CN11 |
| BJ12 | AN2 | BN10 | CN12 |
| BJ13 | AN2 | BN10 | CN13 |
| BJ14 | AN2 | BN10 | CN14 |
| BJ15 | AN2 | BN10 | CN15 |
| BJ16 | AN2 | BN10 | CN16 |
| BJ17 | AN2 | BN10 | CN17 |
| BJ18 | AN2 | BN10 | CN18 |
| BJ19 | AN2 | BN10 | CN19 |
| BJ20 | AN2 | BN10 | CN20 |
| BJ21 | AN2 | BN10 | CN21 |
| BJ22 | AN2 | BN10 | CN22 |
| BJ23 | AN2 | BN10 | CN23 |
| BJ24 | AN2 | BN10 | CN24 |
| BJ25 | AN2 | BN10 | CN25 |
| BJ26 | AN2 | BN10 | CN26 |
| BJ27 | AN2 | BN10 | CN27 |
| BJ28 | AN2 | BN10 | CN28 |
| BJ29 | AN2 | BN10 | CN29 |
| BJ30 | AN2 | BN10 | CN30 |
| BJ31 | AN2 | BN10 | CN31 |
| BJ32 | AN2 | BN10 | CN32 |
| BJ33 | AN2 | BN10 | CN33 |
| BJ34 | AN2 | BN10 | CN34 |
| BJ35 | AN2 | BN10 | CN35 |
| BJ36 | AN2 | BN10 | CN36 |
| BK1 | AN2 | BN11 | CN1 |
| BK2 | AN2 | BN11 | CN2 |
| BK3 | AN2 | BN11 | CN3 |
| BK4 | AN2 | BN11 | CN4 |
| BK5 | AN2 | BN11 | CN5 |
| BK6 | AN2 | BN11 | CN6 |
| BK7 | AN2 | BN11 | CN7 |
| BK8 | AN2 | BN11 | CN8 |
| BK9 | AN2 | BN11 | CN9 |
| BK10 | AN2 | BN11 | CN10 |
| BK11 | AN2 | BN11 | CN11 |
| BK12 | AN2 | BN11 | CN12 |
| BK13 | AN2 | BN11 | CN13 |
| BK14 | AN2 | BN11 | CN14 |
| BK15 | AN2 | BN11 | CN15 |
| BK16 | AN2 | BN11 | CN16 |
| BK17 | AN2 | BN11 | CN17 |
| BK18 | AN2 | BN11 | CN18 |
| BK19 | AN2 | BN11 | CN19 |
| BK20 | AN2 | BN11 | CN20 |
| BK21 | AN2 | BN11 | CN21 |
| BK22 | AN2 | BN11 | CN22 |
| BK23 | AN2 | BN11 | CN23 |
| BK24 | AN2 | BN11 | CN24 |
| BK25 | AN2 | BN11 | CN25 |
| BK26 | AN2 | BN11 | CN26 |
| BK27 | AN2 | BN11 | CN27 |
| BK28 | AN2 | BN11 | CN28 |
| BK29 | AN2 | BN11 | CN29 |
| BK30 | AN2 | BN11 | CN30 |
| BK31 | AN2 | BN11 | CN31 |
| BK32 | AN2 | BN11 | CN32 |
| BK33 | AN2 | BN11 | CN33 |
| BK34 | AN2 | BN11 | CN34 |
| BK35 | AN2 | BN11 | CN35 |
| BK36 | AN2 | BN11 | CN36 |
| BL1 | AN2 | BN12 | CN1 |
| BL2 | AN2 | BN12 | CN2 |
| BL3 | AN2 | BN12 | CN3 |
| BL4 | AN2 | BN12 | CN4 |
| BL5 | AN2 | BN12 | CN5 |
| BL6 | AN2 | BN12 | CN6 |
| BL7 | AN2 | BN12 | CN7 |
| BL8 | AN2 | BN12 | CN8 |
| BL9 | AN2 | BN12 | CN9 |
| BL10 | AN2 | BN12 | CN10 |
| BL11 | AN2 | BN12 | CN11 |
| BL12 | AN2 | BN12 | CN12 |
| BL13 | AN2 | BN12 | CN13 |
| BL14 | AN2 | BN12 | CN14 |
| BL15 | AN2 | BN12 | CN15 |
| BL16 | AN2 | BN12 | CN16 |
| BL17 | AN2 | BN12 | CN17 |
| BL18 | AN2 | BN12 | CN18 |
| BL19 | AN2 | BN12 | CN19 |
| BL20 | AN2 | BN12 | CN20 |
| BL21 | AN2 | BN12 | CN21 |
| BL22 | AN2 | BN12 | CN22 |
| BL23 | AN2 | BN12 | CN23 |
| BL24 | AN2 | BN12 | CN24 |
| BL25 | AN2 | BN12 | CN25 |
| BL26 | AN2 | BN12 | CN26 |
| BL27 | AN2 | BN12 | CN27 |
| BL28 | AN2 | BN12 | CN28 |
| BL29 | AN2 | BN12 | CN29 |
| BL30 | AN2 | BN12 | CN30 |
| BL31 | AN2 | BN12 | CN31 |
| BL32 | AN2 | BN12 | CN32 |
| BL33 | AN2 | BN12 | CN33 |
| BL34 | AN2 | BN12 | CN34 |
| BL35 | AN2 | BN12 | CN35 |
| BL36 | AN2 | BN12 | CN36 |
| BM1 | AN2 | BN13 | CN1 |
| BM2 | AN2 | BN13 | CN2 |
| BM3 | AN2 | BN13 | CN3 |
| BM4 | AN2 | BN13 | CN4 |
| BM5 | AN2 | BN13 | CN5 |
| BM6 | AN2 | BN13 | CN6 |
| BM7 | AN2 | BN13 | CN7 |
| BM8 | AN2 | BN13 | CN8 |
| BM9 | AN2 | BN13 | CN9 |
| BM10 | AN2 | BN13 | CN10 |
| BM11 | AN2 | BN13 | CN11 |
| BM12 | AN2 | BN13 | CN12 |
| BM13 | AN2 | BN13 | CN13 |
| BM14 | AN2 | BN13 | CN14 |
| BM15 | AN2 | BN13 | CN15 |
| BM16 | AN2 | BN13 | CN16 |
| BM17 | AN2 | BN13 | CN17 |
| BM18 | AN2 | BN13 | CN18 |
| BM19 | AN2 | BN13 | CN19 |
| BM20 | AN2 | BN13 | CN20 |
| BM21 | AN2 | BN13 | CN21 |
| BM22 | AN2 | BN13 | CN22 |
| BM23 | AN2 | BN13 | CN23 |
| BM24 | AN2 | BN13 | CN24 |
| BM25 | AN2 | BN13 | CN25 |
| BM26 | AN2 | BN13 | CN26 |
| BM27 | AN2 | BN13 | CN27 |
| BM28 | AN2 | BN13 | CN28 |
| BM29 | AN2 | BN13 | CN29 |
| BM30 | AN2 | BN13 | CN30 |
| BM31 | AN2 | BN13 | CN31 |
| BM32 | AN2 | BN13 | CN32 |
| BM33 | AN2 | BN13 | CN33 |
| BM34 | AN2 | BN13 | CN34 |
| BM35 | AN2 | BN13 | CN35 |
| BM36 | AN2 | BN13 | CN36 |
| BO1 | AN2 | BN14 | CN1 |
| BO2 | AN2 | BN14 | CN2 |
| BO3 | AN2 | BN14 | CN3 |
| BO4 | AN2 | BN14 | CN4 |
| BO5 | AN2 | BN14 | CN5 |
| BO6 | AN2 | BN14 | CN6 |
| BO7 | AN2 | BN14 | CN7 |
| BO8 | AN2 | BN14 | CN8 |
| BO9 | AN2 | BN14 | CN9 |
| BO10 | AN2 | BN14 | CN10 |
| BO11 | AN2 | BN14 | CN11 |
| BO12 | AN2 | BN14 | CN12 |
| BO13 | AN2 | BN14 | CN13 |
| BO14 | AN2 | BN14 | CN14 |
| BO15 | AN2 | BN14 | CN15 |
| BO16 | AN2 | BN14 | CN16 |
| BO17 | AN2 | BN14 | CN17 |
| BO18 | AN2 | BN14 | CN18 |
| BO19 | AN2 | BN14 | CN19 |
| BO20 | AN2 | BN14 | CN20 |
| BO21 | AN2 | BN14 | CN21 |
| BO22 | AN2 | BN14 | CN22 |
| BO23 | AN2 | BN14 | CN23 |
| BO24 | AN2 | BN14 | CN24 |
| BO25 | AN2 | BN14 | CN25 |
| BO26 | AN2 | BN14 | CN26 |
| BO27 | AN2 | BN14 | CN27 |
| BO28 | AN2 | BN14 | CN28 |
| BO29 | AN2 | BN14 | CN29 |
| BO30 | AN2 | BN14 | CN30 |
| BO31 | AN2 | BN14 | CN31 |
| BO32 | AN2 | BN14 | CN32 |
| BO33 | AN2 | BN14 | CN33 |
| BO34 | AN2 | BN14 | CN34 |
| BO35 | AN2 | BN14 | CN35 |
| BO36 | AN2 | BN14 | CN36 |
| BP1 | AN2 | BN15 | CN1 |
| BP2 | AN2 | BN15 | CN2 |
| BP3 | AN2 | BN15 | CN3 |
| BP4 | AN2 | BN15 | CN4 |
| BP5 | AN2 | BN15 | CN5 |
| BP6 | AN2 | BN15 | CN6 |
| BP7 | AN2 | BN15 | CN7 |
| BP8 | AN2 | BN15 | CN8 |
| BP9 | AN2 | BN15 | CN9 |
| BP10 | AN2 | BN15 | CN10 |
| BP11 | AN2 | BN15 | CN11 |
| BP12 | AN2 | BN15 | CN12 |
| BP13 | AN2 | BN15 | CN13 |
| BP14 | AN2 | BN15 | CN14 |
| BP15 | AN2 | BN15 | CN15 |
| BP16 | AN2 | BN15 | CN16 |
| BP17 | AN2 | BN15 | CN17 |
| BP18 | AN2 | BN15 | CN18 |
| BP19 | AN2 | BN15 | CN19 |
| BP20 | AN2 | BN15 | CN20 |
| BP21 | AN2 | BN15 | CN21 |
| BP22 | AN2 | BN15 | CN22 |
| BP23 | AN2 | BN15 | CN23 |
| BP24 | AN2 | BN15 | CN24 |
| BP25 | AN2 | BN15 | CN25 |
| BP26 | AN2 | BN15 | CN26 |
| BP27 | AN2 | BN15 | CN27 |
| BP28 | AN2 | BN15 | CN28 |
| BP29 | AN2 | BN15 | CN29 |
| BP30 | AN2 | BN15 | CN30 |
| BP31 | AN2 | BN15 | CN31 |
| BP32 | AN2 | BN15 | CN32 |
| BP33 | AN2 | BN15 | CN33 |
| BP34 | AN2 | BN15 | CN34 |
| BP35 | AN2 | BN15 | CN35 |
| BP36 | AN2 | BN15 | CN36 |
| BQ1 | AN2 | BN16 | CN1 |
| BQ2 | AN2 | BN16 | CN2 |
| BQ3 | AN2 | BN16 | CN3 |
| BQ4 | AN2 | BN16 | CN4 |
| BQ5 | AN2 | BN16 | CN5 |
| BQ6 | AN2 | BN16 | CN6 |
| BQ7 | AN2 | BN16 | CN7 |
| BQ8 | AN2 | BN16 | CN8 |
| BQ9 | AN2 | BN16 | CN9 |
| BQ10 | AN2 | BN16 | CN10 |
| BQ11 | AN2 | BN16 | CN11 |
| BQ12 | AN2 | BN16 | CN12 |
| BQ13 | AN2 | BN16 | CN13 |
| BQ14 | AN2 | BN16 | CN14 |
| BQ15 | AN2 | BN16 | CN15 |
| BQ16 | AN2 | BN16 | CN16 |
| BQ17 | AN2 | BN16 | CN17 |
| BQ18 | AN2 | BN16 | CN18 |
| BQ19 | AN2 | BN16 | CN19 |
| BQ20 | AN2 | BN16 | CN20 |
| BQ21 | AN2 | BN16 | CN21 |
| BQ22 | AN2 | BN16 | CN22 |
| BQ23 | AN2 | BN16 | CN23 |
| BQ24 | AN2 | BN16 | CN24 |
| BQ25 | AN2 | BN16 | CN25 |
| BQ26 | AN2 | BN16 | CN26 |
| BQ27 | AN2 | BN16 | CN27 |
| BQ28 | AN2 | BN16 | CN28 |
| BQ29 | AN2 | BN16 | CN29 |
| BQ30 | AN2 | BN16 | CN30 |
| BQ31 | AN2 | BN16 | CN31 |
| BQ32 | AN2 | BN16 | CN32 |
| BQ33 | AN2 | BN16 | CN33 |
| BQ34 | AN2 | BN16 | CN34 |
| BQ35 | AN2 | BN16 | CN35 |
| BQ36 | AN2 | BN16 | CN36 |
| BR1 | AN2 | BN17 | CN1 |
| BR2 | AN2 | BN17 | CN2 |
| BR3 | AN2 | BN17 | CN3 |
| BR4 | AN2 | BN17 | CN4 |
| BR5 | AN2 | BN17 | CN5 |
| BR6 | AN2 | BN17 | CN6 |
| BR7 | AN2 | BN17 | CN7 |
| BR8 | AN2 | BN17 | CN8 |
| BR9 | AN2 | BN17 | CN9 |
| BR10 | AN2 | BN17 | CN10 |
| BR11 | AN2 | BN17 | CN11 |
| BR12 | AN2 | BN17 | CN12 |
| BR13 | AN2 | BN17 | CN13 |
| BR14 | AN2 | BN17 | CN14 |
| BR15 | AN2 | BN17 | CN15 |
| BR16 | AN2 | BN17 | CN16 |
| BR17 | AN2 | BN17 | CN17 |
| BR18 | AN2 | BN17 | CN18 |
| BR19 | AN2 | BN17 | CN19 |
| BR20 | AN2 | BN17 | CN20 |
| BR21 | AN2 | BN17 | CN21 |
| BR22 | AN2 | BN17 | CN22 |
| BR23 | AN2 | BN17 | CN23 |
| BR24 | AN2 | BN17 | CN24 |
| BR25 | AN2 | BN17 | CN25 |
| BR26 | AN2 | BN17 | CN26 |
| BR27 | AN2 | BN17 | CN27 |
| BR28 | AN2 | BN17 | CN28 |
| BR29 | AN2 | BN17 | CN29 |
| BR30 | AN2 | BN17 | CN30 |
| BR31 | AN2 | BN17 | CN31 |
| BR32 | AN2 | BN17 | CN32 |
| BR33 | AN2 | BN17 | CN33 |
| BR34 | AN2 | BN17 | CN34 |
| BR35 | AN2 | BN17 | CN35 |
| BR36 | AN2 | BN17 | CN36 |
| BS1 | AN2 | BN18 | CN1 |
| BS2 | AN2 | BN18 | CN2 |
| BS3 | AN2 | BN18 | CN3 |
| BS4 | AN2 | BN18 | CN4 |
| BS5 | AN2 | BN18 | CN5 |
| BS6 | AN2 | BN18 | CN6 |
| BS7 | AN2 | BN18 | CN7 |
| BS8 | AN2 | BN18 | CN8 |
| BS9 | AN2 | BN18 | CN9 |
| BS10 | AN2 | BN18 | CN10 |
| BS11 | AN2 | BN18 | CN11 |
| BS12 | AN2 | BN18 | CN12 |
| BS13 | AN2 | BN18 | CN13 |
| BS14 | AN2 | BN18 | CN14 |
| BS15 | AN2 | BN18 | CN15 |
| BS16 | AN2 | BN18 | CN16 |
| BS17 | AN2 | BN18 | CN17 |
| BS18 | AN2 | BN18 | CN18 |
| BS19 | AN2 | BN18 | CN19 |
| BS20 | AN2 | BN18 | CN20 |
| BS21 | AN2 | BN18 | CN21 |
| BS22 | AN2 | BN18 | CN22 |
| BS23 | AN2 | BN18 | CN23 |
| BS24 | AN2 | BN18 | CN24 |
| BS25 | AN2 | BN18 | CN25 |
| BS26 | AN2 | BN18 | CN26 |
| BS27 | AN2 | BN18 | CN27 |
| BS28 | AN2 | BN18 | CN28 |
| BS29 | AN2 | BN18 | CN29 |
| BS30 | AN2 | BN18 | CN30 |
| BS31 | AN2 | BN18 | CN31 |
| BS32 | AN2 | BN18 | CN32 |
| BS33 | AN2 | BN18 | CN33 |
| BS34 | AN2 | BN18 | CN34 |
| BS35 | AN2 | BN18 | CN35 |
| BS36 | AN2 | BN18 | CN36 |
| BT1 | AN2 | BN19 | CN1 |
| BT2 | AN2 | BN19 | CN2 |
| BT3 | AN2 | BN19 | CN3 |
| BT4 | AN2 | BN19 | CN4 |
| BT5 | AN2 | BN19 | CN5 |
| BT6 | AN2 | BN19 | CN6 |
| BT7 | AN2 | BN19 | CN7 |
| BT8 | AN2 | BN19 | CN8 |
| BT9 | AN2 | BN19 | CN9 |
| BT10 | AN2 | BN19 | CN10 |
| BT11 | AN2 | BN19 | CN11 |
| BT12 | AN2 | BN19 | CN12 |
| BT13 | AN2 | BN19 | CN13 |
| BT14 | AN2 | BN19 | CN14 |
| BT15 | AN2 | BN19 | CN15 |
| BT16 | AN2 | BN19 | CN16 |
| BT17 | AN2 | BN19 | CN17 |
| BT18 | AN2 | BN19 | CN18 |
| BT19 | AN2 | BN19 | CN19 |
| BT20 | AN2 | BN19 | CN20 |
| BT21 | AN2 | BN19 | CN21 |
| BT22 | AN2 | BN19 | CN22 |
| BT23 | AN2 | BN19 | CN23 |
| BT24 | AN2 | BN19 | CN24 |
| BT25 | AN2 | BN19 | CN25 |
| BT26 | AN2 | BN19 | CN26 |
| BT27 | AN2 | BN19 | CN27 |
| BT28 | AN2 | BN19 | CN28 |
| BT29 | AN2 | BN19 | CN29 |
| BT30 | AN2 | BN19 | CN30 |
| BT31 | AN2 | BN19 | CN31 |
| BT32 | AN2 | BN19 | CN32 |
| BT33 | AN2 | BN19 | CN33 |
| BT34 | AN2 | BN19 | CN34 |
| BT35 | AN2 | BN19 | CN35 |
| BT36 | AN2 | BN19 | CN36 |
| BU1 | AN2 | BN20 | CN1 |
| BU2 | AN2 | BN20 | CN2 |
| BU3 | AN2 | BN20 | CN3 |
| BU4 | AN2 | BN20 | CN4 |
| BU5 | AN2 | BN20 | CN5 |
| BU6 | AN2 | BN20 | CN6 |
| BU7 | AN2 | BN20 | CN7 |
| BU8 | AN2 | BN20 | CN8 |
| BU9 | AN2 | BN20 | CN9 |
| BU10 | AN2 | BN20 | CN10 |
| BU11 | AN2 | BN20 | CN11 |
| BU12 | AN2 | BN20 | CN12 |
| BU13 | AN2 | BN20 | CN13 |
| BU14 | AN2 | BN20 | CN14 |
| BU15 | AN2 | BN20 | CN15 |
| BU16 | AN2 | BN20 | CN16 |
| BU17 | AN2 | BN20 | CN17 |
| BU18 | AN2 | BN20 | CN18 |
| BU19 | AN2 | BN20 | CN19 |
| BU20 | AN2 | BN20 | CN20 |
| BU21 | AN2 | BN20 | CN21 |
| BU22 | AN2 | BN20 | CN22 |
| BU23 | AN2 | BN20 | CN23 |
| BU24 | AN2 | BN20 | CN24 |
| BU25 | AN2 | BN20 | CN25 |
| BU26 | AN2 | BN20 | CN26 |
| BU27 | AN2 | BN20 | CN27 |
| BU28 | AN2 | BN20 | CN28 |
| BU29 | AN2 | BN20 | CN29 |
| BU30 | AN2 | BN20 | CN30 |
| BU31 | AN2 | BN20 | CN31 |
| BU32 | AN2 | BN20 | CN32 |
| BU33 | AN2 | BN20 | CN33 |
| BU34 | AN2 | BN20 | CN34 |
| BU35 | AN2 | BN20 | CN35 |
| BU36 | AN2 | BN20 | CN36 |
| BV1 | AN2 | BN21 | CN1 |
| BV2 | AN2 | BN21 | CN2 |
| BV3 | AN2 | BN21 | CN3 |
| BV4 | AN2 | BN21 | CN4 |
| BV5 | AN2 | BN21 | CN5 |
| BV6 | AN2 | BN21 | CN6 |
| BV7 | AN2 | BN21 | CN7 |
| BV8 | AN2 | BN21 | CN8 |
| BV9 | AN2 | BN21 | CN9 |
| BV10 | AN2 | BN21 | CN10 |
| BV11 | AN2 | BN21 | CN11 |
| BV12 | AN2 | BN21 | CN12 |
| BV13 | AN2 | BN21 | CN13 |
| BV14 | AN2 | BN21 | CN14 |
| BV15 | AN2 | BN21 | CN15 |
| BV16 | AN2 | BN21 | CN16 |
| BV17 | AN2 | BN21 | CN17 |
| BV18 | AN2 | BN21 | CN18 |
| BV19 | AN2 | BN21 | CN19 |
| BV20 | AN2 | BN21 | CN20 |
| BV21 | AN2 | BN21 | CN21 |
| BV22 | AN2 | BN21 | CN22 |
| BV23 | AN2 | BN21 | CN23 |
| BV24 | AN2 | BN21 | CN24 |
| BV25 | AN2 | BN21 | CN25 |
| BV26 | AN2 | BN21 | CN26 |
| BV27 | AN2 | BN21 | CN27 |
| BV28 | AN2 | BN21 | CN28 |
| BV29 | AN2 | BN21 | CN29 |
| BV30 | AN2 | BN21 | CN30 |
| BV31 | AN2 | BN21 | CN31 |
| BV32 | AN2 | BN21 | CN32 |
| BV33 | AN2 | BN21 | CN33 |
| BV34 | AN2 | BN21 | CN34 |
| BV35 | AN2 | BN21 | CN35 |
| BV36 | AN2 | BN21 | CN36 |
| BW1 | AN2 | BN22 | CN1 |
| BW2 | AN2 | BN22 | CN2 |
| BW3 | AN2 | BN22 | CN3 |
| BW4 | AN2 | BN22 | CN4 |
| BW5 | AN2 | BN22 | CN5 |
| BW6 | AN2 | BN22 | CN6 |
| BW7 | AN2 | BN22 | CN7 |
| BW8 | AN2 | BN22 | CN8 |
| BW9 | AN2 | BN22 | CN9 |
| BW10 | AN2 | BN22 | CN10 |
| BW11 | AN2 | BN22 | CN11 |
| BW12 | AN2 | BN22 | CN12 |
| BW13 | AN2 | BN22 | CN13 |
| BW14 | AN2 | BN22 | CN14 |
| BW15 | AN2 | BN22 | CN15 |
| BW16 | AN2 | BN22 | CN16 |
| BW17 | AN2 | BN22 | CN17 |
| BW18 | AN2 | BN22 | CN18 |
| BW19 | AN2 | BN22 | CN19 |
| BW20 | AN2 | BN22 | CN20 |
| BW21 | AN2 | BN22 | CN21 |
| BW22 | AN2 | BN22 | CN22 |
| BW23 | AN2 | BN22 | CN23 |
| BW24 | AN2 | BN22 | CN24 |
| BW25 | AN2 | BN22 | CN25 |
| BW26 | AN2 | BN22 | CN26 |
| BW27 | AN2 | BN22 | CN27 |
| BW28 | AN2 | BN22 | CN28 |
| BW29 | AN2 | BN22 | CN29 |
| BW30 | AN2 | BN22 | CN30 |
| BW31 | AN2 | BN22 | CN31 |
| BW32 | AN2 | BN22 | CN32 |
| BW33 | AN2 | BN22 | CN33 |
| BW34 | AN2 | BN22 | CN34 |
| BW35 | AN2 | BN22 | CN35 |
| BW36 | AN2 | BN22 | CN36 |
| CA1 | AN3 | BN1 | CN1 |
| CA2 | AN3 | BN1 | CN2 |
| CA3 | AN3 | BN1 | CN3 |
| CA4 | AN3 | BN1 | CN4 |
| CA5 | AN3 | BN1 | CN5 |
| CA6 | AN3 | BN1 | CN6 |
| CA7 | AN3 | BN1 | CN7 |
| CA8 | AN3 | BN1 | CN8 |
| CA9 | AN3 | BN1 | CN9 |
| CA10 | AN3 | BN1 | CN10 |
| CA11 | AN3 | BN1 | CN11 |
| CA12 | AN3 | BN1 | CN12 |
| CA13 | AN3 | BN1 | CN13 |
| CA14 | AN3 | BN1 | CN14 |
| CA15 | AN3 | BN1 | CN15 |
| CA16 | AN3 | BN1 | CN16 |
| CA17 | AN3 | BN1 | CN17 |
| CA18 | AN3 | BN1 | CN18 |
| CA19 | AN3 | BN1 | CN19 |
| CA20 | AN3 | BN1 | CN20 |
| CA21 | AN3 | BN1 | CN21 |
| CA22 | AN3 | BN1 | CN22 |
| CA23 | AN3 | BN1 | CN23 |
| CA24 | AN3 | BN1 | CN24 |
| CA25 | AN3 | BN1 | CN25 |
| CA26 | AN3 | BN1 | CN26 |
| CA27 | AN3 | BN1 | CN27 |
| CA28 | AN3 | BN1 | CN28 |
| CA29 | AN3 | BN1 | CN29 |
| CA30 | AN3 | BN1 | CN30 |
| CA31 | AN3 | BN1 | CN31 |
| CA32 | AN3 | BN1 | CN32 |
| CA33 | AN3 | BN1 | CN33 |
| CA34 | AN3 | BN1 | CN34 |
| CA35 | AN3 | BN1 | CN35 |
| CA36 | AN3 | BN1 | CN36 |
| CB1 | AN3 | BN2 | CN1 |
| CB2 | AN3 | BN2 | CN2 |
| CB3 | AN3 | BN2 | CN3 |
| CB4 | AN3 | BN2 | CN4 |
| CB5 | AN3 | BN2 | CN5 |
| CB6 | AN3 | BN2 | CN6 |
| CB7 | AN3 | BN2 | CN7 |
| CB8 | AN3 | BN2 | CN8 |
| CB9 | AN3 | BN2 | CN9 |
| CB10 | AN3 | BN2 | CN10 |
| CB11 | AN3 | BN2 | CN11 |
| CB12 | AN3 | BN2 | CN12 |
| CB13 | AN3 | BN2 | CN13 |
| CB14 | AN3 | BN2 | CN14 |
| CB15 | AN3 | BN2 | CN15 |
| CB16 | AN3 | BN2 | CN16 |
| CB17 | AN3 | BN2 | CN17 |
| CB18 | AN3 | BN2 | CN18 |
| CB19 | AN3 | BN2 | CN19 |
| CB20 | AN3 | BN2 | CN20 |
| CB21 | AN3 | BN2 | CN21 |
| CB22 | AN3 | BN2 | CN22 |
| CB23 | AN3 | BN2 | CN23 |
| CB24 | AN3 | BN2 | CN24 |
| CB25 | AN3 | BN2 | CN25 |
| CB26 | AN3 | BN2 | CN26 |
| CB27 | AN3 | BN2 | CN27 |
| CB28 | AN3 | BN2 | CN28 |
| CB29 | AN3 | BN2 | CN29 |
| CB30 | AN3 | BN2 | CN30 |
| CB31 | AN3 | BN2 | CN31 |
| CB32 | AN3 | BN2 | CN32 |
| CB33 | AN3 | BN2 | CN33 |
| CB34 | AN3 | BN2 | CN34 |
| CB35 | AN3 | BN2 | CN35 |
| CB36 | AN3 | BN2 | CN36 |
| CC1 | AN3 | BN3 | CN1 |
| CC2 | AN3 | BN3 | CN2 |
| CC3 | AN3 | BN3 | CN3 |
| CC4 | AN3 | BN3 | CN4 |
| CC5 | AN3 | BN3 | CN5 |
| CC6 | AN3 | BN3 | CN6 |
| CC7 | AN3 | BN3 | CN7 |
| CC8 | AN3 | BN3 | CN8 |
| CC9 | AN3 | BN3 | CN9 |
| CC10 | AN3 | BN3 | CN10 |
| CC11 | AN3 | BN3 | CN11 |
| CC12 | AN3 | BN3 | CN12 |
| CC13 | AN3 | BN3 | CN13 |
| CC14 | AN3 | BN3 | CN14 |
| CC15 | AN3 | BN3 | CN15 |
| CC16 | AN3 | BN3 | CN16 |
| CC17 | AN3 | BN3 | CN17 |
| CC18 | AN3 | BN3 | CN18 |
| CC19 | AN3 | BN3 | CN19 |
| CC20 | AN3 | BN3 | CN20 |
| CC21 | AN3 | BN3 | CN21 |
| CC22 | AN3 | BN3 | CN22 |
| CC23 | AN3 | BN3 | CN23 |
| CC24 | AN3 | BN3 | CN24 |
| CC25 | AN3 | BN3 | CN25 |
| CC26 | AN3 | BN3 | CN26 |
| CC27 | AN3 | BN3 | CN27 |
| CC28 | AN3 | BN3 | CN28 |
| CC29 | AN3 | BN3 | CN29 |
| CC30 | AN3 | BN3 | CN30 |
| CC31 | AN3 | BN3 | CN31 |
| CC32 | AN3 | BN3 | CN32 |
| CC33 | AN3 | BN3 | CN33 |
| CC34 | AN3 | BN3 | CN34 |
| CC35 | AN3 | BN3 | CN35 |
| CC36 | AN3 | BN3 | CN36 |
| CD1 | AN3 | BN4 | CN1 |
| CD2 | AN3 | BN4 | CN2 |
| CD3 | AN3 | BN4 | CN3 |
| CD4 | AN3 | BN4 | CN4 |
| CD5 | AN3 | BN4 | CN5 |
| CD6 | AN3 | BN4 | CN6 |
| CD7 | AN3 | BN4 | CN7 |
| CD8 | AN3 | BN4 | CN8 |
| CD9 | AN3 | BN4 | CN9 |
| CD10 | AN3 | BN4 | CN10 |
| CD11 | AN3 | BN4 | CN11 |
| CD12 | AN3 | BN4 | CN12 |
| CD13 | AN3 | BN4 | CN13 |
| CD14 | AN3 | BN4 | CN14 |
| CD15 | AN3 | BN4 | CN15 |
| CD16 | AN3 | BN4 | CN16 |
| CD17 | AN3 | BN4 | CN17 |
| CD18 | AN3 | BN4 | CN18 |
| CD19 | AN3 | BN4 | CN19 |
| CD20 | AN3 | BN4 | CN20 |
| CD21 | AN3 | BN4 | CN21 |
| CD22 | AN3 | BN4 | CN22 |
| CD23 | AN3 | BN4 | CN23 |
| CD24 | AN3 | BN4 | CN24 |
| CD25 | AN3 | BN4 | CN25 |
| CD26 | AN3 | BN4 | CN26 |
| CD27 | AN3 | BN4 | CN27 |
| CD28 | AN3 | BN4 | CN28 |
| CD29 | AN3 | BN4 | CN29 |
| CD30 | AN3 | BN4 | CN30 |
| CD31 | AN3 | BN4 | CN31 |
| CD32 | AN3 | BN4 | CN32 |
| CD33 | AN3 | BN4 | CN33 |
| CD34 | AN3 | BN4 | CN34 |
| CD35 | AN3 | BN4 | CN35 |
| CD36 | AN3 | BN4 | CN36 |
| CE1 | AN3 | BN5 | CN1 |
| CE2 | AN3 | BN5 | CN2 |
| CE3 | AN3 | BN5 | CN3 |
| CE4 | AN3 | BN5 | CN4 |
| CE5 | AN3 | BN5 | CN5 |
| CE6 | AN3 | BN5 | CN6 |
| CE7 | AN3 | BN5 | CN7 |
| CE8 | AN3 | BN5 | CN8 |
| CE9 | AN3 | BN5 | CN9 |
| CE10 | AN3 | BN5 | CN10 |
| CE11 | AN3 | BN5 | CN11 |
| CE12 | AN3 | BN5 | CN12 |
| CE13 | AN3 | BN5 | CN13 |
| CE14 | AN3 | BN5 | CN14 |
| CE15 | AN3 | BN5 | CN15 |
| CE16 | AN3 | BN5 | CN16 |
| CE17 | AN3 | BN5 | CN17 |
| CE18 | AN3 | BN5 | CN18 |
| CE19 | AN3 | BN5 | CN19 |
| CE20 | AN3 | BN5 | CN20 |
| CE21 | AN3 | BN5 | CN21 |
| CE22 | AN3 | BN5 | CN22 |
| CE23 | AN3 | BN5 | CN23 |
| CE24 | AN3 | BN5 | CN24 |
| CE25 | AN3 | BN5 | CN25 |
| CE26 | AN3 | BN5 | CN26 |
| CE27 | AN3 | BN5 | CN27 |
| CE28 | AN3 | BN5 | CN28 |
| CE29 | AN3 | BN5 | CN29 |
| CE30 | AN3 | BN5 | CN30 |
| CE31 | AN3 | BN5 | CN31 |
| CE32 | AN3 | BN5 | CN32 |
| CE33 | AN3 | BN5 | CN33 |
| CE34 | AN3 | BN5 | CN34 |
| CE35 | AN3 | BN5 | CN35 |
| CE36 | AN3 | BN5 | CN36 |
| CF1 | AN3 | BN6 | CN1 |
| CF2 | AN3 | BN6 | CN2 |
| CF3 | AN3 | BN6 | CN3 |
| CF4 | AN3 | BN6 | CN4 |
| CF5 | AN3 | BN6 | CN5 |
| CF6 | AN3 | BN6 | CN6 |
| CF7 | AN3 | BN6 | CN7 |
| CF8 | AN3 | BN6 | CN8 |
| CF9 | AN3 | BN6 | CN9 |
| CF10 | AN3 | BN6 | CN10 |
| CF11 | AN3 | BN6 | CN11 |
| CF12 | AN3 | BN6 | CN12 |
| CF13 | AN3 | BN6 | CN13 |
| CF14 | AN3 | BN6 | CN14 |
| CF15 | AN3 | BN6 | CN15 |
| CF16 | AN3 | BN6 | CN16 |
| CF17 | AN3 | BN6 | CN17 |
| CF18 | AN3 | BN6 | CN18 |
| CF19 | AN3 | BN6 | CN19 |
| CF20 | AN3 | BN6 | CN20 |
| CF21 | AN3 | BN6 | CN21 |
| CF22 | AN3 | BN6 | CN22 |
| CF23 | AN3 | BN6 | CN23 |
| CF24 | AN3 | BN6 | CN24 |
| CF25 | AN3 | BN6 | CN25 |
| CF26 | AN3 | BN6 | CN26 |
| CF27 | AN3 | BN6 | CN27 |
| CF28 | AN3 | BN6 | CN28 |
| CF29 | AN3 | BN6 | CN29 |
| CF30 | AN3 | BN6 | CN30 |
| CF31 | AN3 | BN6 | CN31 |
| CF32 | AN3 | BN6 | CN32 |
| CF33 | AN3 | BN6 | CN33 |
| CF34 | AN3 | BN6 | CN34 |
| CF35 | AN3 | BN6 | CN35 |
| CF36 | AN3 | BN6 | CN36 |
| CG1 | AN3 | BN7 | CN1 |
| CG2 | AN3 | BN7 | CN2 |
| CG3 | AN3 | BN7 | CN3 |
| CG4 | AN3 | BN7 | CN4 |
| CG5 | AN3 | BN7 | CN5 |
| CG6 | AN3 | BN7 | CN6 |
| CG7 | AN3 | BN7 | CN7 |
| CG8 | AN3 | BN7 | CN8 |
| CG9 | AN3 | BN7 | CN9 |
| CG10 | AN3 | BN7 | CN10 |
| CG11 | AN3 | BN7 | CN11 |
| CG12 | AN3 | BN7 | CN12 |
| CG13 | AN3 | BN7 | CN13 |
| CG14 | AN3 | BN7 | CN14 |
| CG15 | AN3 | BN7 | CN15 |
| CG16 | AN3 | BN7 | CN16 |
| CG17 | AN3 | BN7 | CN17 |
| CG18 | AN3 | BN7 | CN18 |
| CG19 | AN3 | BN7 | CN19 |
| CG20 | AN3 | BN7 | CN20 |
| CG21 | AN3 | BN7 | CN21 |
| CG22 | AN3 | BN7 | CN22 |
| CG23 | AN3 | BN7 | CN23 |
| CG24 | AN3 | BN7 | CN24 |
| CG25 | AN3 | BN7 | CN25 |
| CG26 | AN3 | BN7 | CN26 |
| CG27 | AN3 | BN7 | CN27 |
| CG28 | AN3 | BN7 | CN28 |
| CG29 | AN3 | BN7 | CN29 |
| CG30 | AN3 | BN7 | CN30 |
| CG31 | AN3 | BN7 | CN31 |
| CG32 | AN3 | BN7 | CN32 |
| CG33 | AN3 | BN7 | CN33 |
| CG34 | AN3 | BN7 | CN34 |
| CG35 | AN3 | BN7 | CN35 |
| CG36 | AN3 | BN7 | CN36 |
| CH1 | AN3 | BN8 | CN1 |
| CH2 | AN3 | BN8 | CN2 |
| CH3 | AN3 | BN8 | CN3 |
| CH4 | AN3 | BN8 | CN4 |
| CH5 | AN3 | BN8 | CN5 |
| CH6 | AN3 | BN8 | CN6 |
| CH7 | AN3 | BN8 | CN7 |
| CH8 | AN3 | BN8 | CN8 |
| CH9 | AN3 | BN8 | CN9 |
| CH10 | AN3 | BN8 | CN10 |
| CH11 | AN3 | BN8 | CN11 |
| CH12 | AN3 | BN8 | CN12 |
| CH13 | AN3 | BN8 | CN13 |
| CH14 | AN3 | BN8 | CN14 |
| CH15 | AN3 | BN8 | CN15 |
| CH16 | AN3 | BN8 | CN16 |
| CH17 | AN3 | BN8 | CN17 |
| CH18 | AN3 | BN8 | CN18 |
| CH19 | AN3 | BN8 | CN19 |
| CH20 | AN3 | BN8 | CN20 |
| CH21 | AN3 | BN8 | CN21 |
| CH22 | AN3 | BN8 | CN22 |
| CH23 | AN3 | BN8 | CN23 |
| CH24 | AN3 | BN8 | CN24 |
| CH25 | AN3 | BN8 | CN25 |
| CH26 | AN3 | BN8 | CN26 |
| CH27 | AN3 | BN8 | CN27 |
| CH28 | AN3 | BN8 | CN28 |
| CH29 | AN3 | BN8 | CN29 |
| CH30 | AN3 | BN8 | CN30 |
| CH31 | AN3 | BN8 | CN31 |
| CH32 | AN3 | BN8 | CN32 |
| CH33 | AN3 | BN8 | CN33 |
| CH34 | AN3 | BN8 | CN34 |
| CH35 | AN3 | BN8 | CN35 |
| CH36 | AN3 | BN8 | CN36 |
| CI1 | AN3 | BN9 | CN1 |
| CI2 | AN3 | BN9 | CN2 |
| CI3 | AN3 | BN9 | CN3 |
| CI4 | AN3 | BN9 | CN4 |
| CI5 | AN3 | BN9 | CN5 |
| CI6 | AN3 | BN9 | CN6 |
| CI7 | AN3 | BN9 | CN7 |
| CI8 | AN3 | BN9 | CN8 |
| CI9 | AN3 | BN9 | CN9 |
| CI10 | AN3 | BN9 | CN10 |
| CI11 | AN3 | BN9 | CN11 |
| CI12 | AN3 | BN9 | CN12 |
| CI13 | AN3 | BN9 | CN13 |
| CI14 | AN3 | BN9 | CN14 |
| CI15 | AN3 | BN9 | CN15 |
| CI16 | AN3 | BN9 | CN16 |
| CI17 | AN3 | BN9 | CN17 |
| CI18 | AN3 | BN9 | CN18 |
| CI19 | AN3 | BN9 | CN19 |
| CI20 | AN3 | BN9 | CN20 |
| CI21 | AN3 | BN9 | CN21 |
| CI22 | AN3 | BN9 | CN22 |
| CI23 | AN3 | BN9 | CN23 |
| CI24 | AN3 | BN9 | CN24 |
| CI25 | AN3 | BN9 | CN25 |
| CI26 | AN3 | BN9 | CN26 |
| CI27 | AN3 | BN9 | CN27 |
| CI28 | AN3 | BN9 | CN28 |
| CI29 | AN3 | BN9 | CN29 |
| CI30 | AN3 | BN9 | CN30 |
| CI31 | AN3 | BN9 | CN31 |
| CI32 | AN3 | BN9 | CN32 |
| CI33 | AN3 | BN9 | CN33 |
| CI34 | AN3 | BN9 | CN34 |
| CI35 | AN3 | BN9 | CN35 |
| CI36 | AN3 | BN9 | CN36 |
| CJ1 | AN3 | BN10 | CN1 |
| CJ2 | AN3 | BN10 | CN2 |
| CJ3 | AN3 | BN10 | CN3 |
| CJ4 | AN3 | BN10 | CN4 |
| CJ5 | AN3 | BN10 | CN5 |
| CJ6 | AN3 | BN10 | CN6 |
| CJ7 | AN3 | BN10 | CN7 |
| CJ8 | AN3 | BN10 | CN8 |
| CJ9 | AN3 | BN10 | CN9 |
| CJ10 | AN3 | BN10 | CN10 |
| CJ11 | AN3 | BN10 | CN11 |
| CJ12 | AN3 | BN10 | CN12 |
| CJ13 | AN3 | BN10 | CN13 |
| CJ14 | AN3 | BN10 | CN14 |
| CJ15 | AN3 | BN10 | CN15 |
| CJ16 | AN3 | BN10 | CN16 |
| CJ17 | AN3 | BN10 | CN17 |
| CJ18 | AN3 | BN10 | CN18 |
| CJ19 | AN3 | BN10 | CN19 |
| CJ20 | AN3 | BN10 | CN20 |
| CJ21 | AN3 | BN10 | CN21 |
| CJ22 | AN3 | BN10 | CN22 |
| CJ23 | AN3 | BN10 | CN23 |
| CJ24 | AN3 | BN10 | CN24 |
| CJ25 | AN3 | BN10 | CN25 |
| CJ26 | AN3 | BN10 | CN26 |
| CJ27 | AN3 | BN10 | CN27 |
| CJ28 | AN3 | BN10 | CN28 |
| CJ29 | AN3 | BN10 | CN29 |
| CJ30 | AN3 | BN10 | CN30 |
| CJ31 | AN3 | BN10 | CN31 |
| CJ32 | AN3 | BN10 | CN32 |
| CJ33 | AN3 | BN10 | CN33 |
| CJ34 | AN3 | BN10 | CN34 |
| CJ35 | AN3 | BN10 | CN35 |
| CJ36 | AN3 | BN10 | CN36 |
| CK1 | AN3 | BN11 | CN1 |
| CK2 | AN3 | BN11 | CN2 |
| CK3 | AN3 | BN11 | CN3 |
| CK4 | AN3 | BN11 | CN4 |
| CK5 | AN3 | BN11 | CN5 |
| CK6 | AN3 | BN11 | CN6 |
| CK7 | AN3 | BN11 | CN7 |
| CK8 | AN3 | BN11 | CN8 |
| CK9 | AN3 | BN11 | CN9 |
| CK10 | AN3 | BN11 | CN10 |
| CK11 | AN3 | BN11 | CN11 |
| CK12 | AN3 | BN11 | CN12 |
| CK13 | AN3 | BN11 | CN13 |
| CK14 | AN3 | BN11 | CN14 |
| CK15 | AN3 | BN11 | CN15 |
| CK16 | AN3 | BN11 | CN16 |
| CK17 | AN3 | BN11 | CN17 |
| CK18 | AN3 | BN11 | CN18 |
| CK19 | AN3 | BN11 | CN19 |
| CK20 | AN3 | BN11 | CN20 |
| CK21 | AN3 | BN11 | CN21 |
| CK22 | AN3 | BN11 | CN22 |
| CK23 | AN3 | BN11 | CN23 |
| CK24 | AN3 | BN11 | CN24 |
| CK25 | AN3 | BN11 | CN25 |
| CK26 | AN3 | BN11 | CN26 |
| CK27 | AN3 | BN11 | CN27 |
| CK28 | AN3 | BN11 | CN28 |
| CK29 | AN3 | BN11 | CN29 |
| CK30 | AN3 | BN11 | CN30 |
| CK31 | AN3 | BN11 | CN31 |
| CK32 | AN3 | BN11 | CN32 |
| CK33 | AN3 | BN11 | CN33 |
| CK34 | AN3 | BN11 | CN34 |
| CK35 | AN3 | BN11 | CN35 |
| CK36 | AN3 | BN11 | CN36 |
| CL1 | AN3 | BN12 | CN1 |
| CL2 | AN3 | BN12 | CN2 |
| CL3 | AN3 | BN12 | CN3 |
| CL4 | AN3 | BN12 | CN4 |
| CL5 | AN3 | BN12 | CN5 |
| CL6 | AN3 | BN12 | CN6 |
| CL7 | AN3 | BN12 | CN7 |
| CL8 | AN3 | BN12 | CN8 |
| CL9 | AN3 | BN12 | CN9 |
| CL10 | AN3 | BN12 | CN10 |
| CL11 | AN3 | BN12 | CN11 |
| CL12 | AN3 | BN12 | CN12 |
| CL13 | AN3 | BN12 | CN13 |
| CL14 | AN3 | BN12 | CN14 |
| CL15 | AN3 | BN12 | CN15 |
| CL16 | AN3 | BN12 | CN16 |
| CL17 | AN3 | BN12 | CN17 |
| CL18 | AN3 | BN12 | CN18 |
| CL19 | AN3 | BN12 | CN19 |
| CL20 | AN3 | BN12 | CN20 |
| CL21 | AN3 | BN12 | CN21 |
| CL22 | AN3 | BN12 | CN22 |
| CL23 | AN3 | BN12 | CN23 |
| CL24 | AN3 | BN12 | CN24 |
| CL25 | AN3 | BN12 | CN25 |
| CL26 | AN3 | BN12 | CN26 |
| CL27 | AN3 | BN12 | CN27 |
| CL28 | AN3 | BN12 | CN28 |
| CL29 | AN3 | BN12 | CN29 |
| CL30 | AN3 | BN12 | CN30 |
| CL31 | AN3 | BN12 | CN31 |
| CL32 | AN3 | BN12 | CN32 |
| CL33 | AN3 | BN12 | CN33 |
| CL34 | AN3 | BN12 | CN34 |
| CL35 | AN3 | BN12 | CN35 |
| CL36 | AN3 | BN12 | CN36 |
| CM1 | AN3 | BN13 | CN1 |
| CM2 | AN3 | BN13 | CN2 |
| CM3 | AN3 | BN13 | CN3 |
| CM4 | AN3 | BN13 | CN4 |
| CM5 | AN3 | BN13 | CN5 |
| CM6 | AN3 | BN13 | CN6 |
| CM7 | AN3 | BN13 | CN7 |
| CM8 | AN3 | BN13 | CN8 |
| CM9 | AN3 | BN13 | CN9 |
| CM10 | AN3 | BN13 | CN10 |
| CM11 | AN3 | BN13 | CN11 |
| CM12 | AN3 | BN13 | CN12 |
| CM13 | AN3 | BN13 | CN13 |
| CM14 | AN3 | BN13 | CN14 |
| CM15 | AN3 | BN13 | CN15 |
| CM16 | AN3 | BN13 | CN16 |
| CM17 | AN3 | BN13 | CN17 |
| CM18 | AN3 | BN13 | CN18 |
| CM19 | AN3 | BN13 | CN19 |
| CM20 | AN3 | BN13 | CN20 |
| CM21 | AN3 | BN13 | CN21 |
| CM22 | AN3 | BN13 | CN22 |
| CM23 | AN3 | BN13 | CN23 |
| CM24 | AN3 | BN13 | CN24 |
| CM25 | AN3 | BN13 | CN25 |
| CM26 | AN3 | BN13 | CN26 |
| CM27 | AN3 | BN13 | CN27 |
| CM28 | AN3 | BN13 | CN28 |
| CM29 | AN3 | BN13 | CN29 |
| CM30 | AN3 | BN13 | CN30 |
| CM31 | AN3 | BN13 | CN31 |
| CM32 | AN3 | BN13 | CN32 |
| CM33 | AN3 | BN13 | CN33 |
| CM34 | AN3 | BN13 | CN34 |
| CM35 | AN3 | BN13 | CN35 |
| CM36 | AN3 | BN13 | CN36 |
| CO1 | AN3 | BN14 | CN1 |
| CO2 | AN3 | BN14 | CN2 |
| CO3 | AN3 | BN14 | CN3 |
| CO4 | AN3 | BN14 | CN4 |
| CO5 | AN3 | BN14 | CN5 |
| CO6 | AN3 | BN14 | CN6 |
| CO7 | AN3 | BN14 | CN7 |
| CO8 | AN3 | BN14 | CN8 |
| CO9 | AN3 | BN14 | CN9 |
| CO10 | AN3 | BN14 | CN10 |
| CO11 | AN3 | BN14 | CN11 |
| CO12 | AN3 | BN14 | CN12 |
| CO13 | AN3 | BN14 | CN13 |
| CO14 | AN3 | BN14 | CN14 |
| CO15 | AN3 | BN14 | CN15 |
| CO16 | AN3 | BN14 | CN16 |
| CO17 | AN3 | BN14 | CN17 |
| CO18 | AN3 | BN14 | CN18 |
| CO19 | AN3 | BN14 | CN19 |
| CO20 | AN3 | BN14 | CN20 |
| CO21 | AN3 | BN14 | CN21 |
| CO22 | AN3 | BN14 | CN22 |
| CO23 | AN3 | BN14 | CN23 |
| CO24 | AN3 | BN14 | CN24 |
| CO25 | AN3 | BN14 | CN25 |
| CO26 | AN3 | BN14 | CN26 |
| CO27 | AN3 | BN14 | CN27 |
| CO28 | AN3 | BN14 | CN28 |
| CO29 | AN3 | BN14 | CN29 |
| CO30 | AN3 | BN14 | CN30 |
| CO31 | AN3 | BN14 | CN31 |
| CO32 | AN3 | BN14 | CN32 |
| CO33 | AN3 | BN14 | CN33 |
| CO34 | AN3 | BN14 | CN34 |
| CO35 | AN3 | BN14 | CN35 |
| CO36 | AN3 | BN14 | CN36 |
| CP1 | AN3 | BN15 | CN1 |
| CP2 | AN3 | BN15 | CN2 |
| CP3 | AN3 | BN15 | CN3 |
| CP4 | AN3 | BN15 | CN4 |
| CP5 | AN3 | BN15 | CN5 |
| CP6 | AN3 | BN15 | CN6 |
| CP7 | AN3 | BN15 | CN7 |
| CP8 | AN3 | BN15 | CN8 |
| CP9 | AN3 | BN15 | CN9 |
| CP10 | AN3 | BN15 | CN10 |
| CP11 | AN3 | BN15 | CN11 |
| CP12 | AN3 | BN15 | CN12 |
| CP13 | AN3 | BN15 | CN13 |
| CP14 | AN3 | BN15 | CN14 |
| CP15 | AN3 | BN15 | CN15 |
| CP16 | AN3 | BN15 | CN16 |
| CP17 | AN3 | BN15 | CN17 |
| CP18 | AN3 | BN15 | CN18 |
| CP19 | AN3 | BN15 | CN19 |
| CP20 | AN3 | BN15 | CN20 |
| CP21 | AN3 | BN15 | CN21 |
| CP22 | AN3 | BN15 | CN22 |
| CP23 | AN3 | BN15 | CN23 |
| CP24 | AN3 | BN15 | CN24 |
| CP25 | AN3 | BN15 | CN25 |
| CP26 | AN3 | BN15 | CN26 |
| CP27 | AN3 | BN15 | CN27 |
| CP28 | AN3 | BN15 | CN28 |
| CP29 | AN3 | BN15 | CN29 |
| CP30 | AN3 | BN15 | CN30 |
| CP31 | AN3 | BN15 | CN31 |
| CP32 | AN3 | BN15 | CN32 |
| CP33 | AN3 | BN15 | CN33 |
| CP34 | AN3 | BN15 | CN34 |
| CP35 | AN3 | BN15 | CN35 |
| CP36 | AN3 | BN15 | CN36 |
| CQ1 | AN3 | BN16 | CN1 |
| CQ2 | AN3 | BN16 | CN2 |
| CQ3 | AN3 | BN16 | CN3 |
| CQ4 | AN3 | BN16 | CN4 |
| CQ5 | AN3 | BN16 | CN5 |
| CQ6 | AN3 | BN16 | CN6 |
| CQ7 | AN3 | BN16 | CN7 |
| CQ8 | AN3 | BN16 | CN8 |
| CQ9 | AN3 | BN16 | CN9 |
| CQ10 | AN3 | BN16 | CN10 |
| CQ11 | AN3 | BN16 | CN11 |
| CQ12 | AN3 | BN16 | CN12 |
| CQ13 | AN3 | BN16 | CN13 |
| CQ14 | AN3 | BN16 | CN14 |
| CQ15 | AN3 | BN16 | CN15 |
| CQ16 | AN3 | BN16 | CN16 |
| CQ17 | AN3 | BN16 | CN17 |
| CQ18 | AN3 | BN16 | CN18 |
| CQ19 | AN3 | BN16 | CN19 |
| CQ20 | AN3 | BN16 | CN20 |
| CQ21 | AN3 | BN16 | CN21 |
| CQ22 | AN3 | BN16 | CN22 |
| CQ23 | AN3 | BN16 | CN23 |
| CQ24 | AN3 | BN16 | CN24 |
| CQ25 | AN3 | BN16 | CN25 |
| CQ26 | AN3 | BN16 | CN26 |
| CQ27 | AN3 | BN16 | CN27 |
| CQ28 | AN3 | BN16 | CN28 |
| CQ29 | AN3 | BN16 | CN29 |
| CQ30 | AN3 | BN16 | CN30 |
| CQ31 | AN3 | BN16 | CN31 |
| CQ32 | AN3 | BN16 | CN32 |
| CQ33 | AN3 | BN16 | CN33 |
| CQ34 | AN3 | BN16 | CN34 |
| CQ35 | AN3 | BN16 | CN35 |
| CQ36 | AN3 | BN16 | CN36 |
| CR1 | AN3 | BN17 | CN1 |
| CR2 | AN3 | BN17 | CN2 |
| CR3 | AN3 | BN17 | CN3 |
| CR4 | AN3 | BN17 | CN4 |
| CR5 | AN3 | BN17 | CN5 |
| CR6 | AN3 | BN17 | CN6 |
| CR7 | AN3 | BN17 | CN7 |
| CR8 | AN3 | BN17 | CN8 |
| CR9 | AN3 | BN17 | CN9 |
| CR10 | AN3 | BN17 | CN10 |
| CR11 | AN3 | BN17 | CN11 |
| CR12 | AN3 | BN17 | CN12 |
| CR13 | AN3 | BN17 | CN13 |
| CR14 | AN3 | BN17 | CN14 |
| CR15 | AN3 | BN17 | CN15 |
| CR16 | AN3 | BN17 | CN16 |
| CR17 | AN3 | BN17 | CN17 |
| CR18 | AN3 | BN17 | CN18 |
| CR19 | AN3 | BN17 | CN19 |
| CR20 | AN3 | BN17 | CN20 |
| CR21 | AN3 | BN17 | CN21 |
| CR22 | AN3 | BN17 | CN22 |
| CR23 | AN3 | BN17 | CN23 |
| CR24 | AN3 | BN17 | CN24 |
| CR25 | AN3 | BN17 | CN25 |
| CR26 | AN3 | BN17 | CN26 |
| CR27 | AN3 | BN17 | CN27 |
| CR28 | AN3 | BN17 | CN28 |
| CR29 | AN3 | BN17 | CN29 |
| CR30 | AN3 | BN17 | CN30 |
| CR31 | AN3 | BN17 | CN31 |
| CR32 | AN3 | BN17 | CN32 |
| CR33 | AN3 | BN17 | CN33 |
| CR34 | AN3 | BN17 | CN34 |
| CR35 | AN3 | BN17 | CN35 |
| CR36 | AN3 | BN17 | CN36 |
| CS1 | AN3 | BN18 | CN1 |
| CS2 | AN3 | BN18 | CN2 |
| CS3 | AN3 | BN18 | CN3 |
| CS4 | AN3 | BN18 | CN4 |
| CS5 | AN3 | BN18 | CN5 |
| CS6 | AN3 | BN18 | CN6 |
| CS7 | AN3 | BN18 | CN7 |
| CS8 | AN3 | BN18 | CN8 |
| CS9 | AN3 | BN18 | CN9 |
| CS10 | AN3 | BN18 | CN10 |
| CS11 | AN3 | BN18 | CN11 |
| CS12 | AN3 | BN18 | CN12 |
| CS13 | AN3 | BN18 | CN13 |
| CS14 | AN3 | BN18 | CN14 |
| CS15 | AN3 | BN18 | CN15 |
| CS16 | AN3 | BN18 | CN16 |
| CS17 | AN3 | BN18 | CN17 |
| CS18 | AN3 | BN18 | CN18 |
| CS19 | AN3 | BN18 | CN19 |
| CS20 | AN3 | BN18 | CN20 |
| CS21 | AN3 | BN18 | CN21 |
| CS22 | AN3 | BN18 | CN22 |
| CS23 | AN3 | BN18 | CN23 |
| CS24 | AN3 | BN18 | CN24 |
| CS25 | AN3 | BN18 | CN25 |
| CS26 | AN3 | BN18 | CN26 |
| CS27 | AN3 | BN18 | CN27 |
| CS28 | AN3 | BN18 | CN28 |
| CS29 | AN3 | BN18 | CN29 |
| CS30 | AN3 | BN18 | CN30 |
| CS31 | AN3 | BN18 | CN31 |
| CS32 | AN3 | BN18 | CN32 |
| CS33 | AN3 | BN18 | CN33 |
| CS34 | AN3 | BN18 | CN34 |
| CS35 | AN3 | BN18 | CN35 |
| CS36 | AN3 | BN18 | CN36 |
| CT1 | AN3 | BN19 | CN1 |
| CT2 | AN3 | BN19 | CN2 |
| CT3 | AN3 | BN19 | CN3 |
| CT4 | AN3 | BN19 | CN4 |
| CT5 | AN3 | BN19 | CN5 |
| CT6 | AN3 | BN19 | CN6 |
| CT7 | AN3 | BN19 | CN7 |
| CT8 | AN3 | BN19 | CN8 |
| CT9 | AN3 | BN19 | CN9 |
| CT10 | AN3 | BN19 | CN10 |
| CT11 | AN3 | BN19 | CN11 |
| CT12 | AN3 | BN19 | CN12 |
| CT13 | AN3 | BN19 | CN13 |
| CT14 | AN3 | BN19 | CN14 |
| CT15 | AN3 | BN19 | CN15 |
| CT16 | AN3 | BN19 | CN16 |
| CT17 | AN3 | BN19 | CN17 |
| CT18 | AN3 | BN19 | CN18 |
| CT19 | AN3 | BN19 | CN19 |
| CT20 | AN3 | BN19 | CN20 |
| CT21 | AN3 | BN19 | CN21 |
| CT22 | AN3 | BN19 | CN22 |
| CT23 | AN3 | BN19 | CN23 |
| CT24 | AN3 | BN19 | CN24 |
| CT25 | AN3 | BN19 | CN25 |
| CT26 | AN3 | BN19 | CN26 |
| CT27 | AN3 | BN19 | CN27 |
| CT28 | AN3 | BN19 | CN28 |
| CT29 | AN3 | BN19 | CN29 |
| CT30 | AN3 | BN19 | CN30 |
| CT31 | AN3 | BN19 | CN31 |
| CT32 | AN3 | BN19 | CN32 |
| CT33 | AN3 | BN19 | CN33 |
| CT34 | AN3 | BN19 | CN34 |
| CT35 | AN3 | BN19 | CN35 |
| CT36 | AN3 | BN19 | CN36 |
| CU1 | AN3 | BN20 | CN1 |
| CU2 | AN3 | BN20 | CN2 |
| CU3 | AN3 | BN20 | CN3 |
| CU4 | AN3 | BN20 | CN4 |
| CU5 | AN3 | BN20 | CN5 |
| CU6 | AN3 | BN20 | CN6 |
| CU7 | AN3 | BN20 | CN7 |
| CU8 | AN3 | BN20 | CN8 |
| CU9 | AN3 | BN20 | CN9 |
| CU10 | AN3 | BN20 | CN10 |
| CU11 | AN3 | BN20 | CN11 |
| CU12 | AN3 | BN20 | CN12 |
| CU13 | AN3 | BN20 | CN13 |
| CU14 | AN3 | BN20 | CN14 |
| CU15 | AN3 | BN20 | CN15 |
| CU16 | AN3 | BN20 | CN16 |
| CU17 | AN3 | BN20 | CN17 |
| CU18 | AN3 | BN20 | CN18 |
| CU19 | AN3 | BN20 | CN19 |
| CU20 | AN3 | BN20 | CN20 |
| CU21 | AN3 | BN20 | CN21 |
| CU22 | AN3 | BN20 | CN22 |
| CU23 | AN3 | BN20 | CN23 |
| CU24 | AN3 | BN20 | CN24 |
| CU25 | AN3 | BN20 | CN25 |
| CU26 | AN3 | BN20 | CN26 |
| CU27 | AN3 | BN20 | CN27 |
| CU28 | AN3 | BN20 | CN28 |
| CU29 | AN3 | BN20 | CN29 |
| CU30 | AN3 | BN20 | CN30 |
| CU31 | AN3 | BN20 | CN31 |
| CU32 | AN3 | BN20 | CN32 |
| CU33 | AN3 | BN20 | CN33 |
| CU34 | AN3 | BN20 | CN34 |
| CU35 | AN3 | BN20 | CN35 |
| CU36 | AN3 | BN20 | CN36 |
| CV1 | AN3 | BN21 | CN1 |
| CV2 | AN3 | BN21 | CN2 |
| CV3 | AN3 | BN21 | CN3 |
| CV4 | AN3 | BN21 | CN4 |
| CV5 | AN3 | BN21 | CN5 |
| CV6 | AN3 | BN21 | CN6 |
| CV7 | AN3 | BN21 | CN7 |
| CV8 | AN3 | BN21 | CN8 |
| CV9 | AN3 | BN21 | CN9 |
| CV10 | AN3 | BN21 | CN10 |
| CV11 | AN3 | BN21 | CN11 |
| CV12 | AN3 | BN21 | CN12 |
| CV13 | AN3 | BN21 | CN13 |
| CV14 | AN3 | BN21 | CN14 |
| CV15 | AN3 | BN21 | CN15 |
| CV16 | AN3 | BN21 | CN16 |
| CV17 | AN3 | BN21 | CN17 |
| CV18 | AN3 | BN21 | CN18 |
| CV19 | AN3 | BN21 | CN19 |
| CV20 | AN3 | BN21 | CN20 |
| CV21 | AN3 | BN21 | CN21 |
| CV22 | AN3 | BN21 | CN22 |
| CV23 | AN3 | BN21 | CN23 |
| CV24 | AN3 | BN21 | CN24 |
| CV25 | AN3 | BN21 | CN25 |
| CV26 | AN3 | BN21 | CN26 |
| CV27 | AN3 | BN21 | CN27 |
| CV28 | AN3 | BN21 | CN28 |
| CV29 | AN3 | BN21 | CN29 |
| CV30 | AN3 | BN21 | CN30 |
| CV31 | AN3 | BN21 | CN31 |
| CV32 | AN3 | BN21 | CN32 |
| CV33 | AN3 | BN21 | CN33 |
| CV34 | AN3 | BN21 | CN34 |
| CV35 | AN3 | BN21 | CN35 |
| CV36 | AN3 | BN21 | CN36 |
| CW1 | AN3 | BN22 | CN1 |
| CW2 | AN3 | BN22 | CN2 |
| CW3 | AN3 | BN22 | CN3 |
| CW4 | AN3 | BN22 | CN4 |
| CW5 | AN3 | BN22 | CN5 |
| CW6 | AN3 | BN22 | CN6 |
| CW7 | AN3 | BN22 | CN7 |
| CW8 | AN3 | BN22 | CN8 |
| CW9 | AN3 | BN22 | CN9 |
| CW10 | AN3 | BN22 | CN10 |
| CW11 | AN3 | BN22 | CN11 |
| CW12 | AN3 | BN22 | CN12 |
| CW13 | AN3 | BN22 | CN13 |
| CW14 | AN3 | BN22 | CN14 |
| CW15 | AN3 | BN22 | CN15 |
| CW16 | AN3 | BN22 | CN16 |
| CW17 | AN3 | BN22 | CN17 |
| CW18 | AN3 | BN22 | CN18 |
| CW19 | AN3 | BN22 | CN19 |
| CW20 | AN3 | BN22 | CN20 |
| CW21 | AN3 | BN22 | CN21 |
| CW22 | AN3 | BN22 | CN22 |
| CW23 | AN3 | BN22 | CN23 |
| CW24 | AN3 | BN22 | CN24 |
| CW25 | AN3 | BN22 | CN25 |
| CW26 | AN3 | BN22 | CN26 |
| CW27 | AN3 | BN22 | CN27 |
| CW28 | AN3 | BN22 | CN28 |
| CW29 | AN3 | BN22 | CN29 |
| CW30 | AN3 | BN22 | CN30 |
| CW31 | AN3 | BN22 | CN31 |
| CW32 | AN3 | BN22 | CN32 |
| CW33 | AN3 | BN22 | CN33 |
| CW34 | AN3 | BN22 | CN34 |
| CW35 | AN3 | BN22 | CN35 |
| CW36 | AN3 | BN22 | CN36 |
| DA1 | AN4 | BN1 | CN1 |
| DA2 | AN4 | BN1 | CN2 |
| DA3 | AN4 | BN1 | CN3 |
| DA4 | AN4 | BN1 | CN4 |
| DA5 | AN4 | BN1 | CN5 |
| DA6 | AN4 | BN1 | CN6 |
| DA7 | AN4 | BN1 | CN7 |
| DA8 | AN4 | BN1 | CN8 |
| DA9 | AN4 | BN1 | CN9 |
| DA10 | AN4 | BN1 | CN10 |
| DA11 | AN4 | BN1 | CN11 |
| DA12 | AN4 | BN1 | CN12 |
| DA13 | AN4 | BN1 | CN13 |
| DA14 | AN4 | BN1 | CN14 |
| DA15 | AN4 | BN1 | CN15 |
| DA16 | AN4 | BN1 | CN16 |
| DA17 | AN4 | BN1 | CN17 |
| DA18 | AN4 | BN1 | CN18 |
| DA19 | AN4 | BN1 | CN19 |
| DA20 | AN4 | BN1 | CN20 |
| DA21 | AN4 | BN1 | CN21 |
| DA22 | AN4 | BN1 | CN22 |
| DA23 | AN4 | BN1 | CN23 |
| DA24 | AN4 | BN1 | CN24 |
| DA25 | AN4 | BN1 | CN25 |
| DA26 | AN4 | BN1 | CN26 |
| DA27 | AN4 | BN1 | CN27 |
| DA28 | AN4 | BN1 | CN28 |
| DA29 | AN4 | BN1 | CN29 |
| DA30 | AN4 | BN1 | CN30 |
| DA31 | AN4 | BN1 | CN31 |
| DA32 | AN4 | BN1 | CN32 |
| DA33 | AN4 | BN1 | CN33 |
| DA34 | AN4 | BN1 | CN34 |
| DA35 | AN4 | BN1 | CN35 |
| DA36 | AN4 | BN1 | CN36 |
| DB1 | AN4 | BN2 | CN1 |
| DB2 | AN4 | BN2 | CN2 |
| DB3 | AN4 | BN2 | CN3 |
| DB4 | AN4 | BN2 | CN4 |
| DB5 | AN4 | BN2 | CN5 |
| DB6 | AN4 | BN2 | CN6 |
| DB7 | AN4 | BN2 | CN7 |
| DB8 | AN4 | BN2 | CN8 |
| DB9 | AN4 | BN2 | CN9 |
| DB10 | AN4 | BN2 | CN10 |
| DB11 | AN4 | BN2 | CN11 |
| DB12 | AN4 | BN2 | CN12 |
| DB13 | AN4 | BN2 | CN13 |
| DB14 | AN4 | BN2 | CN14 |
| DB15 | AN4 | BN2 | CN15 |
| DB16 | AN4 | BN2 | CN16 |
| DB17 | AN4 | BN2 | CN17 |
| DB18 | AN4 | BN2 | CN18 |
| DB19 | AN4 | BN2 | CN19 |
| DB20 | AN4 | BN2 | CN20 |
| DB21 | AN4 | BN2 | CN21 |
| DB22 | AN4 | BN2 | CN22 |
| DB23 | AN4 | BN2 | CN23 |
| DB24 | AN4 | BN2 | CN24 |
| DB25 | AN4 | BN2 | CN25 |
| DB26 | AN4 | BN2 | CN26 |
| DB27 | AN4 | BN2 | CN27 |
| DB28 | AN4 | BN2 | CN28 |
| DB29 | AN4 | BN2 | CN29 |
| DB30 | AN4 | BN2 | CN30 |
| DB31 | AN4 | BN2 | CN31 |
| DB32 | AN4 | BN2 | CN32 |
| DB33 | AN4 | BN2 | CN33 |
| DB34 | AN4 | BN2 | CN34 |
| DB35 | AN4 | BN2 | CN35 |
| DB36 | AN4 | BN2 | CN36 |
| DC1 | AN4 | BN3 | CN1 |
| DC2 | AN4 | BN3 | CN2 |
| DC3 | AN4 | BN3 | CN3 |
| DC4 | AN4 | BN3 | CN4 |
| DC5 | AN4 | BN3 | CN5 |
| DC6 | AN4 | BN3 | CN6 |
| DC7 | AN4 | BN3 | CN7 |
| DC8 | AN4 | BN3 | CN8 |
| DC9 | AN4 | BN3 | CN9 |
| DC10 | AN4 | BN3 | CN10 |
| DC11 | AN4 | BN3 | CN11 |
| DC12 | AN4 | BN3 | CN12 |
| DC13 | AN4 | BN3 | CN13 |
| DC14 | AN4 | BN3 | CN14 |
| DC15 | AN4 | BN3 | CN15 |
| DC16 | AN4 | BN3 | CN16 |
| DC17 | AN4 | BN3 | CN17 |
| DC18 | AN4 | BN3 | CN18 |
| DC19 | AN4 | BN3 | CN19 |
| DC20 | AN4 | BN3 | CN20 |
| DC21 | AN4 | BN3 | CN21 |
| DC22 | AN4 | BN3 | CN22 |
| DC23 | AN4 | BN3 | CN23 |
| DC24 | AN4 | BN3 | CN24 |
| DC25 | AN4 | BN3 | CN25 |
| DC26 | AN4 | BN3 | CN26 |
| DC27 | AN4 | BN3 | CN27 |
| DC28 | AN4 | BN3 | CN28 |
| DC29 | AN4 | BN3 | CN29 |
| DC30 | AN4 | BN3 | CN30 |
| DC31 | AN4 | BN3 | CN31 |
| DC32 | AN4 | BN3 | CN32 |
| DC33 | AN4 | BN3 | CN33 |
| DC34 | AN4 | BN3 | CN34 |
| DC35 | AN4 | BN3 | CN35 |
| DC36 | AN4 | BN3 | CN36 |
| DD1 | AN4 | BN4 | CN1 |
| DD2 | AN4 | BN4 | CN2 |
| DD3 | AN4 | BN4 | CN3 |
| DD4 | AN4 | BN4 | CN4 |
| DD5 | AN4 | BN4 | CN5 |
| DD6 | AN4 | BN4 | CN6 |
| DD7 | AN4 | BN4 | CN7 |
| DD8 | AN4 | BN4 | CN8 |
| DD9 | AN4 | BN4 | CN9 |
| DD10 | AN4 | BN4 | CN10 |
| DD11 | AN4 | BN4 | CN11 |
| DD12 | AN4 | BN4 | CN12 |
| DD13 | AN4 | BN4 | CN13 |
| DD14 | AN4 | BN4 | CN14 |
| DD15 | AN4 | BN4 | CN15 |
| DD16 | AN4 | BN4 | CN16 |
| DD17 | AN4 | BN4 | CN17 |
| DD18 | AN4 | BN4 | CN18 |
| DD19 | AN4 | BN4 | CN19 |
| DD20 | AN4 | BN4 | CN20 |
| DD21 | AN4 | BN4 | CN21 |
| DD22 | AN4 | BN4 | CN22 |
| DD23 | AN4 | BN4 | CN23 |
| DD24 | AN4 | BN4 | CN24 |
| DD25 | AN4 | BN4 | CN25 |
| DD26 | AN4 | BN4 | CN26 |
| DD27 | AN4 | BN4 | CN27 |
| DD28 | AN4 | BN4 | CN28 |
| DD29 | AN4 | BN4 | CN29 |
| DD30 | AN4 | BN4 | CN30 |
| DD31 | AN4 | BN4 | CN31 |
| DD32 | AN4 | BN4 | CN32 |
| DD33 | AN4 | BN4 | CN33 |
| DD34 | AN4 | BN4 | CN34 |
| DD35 | AN4 | BN4 | CN35 |
| DD36 | AN4 | BN4 | CN36 |
| DE1 | AN4 | BN5 | CN1 |
| DE2 | AN4 | BN5 | CN2 |
| DE3 | AN4 | BN5 | CN3 |
| DE4 | AN4 | BN5 | CN4 |
| DE5 | AN4 | BN5 | CN5 |
| DE6 | AN4 | BN5 | CN6 |
| DE7 | AN4 | BN5 | CN7 |
| DE8 | AN4 | BN5 | CN8 |
| DE9 | AN4 | BN5 | CN9 |
| DE10 | AN4 | BN5 | CN10 |
| DE11 | AN4 | BN5 | CN11 |
| DE12 | AN4 | BN5 | CN12 |
| DE13 | AN4 | BN5 | CN13 |
| DE14 | AN4 | BN5 | CN14 |
| DE15 | AN4 | BN5 | CN15 |
| DE16 | AN4 | BN5 | CN16 |
| DE17 | AN4 | BN5 | CN17 |
| DE18 | AN4 | BN5 | CN18 |
| DE19 | AN4 | BN5 | CN19 |
| DE20 | AN4 | BN5 | CN20 |
| DE21 | AN4 | BN5 | CN21 |
| DE22 | AN4 | BN5 | CN22 |
| DE23 | AN4 | BN5 | CN23 |
| DE24 | AN4 | BN5 | CN24 |
| DE25 | AN4 | BN5 | CN25 |
| DE26 | AN4 | BN5 | CN26 |
| DE27 | AN4 | BN5 | CN27 |
| DE28 | AN4 | BN5 | CN28 |
| DE29 | AN4 | BN5 | CN29 |
| DE30 | AN4 | BN5 | CN30 |
| DE31 | AN4 | BN5 | CN31 |
| DE32 | AN4 | BN5 | CN32 |
| DE33 | AN4 | BN5 | CN33 |
| DE34 | AN4 | BN5 | CN34 |
| DE35 | AN4 | BN5 | CN35 |
| DE36 | AN4 | BN5 | CN36 |
| DF1 | AN4 | BN6 | CN1 |
| DF2 | AN4 | BN6 | CN2 |
| DF3 | AN4 | BN6 | CN3 |
| DF4 | AN4 | BN6 | CN4 |
| DF5 | AN4 | BN6 | CN5 |
| DF6 | AN4 | BN6 | CN6 |
| DF7 | AN4 | BN6 | CN7 |
| DF8 | AN4 | BN6 | CN8 |
| DF9 | AN4 | BN6 | CN9 |
| DF10 | AN4 | BN6 | CN10 |
| DF11 | AN4 | BN6 | CN11 |
| DF12 | AN4 | BN6 | CN12 |
| DF13 | AN4 | BN6 | CN13 |
| DF14 | AN4 | BN6 | CN14 |
| DF15 | AN4 | BN6 | CN15 |
| DF16 | AN4 | BN6 | CN16 |
| DF17 | AN4 | BN6 | CN17 |
| DF18 | AN4 | BN6 | CN18 |
| DF19 | AN4 | BN6 | CN19 |
| DF20 | AN4 | BN6 | CN20 |
| DF21 | AN4 | BN6 | CN21 |
| DF22 | AN4 | BN6 | CN22 |
| DF23 | AN4 | BN6 | CN23 |
| DF24 | AN4 | BN6 | CN24 |
| DF25 | AN4 | BN6 | CN25 |
| DF26 | AN4 | BN6 | CN26 |
| DF27 | AN4 | BN6 | CN27 |
| DF28 | AN4 | BN6 | CN28 |
| DF29 | AN4 | BN6 | CN29 |
| DF30 | AN4 | BN6 | CN30 |
| DF31 | AN4 | BN6 | CN31 |
| DF32 | AN4 | BN6 | CN32 |
| DF33 | AN4 | BN6 | CN33 |
| DF34 | AN4 | BN6 | CN34 |
| DF35 | AN4 | BN6 | CN35 |
| DF36 | AN4 | BN6 | CN36 |
| DG1 | AN4 | BN7 | CN1 |
| DG2 | AN4 | BN7 | CN2 |
| DG3 | AN4 | BN7 | CN3 |
| DG4 | AN4 | BN7 | CN4 |
| DG5 | AN4 | BN7 | CN5 |
| DG6 | AN4 | BN7 | CN6 |
| DG7 | AN4 | BN7 | CN7 |
| DG8 | AN4 | BN7 | CN8 |
| DG9 | AN4 | BN7 | CN9 |
| DG10 | AN4 | BN7 | CN10 |
| DG11 | AN4 | BN7 | CN11 |
| DG12 | AN4 | BN7 | CN12 |
| DG13 | AN4 | BN7 | CN13 |
| DG14 | AN4 | BN7 | CN14 |
| DG15 | AN4 | BN7 | CN15 |
| DG16 | AN4 | BN7 | CN16 |
| DG17 | AN4 | BN7 | CN17 |
| DG18 | AN4 | BN7 | CN18 |
| DG19 | AN4 | BN7 | CN19 |
| DG20 | AN4 | BN7 | CN20 |
| DG21 | AN4 | BN7 | CN21 |
| DG22 | AN4 | BN7 | CN22 |
| DG23 | AN4 | BN7 | CN23 |
| DG24 | AN4 | BN7 | CN24 |
| DG25 | AN4 | BN7 | CN25 |
| DG26 | AN4 | BN7 | CN26 |
| DG27 | AN4 | BN7 | CN27 |
| DG28 | AN4 | BN7 | CN28 |
| DG29 | AN4 | BN7 | CN29 |
| DG30 | AN4 | BN7 | CN30 |
| DG31 | AN4 | BN7 | CN31 |
| DG32 | AN4 | BN7 | CN32 |
| DG33 | AN4 | BN7 | CN33 |
| DG34 | AN4 | BN7 | CN34 |
| DG35 | AN4 | BN7 | CN35 |
| DG36 | AN4 | BN7 | CN36 |
| DH1 | AN4 | BN8 | CN1 |
| DH2 | AN4 | BN8 | CN2 |
| DH3 | AN4 | BN8 | CN3 |
| DH4 | AN4 | BN8 | CN4 |
| DH5 | AN4 | BN8 | CN5 |
| DH6 | AN4 | BN8 | CN6 |
| DH7 | AN4 | BN8 | CN7 |
| DH8 | AN4 | BN8 | CN8 |
| DH9 | AN4 | BN8 | CN9 |
| DH10 | AN4 | BN8 | CN10 |
| DH11 | AN4 | BN8 | CN11 |
| DH12 | AN4 | BN8 | CN12 |
| DH13 | AN4 | BN8 | CN13 |
| DH14 | AN4 | BN8 | CN14 |
| DH15 | AN4 | BN8 | CN15 |
| DH16 | AN4 | BN8 | CN16 |
| DH17 | AN4 | BN8 | CN17 |
| DH18 | AN4 | BN8 | CN18 |
| DH19 | AN4 | BN8 | CN19 |
| DH20 | AN4 | BN8 | CN20 |
| DH21 | AN4 | BN8 | CN21 |
| DH22 | AN4 | BN8 | CN22 |
| DH23 | AN4 | BN8 | CN23 |
| DH24 | AN4 | BN8 | CN24 |
| DH25 | AN4 | BN8 | CN25 |
| DH26 | AN4 | BN8 | CN26 |
| DH27 | AN4 | BN8 | CN27 |
| DH28 | AN4 | BN8 | CN28 |
| DH29 | AN4 | BN8 | CN29 |
| DH30 | AN4 | BN8 | CN30 |
| DH31 | AN4 | BN8 | CN31 |
| DH32 | AN4 | BN8 | CN32 |
| DH33 | AN4 | BN8 | CN33 |
| DH34 | AN4 | BN8 | CN34 |
| DH35 | AN4 | BN8 | CN35 |
| DH36 | AN4 | BN8 | CN36 |
| DI1 | AN4 | BN9 | CN1 |
| DI2 | AN4 | BN9 | CN2 |
| DI3 | AN4 | BN9 | CN3 |
| DI4 | AN4 | BN9 | CN4 |
| DI5 | AN4 | BN9 | CN5 |
| DI6 | AN4 | BN9 | CN6 |
| DI7 | AN4 | BN9 | CN7 |
| DI8 | AN4 | BN9 | CN8 |
| DI9 | AN4 | BN9 | CN9 |
| DI10 | AN4 | BN9 | CN10 |
| DI11 | AN4 | BN9 | CN11 |
| DI12 | AN4 | BN9 | CN12 |
| DI13 | AN4 | BN9 | CN13 |
| DI14 | AN4 | BN9 | CN14 |
| DI15 | AN4 | BN9 | CN15 |
| DI16 | AN4 | BN9 | CN16 |
| DI17 | AN4 | BN9 | CN17 |
| DI18 | AN4 | BN9 | CN18 |
| DI19 | AN4 | BN9 | CN19 |
| DI20 | AN4 | BN9 | CN20 |
| DI21 | AN4 | BN9 | CN21 |
| DI22 | AN4 | BN9 | CN22 |
| DI23 | AN4 | BN9 | CN23 |
| DI24 | AN4 | BN9 | CN24 |
| DI25 | AN4 | BN9 | CN25 |
| DI26 | AN4 | BN9 | CN26 |
| DI27 | AN4 | BN9 | CN27 |
| DI28 | AN4 | BN9 | CN28 |
| DI29 | AN4 | BN9 | CN29 |
| DI30 | AN4 | BN9 | CN30 |
| DI31 | AN4 | BN9 | CN31 |
| DI32 | AN4 | BN9 | CN32 |
| DI33 | AN4 | BN9 | CN33 |
| DI34 | AN4 | BN9 | CN34 |
| DI35 | AN4 | BN9 | CN35 |
| DI36 | AN4 | BN9 | CN36 |
| DJ1 | AN4 | BN10 | CN1 |
| DJ2 | AN4 | BN10 | CN2 |
| DJ3 | AN4 | BN10 | CN3 |
| DJ4 | AN4 | BN10 | CN4 |
| DJ5 | AN4 | BN10 | CN5 |
| DJ6 | AN4 | BN10 | CN6 |
| DJ7 | AN4 | BN10 | CN7 |
| DJ8 | AN4 | BN10 | CN8 |
| DJ9 | AN4 | BN10 | CN9 |
| DJ10 | AN4 | BN10 | CN10 |
| DJ11 | AN4 | BN10 | CN11 |
| DJ12 | AN4 | BN10 | CN12 |
| DJ13 | AN4 | BN10 | CN13 |
| DJ14 | AN4 | BN10 | CN14 |
| DJ15 | AN4 | BN10 | CN15 |
| DJ16 | AN4 | BN10 | CN16 |
| DJ17 | AN4 | BN10 | CN17 |
| DJ18 | AN4 | BN10 | CN18 |
| DJ19 | AN4 | BN10 | CN19 |
| DJ20 | AN4 | BN10 | CN20 |
| DJ21 | AN4 | BN10 | CN21 |
| DJ22 | AN4 | BN10 | CN22 |
| DJ23 | AN4 | BN10 | CN23 |
| DJ24 | AN4 | BN10 | CN24 |
| DJ25 | AN4 | BN10 | CN25 |
| DJ26 | AN4 | BN10 | CN26 |
| DJ27 | AN4 | BN10 | CN27 |
| DJ28 | AN4 | BN10 | CN28 |
| DJ29 | AN4 | BN10 | CN29 |
| DJ30 | AN4 | BN10 | CN30 |
| DJ31 | AN4 | BN10 | CN31 |
| DJ32 | AN4 | BN10 | CN32 |
| DJ33 | AN4 | BN10 | CN33 |
| DJ34 | AN4 | BN10 | CN34 |
| DJ35 | AN4 | BN10 | CN35 |
| DJ36 | AN4 | BN10 | CN36 |
| DK1 | AN4 | BN11 | CN1 |
| DK2 | AN4 | BN11 | CN2 |
| DK3 | AN4 | BN11 | CN3 |
| DK4 | AN4 | BN11 | CN4 |
| DK5 | AN4 | BN11 | CN5 |
| DK6 | AN4 | BN11 | CN6 |
| DK7 | AN4 | BN11 | CN7 |
| DK8 | AN4 | BN11 | CN8 |
| DK9 | AN4 | BN11 | CN9 |
| DK10 | AN4 | BN11 | CN10 |
| DK11 | AN4 | BN11 | CN11 |
| DK12 | AN4 | BN11 | CN12 |
| DK13 | AN4 | BN11 | CN13 |
| DK14 | AN4 | BN11 | CN14 |
| DK15 | AN4 | BN11 | CN15 |
| DK16 | AN4 | BN11 | CN16 |
| DK17 | AN4 | BN11 | CN17 |
| DK18 | AN4 | BN11 | CN18 |
| DK19 | AN4 | BN11 | CN19 |
| DK20 | AN4 | BN11 | CN20 |
| DK21 | AN4 | BN11 | CN21 |
| DK22 | AN4 | BN11 | CN22 |
| DK23 | AN4 | BN11 | CN23 |
| DK24 | AN4 | BN11 | CN24 |
| DK25 | AN4 | BN11 | CN25 |
| DK26 | AN4 | BN11 | CN26 |
| DK27 | AN4 | BN11 | CN27 |
| DK28 | AN4 | BN11 | CN28 |
| DK29 | AN4 | BN11 | CN29 |
| DK30 | AN4 | BN11 | CN30 |
| DK31 | AN4 | BN11 | CN31 |
| DK32 | AN4 | BN11 | CN32 |
| DK33 | AN4 | BN11 | CN33 |
| DK34 | AN4 | BN11 | CN34 |
| DK35 | AN4 | BN11 | CN35 |
| DK36 | AN4 | BN11 | CN36 |
| DL1 | AN4 | BN12 | CN1 |
| DL2 | AN4 | BN12 | CN2 |
| DL3 | AN4 | BN12 | CN3 |
| DL4 | AN4 | BN12 | CN4 |
| DL5 | AN4 | BN12 | CN5 |
| DL6 | AN4 | BN12 | CN6 |
| DL7 | AN4 | BN12 | CN7 |
| DL8 | AN4 | BN12 | CN8 |
| DL9 | AN4 | BN12 | CN9 |
| DL10 | AN4 | BN12 | CN10 |
| DL11 | AN4 | BN12 | CN11 |
| DL12 | AN4 | BN12 | CN12 |
| DL13 | AN4 | BN12 | CN13 |
| DL14 | AN4 | BN12 | CN14 |
| DL15 | AN4 | BN12 | CN15 |
| DL16 | AN4 | BN12 | CN16 |
| DL17 | AN4 | BN12 | CN17 |
| DL18 | AN4 | BN12 | CN18 |
| DL19 | AN4 | BN12 | CN19 |
| DL20 | AN4 | BN12 | CN20 |
| DL21 | AN4 | BN12 | CN21 |
| DL22 | AN4 | BN12 | CN22 |
| DL23 | AN4 | BN12 | CN23 |
| DL24 | AN4 | BN12 | CN24 |
| DL25 | AN4 | BN12 | CN25 |
| DL26 | AN4 | BN12 | CN26 |
| DL27 | AN4 | BN12 | CN27 |
| DL28 | AN4 | BN12 | CN28 |
| DL29 | AN4 | BN12 | CN29 |
| DL30 | AN4 | BN12 | CN30 |
| DL31 | AN4 | BN12 | CN31 |
| DL32 | AN4 | BN12 | CN32 |
| DL33 | AN4 | BN12 | CN33 |
| DL34 | AN4 | BN12 | CN34 |
| DL35 | AN4 | BN12 | CN35 |
| DL36 | AN4 | BN12 | CN36 |
| DM1 | AN4 | BN13 | CN1 |
| DM2 | AN4 | BN13 | CN2 |
| DM3 | AN4 | BN13 | CN3 |
| DM4 | AN4 | BN13 | CN4 |
| DM5 | AN4 | BN13 | CN5 |
| DM6 | AN4 | BN13 | CN6 |
| DM7 | AN4 | BN13 | CN7 |
| DM8 | AN4 | BN13 | CN8 |
| DM9 | AN4 | BN13 | CN9 |
| DM10 | AN4 | BN13 | CN10 |
| DM11 | AN4 | BN13 | CN11 |
| DM12 | AN4 | BN13 | CN12 |
| DM13 | AN4 | BN13 | CN13 |
| DM14 | AN4 | BN13 | CN14 |
| DM15 | AN4 | BN13 | CN15 |
| DM16 | AN4 | BN13 | CN16 |
| DM17 | AN4 | BN13 | CN17 |
| DM18 | AN4 | BN13 | CN18 |
| DM19 | AN4 | BN13 | CN19 |
| DM20 | AN4 | BN13 | CN20 |
| DM21 | AN4 | BN13 | CN21 |
| DM22 | AN4 | BN13 | CN22 |
| DM23 | AN4 | BN13 | CN23 |
| DM24 | AN4 | BN13 | CN24 |
| DM25 | AN4 | BN13 | CN25 |
| DM26 | AN4 | BN13 | CN26 |
| DM27 | AN4 | BN13 | CN27 |
| DM28 | AN4 | BN13 | CN28 |
| DM29 | AN4 | BN13 | CN29 |
| DM30 | AN4 | BN13 | CN30 |
| DM31 | AN4 | BN13 | CN31 |
| DM32 | AN4 | BN13 | CN32 |
| DM33 | AN4 | BN13 | CN33 |
| DM34 | AN4 | BN13 | CN34 |
| DM35 | AN4 | BN13 | CN35 |
| DM36 | AN4 | BN13 | CN36 |
| DO1 | AN4 | BN14 | CN1 |
| DO2 | AN4 | BN14 | CN2 |
| DO3 | AN4 | BN14 | CN3 |
| DO4 | AN4 | BN14 | CN4 |
| DO5 | AN4 | BN14 | CN5 |
| DO6 | AN4 | BN14 | CN6 |
| DO7 | AN4 | BN14 | CN7 |
| DO8 | AN4 | BN14 | CN8 |
| DO9 | AN4 | BN14 | CN9 |
| DO10 | AN4 | BN14 | CN10 |
| DO11 | AN4 | BN14 | CN11 |
| DO12 | AN4 | BN14 | CN12 |
| DO13 | AN4 | BN14 | CN13 |
| DO14 | AN4 | BN14 | CN14 |
| DO15 | AN4 | BN14 | CN15 |
| DO16 | AN4 | BN14 | CN16 |
| DO17 | AN4 | BN14 | CN17 |
| DO18 | AN4 | BN14 | CN18 |
| DO19 | AN4 | BN14 | CN19 |
| DO20 | AN4 | BN14 | CN20 |
| DO21 | AN4 | BN14 | CN21 |
| DO22 | AN4 | BN14 | CN22 |
| DO23 | AN4 | BN14 | CN23 |
| DO24 | AN4 | BN14 | CN24 |
| DO25 | AN4 | BN14 | CN25 |
| DO26 | AN4 | BN14 | CN26 |
| DO27 | AN4 | BN14 | CN27 |
| DO28 | AN4 | BN14 | CN28 |
| DO29 | AN4 | BN14 | CN29 |
| DO30 | AN4 | BN14 | CN30 |
| DO31 | AN4 | BN14 | CN31 |
| DO32 | AN4 | BN14 | CN32 |
| DO33 | AN4 | BN14 | CN33 |
| DO34 | AN4 | BN14 | CN34 |
| DO35 | AN4 | BN14 | CN35 |
| DO36 | AN4 | BN14 | CN36 |
| DP1 | AN4 | BN15 | CN1 |
| DP2 | AN4 | BN15 | CN2 |
| DP3 | AN4 | BN15 | CN3 |
| DP4 | AN4 | BN15 | CN4 |
| DP5 | AN4 | BN15 | CN5 |
| DP6 | AN4 | BN15 | CN6 |
| DP7 | AN4 | BN15 | CN7 |
| DP8 | AN4 | BN15 | CN8 |
| DP9 | AN4 | BN15 | CN9 |
| DP10 | AN4 | BN15 | CN10 |
| DP11 | AN4 | BN15 | CN11 |
| DP12 | AN4 | BN15 | CN12 |
| DP13 | AN4 | BN15 | CN13 |
| DP14 | AN4 | BN15 | CN14 |
| DP15 | AN4 | BN15 | CN15 |
| DP16 | AN4 | BN15 | CN16 |
| DP17 | AN4 | BN15 | CN17 |
| DP18 | AN4 | BN15 | CN18 |
| DP19 | AN4 | BN15 | CN19 |
| DP20 | AN4 | BN15 | CN20 |
| DP21 | AN4 | BN15 | CN21 |
| DP22 | AN4 | BN15 | CN22 |
| DP23 | AN4 | BN15 | CN23 |
| DP24 | AN4 | BN15 | CN24 |
| DP25 | AN4 | BN15 | CN25 |
| DP26 | AN4 | BN15 | CN26 |
| DP27 | AN4 | BN15 | CN27 |
| DP28 | AN4 | BN15 | CN28 |
| DP29 | AN4 | BN15 | CN29 |
| DP30 | AN4 | BN15 | CN30 |
| DP31 | AN4 | BN15 | CN31 |
| DP32 | AN4 | BN15 | CN32 |
| DP33 | AN4 | BN15 | CN33 |
| DP34 | AN4 | BN15 | CN34 |
| DP35 | AN4 | BN15 | CN35 |
| DP36 | AN4 | BN15 | CN36 |
| DQ1 | AN4 | BN16 | CN1 |
| DQ2 | AN4 | BN16 | CN2 |
| DQ3 | AN4 | BN16 | CN3 |
| DQ4 | AN4 | BN16 | CN4 |
| DQ5 | AN4 | BN16 | CN5 |
| DQ6 | AN4 | BN16 | CN6 |
| DQ7 | AN4 | BN16 | CN7 |
| DQ8 | AN4 | BN16 | CN8 |
| DQ9 | AN4 | BN16 | CN9 |
| DQ10 | AN4 | BN16 | CN10 |
| DQ11 | AN4 | BN16 | CN11 |
| DQ12 | AN4 | BN16 | CN12 |
| DQ13 | AN4 | BN16 | CN13 |
| DQ14 | AN4 | BN16 | CN14 |
| DQ15 | AN4 | BN16 | CN15 |
| DQ16 | AN4 | BN16 | CN16 |
| DQ17 | AN4 | BN16 | CN17 |
| DQ18 | AN4 | BN16 | CN18 |
| DQ19 | AN4 | BN16 | CN19 |
| DQ20 | AN4 | BN16 | CN20 |
| DQ21 | AN4 | BN16 | CN21 |
| DQ22 | AN4 | BN16 | CN22 |
| DQ23 | AN4 | BN16 | CN23 |
| DQ24 | AN4 | BN16 | CN24 |
| DQ25 | AN4 | BN16 | CN25 |
| DQ26 | AN4 | BN16 | CN26 |
| DQ27 | AN4 | BN16 | CN27 |
| DQ28 | AN4 | BN16 | CN28 |
| DQ29 | AN4 | BN16 | CN29 |
| DQ30 | AN4 | BN16 | CN30 |
| DQ31 | AN4 | BN16 | CN31 |
| DQ32 | AN4 | BN16 | CN32 |
| DQ33 | AN4 | BN16 | CN33 |
| DQ34 | AN4 | BN16 | CN34 |
| DQ35 | AN4 | BN16 | CN35 |
| DQ36 | AN4 | BN16 | CN36 |
| DR1 | AN4 | BN17 | CN1 |
| DR2 | AN4 | BN17 | CN2 |
| DR3 | AN4 | BN17 | CN3 |
| DR4 | AN4 | BN17 | CN4 |
| DR5 | AN4 | BN17 | CN5 |
| DR6 | AN4 | BN17 | CN6 |
| DR7 | AN4 | BN17 | CN7 |
| DR8 | AN4 | BN17 | CN8 |
| DR9 | AN4 | BN17 | CN9 |
| DR10 | AN4 | BN17 | CN10 |
| DR11 | AN4 | BN17 | CN11 |
| DR12 | AN4 | BN17 | CN12 |
| DR13 | AN4 | BN17 | CN13 |
| DR14 | AN4 | BN17 | CN14 |
| DR15 | AN4 | BN17 | CN15 |
| DR16 | AN4 | BN17 | CN16 |
| DR17 | AN4 | BN17 | CN17 |
| DR18 | AN4 | BN17 | CN18 |
| DR19 | AN4 | BN17 | CN19 |
| DR20 | AN4 | BN17 | CN20 |
| DR21 | AN4 | BN17 | CN21 |
| DR22 | AN4 | BN17 | CN22 |
| DR23 | AN4 | BN17 | CN23 |
| DR24 | AN4 | BN17 | CN24 |
| DR25 | AN4 | BN17 | CN25 |
| DR26 | AN4 | BN17 | CN26 |
| DR27 | AN4 | BN17 | CN27 |
| DR28 | AN4 | BN17 | CN28 |
| DR29 | AN4 | BN17 | CN29 |
| DR30 | AN4 | BN17 | CN30 |
| DR31 | AN4 | BN17 | CN31 |
| DR32 | AN4 | BN17 | CN32 |
| DR33 | AN4 | BN17 | CN33 |
| DR34 | AN4 | BN17 | CN34 |
| DR35 | AN4 | BN17 | CN35 |
| DR36 | AN4 | BN17 | CN36 |
| DS1 | AN4 | BN18 | CN1 |
| DS2 | AN4 | BN18 | CN2 |
| DS3 | AN4 | BN18 | CN3 |
| DS4 | AN4 | BN18 | CN4 |
| DS5 | AN4 | BN18 | CN5 |
| DS6 | AN4 | BN18 | CN6 |
| DS7 | AN4 | BN18 | CN7 |
| DS8 | AN4 | BN18 | CN8 |
| DS9 | AN4 | BN18 | CN9 |
| DS10 | AN4 | BN18 | CN10 |
| DS11 | AN4 | BN18 | CN11 |
| DS12 | AN4 | BN18 | CN12 |
| DS13 | AN4 | BN18 | CN13 |
| DS14 | AN4 | BN18 | CN14 |
| DS15 | AN4 | BN18 | CN15 |
| DS16 | AN4 | BN18 | CN16 |
| DS17 | AN4 | BN18 | CN17 |
| DS18 | AN4 | BN18 | CN18 |
| DS19 | AN4 | BN18 | CN19 |
| DS20 | AN4 | BN18 | CN20 |
| DS21 | AN4 | BN18 | CN21 |
| DS22 | AN4 | BN18 | CN22 |
| DS23 | AN4 | BN18 | CN23 |
| DS24 | AN4 | BN18 | CN24 |
| DS25 | AN4 | BN18 | CN25 |
| DS26 | AN4 | BN18 | CN26 |
| DS27 | AN4 | BN18 | CN27 |
| DS28 | AN4 | BN18 | CN28 |
| DS29 | AN4 | BN18 | CN29 |
| DS30 | AN4 | BN18 | CN30 |
| DS31 | AN4 | BN18 | CN31 |
| DS32 | AN4 | BN18 | CN32 |
| DS33 | AN4 | BN18 | CN33 |
| DS34 | AN4 | BN18 | CN34 |
| DS35 | AN4 | BN18 | CN35 |
| DS36 | AN4 | BN18 | CN36 |
| DT1 | AN4 | BN19 | CN1 |
| DT2 | AN4 | BN19 | CN2 |
| DT3 | AN4 | BN19 | CN3 |
| DT4 | AN4 | BN19 | CN4 |
| DT5 | AN4 | BN19 | CN5 |
| DT6 | AN4 | BN19 | CN6 |
| DT7 | AN4 | BN19 | CN7 |
| DT8 | AN4 | BN19 | CN8 |
| DT9 | AN4 | BN19 | CN9 |
| DT10 | AN4 | BN19 | CN10 |
| DT11 | AN4 | BN19 | CN11 |
| DT12 | AN4 | BN19 | CN12 |
| DT13 | AN4 | BN19 | CN13 |
| DT14 | AN4 | BN19 | CN14 |
| DT15 | AN4 | BN19 | CN15 |
| DT16 | AN4 | BN19 | CN16 |
| DT17 | AN4 | BN19 | CN17 |
| DT18 | AN4 | BN19 | CN18 |
| DT19 | AN4 | BN19 | CN19 |
| DT20 | AN4 | BN19 | CN20 |
| DT21 | AN4 | BN19 | CN21 |
| DT22 | AN4 | BN19 | CN22 |
| DT23 | AN4 | BN19 | CN23 |
| DT24 | AN4 | BN19 | CN24 |
| DT25 | AN4 | BN19 | CN25 |
| DT26 | AN4 | BN19 | CN26 |
| DT27 | AN4 | BN19 | CN27 |
| DT28 | AN4 | BN19 | CN28 |
| DT29 | AN4 | BN19 | CN29 |
| DT30 | AN4 | BN19 | CN30 |
| DT31 | AN4 | BN19 | CN31 |
| DT32 | AN4 | BN19 | CN32 |
| DT33 | AN4 | BN19 | CN33 |
| DT34 | AN4 | BN19 | CN34 |
| DT35 | AN4 | BN19 | CN35 |
| DT36 | AN4 | BN19 | CN36 |
| DU1 | AN4 | BN20 | CN1 |
| DU2 | AN4 | BN20 | CN2 |
| DU3 | AN4 | BN20 | CN3 |
| DU4 | AN4 | BN20 | CN4 |
| DU5 | AN4 | BN20 | CN5 |
| DU6 | AN4 | BN20 | CN6 |
| DU7 | AN4 | BN20 | CN7 |
| DU8 | AN4 | BN20 | CN8 |
| DU9 | AN4 | BN20 | CN9 |
| DU10 | AN4 | BN20 | CN10 |
| DU11 | AN4 | BN20 | CN11 |
| DU12 | AN4 | BN20 | CN12 |
| DU13 | AN4 | BN20 | CN13 |
| DU14 | AN4 | BN20 | CN14 |
| DU15 | AN4 | BN20 | CN15 |
| DU16 | AN4 | BN20 | CN16 |
| DU17 | AN4 | BN20 | CN17 |
| DU18 | AN4 | BN20 | CN18 |
| DU19 | AN4 | BN20 | CN19 |
| DU20 | AN4 | BN20 | CN20 |
| DU21 | AN4 | BN20 | CN21 |
| DU22 | AN4 | BN20 | CN22 |
| DU23 | AN4 | BN20 | CN23 |
| DU24 | AN4 | BN20 | CN24 |
| DU25 | AN4 | BN20 | CN25 |
| DU26 | AN4 | BN20 | CN26 |
| DU27 | AN4 | BN20 | CN27 |
| DU28 | AN4 | BN20 | CN28 |
| DU29 | AN4 | BN20 | CN29 |
| DU30 | AN4 | BN20 | CN30 |
| DU31 | AN4 | BN20 | CN31 |
| DU32 | AN4 | BN20 | CN32 |
| DU33 | AN4 | BN20 | CN33 |
| DU34 | AN4 | BN20 | CN34 |
| DU35 | AN4 | BN20 | CN35 |
| DU36 | AN4 | BN20 | CN36 |
| DV1 | AN4 | BN21 | CN1 |
| DV2 | AN4 | BN21 | CN2 |
| DV3 | AN4 | BN21 | CN3 |
| DV4 | AN4 | BN21 | CN4 |
| DV5 | AN4 | BN21 | CN5 |
| DV6 | AN4 | BN21 | CN6 |
| DV7 | AN4 | BN21 | CN7 |
| DV8 | AN4 | BN21 | CN8 |
| DV9 | AN4 | BN21 | CN9 |
| DV10 | AN4 | BN21 | CN10 |
| DV11 | AN4 | BN21 | CN11 |
| DV12 | AN4 | BN21 | CN12 |
| DV13 | AN4 | BN21 | CN13 |
| DV14 | AN4 | BN21 | CN14 |
| DV15 | AN4 | BN21 | CN15 |
| DV16 | AN4 | BN21 | CN16 |
| DV17 | AN4 | BN21 | CN17 |
| DV18 | AN4 | BN21 | CN18 |
| DV19 | AN4 | BN21 | CN19 |
| DV20 | AN4 | BN21 | CN20 |
| DV21 | AN4 | BN21 | CN21 |
| DV22 | AN4 | BN21 | CN22 |
| DV23 | AN4 | BN21 | CN23 |
| DV24 | AN4 | BN21 | CN24 |
| DV25 | AN4 | BN21 | CN25 |
| DV26 | AN4 | BN21 | CN26 |
| DV27 | AN4 | BN21 | CN27 |
| DV28 | AN4 | BN21 | CN28 |
| DV29 | AN4 | BN21 | CN29 |
| DV30 | AN4 | BN21 | CN30 |
| DV31 | AN4 | BN21 | CN31 |
| DV32 | AN4 | BN21 | CN32 |
| DV33 | AN4 | BN21 | CN33 |
| DV34 | AN4 | BN21 | CN34 |
| DV35 | AN4 | BN21 | CN35 |
| DV36 | AN4 | BN21 | CN36 |
| DW1 | AN4 | BN22 | CN1 |
| DW2 | AN4 | BN22 | CN2 |
| DW3 | AN4 | BN22 | CN3 |
| DW4 | AN4 | BN22 | CN4 |
| DW5 | AN4 | BN22 | CN5 |
| DW6 | AN4 | BN22 | CN6 |
| DW7 | AN4 | BN22 | CN7 |
| DW8 | AN4 | BN22 | CN8 |
| DW9 | AN4 | BN22 | CN9 |
| DW10 | AN4 | BN22 | CN10 |
| DW11 | AN4 | BN22 | CN11 |
| DW12 | AN4 | BN22 | CN12 |
| DW13 | AN4 | BN22 | CN13 |
| DW14 | AN4 | BN22 | CN14 |
| DW15 | AN4 | BN22 | CN15 |
| DW16 | AN4 | BN22 | CN16 |
| DW17 | AN4 | BN22 | CN17 |
| DW18 | AN4 | BN22 | CN18 |
| DW19 | AN4 | BN22 | CN19 |
| DW20 | AN4 | BN22 | CN20 |
| DW21 | AN4 | BN22 | CN21 |
| DW22 | AN4 | BN22 | CN22 |
| DW23 | AN4 | BN22 | CN23 |
| DW24 | AN4 | BN22 | CN24 |
| DW25 | AN4 | BN22 | CN25 |
| DW26 | AN4 | BN22 | CN26 |
| DW27 | AN4 | BN22 | CN27 |
| DW28 | AN4 | BN22 | CN28 |
| DW29 | AN4 | BN22 | CN29 |
| DW30 | AN4 | BN22 | CN30 |
| DW31 | AN4 | BN22 | CN31 |
| DW32 | AN4 | BN22 | CN32 |
| DW33 | AN4 | BN22 | CN33 |
| DW34 | AN4 | BN22 | CN34 |
| DW35 | AN4 | BN22 | CN35 |
| DW36 | AN4 | BN22 | CN36 |
| EA1 | AN5 | BN1 | CN1 |
| EA2 | AN5 | BN1 | CN2 |
| EA3 | AN5 | BN1 | CN3 |
| EA4 | AN5 | BN1 | CN4 |
| EA5 | AN5 | BN1 | CN5 |
| EA6 | AN5 | BN1 | CN6 |
| EA7 | AN5 | BN1 | CN7 |
| EA8 | AN5 | BN1 | CN8 |
| EA9 | AN5 | BN1 | CN9 |
| EA10 | AN5 | BN1 | CN10 |
| EA11 | AN5 | BN1 | CN11 |
| EA12 | AN5 | BN1 | CN12 |
| EA13 | AN5 | BN1 | CN13 |
| EA14 | AN5 | BN1 | CN14 |
| EA15 | AN5 | BN1 | CN15 |
| EA16 | AN5 | BN1 | CN16 |
| EA17 | AN5 | BN1 | CN17 |
| EA18 | AN5 | BN1 | CN18 |
| EA19 | AN5 | BN1 | CN19 |
| EA20 | AN5 | BN1 | CN20 |
| EA21 | AN5 | BN1 | CN21 |
| EA22 | AN5 | BN1 | CN22 |
| EA23 | AN5 | BN1 | CN23 |
| EA24 | AN5 | BN1 | CN24 |
| EA25 | AN5 | BN1 | CN25 |
| EA26 | AN5 | BN1 | CN26 |
| EA27 | AN5 | BN1 | CN27 |
| EA28 | AN5 | BN1 | CN28 |
| EA29 | AN5 | BN1 | CN29 |
| EA30 | AN5 | BN1 | CN30 |
| EA31 | AN5 | BN1 | CN31 |
| EA32 | AN5 | BN1 | CN32 |
| EA33 | AN5 | BN1 | CN33 |
| EA34 | AN5 | BN1 | CN34 |
| EA35 | AN5 | BN1 | CN35 |
| EA36 | AN5 | BN1 | CN36 |
| EB1 | AN5 | BN2 | CN1 |
| EB2 | AN5 | BN2 | CN2 |
| EB3 | AN5 | BN2 | CN3 |
| EB4 | AN5 | BN2 | CN4 |
| EB5 | AN5 | BN2 | CN5 |
| EB6 | AN5 | BN2 | CN6 |
| EB7 | AN5 | BN2 | CN7 |
| EB8 | AN5 | BN2 | CN8 |
| EB9 | AN5 | BN2 | CN9 |
| EB10 | AN5 | BN2 | CN10 |
| EB11 | AN5 | BN2 | CN11 |
| EB12 | AN5 | BN2 | CN12 |
| EB13 | AN5 | BN2 | CN13 |
| EB14 | AN5 | BN2 | CN14 |
| EB15 | AN5 | BN2 | CN15 |
| EB16 | AN5 | BN2 | CN16 |
| EB17 | AN5 | BN2 | CN17 |
| EB18 | AN5 | BN2 | CN18 |
| EB19 | AN5 | BN2 | CN19 |
| EB20 | AN5 | BN2 | CN20 |
| EB21 | AN5 | BN2 | CN21 |
| EB22 | AN5 | BN2 | CN22 |
| EB23 | AN5 | BN2 | CN23 |
| EB24 | AN5 | BN2 | CN24 |
| EB25 | AN5 | BN2 | CN25 |
| EB26 | AN5 | BN2 | CN26 |
| EB27 | AN5 | BN2 | CN27 |
| EB28 | AN5 | BN2 | CN28 |
| EB29 | AN5 | BN2 | CN29 |
| EB30 | AN5 | BN2 | CN30 |
| EB31 | AN5 | BN2 | CN31 |
| EB32 | AN5 | BN2 | CN32 |
| EB33 | AN5 | BN2 | CN33 |
| EB34 | AN5 | BN2 | CN34 |
| EB35 | AN5 | BN2 | CN35 |
| EB36 | AN5 | BN2 | CN36 |
| EC1 | AN5 | BN3 | CN1 |
| EC2 | AN5 | BN3 | CN2 |
| EC3 | AN5 | BN3 | CN3 |
| EC4 | AN5 | BN3 | CN4 |
| EC5 | AN5 | BN3 | CN5 |
| EC6 | AN5 | BN3 | CN6 |
| EC7 | AN5 | BN3 | CN7 |
| EC8 | AN5 | BN3 | CN8 |
| EC9 | AN5 | BN3 | CN9 |
| EC10 | AN5 | BN3 | CN10 |
| EC11 | AN5 | BN3 | CN11 |
| EC12 | AN5 | BN3 | CN12 |
| EC13 | AN5 | BN3 | CN13 |
| EC14 | AN5 | BN3 | CN14 |
| EC15 | AN5 | BN3 | CN15 |
| EC16 | AN5 | BN3 | CN16 |
| EC17 | AN5 | BN3 | CN17 |
| EC18 | AN5 | BN3 | CN18 |
| EC19 | AN5 | BN3 | CN19 |
| EC20 | AN5 | BN3 | CN20 |
| EC21 | AN5 | BN3 | CN21 |
| EC22 | AN5 | BN3 | CN22 |
| EC23 | AN5 | BN3 | CN23 |
| EC24 | AN5 | BN3 | CN24 |
| EC25 | AN5 | BN3 | CN25 |
| EC26 | AN5 | BN3 | CN26 |
| EC27 | AN5 | BN3 | CN27 |
| EC28 | AN5 | BN3 | CN28 |
| EC29 | AN5 | BN3 | CN29 |
| EC30 | AN5 | BN3 | CN30 |
| EC31 | AN5 | BN3 | CN31 |
| EC32 | AN5 | BN3 | CN32 |
| EC33 | AN5 | BN3 | CN33 |
| EC34 | AN5 | BN3 | CN34 |
| EC35 | AN5 | BN3 | CN35 |
| EC36 | AN5 | BN3 | CN36 |
| ED1 | AN5 | BN4 | CN1 |
| ED2 | AN5 | BN4 | CN2 |
| ED3 | AN5 | BN4 | CN3 |
| ED4 | AN5 | BN4 | CN4 |
| ED5 | AN5 | BN4 | CN5 |
| ED6 | AN5 | BN4 | CN6 |
| ED7 | AN5 | BN4 | CN7 |
| ED8 | AN5 | BN4 | CN8 |
| ED9 | AN5 | BN4 | CN9 |
| ED10 | AN5 | BN4 | CN10 |
| ED11 | AN5 | BN4 | CN11 |
| ED12 | AN5 | BN4 | CN12 |
| ED13 | AN5 | BN4 | CN13 |
| ED14 | AN5 | BN4 | CN14 |
| ED15 | AN5 | BN4 | CN15 |
| ED16 | AN5 | BN4 | CN16 |
| ED17 | AN5 | BN4 | CN17 |
| ED18 | AN5 | BN4 | CN18 |
| ED19 | AN5 | BN4 | CN19 |
| ED20 | AN5 | BN4 | CN20 |
| ED21 | AN5 | BN4 | CN21 |
| ED22 | AN5 | BN4 | CN22 |
| ED23 | AN5 | BN4 | CN23 |
| ED24 | AN5 | BN4 | CN24 |
| ED25 | AN5 | BN4 | CN25 |
| ED26 | AN5 | BN4 | CN26 |
| ED27 | AN5 | BN4 | CN27 |
| ED28 | AN5 | BN4 | CN28 |
| ED29 | AN5 | BN4 | CN29 |
| ED30 | AN5 | BN4 | CN30 |
| ED31 | AN5 | BN4 | CN31 |
| ED32 | AN5 | BN4 | CN32 |
| ED33 | AN5 | BN4 | CN33 |
| ED34 | AN5 | BN4 | CN34 |
| ED35 | AN5 | BN4 | CN35 |
| ED36 | AN5 | BN4 | CN36 |
| EE1 | AN5 | BN5 | CN1 |
| EE2 | AN5 | BN5 | CN2 |
| EE3 | AN5 | BN5 | CN3 |
| EE4 | AN5 | BN5 | CN4 |
| EE5 | AN5 | BN5 | CN5 |
| EE6 | AN5 | BN5 | CN6 |
| EE7 | AN5 | BN5 | CN7 |
| EE8 | AN5 | BN5 | CN8 |
| EE9 | AN5 | BN5 | CN9 |
| EE10 | AN5 | BN5 | CN10 |
| EE11 | AN5 | BN5 | CN11 |
| EE12 | AN5 | BN5 | CN12 |
| EE13 | AN5 | BN5 | CN13 |
| EE14 | AN5 | BN5 | CN14 |
| EE15 | AN5 | BN5 | CN15 |
| EE16 | AN5 | BN5 | CN16 |
| EE17 | AN5 | BN5 | CN17 |
| EE18 | AN5 | BN5 | CN18 |
| EE19 | AN5 | BN5 | CN19 |
| EE20 | AN5 | BN5 | CN20 |
| EE21 | AN5 | BN5 | CN21 |
| EE22 | AN5 | BN5 | CN22 |
| EE23 | AN5 | BN5 | CN23 |
| EE24 | AN5 | BN5 | CN24 |
| EE25 | AN5 | BN5 | CN25 |
| EE26 | AN5 | BN5 | CN26 |
| EE27 | AN5 | BN5 | CN27 |
| EE28 | AN5 | BN5 | CN28 |
| EE29 | AN5 | BN5 | CN29 |
| EE30 | AN5 | BN5 | CN30 |
| EE31 | AN5 | BN5 | CN31 |
| EE32 | AN5 | BN5 | CN32 |
| EE33 | AN5 | BN5 | CN33 |
| EE34 | AN5 | BN5 | CN34 |
| EE35 | AN5 | BN5 | CN35 |
| EE36 | AN5 | BN5 | CN36 |
| EF1 | AN5 | BN6 | CN1 |
| EF2 | AN5 | BN6 | CN2 |
| EF3 | AN5 | BN6 | CN3 |
| EF4 | AN5 | BN6 | CN4 |
| EF5 | AN5 | BN6 | CN5 |
| EF6 | AN5 | BN6 | CN6 |
| EF7 | AN5 | BN6 | CN7 |
| EF8 | AN5 | BN6 | CN8 |
| EF9 | AN5 | BN6 | CN9 |
| EF10 | AN5 | BN6 | CN10 |
| EF11 | AN5 | BN6 | CN11 |
| EF12 | AN5 | BN6 | CN12 |
| EF13 | AN5 | BN6 | CN13 |
| EF14 | AN5 | BN6 | CN14 |
| EF15 | AN5 | BN6 | CN15 |
| EF16 | AN5 | BN6 | CN16 |
| EF17 | AN5 | BN6 | CN17 |
| EF18 | AN5 | BN6 | CN18 |
| EF19 | AN5 | BN6 | CN19 |
| EF20 | AN5 | BN6 | CN20 |
| EF21 | AN5 | BN6 | CN21 |
| EF22 | AN5 | BN6 | CN22 |
| EF23 | AN5 | BN6 | CN23 |
| EF24 | AN5 | BN6 | CN24 |
| EF25 | AN5 | BN6 | CN25 |
| EF26 | AN5 | BN6 | CN26 |
| EF27 | AN5 | BN6 | CN27 |
| EF28 | AN5 | BN6 | CN28 |
| EF29 | AN5 | BN6 | CN29 |
| EF30 | AN5 | BN6 | CN30 |
| EF31 | AN5 | BN6 | CN31 |
| EF32 | AN5 | BN6 | CN32 |
| EF33 | AN5 | BN6 | CN33 |
| EF34 | AN5 | BN6 | CN34 |
| EF35 | AN5 | BN6 | CN35 |
| EF36 | AN5 | BN6 | CN36 |
| EG1 | AN5 | BN7 | CN1 |
| EG2 | AN5 | BN7 | CN2 |
| EG3 | AN5 | BN7 | CN3 |
| EG4 | AN5 | BN7 | CN4 |
| EG5 | AN5 | BN7 | CN5 |
| EG6 | AN5 | BN7 | CN6 |
| EG7 | AN5 | BN7 | CN7 |
| EG8 | AN5 | BN7 | CN8 |
| EG9 | AN5 | BN7 | CN9 |
| EG10 | AN5 | BN7 | CN10 |
| EG11 | AN5 | BN7 | CN11 |
| EG12 | AN5 | BN7 | CN12 |
| EG13 | AN5 | BN7 | CN13 |
| EG14 | AN5 | BN7 | CN14 |
| EG15 | AN5 | BN7 | CN15 |
| EG16 | AN5 | BN7 | CN16 |
| EG17 | AN5 | BN7 | CN17 |
| EG18 | AN5 | BN7 | CN18 |
| EG19 | AN5 | BN7 | CN19 |
| EG20 | AN5 | BN7 | CN20 |
| EG21 | AN5 | BN7 | CN21 |
| EG22 | AN5 | BN7 | CN22 |
| EG23 | AN5 | BN7 | CN23 |
| EG24 | AN5 | BN7 | CN24 |
| EG25 | AN5 | BN7 | CN25 |
| EG26 | AN5 | BN7 | CN26 |
| EG27 | AN5 | BN7 | CN27 |
| EG28 | AN5 | BN7 | CN28 |
| EG29 | AN5 | BN7 | CN29 |
| EG30 | AN5 | BN7 | CN30 |
| EG31 | AN5 | BN7 | CN31 |
| EG32 | AN5 | BN7 | CN32 |
| EG33 | AN5 | BN7 | CN33 |
| EG34 | AN5 | BN7 | CN34 |
| EG35 | AN5 | BN7 | CN35 |
| EG36 | AN5 | BN7 | CN36 |
| EH1 | AN5 | BN8 | CN1 |
| EH2 | AN5 | BN8 | CN2 |
| EH3 | AN5 | BN8 | CN3 |
| EH4 | AN5 | BN8 | CN4 |
| EH5 | AN5 | BN8 | CN5 |
| EH6 | AN5 | BN8 | CN6 |
| EH7 | AN5 | BN8 | CN7 |
| EH8 | AN5 | BN8 | CN8 |
| EH9 | AN5 | BN8 | CN9 |
| EH10 | AN5 | BN8 | CN10 |
| EH11 | AN5 | BN8 | CN11 |
| EH12 | AN5 | BN8 | CN12 |
| EH13 | AN5 | BN8 | CN13 |
| EH14 | AN5 | BN8 | CN14 |
| EH15 | AN5 | BN8 | CN15 |
| EH16 | AN5 | BN8 | CN16 |
| EH17 | AN5 | BN8 | CN17 |
| EH18 | AN5 | BN8 | CN18 |
| EH19 | AN5 | BN8 | CN19 |
| EH20 | AN5 | BN8 | CN20 |
| EH21 | AN5 | BN8 | CN21 |
| EH22 | AN5 | BN8 | CN22 |
| EH23 | AN5 | BN8 | CN23 |
| EH24 | AN5 | BN8 | CN24 |
| EH25 | AN5 | BN8 | CN25 |
| EH26 | AN5 | BN8 | CN26 |
| EH27 | AN5 | BN8 | CN27 |
| EH28 | AN5 | BN8 | CN28 |
| EH29 | AN5 | BN8 | CN29 |
| EH30 | AN5 | BN8 | CN30 |
| EH31 | AN5 | BN8 | CN31 |
| EH32 | AN5 | BN8 | CN32 |
| EH33 | AN5 | BN8 | CN33 |
| EH34 | AN5 | BN8 | CN34 |
| EH35 | AN5 | BN8 | CN35 |
| EH36 | AN5 | BN8 | CN36 |
| EI1 | AN5 | BN9 | CN1 |
| EI2 | AN5 | BN9 | CN2 |
| EI3 | AN5 | BN9 | CN3 |
| EI4 | AN5 | BN9 | CN4 |
| EI5 | AN5 | BN9 | CN5 |
| EI6 | AN5 | BN9 | CN6 |
| EI7 | AN5 | BN9 | CN7 |
| EI8 | AN5 | BN9 | CN8 |
| EI9 | AN5 | BN9 | CN9 |
| EI10 | AN5 | BN9 | CN10 |
| EI11 | AN5 | BN9 | CN11 |
| EI12 | AN5 | BN9 | CN12 |
| EI13 | AN5 | BN9 | CN13 |
| EI14 | AN5 | BN9 | CN14 |
| EI15 | AN5 | BN9 | CN15 |
| EI16 | AN5 | BN9 | CN16 |
| EI17 | AN5 | BN9 | CN17 |
| EI18 | AN5 | BN9 | CN18 |
| EI19 | AN5 | BN9 | CN19 |
| EI20 | AN5 | BN9 | CN20 |
| EI21 | AN5 | BN9 | CN21 |
| EI22 | AN5 | BN9 | CN22 |
| EI23 | AN5 | BN9 | CN23 |
| EI24 | AN5 | BN9 | CN24 |
| EI25 | AN5 | BN9 | CN25 |
| EI26 | AN5 | BN9 | CN26 |
| EI27 | AN5 | BN9 | CN27 |
| EI28 | AN5 | BN9 | CN28 |
| EI29 | AN5 | BN9 | CN29 |
| EI30 | AN5 | BN9 | CN30 |
| EI31 | AN5 | BN9 | CN31 |
| EI32 | AN5 | BN9 | CN32 |
| EI33 | AN5 | BN9 | CN33 |
| EI34 | AN5 | BN9 | CN34 |
| EI35 | AN5 | BN9 | CN35 |
| EI36 | AN5 | BN9 | CN36 |
| EJ1 | AN5 | BN10 | CN1 |
| EJ2 | AN5 | BN10 | CN2 |
| EJ3 | AN5 | BN10 | CN3 |
| EJ4 | AN5 | BN10 | CN4 |
| EJ5 | AN5 | BN10 | CN5 |
| EJ6 | AN5 | BN10 | CN6 |
| EJ7 | AN5 | BN10 | CN7 |
| EJ8 | AN5 | BN10 | CN8 |
| EJ9 | AN5 | BN10 | CN9 |
| EJ10 | AN5 | BN10 | CN10 |
| EJ11 | AN5 | BN10 | CN11 |
| EJ12 | AN5 | BN10 | CN12 |
| EJ13 | AN5 | BN10 | CN13 |
| EJ14 | AN5 | BN10 | CN14 |
| EJ15 | AN5 | BN10 | CN15 |
| EJ16 | AN5 | BN10 | CN16 |
| EJ17 | AN5 | BN10 | CN17 |
| EJ18 | AN5 | BN10 | CN18 |
| EJ19 | AN5 | BN10 | CN19 |
| EJ20 | AN5 | BN10 | CN20 |
| EJ21 | AN5 | BN10 | CN21 |
| EJ22 | AN5 | BN10 | CN22 |
| EJ23 | AN5 | BN10 | CN23 |
| EJ24 | AN5 | BN10 | CN24 |
| EJ25 | AN5 | BN10 | CN25 |
| EJ26 | AN5 | BN10 | CN26 |
| EJ27 | AN5 | BN10 | CN27 |
| EJ28 | AN5 | BN10 | CN28 |
| EJ29 | AN5 | BN10 | CN29 |
| EJ30 | AN5 | BN10 | CN30 |
| EJ31 | AN5 | BN10 | CN31 |
| EJ32 | AN5 | BN10 | CN32 |
| EJ33 | AN5 | BN10 | CN33 |
| EJ34 | AN5 | BN10 | CN34 |
| EJ35 | AN5 | BN10 | CN35 |
| EJ36 | AN5 | BN10 | CN36 |
| EK1 | AN5 | BN11 | CN1 |
| EK2 | AN5 | BN11 | CN2 |
| EK3 | AN5 | BN11 | CN3 |
| EK4 | AN5 | BN11 | CN4 |
| EK5 | AN5 | BN11 | CN5 |
| EK6 | AN5 | BN11 | CN6 |
| EK7 | AN5 | BN11 | CN7 |
| EK8 | AN5 | BN11 | CN8 |
| EK9 | AN5 | BN11 | CN9 |
| EK10 | AN5 | BN11 | CN10 |
| EK11 | AN5 | BN11 | CN11 |
| EK12 | AN5 | BN11 | CN12 |
| EK13 | AN5 | BN11 | CN13 |
| EK14 | AN5 | BN11 | CN14 |
| EK15 | AN5 | BN11 | CN15 |
| EK16 | AN5 | BN11 | CN16 |
| EK17 | AN5 | BN11 | CN17 |
| EK18 | AN5 | BN11 | CN18 |
| EK19 | AN5 | BN11 | CN19 |
| EK20 | AN5 | BN11 | CN20 |
| EK21 | AN5 | BN11 | CN21 |
| EK22 | AN5 | BN11 | CN22 |
| EK23 | AN5 | BN11 | CN23 |
| EK24 | AN5 | BN11 | CN24 |
| EK25 | AN5 | BN11 | CN25 |
| EK26 | AN5 | BN11 | CN26 |
| EK27 | AN5 | BN11 | CN27 |
| EK28 | AN5 | BN11 | CN28 |
| EK29 | AN5 | BN11 | CN29 |
| EK30 | AN5 | BN11 | CN30 |
| EK31 | AN5 | BN11 | CN31 |
| EK32 | AN5 | BN11 | CN32 |
| EK33 | AN5 | BN11 | CN33 |
| EK34 | AN5 | BN11 | CN34 |
| EK35 | AN5 | BN11 | CN35 |
| EK36 | AN5 | BN11 | CN36 |
| EL1 | AN5 | BN12 | CN1 |
| EL2 | AN5 | BN12 | CN2 |
| EL3 | AN5 | BN12 | CN3 |
| EL4 | AN5 | BN12 | CN4 |
| EL5 | AN5 | BN12 | CN5 |
| EL6 | AN5 | BN12 | CN6 |
| EL7 | AN5 | BN12 | CN7 |
| EL8 | AN5 | BN12 | CN8 |
| EL9 | AN5 | BN12 | CN9 |
| EL10 | AN5 | BN12 | CN10 |
| EL11 | AN5 | BN12 | CN11 |
| EL12 | AN5 | BN12 | CN12 |
| EL13 | AN5 | BN12 | CN13 |
| EL14 | AN5 | BN12 | CN14 |
| EL15 | AN5 | BN12 | CN15 |
| EL16 | AN5 | BN12 | CN16 |
| EL17 | AN5 | BN12 | CN17 |
| EL18 | AN5 | BN12 | CN18 |
| EL19 | AN5 | BN12 | CN19 |
| EL20 | AN5 | BN12 | CN20 |
| EL21 | AN5 | BN12 | CN21 |
| EL22 | AN5 | BN12 | CN22 |
| EL23 | AN5 | BN12 | CN23 |
| EL24 | AN5 | BN12 | CN24 |
| EL25 | AN5 | BN12 | CN25 |
| EL26 | AN5 | BN12 | CN26 |
| EL27 | AN5 | BN12 | CN27 |
| EL28 | AN5 | BN12 | CN28 |
| EL29 | AN5 | BN12 | CN29 |
| EL30 | AN5 | BN12 | CN30 |
| EL31 | AN5 | BN12 | CN31 |
| EL32 | AN5 | BN12 | CN32 |
| EL33 | AN5 | BN12 | CN33 |
| EL34 | AN5 | BN12 | CN34 |
| EL35 | AN5 | BN12 | CN35 |
| EL36 | AN5 | BN12 | CN36 |
| EM1 | AN5 | BN13 | CN1 |
| EM2 | AN5 | BN13 | CN2 |
| EM3 | AN5 | BN13 | CN3 |
| EM4 | AN5 | BN13 | CN4 |
| EM5 | AN5 | BN13 | CN5 |
| EM6 | AN5 | BN13 | CN6 |
| EM7 | AN5 | BN13 | CN7 |
| EM8 | AN5 | BN13 | CN8 |
| EM9 | AN5 | BN13 | CN9 |
| EM10 | AN5 | BN13 | CN10 |
| EM11 | AN5 | BN13 | CN11 |
| EM12 | AN5 | BN13 | CN12 |
| EM13 | AN5 | BN13 | CN13 |
| EM14 | AN5 | BN13 | CN14 |
| EM15 | AN5 | BN13 | CN15 |
| EM16 | AN5 | BN13 | CN16 |
| EM17 | AN5 | BN13 | CN17 |
| EM18 | AN5 | BN13 | CN18 |
| EM19 | AN5 | BN13 | CN19 |
| EM20 | AN5 | BN13 | CN20 |
| EM21 | AN5 | BN13 | CN21 |
| EM22 | AN5 | BN13 | CN22 |
| EM23 | AN5 | BN13 | CN23 |
| EM24 | AN5 | BN13 | CN24 |
| EM25 | AN5 | BN13 | CN25 |
| EM26 | AN5 | BN13 | CN26 |
| EM27 | AN5 | BN13 | CN27 |
| EM28 | AN5 | BN13 | CN28 |
| EM29 | AN5 | BN13 | CN29 |
| EM30 | AN5 | BN13 | CN30 |
| EM31 | AN5 | BN13 | CN31 |
| EM32 | AN5 | BN13 | CN32 |
| EM33 | AN5 | BN13 | CN33 |
| EM34 | AN5 | BN13 | CN34 |
| EM35 | AN5 | BN13 | CN35 |
| EM36 | AN5 | BN13 | CN36 |
| EO1 | AN5 | BN14 | CN1 |
| EO2 | AN5 | BN14 | CN2 |
| EO3 | AN5 | BN14 | CN3 |
| EO4 | AN5 | BN14 | CN4 |
| EO5 | AN5 | BN14 | CN5 |
| EO6 | AN5 | BN14 | CN6 |
| EO7 | AN5 | BN14 | CN7 |
| EO8 | AN5 | BN14 | CN8 |
| EO9 | AN5 | BN14 | CN9 |
| EO10 | AN5 | BN14 | CN10 |
| EO11 | AN5 | BN14 | CN11 |
| EO12 | AN5 | BN14 | CN12 |
| EO13 | AN5 | BN14 | CN13 |
| EO14 | AN5 | BN14 | CN14 |
| EO15 | AN5 | BN14 | CN15 |
| EO16 | AN5 | BN14 | CN16 |
| EO17 | AN5 | BN14 | CN17 |
| EO18 | AN5 | BN14 | CN18 |
| EO19 | AN5 | BN14 | CN19 |
| EO20 | AN5 | BN14 | CN20 |
| EO21 | AN5 | BN14 | CN21 |
| EO22 | AN5 | BN14 | CN22 |
| EO23 | AN5 | BN14 | CN23 |
| EO24 | AN5 | BN14 | CN24 |
| EO25 | AN5 | BN14 | CN25 |
| EO26 | AN5 | BN14 | CN26 |
| EO27 | AN5 | BN14 | CN27 |
| EO28 | AN5 | BN14 | CN28 |
| EO29 | AN5 | BN14 | CN29 |
| EO30 | AN5 | BN14 | CN30 |
| EO31 | AN5 | BN14 | CN31 |
| EO32 | AN5 | BN14 | CN32 |
| EO33 | AN5 | BN14 | CN33 |
| EO34 | AN5 | BN14 | CN34 |
| EO35 | AN5 | BN14 | CN35 |
| EO36 | AN5 | BN14 | CN36 |
| EP1 | AN5 | BN15 | CN1 |
| EP2 | AN5 | BN15 | CN2 |
| EP3 | AN5 | BN15 | CN3 |
| EP4 | AN5 | BN15 | CN4 |
| EP5 | AN5 | BN15 | CN5 |
| EP6 | AN5 | BN15 | CN6 |
| EP7 | AN5 | BN15 | CN7 |
| EP8 | AN5 | BN15 | CN8 |
| EP9 | AN5 | BN15 | CN9 |
| EP10 | AN5 | BN15 | CN10 |
| EP11 | AN5 | BN15 | CN11 |
| EP12 | AN5 | BN15 | CN12 |
| EP13 | AN5 | BN15 | CN13 |
| EP14 | AN5 | BN15 | CN14 |
| EP15 | AN5 | BN15 | CN15 |
| EP16 | AN5 | BN15 | CN16 |
| EP17 | AN5 | BN15 | CN17 |
| EP18 | AN5 | BN15 | CN18 |
| EP19 | AN5 | BN15 | CN19 |
| EP20 | AN5 | BN15 | CN20 |
| EP21 | AN5 | BN15 | CN21 |
| EP22 | AN5 | BN15 | CN22 |
| EP23 | AN5 | BN15 | CN23 |
| EP24 | AN5 | BN15 | CN24 |
| EP25 | AN5 | BN15 | CN25 |
| EP26 | AN5 | BN15 | CN26 |
| EP27 | AN5 | BN15 | CN27 |
| EP28 | AN5 | BN15 | CN28 |
| EP29 | AN5 | BN15 | CN29 |
| EP30 | AN5 | BN15 | CN30 |
| EP31 | AN5 | BN15 | CN31 |
| EP32 | AN5 | BN15 | CN32 |
| EP33 | AN5 | BN15 | CN33 |
| EP34 | AN5 | BN15 | CN34 |
| EP35 | AN5 | BN15 | CN35 |
| EP36 | AN5 | BN15 | CN36 |
| EQ1 | AN5 | BN16 | CN1 |
| EQ2 | AN5 | BN16 | CN2 |
| EQ3 | AN5 | BN16 | CN3 |
| EQ4 | AN5 | BN16 | CN4 |
| EQ5 | AN5 | BN16 | CN5 |
| EQ6 | AN5 | BN16 | CN6 |
| EQ7 | AN5 | BN16 | CN7 |
| EQ8 | AN5 | BN16 | CN8 |
| EQ9 | AN5 | BN16 | CN9 |
| EQ10 | AN5 | BN16 | CN10 |
| EQ11 | AN5 | BN16 | CN11 |
| EQ12 | AN5 | BN16 | CN12 |
| EQ13 | AN5 | BN16 | CN13 |
| EQ14 | AN5 | BN16 | CN14 |
| EQ15 | AN5 | BN16 | CN15 |
| EQ16 | AN5 | BN16 | CN16 |
| EQ17 | AN5 | BN16 | CN17 |
| EQ18 | AN5 | BN16 | CN18 |
| EQ19 | AN5 | BN16 | CN19 |
| EQ20 | AN5 | BN16 | CN20 |
| EQ21 | AN5 | BN16 | CN21 |
| EQ22 | AN5 | BN16 | CN22 |
| EQ23 | AN5 | BN16 | CN23 |
| EQ24 | AN5 | BN16 | CN24 |
| EQ25 | AN5 | BN16 | CN25 |
| EQ26 | AN5 | BN16 | CN26 |
| EQ27 | AN5 | BN16 | CN27 |
| EQ28 | AN5 | BN16 | CN28 |
| EQ29 | AN5 | BN16 | CN29 |
| EQ30 | AN5 | BN16 | CN30 |
| EQ31 | AN5 | BN16 | CN31 |
| EQ32 | AN5 | BN16 | CN32 |
| EQ33 | AN5 | BN16 | CN33 |
| EQ34 | AN5 | BN16 | CN34 |
| EQ35 | AN5 | BN16 | CN35 |
| EQ36 | AN5 | BN16 | CN36 |
| ER1 | AN5 | BN17 | CN1 |
| ER2 | AN5 | BN17 | CN2 |
| ER3 | AN5 | BN17 | CN3 |
| ER4 | AN5 | BN17 | CN4 |
| ER5 | AN5 | BN17 | CN5 |
| ER6 | AN5 | BN17 | CN6 |
| ER7 | AN5 | BN17 | CN7 |
| ER8 | AN5 | BN17 | CN8 |
| ER9 | AN5 | BN17 | CN9 |
| ER10 | AN5 | BN17 | CN10 |
| ER11 | AN5 | BN17 | CN11 |
| ER12 | AN5 | BN17 | CN12 |
| ER13 | AN5 | BN17 | CN13 |
| ER14 | AN5 | BN17 | CN14 |
| ER15 | AN5 | BN17 | CN15 |
| ER16 | AN5 | BN17 | CN16 |
| ER17 | AN5 | BN17 | CN17 |
| ER18 | AN5 | BN17 | CN18 |
| ER19 | AN5 | BN17 | CN19 |
| ER20 | AN5 | BN17 | CN20 |
| ER21 | AN5 | BN17 | CN21 |
| ER22 | AN5 | BN17 | CN22 |
| ER23 | AN5 | BN17 | CN23 |
| ER24 | AN5 | BN17 | CN24 |
| ER25 | AN5 | BN17 | CN25 |
| ER26 | AN5 | BN17 | CN26 |
| ER27 | AN5 | BN17 | CN27 |
| ER28 | AN5 | BN17 | CN28 |
| ER29 | AN5 | BN17 | CN29 |
| ER30 | AN5 | BN17 | CN30 |
| ER31 | AN5 | BN17 | CN31 |
| ER32 | AN5 | BN17 | CN32 |
| ER33 | AN5 | BN17 | CN33 |
| ER34 | AN5 | BN17 | CN34 |
| ER35 | AN5 | BN17 | CN35 |
| ER36 | AN5 | BN17 | CN36 |
| ES1 | AN5 | BN18 | CN1 |
| ES2 | AN5 | BN18 | CN2 |
| ES3 | AN5 | BN18 | CN3 |
| ES4 | AN5 | BN18 | CN4 |
| ES5 | AN5 | BN18 | CN5 |
| ES6 | AN5 | BN18 | CN6 |
| ES7 | AN5 | BN18 | CN7 |
| ES8 | AN5 | BN18 | CN8 |
| ES9 | AN5 | BN18 | CN9 |
| ES10 | AN5 | BN18 | CN10 |
| ES11 | AN5 | BN18 | CN11 |
| ES12 | AN5 | BN18 | CN12 |
| ES13 | AN5 | BN18 | CN13 |
| ES14 | AN5 | BN18 | CN14 |
| ES15 | AN5 | BN18 | CN15 |
| ES16 | AN5 | BN18 | CN16 |
| ES17 | AN5 | BN18 | CN17 |
| ES18 | AN5 | BN18 | CN18 |
| ES19 | AN5 | BN18 | CN19 |
| ES20 | AN5 | BN18 | CN20 |
| ES21 | AN5 | BN18 | CN21 |
| ES22 | AN5 | BN18 | CN22 |
| ES23 | AN5 | BN18 | CN23 |
| ES24 | AN5 | BN18 | CN24 |
| ES25 | AN5 | BN18 | CN25 |
| ES26 | AN5 | BN18 | CN26 |
| ES27 | AN5 | BN18 | CN27 |
| ES28 | AN5 | BN18 | CN28 |
| ES29 | AN5 | BN18 | CN29 |
| ES30 | AN5 | BN18 | CN30 |
| ES31 | AN5 | BN18 | CN31 |
| ES32 | AN5 | BN18 | CN32 |
| ES33 | AN5 | BN18 | CN33 |
| ES34 | AN5 | BN18 | CN34 |
| ES35 | AN5 | BN18 | CN35 |
| ES36 | AN5 | BN18 | CN36 |
| ET1 | AN5 | BN19 | CN1 |
| ET2 | AN5 | BN19 | CN2 |
| ET3 | AN5 | BN19 | CN3 |
| ET4 | AN5 | BN19 | CN4 |
| ET5 | AN5 | BN19 | CN5 |
| ET6 | AN5 | BN19 | CN6 |
| ET7 | AN5 | BN19 | CN7 |
| ET8 | AN5 | BN19 | CN8 |
| ET9 | AN5 | BN19 | CN9 |
| ET10 | AN5 | BN19 | CN10 |
| ET11 | AN5 | BN19 | CN11 |
| ET12 | AN5 | BN19 | CN12 |
| ET13 | AN5 | BN19 | CN13 |
| ET14 | AN5 | BN19 | CN14 |
| ET15 | AN5 | BN19 | CN15 |
| ET16 | AN5 | BN19 | CN16 |
| ET17 | AN5 | BN19 | CN17 |
| ET18 | AN5 | BN19 | CN18 |
| ET19 | AN5 | BN19 | CN19 |
| ET20 | AN5 | BN19 | CN20 |
| ET21 | AN5 | BN19 | CN21 |
| ET22 | AN5 | BN19 | CN22 |
| ET23 | AN5 | BN19 | CN23 |
| ET24 | AN5 | BN19 | CN24 |
| ET25 | AN5 | BN19 | CN25 |
| ET26 | AN5 | BN19 | CN26 |
| ET27 | AN5 | BN19 | CN27 |
| ET28 | AN5 | BN19 | CN28 |
| ET29 | AN5 | BN19 | CN29 |
| ET30 | AN5 | BN19 | CN30 |
| ET31 | AN5 | BN19 | CN31 |
| ET32 | AN5 | BN19 | CN32 |
| ET33 | AN5 | BN19 | CN33 |
| ET34 | AN5 | BN19 | CN34 |
| ET35 | AN5 | BN19 | CN35 |
| ET36 | AN5 | BN19 | CN36 |
| EU1 | AN5 | BN20 | CN1 |
| EU2 | AN5 | BN20 | CN2 |
| EU3 | AN5 | BN20 | CN3 |
| EU4 | AN5 | BN20 | CN4 |
| EU5 | AN5 | BN20 | CN5 |
| EU6 | AN5 | BN20 | CN6 |
| EU7 | AN5 | BN20 | CN7 |
| EU8 | AN5 | BN20 | CN8 |
| EU9 | AN5 | BN20 | CN9 |
| EU10 | AN5 | BN20 | CN10 |
| EU11 | AN5 | BN20 | CN11 |
| EU12 | AN5 | BN20 | CN12 |
| EU13 | AN5 | BN20 | CN13 |
| EU14 | AN5 | BN20 | CN14 |
| EU15 | AN5 | BN20 | CN15 |
| EU16 | AN5 | BN20 | CN16 |
| EU17 | AN5 | BN20 | CN17 |
| EU18 | AN5 | BN20 | CN18 |
| EU19 | AN5 | BN20 | CN19 |
| EU20 | AN5 | BN20 | CN20 |
| EU21 | AN5 | BN20 | CN21 |
| EU22 | AN5 | BN20 | CN22 |
| EU23 | AN5 | BN20 | CN23 |
| EU24 | AN5 | BN20 | CN24 |
| EU25 | AN5 | BN20 | CN25 |
| EU26 | AN5 | BN20 | CN26 |
| EU27 | AN5 | BN20 | CN27 |
| EU28 | AN5 | BN20 | CN28 |
| EU29 | AN5 | BN20 | CN29 |
| EU30 | AN5 | BN20 | CN30 |
| EU31 | AN5 | BN20 | CN31 |
| EU32 | AN5 | BN20 | CN32 |
| EU33 | AN5 | BN20 | CN33 |
| EU34 | AN5 | BN20 | CN34 |
| EU35 | AN5 | BN20 | CN35 |
| EU36 | AN5 | BN20 | CN36 |
| EV1 | AN5 | BN21 | CN1 |
| EV2 | AN5 | BN21 | CN2 |
| EV3 | AN5 | BN21 | CN3 |
| EV4 | AN5 | BN21 | CN4 |
| EV5 | AN5 | BN21 | CN5 |
| EV6 | AN5 | BN21 | CN6 |
| EV7 | AN5 | BN21 | CN7 |
| EV8 | AN5 | BN21 | CN8 |
| EV9 | AN5 | BN21 | CN9 |
| EV10 | AN5 | BN21 | CN10 |
| EV11 | AN5 | BN21 | CN11 |
| EV12 | AN5 | BN21 | CN12 |
| EV13 | AN5 | BN21 | CN13 |
| EV14 | AN5 | BN21 | CN14 |
| EV15 | AN5 | BN21 | CN15 |
| EV16 | AN5 | BN21 | CN16 |
| EV17 | AN5 | BN21 | CN17 |
| EV18 | AN5 | BN21 | CN18 |
| EV19 | AN5 | BN21 | CN19 |
| EV20 | AN5 | BN21 | CN20 |
| EV21 | AN5 | BN21 | CN21 |
| EV22 | AN5 | BN21 | CN22 |
| EV23 | AN5 | BN21 | CN23 |
| EV24 | AN5 | BN21 | CN24 |
| EV25 | AN5 | BN21 | CN25 |
| EV26 | AN5 | BN21 | CN26 |
| EV27 | AN5 | BN21 | CN27 |
| EV28 | AN5 | BN21 | CN28 |
| EV29 | AN5 | BN21 | CN29 |
| EV30 | AN5 | BN21 | CN30 |
| EV31 | AN5 | BN21 | CN31 |
| EV32 | AN5 | BN21 | CN32 |
| EV33 | AN5 | BN21 | CN33 |
| EV34 | AN5 | BN21 | CN34 |
| EV35 | AN5 | BN21 | CN35 |
| EV36 | AN5 | BN21 | CN36 |
| EW1 | AN5 | BN22 | CN1 |
| EW2 | AN5 | BN22 | CN2 |
| EW3 | AN5 | BN22 | CN3 |
| EW4 | AN5 | BN22 | CN4 |
| EW5 | AN5 | BN22 | CN5 |
| EW6 | AN5 | BN22 | CN6 |
| EW7 | AN5 | BN22 | CN7 |
| EW8 | AN5 | BN22 | CN8 |
| EW9 | AN5 | BN22 | CN9 |
| EW10 | AN5 | BN22 | CN10 |
| EW11 | AN5 | BN22 | CN11 |
| EW12 | AN5 | BN22 | CN12 |
| EW13 | AN5 | BN22 | CN13 |
| EW14 | AN5 | BN22 | CN14 |
| EW15 | AN5 | BN22 | CN15 |
| EW16 | AN5 | BN22 | CN16 |
| EW17 | AN5 | BN22 | CN17 |
| EW18 | AN5 | BN22 | CN18 |
| EW19 | AN5 | BN22 | CN19 |
| EW20 | AN5 | BN22 | CN20 |
| EW21 | AN5 | BN22 | CN21 |
| EW22 | AN5 | BN22 | CN22 |
| EW23 | AN5 | BN22 | CN23 |
| EW24 | AN5 | BN22 | CN24 |
| EW25 | AN5 | BN22 | CN25 |
| EW26 | AN5 | BN22 | CN26 |
| EW27 | AN5 | BN22 | CN27 |
| EW28 | AN5 | BN22 | CN28 |
| EW29 | AN5 | BN22 | CN29 |
| EW30 | AN5 | BN22 | CN30 |
| EW31 | AN5 | BN22 | CN31 |
| EW32 | AN5 | BN22 | CN32 |
| EW33 | AN5 | BN22 | CN33 |
| EW34 | AN5 | BN22 | CN34 |
| EW35 | AN5 | BN22 | CN35 |
| EW36 | AN5 | BN22 | CN36. |
